(19) United States
(12) Patent Application Publication
(10) Pub. No.: US 2004/0152739 A1
(43) Pub. Date: Aug. 5, 2004

(54) Acyclic pyrazole compounds for the inhibition of mitogen activated protein kinase-activated protein kinase-2

(75) Inventors: Cathleen E. Hanau, Chesterfield, MO (US); Serena Marie Mershon, Bessemer, MI (US); Matthew J. Graneto, Chesterfield, MO (US); Marvin J. Meyers, St. Charles, MO (US); Shridhar G. Hegde, Ballwin, MO (US); Ingrid P. Buchler, South University City, MO (US); Kun K. Wu, Chesterfield, MO (US); Shuang Liu, Schenectady, NY (US); Kassoum Nacro, Guiderland, NY (US)

Correspondence Address: Charles E. Dunlap, P.O. Box 11070, Columbia, SC, 29211, US

(73) Assignee: Pharmacia Corporation, St. Louis, MO

(21) Appl. No.: 10/742,494

(22) Filed: Dec. 19, 2003

Related U.S. Application Data
(60) Provisional application No. 60/434,962, filed on Dec. 20, 2002.

(51) Int. Cl. A61K 31/4439; A61K 31/415

(52) U.S. Cl. 514/341; 514/406; 546/275.4; 548/377.1

(57) ABSTRACT

Compounds are described which inhibit mitogen activated protein kinase-activated protein kinase-2 (MK-2). Methods of making such compounds are described, as well as a method of using them for the inhibition of MK-2, and for the prevention or treatment of a disease or disorder that is mediated by TNFα, where the method involves administering to the subject an MK-2 inhibiting compound of the present invention. Pharmaceutical compositions and kits which contain the present MK-2 inhibiting compounds are also described.

### CROSS REFERENCE TO RELATED PATENTS AND PATENT APPLICATIONS

This application is related to and claims the benefit of U.S. Provisional Patent Application Serial No. 60/434,962, filed Dec. 20, 2002, which is incorporated by reference herein in its entirety.

### BACKGROUND OF THE INVENTION

(1) Field of the Invention

The present invention relates to certain cyclic and heterocyclic compounds which inhibit mitogen-activated protein kinase-activated protein kinase-2 (MAPKAP kinase-2, or MK-2), and also to methods of using such compounds to inhibit MK-2 and for the prevention and treatment of TNFα mediated diseases or disorders in subjects that are in need of such prevention and/or treatment.

(2) Description of the Related Art

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif with the sequence Thr-X-Tyr by mitogen-activated protein kinase kinases (MAPKKs). In higher eukaryotes, the physiological role of MAPK signaling has been correlated with cellular events such as proliferation, oncogenesis, development and differentiation. Accordingly, the ability to regulate signal transduction via these pathways could lead to the development of treatments and preventive therapies for human diseases associated with MAPK signaling, such as inflammatory diseases, autoimmune diseases and cancer.

In mammalian cells, three parallel MAPK pathways have been described. The best characterized pathway leads to the activation of the extracellular-signal-regulated kinase (ERK). Less well understood are the signal transduction pathways leading to the activation of the cJun N-terminal kinase (JNK) and the p38 MAPK. See, e.g., Davis, Trends Biochem. Sci. 19:470-473 (1994); Cano, et al., Trends Biochem. Sci. 20:117-122(1995).

The p38 MAPK pathway is potentially activated by a wide variety of stresses and cellular insults. These stresses and cellular insults include heat shock, UV irradiation, inflammatory cytokines (such as TNF and IL-1), tunicamycin, chemotherapeutic drugs (i.e., cisplatinum), anisomycin, sorbitol/hyperosmolarity, gamma irradiation, sodium arsenite, and ischaemia. See, Ono, K., et al, Cellular Signalling 12, 1-13 (2000). Activation of the p38 pathway is involved in (1) production of proinflammatory cytokines, such as TNF-α; (2) induction of enzymes, such as Cox-2; (3) expression of an intracellular enzyme, such as iNOS, which plays an important role in the regulation of oxidation; (4) induction of adherent proteins, such as VCAM-1 and many other inflammatory-related molecules. Furthermore, the p38 pathway functions as a regulator in the proliferation and differentiation of cells of the immune system. See, Ono, K., et al., Id. at 7.

The p38 kinase is an upstream kinase of mitogen-activated protein kinase-activated protein kinase-2 (MAPKAP kinase-2 or MK-2). (See, Freshney, N. W., et al., J. Cell, 78:1039-1049 (1994)). MK-2 is a protein that appears to be predominantly regulated by p38 in cells. Indeed, MK-2 was the first substrate of p38α to be identified. For example, in vitro phosphorylation of MK-2 by p38α activates MK-2. The substrates that MK-2 acts upon, in turn, include heat shock protein 27, lymphocyte-specific protein 1 (LAP1), cAMP response element-binding protein (CREB), ATF1, serum response factor (SRF), and tyrosine hydroxylase. The substrate of MK-2 that has been best characterized is small heat shock protein 27 (hsp27).

The role of the p38 pathway in inflammatory-related diseases has been studied in several animal models. The pyridinyl imidazole compound SB203580 has been shown to be a specific inhibitor of p38 in vivo, and also has been shown to inhibit activation of MK-2, (See, Rouse, J., et al, Cell, 78:1027-1037 (1994); Cuenda, A., et al, Biochem. J., 333:11-15 (1998)), as well as a MAP kinase homologue termed reactivating kinase (RK). (See, Cuenda, A., et al., FEBS Lett., 364(2):229-233 (1995)). Inhibition of p38 by SB203580 can reduce mortality in a murine model of endotoxin-induced shock and inhibit the development of mouse collagen-induced arthritis and rat adjuvant arthritis. See, e.g., Badger, A. M., et al., J. Pharmacol Exp. Ther., 279:1453-1461 (1996). Another p38 inhibitor that has been utilized in an animal model that is believed to be more potent than SB203580 in its inhibitory effect on p38 is SB 220025. A recent animal study has demonstrated that SB 220025 caused a significant dose-dependent decrease in vascular density of granulomas in laboratory rats. (See, Jackson, J. R., et al, J. Pharmacol. Exp. Ther., 284:687-692 (1998)). The results of these animal studies indicated that p38, or the components of the p38 pathway, can be useful therapeutic targets for the prevention or treatment of inflammatory disease.

Due to its integral role in the p38 signaling pathway, MK-2 has been used as a monitor for measuring the level of activation in the pathway. Because of its downstream location in the pathway, relative to p38, MK-2 has been measured as a more convenient, albeit indirect, method of assessing p38 activation. However, so far, research efforts exploring therapeutic strategies associated with the modulation of this pathway have focused mainly on the inhibition of p38 kinase.

Several compounds that inhibit the activity of p38 kinase have been described in U.S. Pat. Nos. 6,046,208, 6,251,914, and 6,335,340. These compounds have been suggested to be useful for the treatment of CSBP/RK/p38 kinase mediated disease. Commercial efforts to apply p38 inhibitors have centered around two p38 inhibitors, the pyridinylimidazole inhibitor SKF 86002, and the 2,4,5 triaryl imidazole inhibitor SB203580. See, Lee, J. C., et al, Immunopharmacology 47, 185-192 (2000). Compounds possessing a similar structure have also been investigated as potential p38 inhibitors. Indeed, p38 MSP kinase's role in various disease states has been elucidated through the use of inhibitors.

Kotlyarov, A. et al, in Nat. Cell Biol., 1(2):94-97 (1999) introduced a targeted mutation into a mouse MK-2 gene, resulting in MK-2-deficient mice. It was shown that mice lacking MK-2 possessed increased stress resistance and survived LPS-induced endotoxic shock better than MK-2⁺ mice. The authors concluded that MK-2 was an essential component in the inflammatory response that regulates biosynthesis of TNFα at a post-transcriptional level. More recently, Lehner, M. D., et al, in J. Immunol., 168(9):4667-4673 (2002), reported that MK-2-deficient mice showed increased susceptibility to Listeria monocytogenes infection, and concluded that MK-2 had an essential role in host defense against intracellular bacteria, probably via regulation of TNF and IFN-gamma production required for activation of antibacterial effector mechanisms.

The location of MK-2 in the p38 signaling pathway at a point that is downstream of p38 offers the potential that MK-2 could act as a focal point for modulating the pathway without affecting as many substrates as would the regulation of an enzyme further upstream in the signaling cascade—such as p38 MAP kinase.

Accordingly, it would be useful to provide compounds and methods that could serve to modulate the activity of MK-2—in particular, to act as inhibitors of MK-2 activity. Such compounds and methods would be useful for the provision of benefits similar to p38 MAP kinase inhibitors, which benefits include the prevention and treatment of diseases and disorders that are mediated by TNFα. It would be even more useful to provide MK-2 inhibitors having improved potency and reduced undesirable side effects, relative to p38 inhibitors.

### SUMMARY OF THE INVENTION

Briefly therefore, the present invention is directed to a novel compound having the structure of formula I:

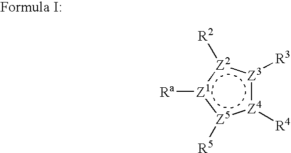

wherein:

Z² and Z³ are nitrogen, Z¹, Z⁴ and Z⁵ are carbon, and join with Z² and Z³ to form a pyrazole ring, or optionally, Z⁴ and Z⁵ are nitrogen, Z¹, Z² and Z³ are carbon and join with Z⁴ and Z⁵ to form a pyrazole ring;

R^{a} is selected from:

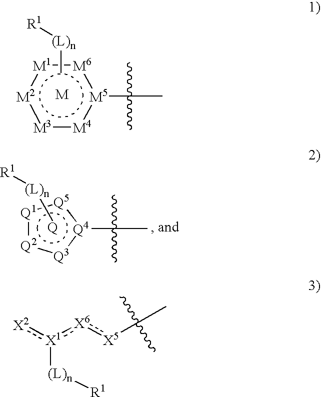

where dashed lines indicate optional single or double bonds;

when R^{a} is ring M and ring M is aromatic, M¹ is carbon and is substituted with (L)ₙR¹, M⁵ is carbon, and each of M², M³, M⁴ and M⁶ is independently selected from carbon and nitrogen and is unsubstituted or substituted with (L)ₙR¹;

when ring M is partially saturated, M¹ is carbon and is mono- or di-substituted with (L)ₙR¹, M⁵ is carbon, and each of M², M³, M⁴ and M⁶ is independently selected from carbon, nitrogen, oxygen and sulfur, and when M², M³, M⁴, or M⁶ is oxygen or sulfur, it is unsubstituted, and when M², M³, M⁴ or M⁶ is carbon or nitrogen, it is optionally unsubstituted; or mono- or di-substituted with (L)ₙR¹;

when R^{a} is ring Q and ring Q is aromatic, Q¹ is selected from carbon and nitrogen, and when Q¹ is carbon, it is substituted with (L)ₙR¹, and when Q¹ is nitrogen, it is unsubstituted, Q⁴ is selected from nitrogen and carbon, and each of Q², Q³ and Q⁵ is independently selected from nitrogen and carbon, and if carbon, it is substituted with (L)ₙR¹;

optionally when ring Q is aromatic, Q¹ is carbon and is substituted with (L)ₙR¹, Q⁴ is carbon, and one of Q², Q³ and Q⁵ is optionally oxygen or sulfur, and the remainder of Q², Q³ and Q⁵ are independently selected from nitrogen and carbon, and if carbon, are substituted with (L)ₙR¹;

when ring Q is partially saturated, Q¹ is selected from carbon and nitrogen, and if carbon, it is mono- or di-substituted with (L)ₙR¹, and if nitrogen, it is unsubstituted or substituted with (L)ₙR¹, Q⁴ is selected from carbon and nitrogen, but only one of Q¹ and Q⁴ can be nitrogen, each of Q², Q³ and Q⁵ is independently selected from carbon, nitrogen, oxygen and sulfur, and if oxygen or sulfur, it is unsubstituted, and if carbon, it is mono- or di-substituted with (L)ₙR¹, and if nitrogen, it is unsubstituted or substituted with (L)ₙR¹;

when R^{a} is structure 3, it is fully conjugated, X² is selected from oxygen or nitrogen substituted with (L)ₙR¹, X¹ is carbon and is substituted with (L)ₙR¹, and each of X⁵ and X⁶ is independently selected from nitrogen and carbon, and if carbon, it is substituted with (L)ₙR¹;

R¹ is selected from —H, C₁-C₆ alkyl, C₂-C₆ alkenyl, C₂-C₆ alkynyl, C₁-C₆ alkyl-R¹¹, C₂-C₆ alkenyl-R¹¹, C₂-C₆ alkynyl-R¹¹, C₁-C₆ alkyl-(R¹¹)₂, C₂-C₆ alkenyl-(R¹¹)₂, CSR¹¹, amino, CONHR¹¹, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkylC(R¹¹)═N—N(R⁸)(R⁹), (C₁-C₄)alkyl-N═N(R⁷), (C₁-C₄)alkyl-N(R⁷)—N═C(R⁸), nitro, cyano, CO₂R¹¹, O—R¹⁰, C₁-C₄ alkyl-OR¹⁰, COR¹¹, SR¹⁰, SSR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, Si(R¹¹)₃, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;

R⁷, R⁸ and R⁹ are each independently selected from —H, C₁-C₆ alkyl, C₂-C₆ alkenyl, C₂-C₆ alkynyl, C₁-C₄ alkyl-R¹¹, C₁-C₆ alkyl-NHR¹³, C₁-C₆ alkyl-NR¹³R⁴, O—R⁵, C₁-C₄ alkyl-OR¹⁵, CO₂R¹⁵, C(S)OR¹⁵, C(O)SR¹⁵, C(O)R¹⁷, C(S)R¹⁷, CONHR¹⁶, C(S)NHR¹⁶, CON(R¹⁶)₂, C(S)N(R¹⁶)₂, SR¹⁵, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁₋₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹⁰ is selected from —H, C₁-C₁₀ alkyl, C₂-C₆ alkenyl, C₂-C₆ alkynyl, C₁-C₆ alkyl-NHR³, C₁-C₆ alkyl-NR¹³R¹⁴, C₁-C₄ alkyl-OR¹⁵, CSR¹¹, CO₂R¹⁵, C(S)OR¹⁵, C(O)SR¹⁵, COR¹⁷, C(S)R¹⁷, CONHR¹⁶, C₁-C₄ alkyl-R¹¹, C₁-C_{4 alkyl-NH}₂R¹³, C(S)NHR¹⁶, O—R¹⁵, CON(R¹⁶)₂, C(S)N(R¹⁶)₂, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, Si(R¹³)₂R¹⁷, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹¹ is selected from —H, C₁-C₆ alkyl, C₁-C₆ alkoxy, C₂-C₆ alkenyl, C₂-C₆ alkynyl, amino, NHR¹³, NR¹³, R¹⁴, N═NR¹³, C₁-C₆ alkyl-NHR¹³, C₁-C₆ alkyl-NR¹³R¹⁴, O—R¹⁵, C₁-C₄ alkyl-OR¹⁵, SR¹⁵, COR¹³, CO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹² is selected from —H, OH, oxo, C₁-C₁₀ alkyl, C₂-C₁₀ alkenyl, C₂-C₁₀ alkynyl, C₁-C₁₀ alkyl-R¹¹, C₂-C₁₀ alkenyl-R¹¹, C₂-C₁₀ alkynyl-R¹¹, C₁-C₁₀ alkyl-(R¹¹)₂, C₂-C₁₀ alkenyl-(R¹¹)₂, CSR¹¹, hydroxyl C₁-C₆ alkyl-R¹¹, amino C₁-C₄ alkyl-R⁷, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₁₀ alkyl-NHR⁷, C₁-C₁₀ alkyl-NR⁸R⁹, (C₁-C₁₀)alkyl-N(R⁷)—N(R⁸)(R⁹), (C₁-C₁₀)alkylC(R¹¹)═N—N(R⁸)(R⁹), (C₁-C₁₀)alkyl-N═N(R⁷), (C₁-C₁₀)alkyl-N(R⁷)—N═C(R)), SCN, NCS, C₁-C₁₀ alkyl SCN, C₁-C₁₀ alkyl NCS, nitro, cyano, O—R¹⁰, C₁-C₁₀ alkyl-OR¹⁰, COR¹¹, CO₂R¹¹, SR¹⁰, SSR¹⁰, SOR¹¹, SO₂R¹¹, C₁-C₁₀ alkyl-COR¹¹, C₁-C₁₀ alkyl-SR¹⁰, C₁-C₁₀ alkyl-SOR¹¹, C₁-C₁₀ alkyl-SO₂R¹¹, halo, Si(R¹¹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, C₂-C₆ alkenyl, C₂-C₆ alkynyl, C₁-C₄ alkyl-R²³, C₁-C₆ alkyl-NHR¹⁹, C₁-C₆ alkyl-NR¹⁹R²⁰, O—R²¹, C₁-C₄ alkyl-OR²¹, CO₂R²¹, COR²¹, C(S)OR²¹, C(O)SR²¹, C(O)R²³, C(S)R²³, CONHR²², C(S)NHR, CON(R²²)₂, C(S)N(R²²)₂, SR²¹, SOR²³, SO₂R²³, C₁-C₆ alkyl-CO₂R²¹ C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁵ and R¹⁶ are independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CSR¹¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁷ is selected from —H, C₁-C₆ alkyl, C₂-C₆ alkenyl, C₂-C₆ alkenyl-R¹⁹, C₁-C₆ alkyl-R¹⁹, C₂-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁸ is selected from —H, oxo, OH, C₁-C₁₀ alkyl, C₂-C₁₀ alkenyl, C₂-C₁₀ alkynyl, C₁-C₁₀ alkyl-R²³, C₂-C₁₀ alkenyl-R²³, C₂-C₁₀ alkynyl-R²³, C₁-C₁₀ alkyl-(R²³)₂, C₂-C₁₀ alkenyl-(R²³)₂, CSR²³, amino, NHR¹⁹, NR²⁰R²⁰, N(R¹⁹)—N(R²⁰)(R²⁰), C(R²³)═N—N(R²⁰)(R²⁰), N═N(R¹⁹), N(R¹⁹)—N═C(R²⁰), C(R²³)═N—O(R²¹), ON═C(R²³), C₁-C₁₀ alkyl-NHR¹⁹, C₁-C₁₀ alkyl-NR²OR²⁰, (C₁-C₁₀)alkyl-N(R¹⁹)—N(R²⁰) (R²⁰), (C₁-C₁₀)alkylC(R²³)═N—N(R²⁰) (R²⁰), (C₁-C₁₀)alkyl-N═N(R¹⁹), (C₁-C₁₀)alkyl-N(R¹⁹)—N═C(R²⁰), SCN, NCS, C₁-C₁₀ alkyl SCN, C₁-C₁₀ alkyl NCS, nitro, cyano, O—R²¹, C₁-C₁₀ alkyl-OR²¹, COR²³ CO₂R²³SR²¹, SSR²¹, SOR²³, SO₂R²³, C₁-C₁₀ alkyl-COR²³, C₁-C₁₀ alkyl-SR²¹, C₁-C₁₀ alkyl-SOR²³, C₁-C₁₀ alkyl-SO₂R²³, halo, Si(R²³)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁹ and R²⁰ are each independently selected from —H, C₁-C₆ alkyl, C₂-C₆ alkenyl, C₂-C₆ alkynyl, C₁-C₄ alkyl-R²⁹, C₁-C₆ alkyl-NHR²⁵, C₁-C₆ alkyl-NR²⁵R²⁶, O—R²⁷, C₁-C₄ alkyl-OR²⁷, CO₂R²⁷, C(S)OR²⁷, C(O)SR²⁷, C(O)R²⁹, C(S)R²⁹, CONHR²⁷, C(S)NHR²⁷, CON(R²⁸)₂, C(S)N(R²⁸)₂, SR²⁷, SOR²⁹, SO₂R²⁹, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₂-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²¹ and R²² are independently selected from —H, C₁-C₁₁ alkyl, C₂-C₆ alkenyl, C₂-C₆ alkynyl, C₁-C₆ alkyl-NHR²⁵, C₁-C₆ alkyl-NR²⁵R²⁶, C₁-C₄ alkyl-OR²⁷, CSR¹¹, CO₂R²⁸, COR²⁹, CONHR², CON(R²)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²³ is selected from —H, C₁-C₆ alkyl, C₂-C₆ alkenyl, C₂-C₆ alkenyl-R²⁵, C₁-C₆ alkyl-R²⁵, C₂-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁴ is selected from —H, OH, C₁-C₁₀ alkyl, C₂-C₁₀ alkenyl, C₂-C₁₀ alkynyl, C₁-C₁₀ alkyl-R²⁹, C₂-C₁₀ alkenyl-R²⁹, C₂-C₁₀ alkynyl-R²⁹, C₁-C₁₀ alkyl-(R²⁹)₂, C₂-C₁₀ alkenyl-(R²⁹)₂, CSR²⁹, amino, NHR²⁵, NR²⁶R²⁶, N(R²⁵)—N(R²⁶)(R²⁶), C(R²⁹)═N—N(R²⁶)(R²⁶), N═N(R²⁵), N(R²⁵)—N═C(R²⁶), C(R²⁹)═N—O(R²⁷), ON═C(R²⁹), C₁-C₁₀ alkyl-NHR²⁵, C₁-C₁₀ alkyl-NR²⁶R²⁶, (C₁-C₁₀)alkyl-N(R²⁵)—N(R²⁶)(R²⁶), (C₁-C₁₀)alkylC(R²⁹)═N—N(R²⁶)(R²⁶), (C₁-C₁₀)alkyl-N═N(R²⁵), (C₁-C₁₀)alkyl-N(R²⁵)—N═C(R²⁶), SCN, NCS, C₁-C₁₀ alkyl SCN, C₁-C₁₀ alkyl NCS, nitro, cyano, O—R²⁷, C₁-C₁₀ alkyl-OR²⁷, CO₂R²⁹, COR²⁹, SR²⁷, SSR²⁷, SOR²⁹, SO₂R²⁹, C₁-C₁₀ alkyl-COR²⁹ C₁-C₁₀ alkyl-SR²⁷, C₁-C₁₀ alkyl-SOR²⁹, C₁-C₁₀ alkyl-SO₂R²⁹, halo, Si(R²⁹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁵ and R²⁶ are each independently selected from —H, C₁-C₆ alkyl, C₂-C₆ alkenyl, C₂-C₆ alkynyl, C₁-C₄ alkyl-R³⁵, C₁-C₆ alkyl-NHR³, C₁-C₆ alkyl-NR³¹R³², O—R³³, C₁-C₄ alkyl-OR³³, CO₂R³³, C(S)OR³³, C(O)SR³³, C(O)R³⁵, C(S)R³⁵, CONHR³⁴, C(S)NHR³⁴, CON(R³⁴)₂, C(S)N(R³⁴)₂, SR³³ SOR³⁵, SO₂R³⁵, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁷ and R²⁸ are independently selected from —H, C₁-C₆ alkyl, C₂-C₆ alkenyl, C₂-C₆ alkynyl, C₁-C₆ alkyl-NHR³, C₁-C₆ alkyl-NR³¹R³², C₁-C₄ alkyl-OR³³, CSR¹¹, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁹ is selected from —H, C₁-C₆ alkyl, C₂-C₆ alkenyl, C₂-C₆ alkenyl-R³¹, C₁-C₆ alkyl-R³¹, C₂-C₆ alkynyl, amino, NHR³¹, NR³¹R³², C₁-C₆ alkyl-NHR³¹, C₁-C₆ alkyl-NR³¹, R³², O—R³³, C₁-C₄ alkyl-OR³³, SR³³, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁰ is selected from —H, OH, C₁-C₁₀ alkyl, C₂-C₁₀ alkenyl, C₂-C₁₀ alkynyl, C₁-C₁₀ alkyl-R³⁵, C₂-C₁₀ alkenyl-R³⁵, C₂-C₁₀ alkynyl-R³⁵, C₁-C₁₀ alkyl-(R³⁵)₂, C₂-C₁₀ alkenyl-(R³⁵)₂, CSR³⁵, amino, NHR³¹, NR³²R³², N(R³¹)—N(R³²)(R³²), C(R³⁵)═N—N(R³²)(R³²), N═N(R³¹), N(R³¹)—N═C(R³²), C(R³⁵)═N—O(R³³), ON═C(R³⁵), C₁-C₁₀ alkyl-NHR³¹, C₁-C₁₀ alkyl-NR³²R³², (C₁-C₁₀)alkyl-N(R³¹)—N(R³²)(R³²), (C₁-C₁₀)alkylC(R³⁵)═N—N(R³²)(R³²), (C₁-C₁₀)alkyl-N═N(R³¹), (C₁-C₁₀)alkyl-N(R³¹)—N═C(R³²), SCN, NCS, C₁-C₁₀ alkyl SCN, C₁-C₁₀ alkyl NCS, nitro, cyano, O—R³³, C₁-C₁₀ alkyl-OR³³, COR³⁵, SR³³, SSR³³, SOR³⁵, SO₂R³⁵, C₁-C₁₀ alkyl-COR³⁵, C₁-C₁₀ alkyl-SR³³, C₁-C₁₀ alkyl-SOR³⁵, C₁-C₁₀ alkyl-SO₂R³⁵, halo, Si(R³⁵)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁶ is selected from —H, alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;

R², R³, R⁴, R⁵, R³⁷ and R³⁸ are each independently absent, or selected from an R¹ group;

n is 0; and

R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

The present invention is also directed to a novel compound having the structure of formula II:

Formula II.

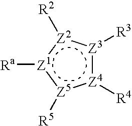

wherein:

Z² and Z³ are nitrogen, Z¹, Z⁴ and Z⁵ are carbon, and join with Z² and Z³ to form a pyrazole ring, or optionally, Z⁴ and Z⁵ are nitrogen, Z¹, Z² and Z³ are carbon and join with Z⁴ and Z⁵ to form a pyrazole ring;

R^{a} is selected from:

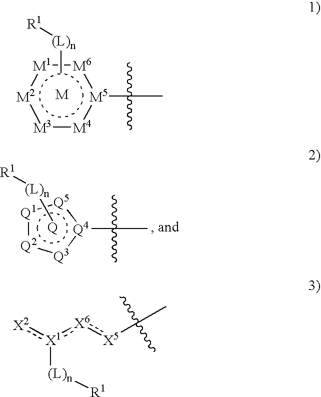

where dashed lines indicate optional single or double bonds;

when R^{a} is ring M and ring M is aromatic, M¹ is carbon and is substituted with (L)ₙR¹, M⁵ is carbon, and each of M², M³, M⁴ and M⁶ is independently selected from carbon and nitrogen and is unsubstituted or substituted with (L)ₙR¹;

when ring M is partially saturated, M¹ is carbon and is mono- or di-substituted with (L)ₙR¹, M⁵ is carbon, and each of M², M³, M⁴ and M⁶ is independently selected from carbon, nitrogen, oxygen and sulfur, and when M², M³, M⁴, or M⁶ is oxygen or sulfur, it is unsubstituted, and when M², M³, M⁴ or M⁶ is carbon or nitrogen, it is optionally unsubstituted; or mono- or di-substituted with (L)ₙR¹;

when R^{a} is ring Q and ring Q is aromatic, Q¹ is selected from carbon and nitrogen, and when Q¹ is carbon, it is substituted with (L)ₙR¹, and when Q¹ is nitrogen, it is unsubstituted, Q⁴ is selected from nitrogen and carbon, and each of Q², Q³ and Q⁵ is independently selected from nitrogen and carbon, and if carbon, it is substituted with (L)ₙR¹;

optionally when ring Q is aromatic, Q¹ is carbon and is substituted with (L)ₙR¹, Q⁴ is carbon, and one of Q², Q³ and Q⁵ is optionally oxygen or sulfur, and the remainder of Q², Q³ and Q⁵ are independently selected from nitrogen and carbon, and if carbon, are substituted with (L)ₙR¹;

when ring Q is partially saturated, Q¹ is selected from carbon and nitrogen, and if carbon, it is mono- or di-substituted with (L)ₙR¹, and if nitrogen, it is unsubstituted or substituted with (L)ₙR¹, Q⁴ is selected from carbon and nitrogen, but only one of Q¹ and Q⁴ can be nitrogen, each of Q², Q³ and Q⁵ is independently selected from carbon, nitrogen, oxygen and sulfur, and if oxygen or sulfur, it is unsubstituted, and if carbon, it is mono- or di-substituted with (L)ₙR¹, and if nitrogen, it is unsubstituted or substituted with (L)ₙR¹;

when R^{a} is structure 3, it is fully conjugated, X² is selected from oxygen or nitrogen substituted with (L)ₙR¹, X¹ is carbon and is substituted with (L)ₙR¹, and each of X⁵ and X⁶ is independently selected from nitrogen and carbon, and if carbon, it is substituted with (L)ₙR¹;

R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR⁷, CO₂R⁷, CONHR⁷, N(R⁸)₂, amino C₁-C₄ alkyl, hydroxy C₁-C₄ alkyl, amino, amino C₁-C₄ alkyl-R⁷, halo C₁-C₄ alkyl, C₁-C₆ alkyl-NHR⁷, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R⁷ and R⁸ are each independently selected from —H, C₁-C₆ alkyl, C₁-C₄ alkyl-R¹¹, C₁-C₆ alkyl-N(R¹³)₂, CO₂R¹⁶, COR¹⁷, aryl, and arylalkyl, wherein aryl and arylalkyl, are optionally substituted with one or more of the groups defined by R¹⁸;

R⁹ and R¹⁰ are each independently selected from —H, hydroxyl, C₁-C₆ alkyl, C₁-C₆ alkyl-R¹⁷, C₁-C₆ alkyl-NH₂R¹³, CO₂R¹, COR¹⁷, C₁-C₆ alkyl-CO₂R¹⁶, C₁-C₆ alkyl-CONH—R¹⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;

R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₁-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;

R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R³)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, COR²³, and aryl;

R¹⁵ and R¹⁶ are each independently selected from —H, aryl, arylalkyl, wherein aryl, arylalkyl, are optionally substituted with one or more of the groups defined by R²⁴;

R¹⁷ is selected from —H, C₁-C₆ alkyl, C₁-C₆ alkyl-R¹⁹, NHR¹⁹, aryl, heteroarylalkyl, and heterocyclylalkyl, wherein aryl is optionally substituted with one or more of the groups defined by R²⁴;

R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;

R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;

R²¹ and R²² are each independently selected from —H and C₁-C₆ alkyl;

R²³ is selected from —H and C₁-C₆ alkyl;

R²⁴ is selected from —H, C₁-C₆ alkyl, C₁-C₆ alkoxy, CO₂R²⁹, halo, and halo C₁-C₄ alkyl;

R²⁹ is selected from —H, and C₁-C₆ alkyl;

R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;

R³⁶ is selected from —H and halo;

R², R³, R⁴, R³⁷ and R³⁸ are each independently selected from an R¹ group;

n is 0; and

R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

The present invention is also directed to a novel MK-2 inhibiting compound that is listed in Table I or Table II, below.

The present invention is also directed to a novel method of inhibiting MK-2, the method comprising contacting MK-2 with at least one compound that is described in Table I or Table II, below.

The present invention is also directed to a novel method of preventing or treating a TNFα mediated disease or disorder in a subject, the method comprising administering to the subject an effective amount of an MK-2 inhibiting compound having the structure described in formula I.

The present invention is also directed to a novel method of preventing or treating a TNFα mediated disease or disorder in a subject, the method comprising administering to the subject at least one MK-2 inhibiting compound that is described in Table I or Table II, below.

The present invention is also directed to a novel therapeutic composition comprising a compound having the structure described in formula I.

The present invention is also directed to a novel therapeutic composition comprising at least one MK-2 inhibitory compound that is described in Table I or Table II.

The present invention is also directed to a novel pharmaceutical composition comprising a pharmaceutically acceptable carrier and at least one MK-2 inhibitory compound having the structure described in formula I.

The present invention is also directed to a novel comprising a dosage form that includes a therapeutically effective amount of at least one MK-2 inhibitory compound having a structure described in formula I.

Among the several advantages found to be achieved by the present invention, therefore, may be noted the provision of a method that could serve to modulate the activity of MK-2—in particular, to inhibit MK-2 activity—and the provision of a method for the prevention and treatment of diseases and disorders that are mediated by TNFα.

### BRIEF DESCRIPTION OF THE DRAWINGS

FIG. 1 is a graph showing paw thickness as a function of time from day 0 to day 7 for MK2 (+/+) and MK2 (−/−) mice, which have received serum injection; and

FIG. 2 is a bar chart showing paw thickness at seven days after injection for normal mice, MK2 (+/+) mice receiving serum, MK2 (−/−) mice receiving serum, and MK2 (+/+) mice receiving serum and anti-TNF antibody;

### DETAILED DESCRIPTION OF THE PREFERRED EMBODIMENTS

In accordance with the present invention, it has been discovered that certain compounds can inhibit the activity of MAPKAP kinase-2. Many of these compounds exhibit their inhibitory effect at low concentrations—having in vitro MK-2 inhibition IC₅₀ values of under 1.0 μM, and with some having IC₅₀ values of under about 0.1 μM, and even as low as about 0.02 μM. Accordingly, these compounds can be potent and effective drugs for use in the inhibition of MK-2, and of special value in subjects where such inhibition would be useful. In particular, these compounds would be useful in methods to prevent or treat diseases and disorders that are mediated by TNFα. For example, they can be used for the prevention or treatment of arthritis.

Compounds that have a high degree of MK-2 inhibiting activity offer advantages in therapeutic uses, because therapeutic benefits can be obtained by the administration of lower amounts of the present compounds than with less active compounds. Such highly active compounds also result in fewer side effects, and in some embodiments, demonstrate a selectivity for MK-2 inhibition over the inhibition of other related kinases.

The present MK-2 inhibitory compounds inhibit the activity of the MK-2 enzyme. When it is said that a subject compound inhibits MK-2, it is meant that the MK-2 enzymatic activity is lower in the presence of the compound than it is under the same conditions in the absence of such compound. One method of expressing the potency of a compound as an MK-2 inhibitor is to measure the “IC₅₀” value of the compound. The IC₅₀ value of an MK-2 inhibitor is the concentration of the compound that is required to decrease the MK-2 enzymatic activity by one-half. Accordingly, a compound having a lower IC₅₀ value is considered to be a more potent inhibitor than a compound having a higher IC₅₀ value. As used herein, compounds that inhibit MK-2 can be referred to as MK-2 inhibitors, or MK-2 inhibiting compounds or MK-2 inhibiting agents.

In practice, the selectivity of an MK-2 inhibitor varies depending upon the condition under which the test is performed and on the inhibitors being tested. However, for the purposes of this specification, the selectivity of an MK-2 inhibitor can be measured as a ratio of the in vitro or in vivo IC₅₀ value for inhibition of MK-3, divided by the IC₅₀ value for inhibition of MK-2 (IC₅₀ MK-3/IC₅₀ MK-2). As used herein, the term “IC₅₀” refers to the concentration of a compound that is required to produce 50% inhibition of MK-2 or MK-3 activity. An MK-2 selective inhibitor is any inhibitor for which the ratio of IC₅₀ MK-3 to IC₅₀ MK-2 is greater than 1. In preferred embodiments, this ratio is greater than 2, more preferably greater than 5, yet more preferably greater than 10, still more preferably greater than 50, and more preferably still, is greater than 100. Such preferred selectivity may indicate an ability to reduce the incidence of side effects incident to the administration of an MK-2 inhibitor to a subject.

Compounds that are useful in the present method include those having the structure shown in formula I:

Formula I:

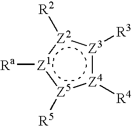

wherein:

Z² and Z³ are nitrogen, Z¹, Z⁴ and Z⁵ are carbon, and join with Z² and Z³ to form a pyrazole ring, or optionally, Z⁴ and Z⁵ are nitrogen, Z¹, Z² and Z³ are carbon and join with Z⁴ and Z⁵ to form a pyrazole ring;

R^{a} is selected from:

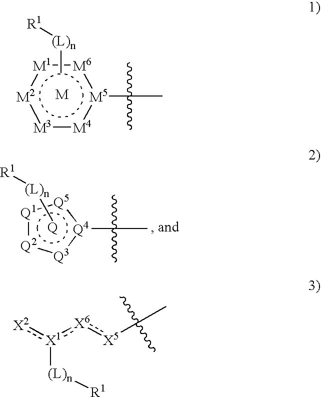

where dashed lines indicate optional single or double bonds;

when R^{a} is ring M and ring M is aromatic, M¹ is carbon and is substituted with (L)ₙR¹, M⁵ is carbon, and each of M², M³, M⁴ and M⁶ is independently selected from carbon and nitrogen and is unsubstituted or substituted with (L)ₙR¹;

when ring M is partially saturated, M¹ is carbon and is mono- or di-substituted with (L)ₙR¹, M⁵ is carbon, and each of M², M³, M⁴ and M⁶ is independently selected from carbon, nitrogen, oxygen and sulfur, and when M², M³, M⁴, or M⁶ is oxygen or sulfur, it is unsubstituted, and when M², M³, M⁴ or M⁶ is carbon or nitrogen, it is optionally unsubstituted; or mono- or di-substituted with (L)ₙR¹;

when R^{a} is ring Q and ring Q is aromatic, Q¹ is selected from carbon and nitrogen, and when Q¹ is carbon, it is substituted with (L)ₙR¹, and when Q¹ is nitrogen, it is unsubstituted, Q⁴ is selected from nitrogen and carbon, and each of Q², Q³ and Q⁵ is independently selected from nitrogen and carbon, and if carbon, it is substituted with (L)ₙR¹;

optionally when ring Q is aromatic, Q¹ is carbon and is substituted with (L)ₙR¹, Q⁴ is carbon, and one of Q², Q³ and Q⁵ is optionally oxygen or sulfur, and the remainder of Q², Q³ and Q⁵ are independently selected from nitrogen and carbon, and if carbon, are substituted with (L)ₙR¹;

when ring Q is partially saturated, Q¹ is selected from carbon and nitrogen, and if carbon, it is mono- or di-substituted with (L)ₙR¹, and if nitrogen, it is unsubstituted or substituted with (L)ₙR¹, Q⁴ is selected from carbon and nitrogen, but only one of Q¹ and Q⁴ can be nitrogen, each of Q², Q³ and Q⁵ is independently selected from carbon, nitrogen, oxygen and sulfur, and if oxygen or sulfur, it is unsubstituted, and if carbon, it is mono- or di-substituted with (L)ₙR¹, and if nitrogen, it is unsubstituted or substituted with (L)ₙR¹;

when R^{a} is structure 3, it is fully conjugated, X² is selected from oxygen or nitrogen substituted with (L)ₙR¹, X¹ is carbon and is substituted with (L)ₙR¹, and each of X⁵ and X⁶ is independently selected from nitrogen and carbon, and if carbon, it is substituted with (L)ₙR¹;

R¹ is selected from —H, C₁-C₆ alkyl, C₂-C₆ alkenyl, C₂-C₆ alkynyl, C₁-C₆ alkyl-R¹, C₂-C₆ alkenyl-R¹¹, C₂-C₆ alkynyl-R¹, C₁-C₆ alkyl-(R¹)₂, C₂-C₆ alkenyl-(R¹¹)₂, CSR¹¹, amino, CONHR¹¹, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹)═N—O(R¹⁰), ON═C(R¹¹), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkylC(R¹¹)═N—N(R⁸)(R⁹), (C₁-C₄)alkyl-N═N(R⁷), (C₁-C₄)alkyl-N(R⁷)—N═C(R⁸), nitro, cyano, CO₂R¹¹, O—R¹⁰, C₁-C₄ alkyl-OR¹⁰, COR¹¹, SR¹⁰, SSR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, Si(R¹¹)₃, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;

R⁷, R⁸ and R⁹ are each independently selected from —H, C₁-C₆ alkyl, C₂-C₆ alkenyl, C₂-C₆ alkynyl, C₁-C₄ alkyl-R¹¹, C₁-C₆ alkyl-NHR¹³, C₁-C₆ alkyl-NR¹³R¹⁴, O—R¹⁵, C₁-C₄ alkyl-OR¹⁵, CO₂R¹⁵, C(S)OR¹⁵, C(O)SR¹⁵, C(O)R¹⁷, C(S)R¹⁷, CONHR¹⁶, C(S)NHR¹⁶, CON(R¹⁶)₂, C(S)N(R¹⁶)₂, SR¹⁵, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹⁰ is selected from —H, C₁-C₁₀ alkyl, C₂-C₆ alkenyl, C₂-C₆ alkynyl, C₁-C₆ alkyl-NHR¹³, C₁-C₆ alkyl-NR¹³R¹⁴, C₁-C₄ alkyl-OR¹⁵, CSR¹¹, CO₂R¹⁵, C(S)OR¹⁵, C(O)SR¹⁵, COR¹⁷, C(S)R¹⁷, CONHR¹⁶ C₁₀ alkyl-R¹¹ C₁-C₄ alkyl-NH₂R¹³, C(S)NHR¹⁶, O—R¹⁵, CON(R¹⁶)₂, C(S)N(R¹⁶)₂, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, Si(R¹³)₂R¹⁷, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹¹ is selected from —H, C₁-C₆ alkyl, C₁-C₆ alkoxy, C₂-C₆ alkenyl, C₂-C₆ alkynyl, amino, NHR¹³, NR¹³R¹⁴, N═NR¹³, C₁-C₆ alkyl-NHR¹³, C₁-C₆ alkyl-NR¹³R¹⁴, O—R¹⁵, C₁-C₄ alkyl-OR¹⁵, SR¹⁵, COR¹³, CO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹² is selected from —H, OH, oxo, C₁-C₁₀ alkyl, C₂-C₁₀ alkenyl, C₂-C₁₀ alkynyl, C₁-C₁₀ alkyl-R¹¹, C₂-C₁₀ alkenyl-R¹¹, C₂-C₁₀ alkynyl-R¹¹, C₁-C₁₀ alkyl-(R¹¹)₂, C₂-C₁₀ alkenyl-(R¹¹)₂, CSR¹¹, hydroxyl C₁-C₆ alkyl-R¹¹, amino C₁-C₄ alkyl-R⁷, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₁₀ alkyl-NHR⁷, C₁-C₁₀ alkyl-NR⁸R⁹, (C₁-C₁₀)alkyl-N(R⁷)—N(R⁸)(R⁹), (C₁-C₁₀)alkylC(R¹¹)═N—N(R⁸)(R⁹), (C₁-C₁₀)alkyl-N═N(R⁷), (C₁-C₁₀)alkyl-N(R⁷)—N═C(R⁸), SCN, NCS, C₁-C₁₀ alkyl SCN, C₁-C₁₀ alkyl NCS, nitro, cyano, O—R¹⁰, C₁-C₁₀ alkyl-OR¹⁰, COR¹¹, CO₂R¹¹, SR¹⁰, SSR¹⁰, SOR¹¹, SO₂R¹¹, C₁-C₁₀ alkyl-COR¹¹, C₁-C₁₀ alkyl-SR¹⁰, C₁-C₁₀ alkyl-SOR¹¹, C₁-C₁₀ alkyl-SO₂R¹¹, halo, Si(R¹¹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, C₂-C₆ alkenyl, C₂-C₆ alkynyl, C₁-C₄ alkyl-R²³, C₁₀₆ alkyl-NHR¹⁹, C₁-C₆ alkyl-NR¹⁹R²⁰, O—R²¹, C₁-C₄ alkyl-OR²¹, CO₂R²¹, COR²¹, C(S)OR²¹, C(O)SR²¹, C(O)R²³, C(S)R²³, CONHR²², C(S)NHR²², CON(R²²)₂, C(S)N(R²²)₂, SR²¹, SOR²³, SO₂R²³, C₁-C₆ alkyl-CO₂R²¹ C₁-C₆ alkyl C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁵ and R¹⁶ are independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CSR¹¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁷ is selected from —H, C₁-C₆ alkyl, C₂-C₆ alkenyl, C₂-C₆ alkenyl-R¹⁹, C₁-C₆ alkyl-R¹⁹, C₂-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁸ is selected from —H, oxo, OH, C₁-C₁₀ alkyl, C₂-C₁₀ alkenyl, C₂-C₁₀ alkynyl, C₁-C₁₀ alkyl-R²³, C₂-C₁₀ alkenyl-R²³, C₂-C₁₀ alkynyl-R²³, C₁-C₁₀ alkyl-(R²³)₂, C₂-C₁₀ alkenyl-(R²³)₂, CSR²³, amino, NHR¹⁹, NR²OR²⁰, N(R¹⁹)—N(R²⁰)(R²⁰), C(R²³)═N—N(R²⁰)(R²⁰), N═N(R¹⁹), N(R¹⁹)—N═C(R²⁰), C(R²³)═N—O(R²¹), ON═C(R²³), C₁-C₁₀ alkyl-NHR¹⁹, C₁-C₁₀ alkyl-NR²OR²⁰, (C₁-C₁₀)alkyl-N(R¹⁹)—N(R²⁰) (R²⁰), (C₁-C₁₀)alkylC(R²³)═N—N(R²⁰) (R²⁰), (C₁-C₁₀)alkyl-N═N(R¹⁹), (C₁-C₁₀)alkyl-N(R¹⁹)—N═C(R²⁰), SCN, NCS, C₁-C₁₀ alkyl SCN, C₁-C₁₀ alkyl NCS, nitro, cyano, O—R², C₁-C₁₀ alkyl-OR²¹, COR²³, C₁₀R²³ SR²¹, SSR², SOR²³, SO₂R²³, C₁-C₁₀ alkyl-COR²³, C₁-C₁₀ alkyl-SR²¹, C₁-C₁₀ alkyl-SOR²³, C₁-C₁₀ alkyl-SO₂R²³, halo, Si(R²³)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁹ and R²⁰ are each independently selected from —H, C₁-C₆ alkyl, C₂-C₆ alkenyl, C₂-C₆ alkynyl, C₁-C₄ alkyl-R²⁹, C₁-C₆ alkyl-NHR²⁵, C₁-C₆ alkyl-NR²⁵R²⁶, O—R²⁷, C₁-C₄ alkyl-OR²⁷, CO₂R²⁷, C(S)OR²⁷, C(O)SR²⁷, C(O)R²⁹, C(S)R²⁹, CONHR²⁸, C(S)NHR²⁸, CON(R²⁸)₂, C(S)N(R²⁷)₂, SR²⁷, SOR²⁹, SO₂R²⁹, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²¹ and R²² are independently selected from —H, C₁-C₁₀ alkyl, C₂-C₆ alkenyl, C₂-C₆ alkynyl, C₁-C₆ alkyl-NHR²⁵, C₁-C₆ alkyl-NR²⁵R²⁶, C₁₀₄ alkyl-OR²⁷, CSR¹¹, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²³ is selected from —H, C₁-C₆ alkyl, C₂-C₆ alkenyl, C₂-C₆ alkenyl-R²⁵, C₁-C₆ alkyl-R²⁵, C₂-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C_{1-C}₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₁ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁴ is selected from —H, OH, C₁-C₁₀ alkyl, C₂-C₁₀ alkenyl, C₂-C₁₀ alkynyl, C₁-C₁₀ alkyl-R²⁹, C₂-C₁₀ alkenyl-R²⁹, C₂-C₁₀ alkynyl-R²⁹, C₁C₁₀ alkyl-(R²⁹)₂, C₂-C₁₀ alkenyl-(R²⁹)₂, CSR²⁹, amino, NHR²⁵, NR²⁶R²⁶, N(R²⁵)—N(R²⁶)(R²⁶), C(R²⁹)═N—N(R²⁶)(R²⁶), N═N(R²⁵), N(R²⁵)—N═C(R²⁶), C(R²⁹)═N—O(R²⁷), ON═C(R²⁹), C₁-C₁₀ alkyl-NHR²⁵, C₁-C₁₀ alkyl-NR²⁶R²⁶, (C₁-C₁₀)alkyl-N(R²⁵)—N(R²⁶)(R²⁶), (C₁-C₁₀)alkylC(R²⁹)═N—N(R²⁶)(R²⁶), (C₁-C₁₀)alkyl-N═N(R²⁵), (C₁-C₁₀)alkyl-N(R²⁵)—N═C(R²⁶), SCN, NCS, C₁-C₁₀ alkyl SCN, C₁-C₁₀ alkyl NCS, nitro, cyano, O—R²⁷, C₁-C₁₀ alkyl-OR²⁷, CO₂R²⁹, COR²⁹, SR²⁷, SSR²⁷, SOR²⁹, SO₂R²⁹, C₁-C₁₀ alkyl-COR²⁹, C_-C₁₀ alkyl-SR²⁷, C₁-C₁₀ alkyl-SOR²⁹, C₁-C₁₀ alkyl-SO₂R²⁹, halo, Si(R²⁹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁵ and R²⁶ are each independently selected from —H, C₁-C₆ alkyl, C₂-C₆ alkenyl, C₂-C₆ alkynyl, C₁-C₄ alkyl-R³⁵, C₁-C₆ alkyl-NHR³¹, C₁-C₆ alkyl-NR³¹R³², O—R³³, C₁-C₄ alkyl-OR³³, CO₂R³³, C(S)OR³³, C(O)SR³³, C(O)R, C(S)R, CONHR³⁴, C(S)NHR³⁴, CON(R³⁴)₂, C(S)N(R³⁴)₂, SR³³ SOR³⁵, SO₂R³⁵, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁷ and R²⁸ are independently selected from —H, C₁-C₆ alkyl, C₂-C₆ alkenyl, C₂-C₆ alkynyl, C₁-C₆ alkyl-NHR³¹, C₁-C₆ alkyl-NR³¹R³², C₁-C₄ alkyl-OR³³, CSR¹¹, CO₂R³⁴, COR³⁵, CONHR³⁴, CON(R₂, SOR³⁵, SO₂R³⁵, C₁-C₆ alkyl-CO₂R³⁴, C—C₆ alkyl-COR³⁵, C₁-C₆ alkyl-CONHR³, C₁-C₆ alkyl-CON(R³⁴)₂, C₁-C₆ alkyl-SR³³, C₁-C₆ alkyl-SOR³⁵, C₁-C₆ alkyl -SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁹ is selected from —H, C₁-C₆ alkyl, C₂-C₆ alkenyl, C₂-C₆ alkenyl-R³¹, C₁-C₆ alkyl-R³¹, C₂-C₆ alkynyl, amino, NHR³¹, NR³¹R³², C₁-C₆ alkyl-NHR³¹, C₁-C₆ alkyl-NR³¹, R³², O—R³³, C₁-C₄ alkyl-OR³³, SR³³, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁰ is selected from —H, OH, C₁-C₁₀ alkyl, C₂-C₁₀ alkenyl, C₂-C₁₀ alkynyl, C₁-C₁₀ alkyl-R³⁵, C₂-C₁₀ alkenyl-R³⁵, C₂-C₁₀ alkynyl-R³⁵, C₁-C₁₀ alkyl-(R³⁵)₂, C₂-C₁₀ alkenyl-(R³⁵)₂, CSR³⁵, amino, NHR³¹, NR³²R³², N(R³¹)—N(R³²)(R³²), C(R³⁵)═N—N(R³²)(R³²), N═N(R³¹), N(R³¹)—N═C(R³²), C(R³⁵)═N—O(R³³), ON═C(R³⁵), C₁-C₁₀ alkyl-NHR³¹, C₁-C₁₀ alkyl-NR³²R³², (C₁-C₁₀)alkyl-N(R³¹)—N(R³²)(R³²), (C₁-C₁₀)alkylC(R³⁵)═N—N(R³²)(R³²), (C₁-C₁₀)alkyl-N═N(R³¹), (C₁-C₁₀)alkyl-N(R³¹)—N═C(R³²), SCN, NCS, C₁-C₁₀ alkyl SCN, C₁-C₁₀ alkyl NCS, nitro, cyano, O—R³³, C₁-C₁₀ alkyl-OR³³, COR³⁵, SR³³, SSR³³, SOR³⁵, SO₂R³⁵, C₁-C₁₀ alkyl-COR³⁵, C₁-C₁₀ alkyl-SR³³, C₁-C₁₀ alkyl-SOR³⁵, C₁-C₁₀ alkyl-SO₂R³⁵, halo, Si(R³⁵)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C—CO₁₀ mono- and bicyclic cycloalkyl are optionally substituted with one or more of the groups defined by R³⁶;

R³¹ R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁶ is selected from —H, alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;

R², R³, R⁴, R⁵, R³⁷ and R³⁸ are each independently absent, or selected from an R¹ group;

n is 0; and

R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

The “M” ring and the “Q” ring of the structure of formula I can have any number of R¹-Lₙ- substituent groups, ranging from zero to one or more per ring atom, and such substituent groups can be located on any atom of the ring having a valence suitable for the addition of a substituent group(s). Each such substituent group can have any number of R¹ groups per L group, ranging from zero to 5. A preferred structure is the presence of either 0 or 1 R¹-Lₙ- substituent groups on the ring. It is also preferred that the R¹-Lₙ- substituent group is attached to the ring at the M¹ or the Q¹ location, respectively.

The meaning of any substituent at any one occurrence in Formula I, or any other general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1 yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like. The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical. The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical. The term “hydroxyalkyl” embraces linear or branched alkyl radicals having one to about ten carbon atoms any one of which may be substituted with one or more hydroxyl radicals. The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain. The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as:

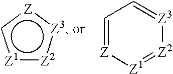

where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures:

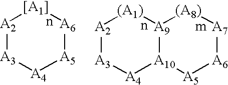

where:

when n=1, m=1 and A₁-A₈ are each CR^{x} or N, A₉ and A₁₀ are carbon;

when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A⁹ and A₁₀ are carbon;

when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;

when n is greater than or equal to 0, and m greater than or equal to 0, atoms separated by 2 atoms (i.e., A₁ and A₄) are Sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical. The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂ (—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl. The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively. The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as:

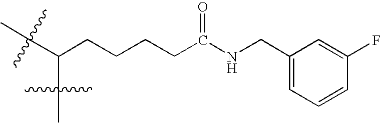

may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

The present invention also comprises MK-2 inhibiting compounds having the structure shown in formula II:

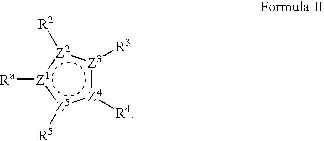

wherein:

Z² and Z³ are nitrogen, Z¹, Z⁴ and Z⁵ are carbon, and join with Z² and Z³ to form a pyrazole ring, or optionally, Z⁴ and Z⁵ are nitrogen, Z¹, Z² and Z³ are carbon and join with Z⁴ and Z⁵ to form a pyrazole ring;

R^{a} is selected from:

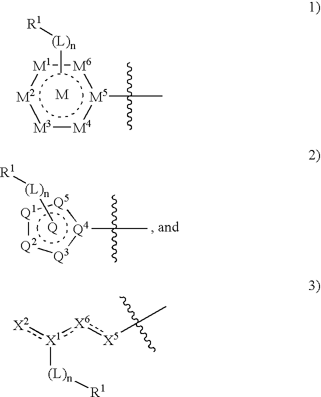

where dashed lines indicate optional single or double bonds;

when R^{a} is ring M and ring M is aromatic, M¹ is carbon and is substituted with (L)ₙR¹, M⁵ is carbon, and each of M², M³, M⁴ and M⁶ is independently selected from carbon and nitrogen and is unsubstituted or substituted with (L)ₙR¹;

when ring M is partially saturated, M¹ is carbon and is mono- or di-substituted with (L)ₙR¹, M⁵ is carbon, and each of M², M³, M⁴ and M⁶ is independently selected from carbon, nitrogen, oxygen and sulfur, and when M², M³, M⁴, or M⁶ is oxygen or sulfur, it is unsubstituted, and when M², M³, M⁴ or M⁶ is carbon or nitrogen, it is optionally unsubstituted; or mono- or di-substituted with (L)ₙR¹;

when R^{a} is ring Q and ring Q is aromatic, Q¹ is selected from carbon and nitrogen, and when Q¹ is carbon, it is substituted with (L)ₙR¹, and when Q¹ is nitrogen, it is unsubstituted, Q⁴ is selected from nitrogen and carbon, and each of Q², Q³ and Q⁵ is independently selected from nitrogen and carbon, and if carbon, it is substituted with (L)ₙR¹;

optionally when ring Q is aromatic, Q¹ is carbon and is substituted with (L)ₙR¹, Q⁴ is carbon, and one of Q², Q³ and Q⁵ is optionally oxygen or sulfur, and the remainder of Q², Q³ and Q⁵ are independently selected from nitrogen and carbon, and if carbon, are substituted with (L)ₙR¹;

when ring Q is partially saturated, Q¹ is selected from carbon and nitrogen, and if carbon, it is mono- or di-substituted with (L)ₙR¹, and if nitrogen, it is unsubstituted or substituted with (L)ₙR¹, Q⁴ is selected from carbon and nitrogen, but only one of Q¹ and Q⁴ can be nitrogen, each of Q², Q³ and Q⁵ is independently selected from carbon, nitrogen, oxygen and sulfur, and if oxygen or sulfur, it is unsubstituted, and if carbon, it is mono- or di-substituted with (L)ₙR¹, and if nitrogen, it is unsubstituted or substituted with (L)ₙR¹;

when R^{a} is structure 3, it is fully conjugated, X² is selected from oxygen or nitrogen substituted with (L)ₙR¹, X¹ is carbon and is substituted with (L)ₙR¹, and each of X⁵ and X⁶ is independently selected from nitrogen and carbon, and if carbon, it is substituted with (L)ₙR¹;

R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR¹⁷, CO₂R¹⁷, CONHR¹⁷, N(R⁸)₂, amino C₁-C₄ alkyl, hydroxy C₁-C₄ alkyl, amino, amino C₁-C₄ alkyl-R⁷, C₁-C₆ alkyl-NHR⁷, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R⁷ and R⁸ are each independently selected from —H, C₁-C₆ alkyl, C₁-C₄ alkyl-R¹¹, C₁-C₆ alkyl-N(R¹³)₂, CO₂R¹⁶, COR¹⁷, aryl, and arylalkyl, wherein aryl and arylalkyl, are optionally substituted with one or more of the groups defined by R¹⁸;

R⁹ and R¹⁰ are each independently selected from —H, hydroxyl, C₁-C₆ alkyl, C₁-C₆ alkyl-R¹⁷, C₁-C₆ alkyl-NH₂R¹³, CO₂R⁶, COR⁷, C₁-C₆ alkyl-CO₂R¹⁶, C₁-C₆ alkyl-CONH—R¹⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;

R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₁-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;

R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R⁸)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, COR²³, and aryl;

R¹⁵ and R¹⁶ are each independently selected from —H, aryl, arylalkyl, wherein aryl, arylalkyl, are optionally substituted with one or more of the groups defined by R²⁴;

R¹⁷ is selected from —H, C₁-C₆ alkyl, C₁-C₆ alkyl-R¹⁹, NHR¹⁹, aryl, heteroarylalkyl, and heterocyclylalkyl, wherein aryl is optionally substituted with one or more of the groups defined by R²⁴;

R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;

R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;

R²¹ and R²² are each independently selected from —H and C₁-C₆ alkyl;

R²³ is selected from —H and C₁-C₆ alkyl;

R²⁴ is selected from —H, C₁-C₆ alkyl, C₁-C₆ alkoxy, CO₂R²⁹, halo, and halo C₁-C₄ alkyl;

R²⁹ is selected from —H, and C₁-C₆ alkyl;

R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;

R³⁶ is selected from —H and halo;

R², R³, R⁴, R³⁷ and R³⁸ are each independently selected from an R¹ group;

n is 0; and

R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

Table I and Table II show examples of MK-2 inhibiting compounds of the present invention, and also shows the chemical name and, where available, the IC₅₀ value of the compound for MK-2 inhibition. It is believed that any of the compounds that are listed in Table I and Table II are MK-2 inhibiting compounds that can be used in the method of the present invention. However, neither the novel MK-2 inhibiting compounds, nor the uses of an MK-2 inhibiting compound that are described herein are intended to be limited to the compounds that are presented in the following Tables.

TABLE I
MK-2 Inhibiting Compounds

| | | | MK-2 Avg. |
|---|---|---|---|
| No. | Structure^{a} | Compound Name(s)^{b} | IC₅₀ (uM) |
| 1 | 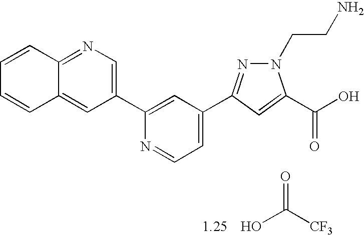 | 1-(2-aminoethyl)-3-(2-quinolin-3-ylpyridin-4- yl)-1H-pyrazole-5-carboxylic acid trifluoroacetate | 0.0269 |
| 2 | 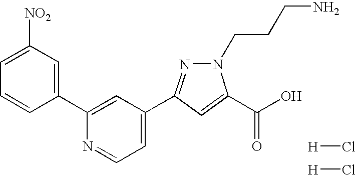 | 1-(3-aminopropyl)-3-[2-(3- nitrophenyl)pyridin-4-yl]-1H-pyrazole-5- carboxylic acid dihydrochloride | 0.0397 |
| 3 | 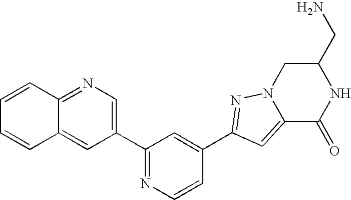 | 6-(aminomethyl)-2-(2-quinolin-3-ylpyridin-4- yl)-6,7-dihydropyrazolo[1,5-a]pyrazin-4(5H)- one | 0.0477 |
| 4 | 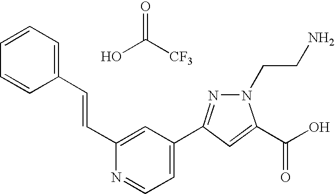 | 1-(2-aminoethyl)-3-{2-[(E)-2- phenylethenyl]pyridin-4-yl}-1H-pyrazole-5- carboxylic acid trifluoroacetate | 0.0505 |
| 5 | 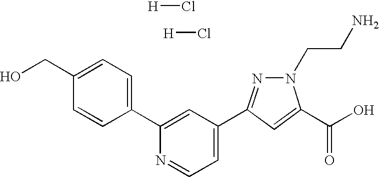 | 1-(2-aminoethyl)-3-{2-[4- (hydroxymethyl)phenyl]pyridin-4-yl}-1H- pyrazole-5-carboxylic acid dihydrochloride | 0.0533 |
| 6 | 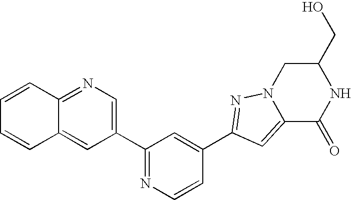 | 6-(hydroxymethyl)-2-(2-quinolin-3-ylpyridin- 4-yl)-6,7-dihydropyrazolo[1,5-a]pyrazin- 4(5H)-one | 0.0615 |
| 7 | 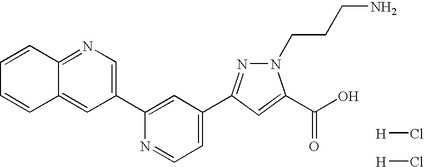 | 1-(3-aminopropyl)-3-(2-quinolin-3-ylpyridin- 4-yl)-1H-pyrazole-5-carboxylic acid dihydrochloride | 0.0686 |
| 8 | 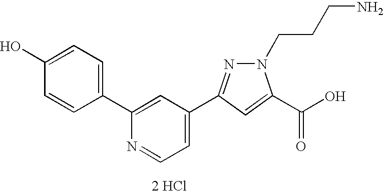 | 1-(3-aminopropyl)-3-[2-(4- hydroxyphenyl)pyridin-4-yl]-1H-pyrazole-5- carboxylic acid hydrochloride | 0.102 |
| 9 | 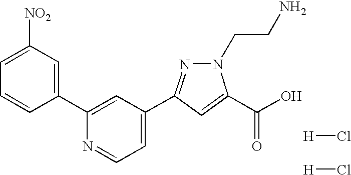 | 1-(2-aminoethyl)-3-[2-(3-nitrophenyl)pyridin- 4-yl]-1H-pyrazole-5-carboxylic acid dihydrochloride | 0.109 |
| 10 | 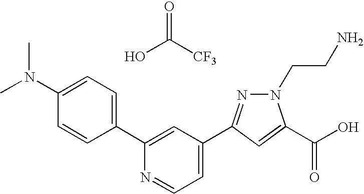 | 1-(2-aminoethyl)-3-{2-[4- (dimethylamino)phenyl]pyridin-4-yl}-1H- pyrazole-5-carboxylic acid trifluoroacetate | 0.117 |
| 11 | 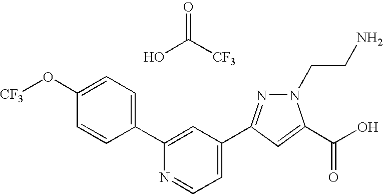 | 1-(2-aminoethyl)-3-{2-[4- (trifluoromethoxy)phenyl]pyridin-4-yl}-1H- pyrazole-5-carboxylic acid trifluoroacetate | 0.161 |
| 12 | 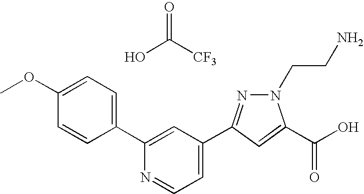 | 1-(2-aminoethyl)-3-[2-(4- methoxyphenyl)pyridin-4-yl]-1H-pyrazole-5- carboxylic acid trifluoroacetate | 0.168 |
| 13 | 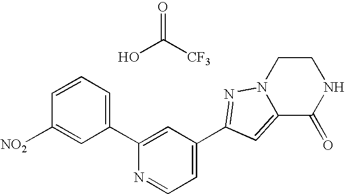 | 2-[2-(3-nitrophenyl)pyridin-4-yl]-6,7- dihydropyrazolo[1,5-a]pyrazin-4(5H)-one trifluoroacetate | 0.171 |
| 14 | 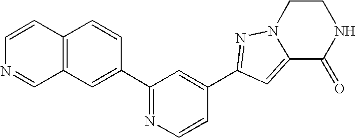 | 2-(2-isoquinolin-7-ylpyridin-4-yl)-6,7- dihydropyrazolo[1,5-a]pyrazin-4(5H)-one | 0.194 |
| 15 | 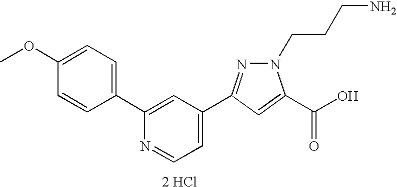 | 1-(3-aminopropyl)-3-[2-(4- methoxyphenyl)pyridin-4-yl]-1H-pyrazole-5- carboxylic acid hydrochloride | 0.196 |
| 16 | 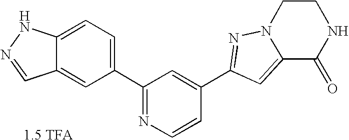 | 2-[2-(1H-indazol-5-yl)pyridin-4-yl]-6,7- dihydropyrazolo[1,5-a]pyrazin-4(5H)-one trifluoroacetate | 0.199 |
| 17 | 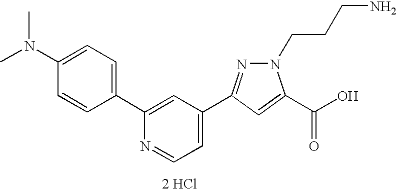 | 1-(3-aminopropyl)-3-{2-[4- (dimethylamino)phenyl]pyridin-4-yl}-1H- pyrazole-5-carboxylic acid hydrochloride | 0.203 |
| 18 | 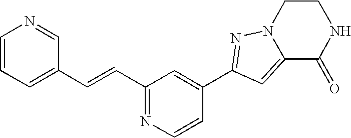 | 2-{2-[(E)-2-pyridin-3-ylethenyl]pyridin-4-yl}- 6,7-dihydropyrazolo[1,5-a]pyrazin-4(5H)- one | 0.212 |
| 19 | 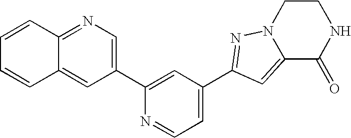 | 2-(2-quinolin-3-ylpyridin-4-yl)-6,7- dihydropyrazolo[1,5-a]pyrazin-4(5H)-one | 0.216 |
| 20 | 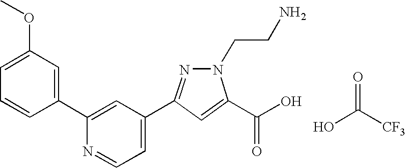 | 1-(2-aminoethyl)-3-[2-(3- methoxyphenyl)pyridin-4-yl]-1H-pyrazole-5- carboxylic acid trifluoroacetate | 0.217 |
| 21 | 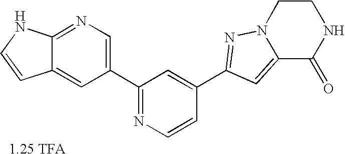 | 2-[2-(1H-pyrrolo[2,3-b]pyridin-5-yl)pyridin-4- yl]-6,7-dihydropyrazolo[1,5-a]pyrazin-4(5H)- one trifluoroacetate | 0.228 |
| 22 | 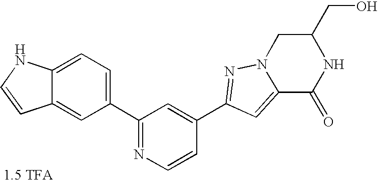 | 6-(hydroxymethyl)-2-[2-(1H-indol-5- yl)pyridin-4-yl]-6,7-dihydropyrazolo[1,5- a]pyrazin-4(5H)-one trifluoroacetate | 0.25 |
| 23 | 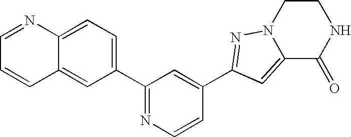 | 2-(2-quinolin-6-ylpyridin-4-yl)-6,7- dihydropyrazolo[1,5-a]pyrazin-4(5H)-one | 0.298 |
| 24 | 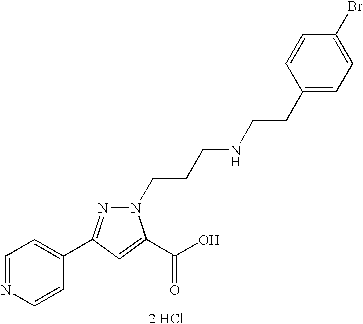 | 1-(3-{[2-(4- bromophenyl)ethyl]amino}propyl)-3-pyridin- 4-yl-1H-pyrazole-5-carboxylic acid hydrochloride | 0.3 |
| 25 | 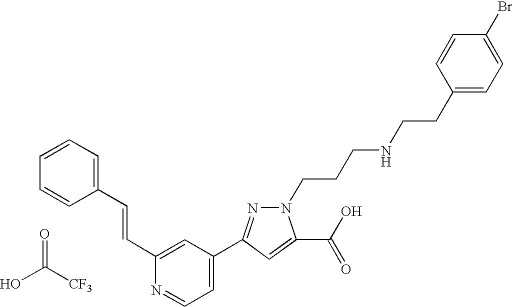 | 1-(3-{[2-(4- bromophenyl)ethyl]amino}propyl)-3-{2-[(E)- 2-phenylethenyl]pyridin-4-yl}-1H-pyrazole-5- carboxylic acid trifluoroacetate | 0.315 |
| 26 | 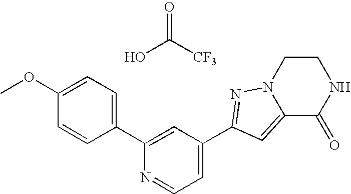 | 2-[2-(4-methoxyphenyl)pyridin-4-yl]-6,7- dihydropyrazolo[1,5-a]pyrazin-4(5H)-one trifluoroacetate | 0.318 |
| 27 | 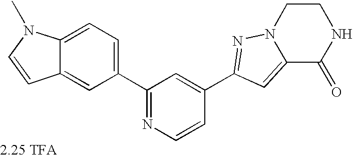 | 2-[2-(1-methyl-1H-indol-5-yl)pyridin-4-yl]- 6,7-dihydropyrazolo[1,5-a]pyrazin-4(5H)- one trifluoroacetate | 0.322 |
| 28 | 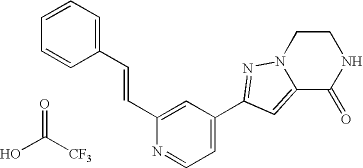 | 2-{2-[(E)-2-phenylvinyl]pyridin-4-yl}-6,7- dihydropyrazolo[1,5-a]pyrazin-4(5H)-one trifluoroacetate | 0.371 |
| 29 | 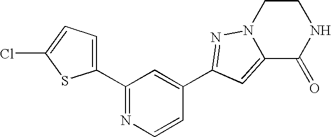 | 2-[2-(5-chlorothien-2-yl)pyridin-4-yl]-6,7- dihydropyrazolo[1,5-a]pyrazin-4(5H)-one | 0.444 |
| 30 | 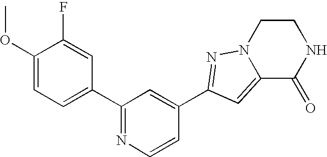 | 2-[2-(3-fluoro-4-methoxyphenyl)pyridin-4- yl]-6,7-dihydropyrazolo[1,5-a]pyrazin-4(5H)- one | 0.471 |
| 31 | 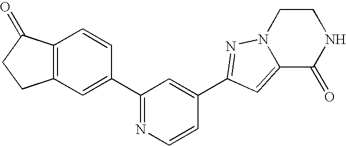 | 2-[2-(1-oxo-2,3-dihydro-1H-inden-5- yl)pyridin-4-y]-6,7-dihydropyrazolo[1,5- a]pyrazin-4(5H)-one | 0.478 |
| 32 | 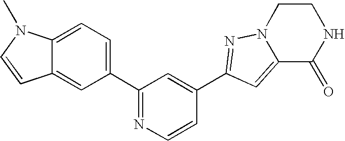 | 2-[2-(1-methyl-1H-indol-5-yl)pyridin-4-yl]- 6,7-dihydropyrazolo[1,5-a]pyrazin-4(5H)- one | 0.479 |
| 33 | 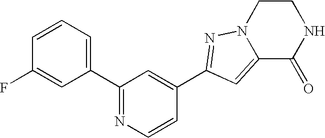 | 2-[2-(3-fluorophenyl)pyridin-4-yl]-6,7- dihydropyrazolo[1,5-a]pyrazin-4(5H)-one | 0.505 |
| 34 | 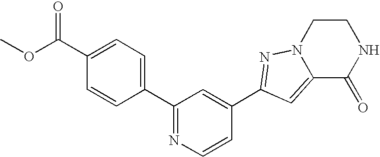 | methyl 4-[4-(4-oxo-4,5,6,7- tetrahydropyrazolo[1,5-a]pyrazin-2- yl)pyridin-2-yl]benzoate | 0.511 |
| 35 | 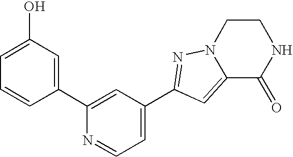 | 2-[2-(3-hydroxyphenyl)pyridin-4-yl]-6,7- dihydropyrazolo[1,5-a]pyrazin-4(5H)-one | 0.52 |
| 36 | 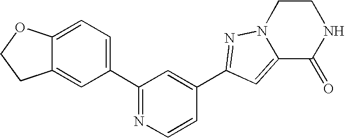 | 2-[2-(2,3-dihydro-1-benzofuran-5-yl)pyridin- 4-yl]-6,7-dihydropyrazolo[1,5-a]pyrazin- 4(5H)-one | 0.521 |
| 37 | 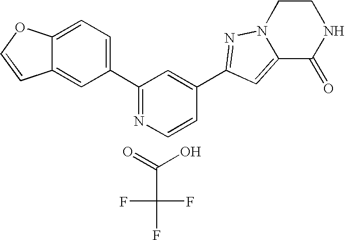 | 2-[2-(1-benzofuran-5-yl)pyridin-4-yl]-6,7- dihydropyrazolo[1,5-a]pyrazin-4(5H)-one trifluoroacetate | 0.543 |
| 38 | 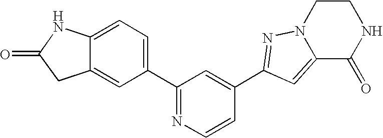 | 2-[2-(2-oxo-2,3-dihydro-1H-indol-5- yl)pyridin-4-yl]-6,7-dihydropyrazolo[1,5- a]pyrazin-4(5H)-one | 0.554 |
| 39 | 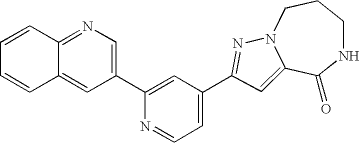 | 2-(2-quinolin-3-ylpyridin-4-yl)-5,6,7,8- tetrahydro-4H-pyrazolo[1,5-a][1,4]diazepin- 4-one | 0.577 |
| 40 | 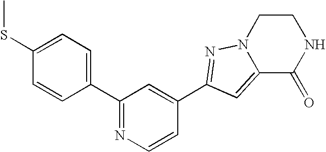 | 2-{2-[4-(methylthio)phenyl]pyridin-4-yl}-6,7- dihydropyrazolo[1,5-a]pyrazin-4(5H)-one | 0.581 |
| 41 | 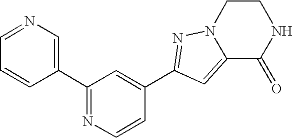 | 2-(2,3′-bipyridin-4-yl)-6,7- dihydropyrazolo[1,5-a]pyrazin-4(5H)-one | 0.586 |
| 42 | 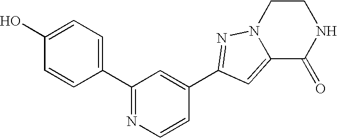 | 2-[2-(4-hydroxyphenyl)pyridin-4-yl]-6,7- dihydropyrazolo[1,5-a]pyrazin-4(5H)-one | 0.611 |
| 43 | 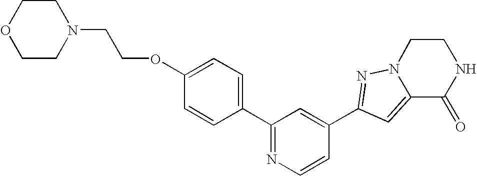 | 2-{2-[4-(2-morpholin-4- ylethoxy)phenyl]pyridin-4-yl}-6,7- dihydropyrazolo[1,5-a]pyrazin-4(5H)-one | 0.63 |
| 44 | 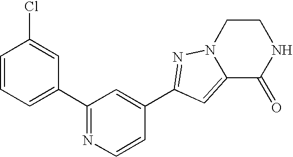 | 2-[2-(3-chlorophenyl)pyridin-4-yl]-6,7- dihydropyrazolo[1,5-a]pyrazin-4(5H)-one | 0.638 |
| 45 | 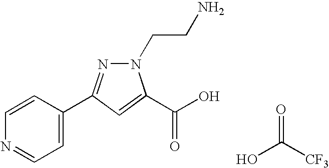 | 1-(2-aminoethyl)-3-pyridin-4-yl-1H-pyrazole- 5-carboxylic acid trifluoroacetate | 0.735 |
| 46 | 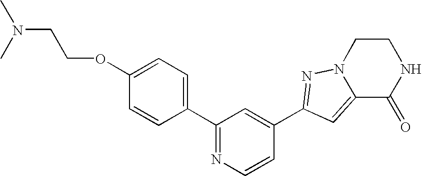 | 2-(2-{4-[2- (dimethylamino)ethoxy]phenyl}pyridin-4-yl)- 6,7-dihydropyrazolo[1,5-a]pyrazin-4(5H)- one | 0.766 |
| 47 | 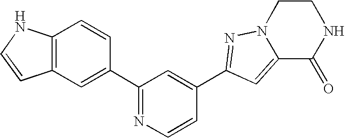 | 2-[2-(1H-indol-5-yl)pyridin-4-yl]-6,7- dihydropyrazolo[1,5-a]pyrazin-4(5H)-one | 0.808 |
| 48 | 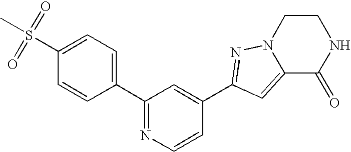 | 2-{2-[4-(methylsulfonyl)phenyl]pyridin-4-yl}- 6,7-dihydropyrazolo[1,5-a]pyrazin-4(5H)- one | 0.821 |
| 49 | 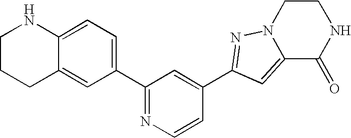 | 2-[2-(1,2,3,4-tetrahydroquinolin-6-yl)pyridin- 4-yl]-6,7-dihydropyrazolo[1,5-a]pyrazin- 4(5H)-one | 0.835 |
| 50 | 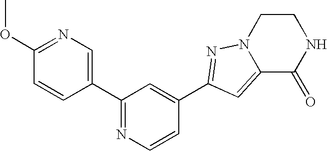 | 2-(6′-methoxy-2,3′-bipyridin-4-yl)-6,7- dihydropyrazolo[1,5-a]pyrazin-4(5H)-one | 0.866 |
| 51 | 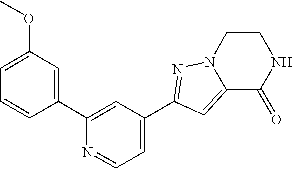 | 2-[2-(3-methoxyphenyl)pyridin-4-yl]-6,7- dihydropyrazolo[1,5-a]pyrazin-4(5H)-one | 0.906 |
| 52 | 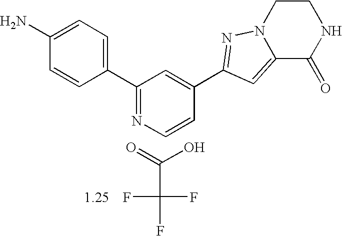 | 2-[2-(4-aminophenyl)pyridin-4-yl]-6,7- dihydropyrazolo[1,5-a]pyrazin-4(5H)-one trifluoroacetate | 0.914 |
| 53 | 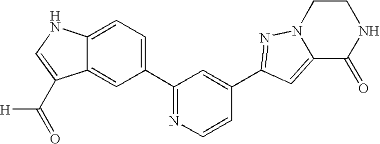 | 5-[4-(4-oxo-4,5,6,7-tetrahydropyrazolo[1,5- a]pyrazin-2-yl)pyridin-2-yl]-1H-indole-3- carbaldehyde | 0.923 |
| 54 | 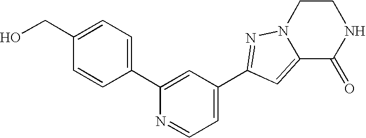 | 2-{2-[4-(hydroxymethyl)phenyl]pyridin-4-yl}- 6,7-dihydropyrazolo[1,5-a]pyrazin-4(5H)- one | 0.929 |
| 55 | 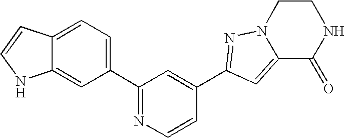 | 2-[2-(1H-indol-6-yl)pyridin-4-yl]-6,7- dihydropyrazolo[1,5-a]pyrazin-4(5H)-one | 0.93 |
| 56 | 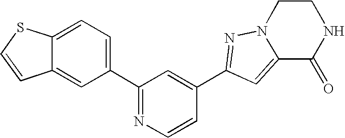 | 2-[2-(1-benzothien-5-yl)pyridin-4-yl]-6,7- dihydropyrazolo[1,5-a]pyrazin-4(5H)-one | 0.965 |
| 57 | 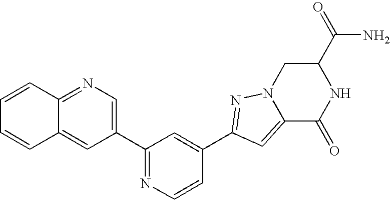 | 4-oxo-2-(2-quinolin-3-ylpyridin-4-yl)-4,5,6,7- tetrahydropyrazolo[1,5-a]pyrazine-6- carboxamide | 1.01 |
| 58 | 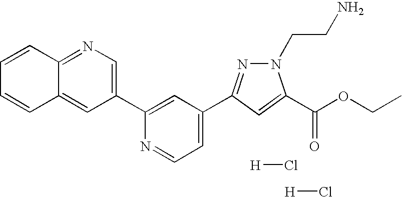 | ethyl 1-(2-aminoethyl)-3-(2-quinolin-3- ylpyridin-4-yl)-1H-pyrazole-5-carboxylate dihydrochloride | 1.03 |
| 59 | 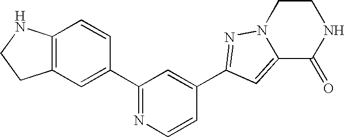 | 2-[2-(2,3-dihydro-1H-indol-5-yl)pyridin-4-yl]- 6,7-dihydropyrazolo[1,5-a]pyrazin-4(5H)- one | 1.07 |
| 60 | 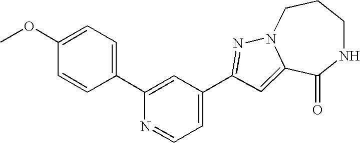 | 2-[2-(4-methoxyphenyl)pyridin-4-yl]-5,6,7,8- tetrahydro-4H-pyrazolo[1,5-a][1,4]diazepin- 4-one | 1.09 |
| 61 | 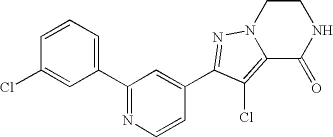 | 3-chloro-2-[2-(3-chlorophenyl)pyridin-4-yl]- 6,7-dihydropyrazolo[1,5-a]pyrazin-4(5H)- one | 1.13 |
| 62 | 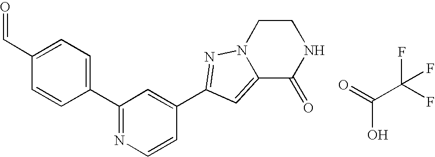 | 4-[4-(4-oxo-4,5,6,7-tetrahydropyrazolo[1,5- a]pyrazin-2-yl)pyridin-2-yl]benzaldehyde trifluoroacetate | 1.15 |
| 63 | 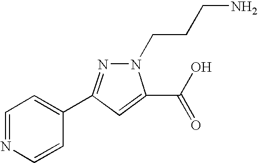 | 1-(3-aminopropyl)-3-pyridin-4-yl-1H- pyrazole-5-carboxylic acid | 1.16 |
| 64 | 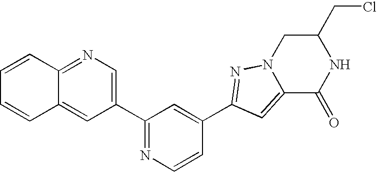 | 13012 or 6-(chloromethyl)-2-(2-quinolin-3- ylpyridin-4-yl)-6,7-dihydropyrazolo[1,5- a]pyrazin-4(5H)-one | 1.17 |
| 65 | 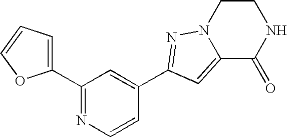 | 2-[2-(2-furyl)pyridin-4-yl]-6,7- dihydropyrazolo[1,5-a]pyrazin-4(5H)-one | 1.2 |
| 66 | 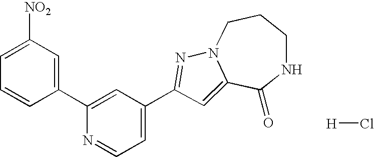 | 2-[2-(3-nitrophenyl)pyridin-4-yl]-5,6,7,8- tetrahydro-4H-pyrazolo[1,5-a][1,4]diazepin- 4-one hydrochloride | 1.26 |
| 67 | 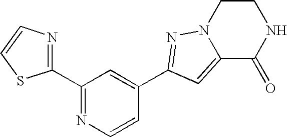 | 2-[2-(1,3-thiazol-2-yl)pyridin-4-yl]-6,7- dihydropyrazolo[1,5-a]pyrazin-4(5H)-one | 1.29 |
| 68 | 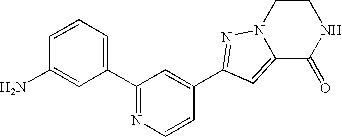 | 2-[2-(3-aminophenyl)pyridin-4-yl]-6,7- dihydropyrazolo[1,5-a]pyrazin-4(5H)-one | 1.3 |
| 69 | 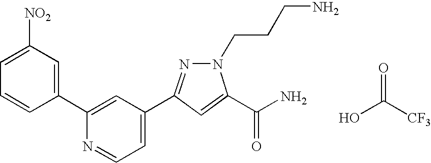 | 1-(3-aminopropyl)-3-[2-(3- nitrophenyl)pyridin-4-yl]-1H-pyrazole-5- carboxamide trifluoroacetate | 1.32 |
| 70 | 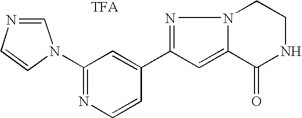 | 2-[2-(1H-imidazol-1-yl)pyridin-4-yl]-6,7- dihydropyrazolo[1,5-a]pyrazin-4(5H)-one trifluoroacetate | 1.36 |
| 71 | 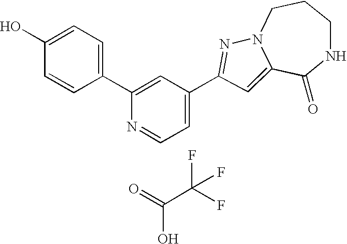 | 2-[2-(4-hydroxyphenyl)pyridin-4-yl]-5,6,7,8- tetrahydro-4H-pyrazolo[1,5-a][1,4]diazepin- 4-one trifluoroacetate | 1.37 |
| 72 | 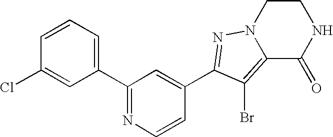 | 3-bromo-2-[2-(3-chlorophenyl)pyridin-4-yl]- 6,7-dihydropyrazolo[1,5-a]pyrazin-4(5H)- one | 1.39 |
| 73 | 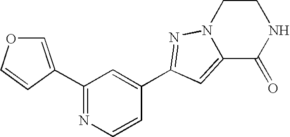 | 2-[2-(3-furyl)pyridin-4-yl]-6,7- dihydropyrazolo[1,5-a]pyrazin-4(5H)-one | 1.46 |
| 74 | 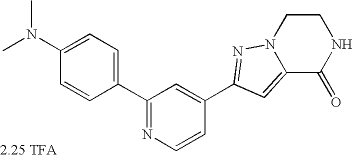 | 2-{2-[4-(dimethylamino)phenyl]pyridin-4-yl}- 6,7-dihydropyrazolo[1,5-a]pyrazin-4(5H)- one trifluoroacetate | 1.53 |
| 75 | 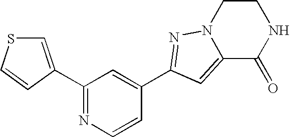 | 2-(2-thien-3-ylpyridin-4-yl)-6,7- dihydropyrazolo[1,5-a]pyrazin-4(5H)-one | 1.55 |
| 76 | 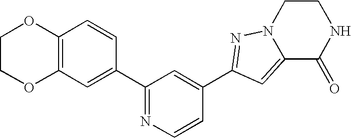 | 2-[2-(2,3-dihydro-1,4-benzodioxin-6- yl)pyridin-4-yl]-6,7-dihydropyrazolo[1,5- a]pyrazin-4(5H)-one | 1.56 |
| 77 | 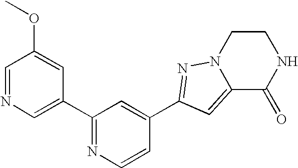 | 2-(5′-methoxy-2,3′-bipyridin-4-yl)-6,7- dihydropyrazolo[1,5-a]pyrazin-4(5H)-one | 1.69 |
| 78 | 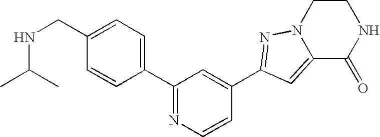 | 2-(2-{4- [(isopropylamino)methyl]phenyl}pyridin-4- yl)-6,7-dihydropyrazolo[1,5-a]pyrazin-4(5H)- one | 1.73 |
| 79 | 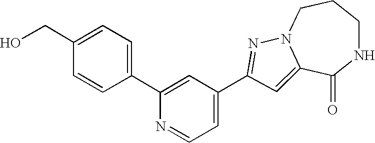 | 2-{2-[4-(hydroxymethyl)phenyl]pyridin-4-yl}- 5,6,7,8-tetrahydro-4H-pyrazolo[1,5- a][1,4]diazepin-4-one | 1.75 |
| 80 | 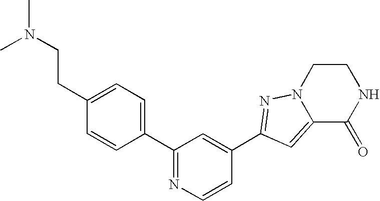 | 2-(2-{4-[2- (dimethylamino)ethyl]phenyl}pyridin-4-yl)- 6,7-dihydropyrazolo[1,5-a]pyrazin-4(5H)- one | 1.98 |
| 81 | 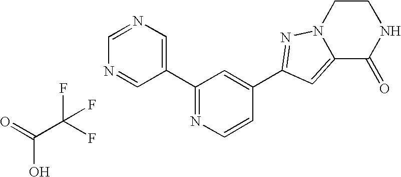 | 2-(2-pyrimidin-5-ylpyridin-4-yl)-6,7- dihydropyrazolo[1,5-a]pyrazin-4(5H)-one trifluoroacetate | 1.99 |
| 82 | 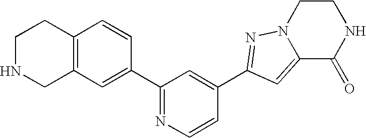 | 2-[2-(1,2,3,4-tetrahydroisoquinolin-7- yl)pyridin-4-yl]-6,7-dihydropyrazolo[1,5- a]pyrazin-4(5H)-one | 2.08 |
| 83 | 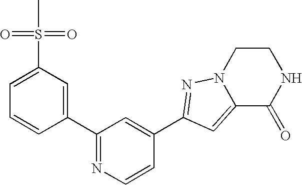 | 2-{2-[3-(methylsulfonyl)phenyl]pyridin-4-yl}- 6,7-dihydropyrazolo[1,5-a]pyrazin-4(5H)- one | 2.1 |
| 84 | 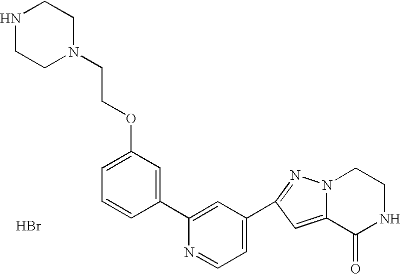 | 2-{2-[3-(2-piperazin-1- ylethoxy)phenyl]pyridin-4-yl}-6,7- dihydropyrazolo[1,5-a]pyrazin-4(5H)-one hydrobromide | 2.13 |
| 85 | 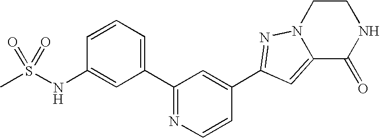 | N-{3-[4-(4-oxo-4,5,6,7- tetrahydropyrazolo[1,5-a]pyrazin-2- yl)pyridin-2-yl]phenyl}methanesulfonamide | 2.29 |
| 86 | 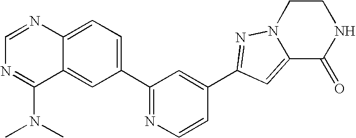 | 2-{2-[4-(dimethylamino)quinazolin-6- yl]pyridin-4-yl}-6,7-dihydropyrazolo[1,5- a]pyrazin-4(5H)-one | 2.4 |
| 87 | 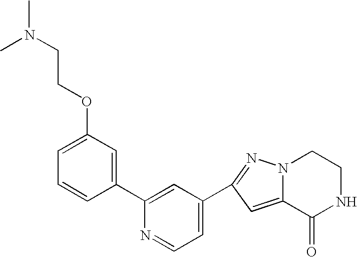 | 2-(2-{3-[2- (dimethylamino)ethoxy]phenyl}pyridin-4-yl)- 6,7-dihydropyrazolo[1,5-a]pyrazin-4(5H)- one | 2.4 |
| 88 | 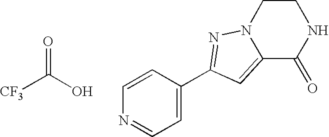 | 2-pyridin-4-yl-6,7-dihydropyrazolo[1,5- a]pyrazin-4(5H)-one trifluoroacetate | 2.6 |
| 89 | 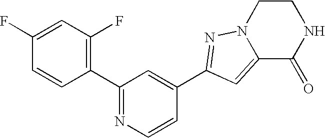 | 2-[2-(2,4-difluorophenyl)pyridin-4-yl]-6,7- dihydropyrazolo[1,5-a]pyrazin-4(5H)-one | 2.62 |
| 90 | 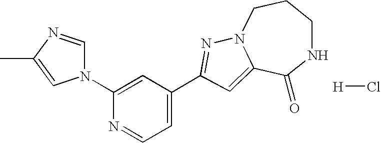 | 2-[2-(4-methyl-1H-imidazol-1-yl)pyridin-4- yl]-5,6,7,8-tetrahydro-4H-pyrazolo[1,5- a][1,4]diazepin-4-one hydrochloride | 2.76 |
| 91 | 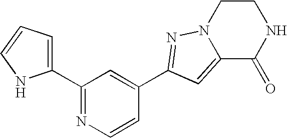 | 2-[2-(1H-pyrrol-2-yl)pyridin-4-yl]-6,7- dihydropyrazolo[1,5-a]pyrazin-4(5H)-one | 2.79 |
| 92 | 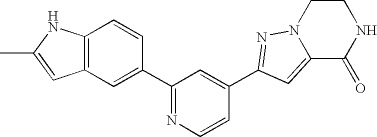 | 2-[2-(2-methyl-1H-indol-5-yl)pyeidin-4-yl]- 6,7-dihydropyrazolo[1,5-a]pyrazin-4(5H)- one | 2.83 |
| 93 | 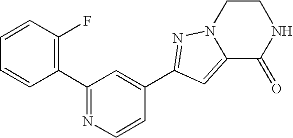 | 2-[2-(2-fluorophenyl)pyridin-4-yl]-6,7- dihydropyrazolo[1,5-a]pyrazin-4(5H)-one | 2.95 |
| 94 | 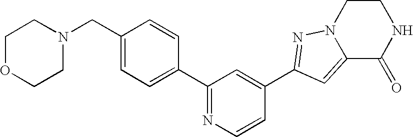 | 2-{2-[4-(morpholin-4- ylmethyl)phenyl]pyridin-4-yl}-6,7- dihydropyrazolo[1,5-a]pyrazin-4(5H)-one | 2.99 |
| 95 | 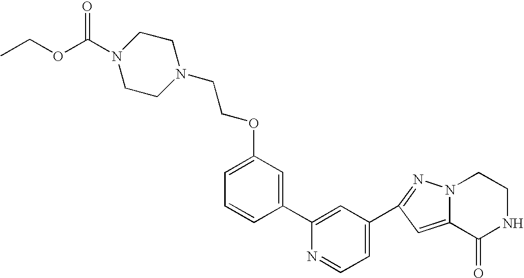 | ethyl 4-(2-{3-[4-(4-oxo-4,5,6,7- tetrahydropyrazolo[1,5-a]pyrazin-2- yl)pyridin-2-yl]phenoxy}ethyl)piperazine-1- carboxylate | 3.02 |
| 96 | 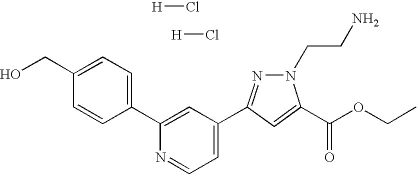 | ethyl 1-(2-aminoethyl)-3-{2-[4- (hydroxymethyl)phenyl]pyridin-4-yl}-1H- pyrazole-5-carboxylate dihydrochloride | 3.25 |
| 97 | 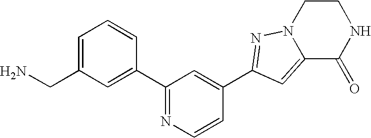 | 2-{2-[3-(aminomethyl)phenyl]pyridin-4-yl}- 6,7-dihydropyrazolo[1,5-a]pyrazin-4(5H)- one | 3.25 |
| 98 | 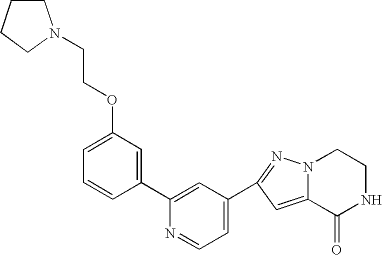 | 2-{2-[3-(2-pyrrolidin-1- ylethoxy)phenyl]pyridin-4-yl}-6,7- dihydropyrazolo[1,5-a]pyrazin-4(5H)-one | 3.27 |
| 99 | 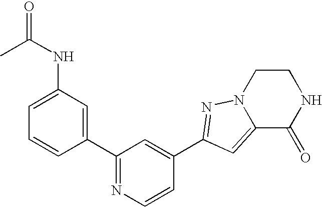 | N-{3-[4-(4-oxo-4,5,6,7- tetrahydropyrazolo[1,5-a]pyrazin-2- yl)pyridin-2-yl]phenyl}acetamide | 3.31 |
| 100 | 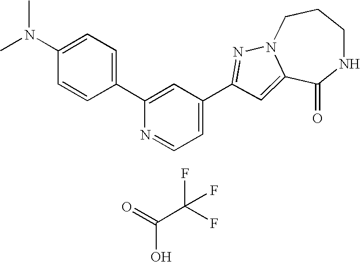 | 2-{2-[4-(dimethylamino)phenyl]pyridin-4-yl}- 5,6,7,8-tetrahydro-4H-pyrazolo[1,5- a][1,4]diazepin-4-one trifluoroacetate | 3.44 |
| 101 | 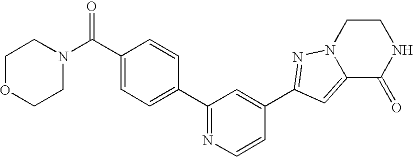 | 2-{2-[4-(morpholin-4- ylcarbonyl)phenyl]pyridin-4-yl}-6,7- dihydropyrazolo[1,5-a]pyrazin-4(5H)-one | 3.65 |
| 102 | 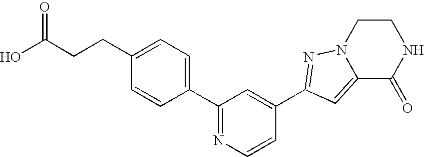 | 3-{4-[4-(4-oxo-4,5,6,7- tetrahydropyrazolo[1,5-a]pyrazin-2- yl)pyridin-2-yl]phenyl}propanoic acid | 4.19 |
| 103 | 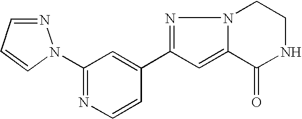 | 2-[2-(1H-pyrazol-1-yl)pyridin-4-yl]-6,7- dihydropyrazolo[1,5-a]pyrazin-4(5H)-one | 4.2 |
| 104 | 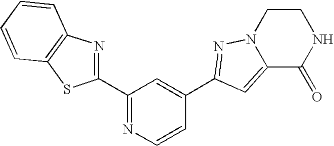 | 2-[2-(1,3-benzothiazol-2-yl)pyndin-4-yl]-6,7- dihydropyrazolo[1,5-a]pyrazin-4(5H)-one | 4.27 |
| 105 | 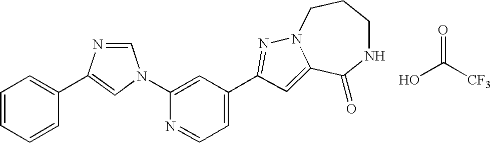 | 2-[2-(4-phenyl-1H-imidazol-1-yl)pyridin-4- yl]-5,6,7,8-tetrahydro-4H-pyrazolo[1,5- a][1,4]diazepin-4-one trifluoroacetate | 4.31 |
| 106 | 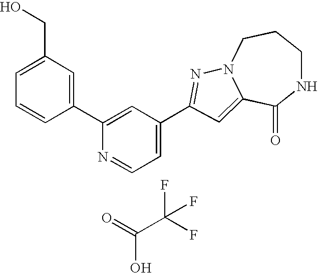 | 2-{2-[3-(hydroxymethyl)phenyl]pyridin-4-yl}- 5,6,7,8-tetrahydro-4H-pyrazolo[1,5- a][1,4]diazepin-4-one trifluoroacetate | 4.54 |
| 107 | 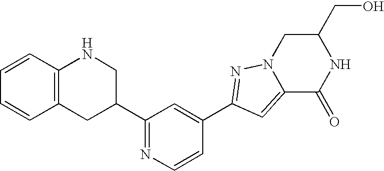 | 6-(hydroxymethyl)-2-[2-(1,2,3,4- tetrahydroquinolin-3-yl)pyridin-4-yl]-6,7- dihydropyrazolo[1,5-a]pyrazin-4(5H)-one | 4.9 |
| 108 | 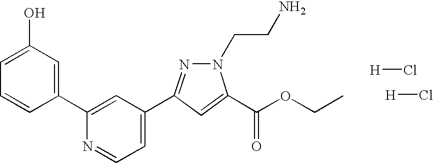 | ethyl 1-(2-aminoethyl)-3-[2-(3- hydroxyphenyl)pyridin-4-yl]-1H-pyrazole-5- carboxylate dihydrochloride | 4.98 |
| 109 | 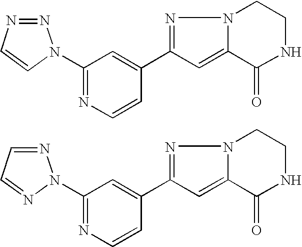 | 2-[2-(2H-1,2,3-triazol-2-yl)pyridin-4-yl]-6,7- dihydropyrazolo[1,5-a]pyrazin-4(5H)-one compound with 2-[2-(1H-1,2,3-triazol-1- yl)pyridin-4-yl]-6,7-dihydropyrazolo[1,5- a]pyrazin-4(5H)-one (1:1) | 5.21 |
| 110 | 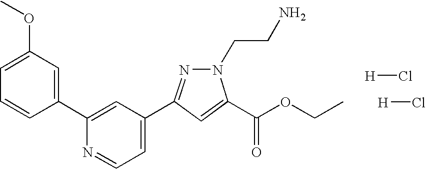 | ethyl 1-(2-aminoethyl)-3-[2-(3- methoxyphenyl)pyridin-4-yl]-1H-pyrazole-5- carboxylate dihydrochloride | 5.23 |
| 111 | 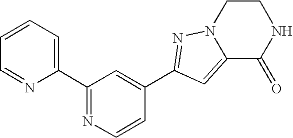 | 2-(2,2′-bipyridin-4-yl)-6,7- dihydropyrazolo[1,5-a]pyrazin-4(5H)-one | 5.34 |
| 112 | 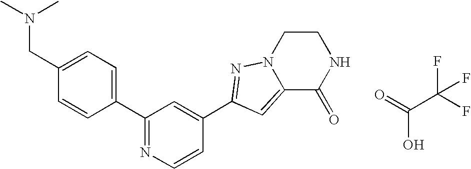 | 2-(2-{4- [(dimethylamino)methyl]phenyl}pyridin-4-yl)- 6,7-dihydropyrazolo[1,5-a]pyrazin-4(5H)- one trifluoroacetate | 5.35 |
| 113 | 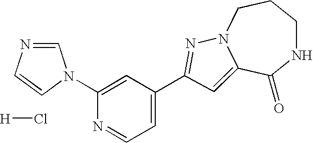 | 2-[2-(1H-imidazol-1-yl)pyridin-4-yl]-5,6,7,8- tetrahydro-4H-pyrazolo[1,5-a][1,4]diazepin- 4-one hydrochloride | 5.66 |
| 114 | 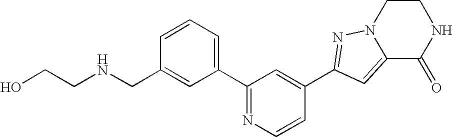 | 2-[2-(3-{[(2- hydroxyethyl)amino]methyl}phenyl)pyridin- 4-yl]-6,7-dihydropyrazolo[1,5-a]pyrazin- 4(5H)-one | 5.78 |
| 115 | 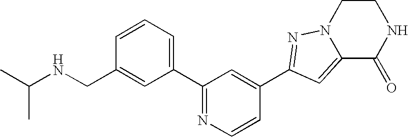 | 2-(2-{3- [(isopropylamino)methyl]phenyl}pyridin-4- yl)-6,7-dihydropyrazolo[1,5-a]pyrazin-4(5H)- one | 6.27 |
| 116 | 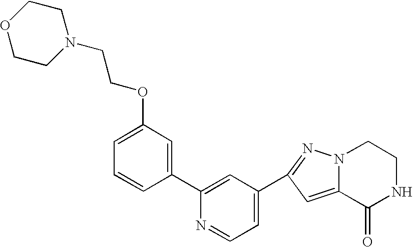 | 2-{2-[3-(2-morpholin-4- ylethoxy)phenyl]pyridin-4-yl}-6,7- dihydropyrazolo[1,5-a]pyrazin-4(5H)-one | 6.73 |
| 117 | 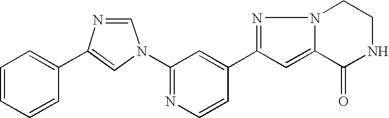 | 2-[2-(4-phenyl-1H-imidazol-1-yl)pyridin-4- yl]-6,7-dihydropyrazolo[1,5-a]pyrazin-4(5H)- one | 7.04 |
| 118 | 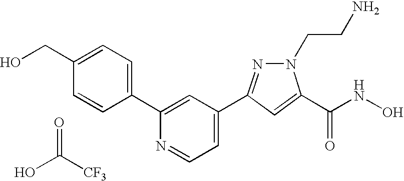 | 1-(2-aminoethy)-N-hydroxy-3-{2-[4- (hydroxymethyl)phenyl]pyridin-4-yl}-1H- pyrazole-5-carboxamide trifluoroacetate | 7.91 |
| 119 | 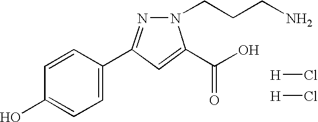 | 1-(3-aminopropyl)-3-(4-hydroxyphenyl)-1H- pyrazole-5-carboxylic acid dihydrochloride | 8.11 |
| 120 | 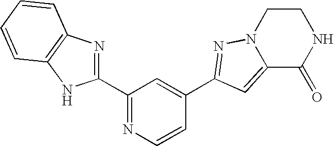 | 2-[2-(1H-benzimidazol-2-yl)pyndin-4-yl]-6,7- dihydropyrazolo[1,5-a]pyrazin-4(5H)-one | 8.92 |
| 121 | 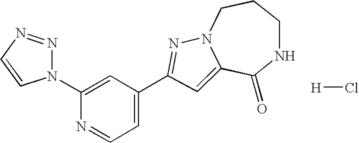 | 2-[2-(1H-1,2,3-triazol-1-yl)pyridin-4-yl]- 5,6,7,8-tetrahydro-4H-pyrazolo[1,5- a][1,4]diazepin-4-one hydrochloride | 9.05 |
| 122 | 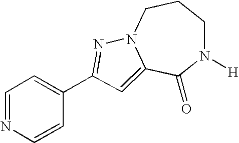 | 2-pyridin-4-yl-5,6,7,8-tetrahydro-4H- pyrazolo[1,5-a][1,4]diazepin-4-one | 10.1 |
| 123 | 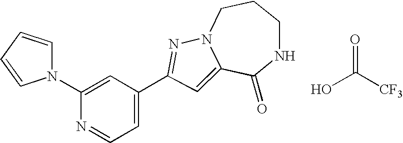 | 2-[2-(1H-pyrrol-1-yl)pyridin-4-yl]-5,6,7,8- tetrahydro-4H-pyrazolo[1,5-a][1,4]diazepin- 4-one trifluoroacetate | 10.6 |
| 124 | 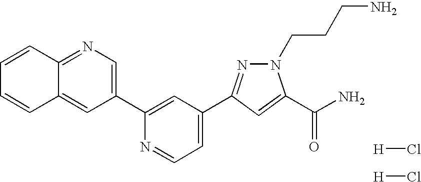 | 1-(3-aminopropyl)-3-(2-quinolin-3-ylpyridin- 4-yl)-1H-pyrazole-5-carboxamide dihydrochloride | 10.7 |
| 125 | 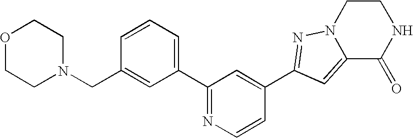 | 2-{2-[3-(morpholin-4- ylmethyl)phenyl]pyridin-4-yl]-6,7- dihydropyrazolo[1,5-a]pyrazin-4(5H)-one | 11.4 |
| 126 | 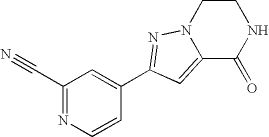 | 4-(4-oxo-4,5,6,7-tetrahydropyrazolo[1,5- a]pyrazin-2-yl)pyridine-2-carbonitrile | 13.8 |
| 127 | 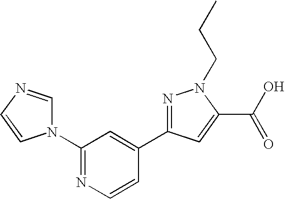 | 3-[2-(1H-imidazol-1-yl)pyridin-4-yl]-1-propyl- 1H-pyrazole-5-carboxylic acid | 14.3 |
| 128 | 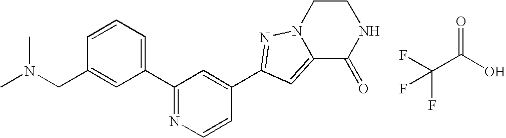 | 2-(2-{3- [(dimethylamino)methyl]phenyl}pyridin-4-yl)- 6,7-dihydropyrazolo[1,5-a]pyrazin-4(5H)- one trifluoroacetate | 15.4 |
| 129 | 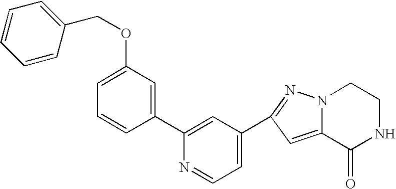 | 2-{2-[3-(benzyloxy)phenyl]pyridin-4-yl}-6,7- dihydropyrazolo[1,5-a]pyrazin-4(5H)-one | 15.5 |
| 130 | 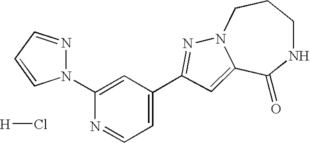 | 2-[2-(1H-pyrazol-1-yl)pyridin-4-yl]-5,6,7,8- tetrahydro-4H-pyrazolo[1,5-a][1,4]diazepin- 4-one hydrochloride | 16.2 |
| 131 | 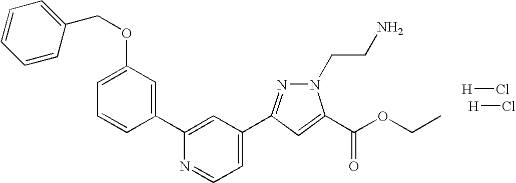 | ethyl 1-(2-aminoethyl)-3-{2-[3- (benzyloxy)phenyl]pyridin-4-yl}-1H- pyrazole-5-carboxylate dihydrochloride | 20 |
| 132 | 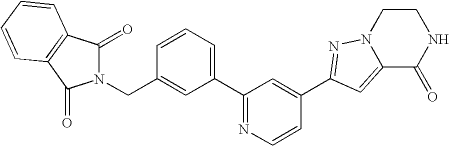 | 2-{3-[4-(4-oxo-4,5,6,7- tetrahydropyrazolo[1,5-a]pyrazin-2- yl)pyridin-2-yl]benzyl}-1H-isoindole-1,3(2H)- dione | 20 |
| 133 | 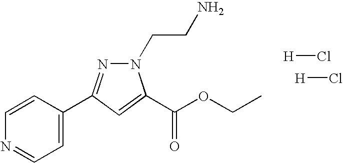 | ethyl 1-(2-aminoethyl)-3-pyridin-4-yl-1H- pyrazole-5-carboxylate dihydrochloride | 24.5 |
| 134 | 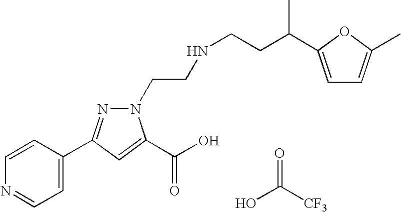 | 1-(2-{[3-(5-methyl-2-furyl)butyl]amino}ethyl)- 3-pyridin-4-yl-1H-pyrazole-5-carboxylic acid trifluoroacetate | 27.2 |
| 135 | 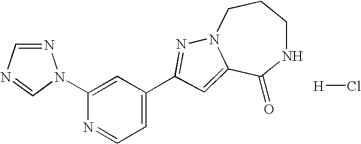 | 2-[2-(1H-1,2,4-triazol-1-yl)pyridin-4-yl]- 5,6,7,8-tetrahydro-4H-pyrazolo[1,5- a][1,4]diazepin-4-one hydrochloride | 28.2 |
| 136 | 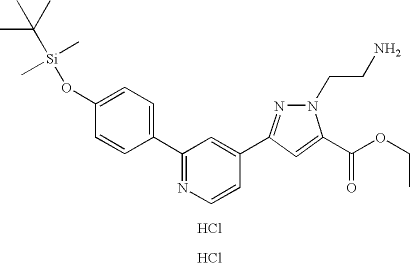 | ethyl 1-(2-aminoethyl)-3-[2-(4-{[tert- butyl(dimethyl)silyl]oxy}phenyl)pyridin-4-yl]- 1H-pyrazole-5-carboxylate dihydrochloride | 28.3 |
| 137 | 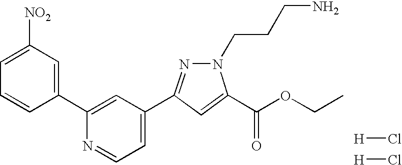 | ethyl 1-(3-aminopropyl)-3-[2-(3- nitrophenyl)pyridin-4-yl]-1H-pyrazole-5- carboxylate dihydrochloride | 31.6 |
| 138 | 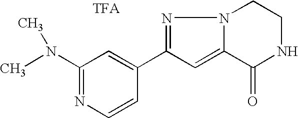 | 2-[2-(dimethylamino)pyridin-4-yl]-6,7- dihydropyrazolo[1,5-a]pyrazin-4(5H)-one trifluoroacetate | 37.2 |
| 139 | 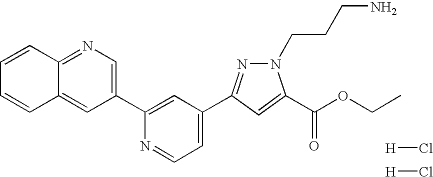 | ethyl 1-(3-aminopropyl)-3-(2-quinolin-3- ylpyridin-4-yl)-1H-pyrazole-5-carboxylate dihydrochloride | 38.6 |
| 140 | 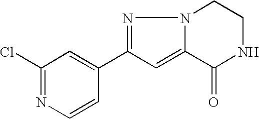 | 2-(2-chloropyridin-4-yl)-6,7- dihydropyrazolo[1,5-a]pyrazin-4(5H)-one | 44.8 |
| 141 | 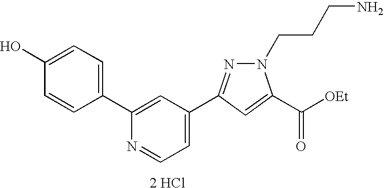 | ethyl 1-(3-aminopropyl)-3-[2-(4- hydroxyphenyl)pyridin-4-yl]-1H-pyrazole-5- carboxylate hydrochloride | 47.5 |
| 142 | 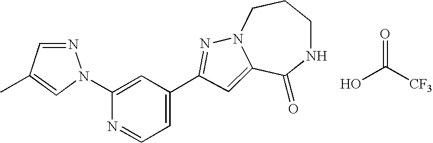 | 2-[2-(4-methyl-1H-pyrazol-1-yl)pyridin-4-yl]- 5,6,7,8-tetrahydro-4H-pyrazolo[1,5- a][1,4]diazepin-4-one trifluoroacetate | 53.4 |
| 143 | 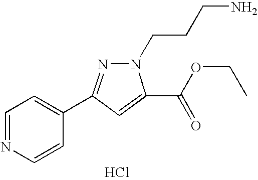 | ethyl 1-(3-aminopropyl)-3-pyridin-4-yl-1H- pyrazole-5-carboxylate hydrochloride | 60.8 |
| 144 | 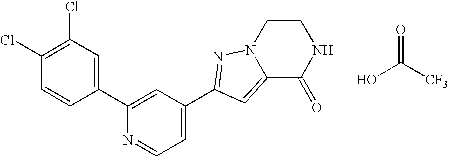 | 2-[2-(3,4-dichlorophenyl)pyridin-4-yl]-6,7- dihydropyrazolo[1,5-a]pyrazin-4(5H)-one trifluoroacetate | 65.5 |
| 145 | 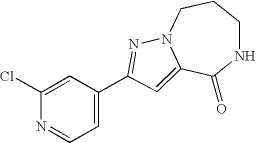 | 2-(2-chloropyridin-4-yl)-5,6,7,8-tetrahydro- 4H-pyrazolo[1,5-a][1,4]diazepin-4-one | 80.4 |
| 146 | 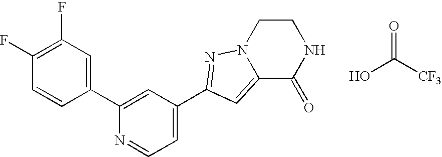 | 2-[2-(3,4-difluorophenyl)pyridin-4-yl]-6,7- dihydropyrazolo[1,5-a]pyrazin-4(5H)-one trifluoroacetate | 88.9 |
| 147 | 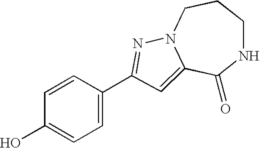 | 2-(4-hydroxyphenyl)-5,6,7,8-tetrahydro-4H- pyrazolo[1,5-a][1,4]diazepin-4-one | 118 |
| 148 | 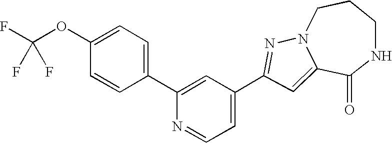 | 2-{2-[4-(trifluoromethoxy)phenyl]pyridin-4- yl}-5,6,7,8-tetrahydro-4H-pyrazolo[1,5- a][1,4]diazepin-4-one | 124 |
| 149 | 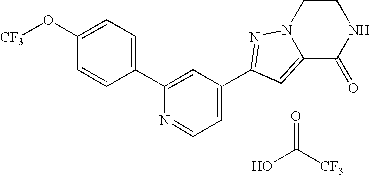 | 2-{2-[4-(trifluoromethoxy)phenyl]pyridin-4- yl}-6,7-dihydropyrazolo[1,5-a]pyrazin-4(5H)- one trifluoroacetate | 131 |
| 150 | 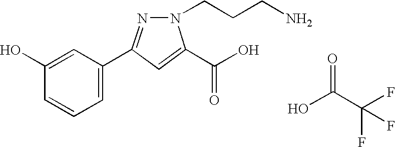 | 1-(3-aminopropyl)-3-(3-hydroxyphenyl)-1H- pyrazole-5-carboxylic acid trifluoroacetate | 153 |
| 151 | 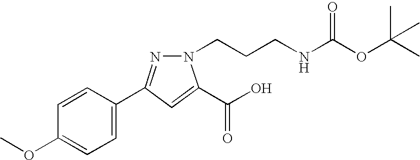 | 1-{3-[(tert-butoxycarbonyl)amino]propyl}-3- (4-methoxyphenyl)-1H-pyrazole-5- carboxylic acid | 155 |
| 152 | 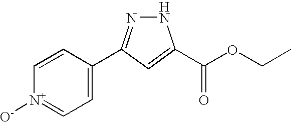 | ethyl 3-(1-oxidopyridin-4-yl)-1H-pyrazole-5- carboxylate | 200 |
| 153 | 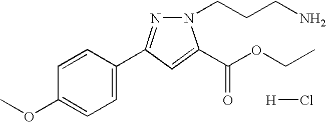 | ethyl 1-(3-aminopropyl)-3-(4- methoxyphenyl)-1H-pyrazole-5-carboxylate hydrochloride | 200 |
| 154 | 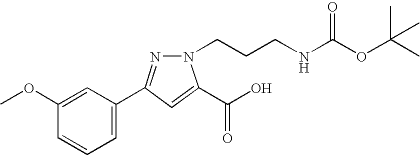 | 1-{3-[(tert-butoxycarbonyl)amino]propyl}-3- (3-methoxyphenyl)-1H-pyrazole-5- carboxylic acid | 200 |
| 155 | 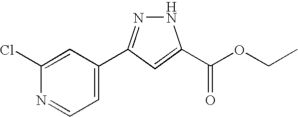 | ethyl 3-(2-chloropyridin-4-yl)-1H-pyrazole-5- carboxylate | 200 |
| 156 | 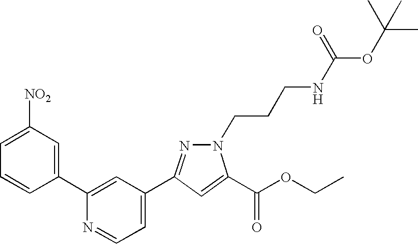 | ethyl 1-{3-[(tert- butoxycarbonyl)amino]propyl}-3-[2-(3- nitrophenyl)pyridin-4-yl]-1H-pyrazole-5- carboxylate | 200 |
| 157 | 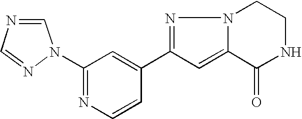 | 2-[2-(1H-1,2,4-triazol-1-yl)pyridin-4-yl]-6,7- dihydropyrazolo[1,5-a]pyrazin-4(5H)-one | 200 |
| 158 | 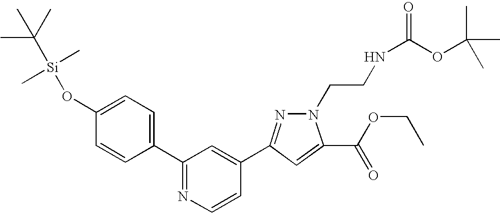 | ethyl 1-{2-[(tert- butoxycarbonyl)amino]ethyl}-3-[2-(4-{[tert- butyl(dimethyl)silyl]oxy}phenyl)pyridin-4-yl]- 1H-pyrazole-5-carboxylate | 200 |
| 159 | 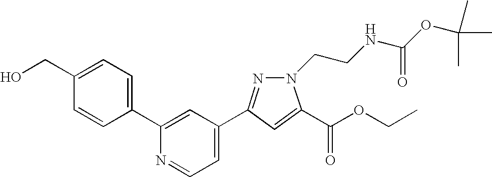 | ethyl 1-{2-[(tert- butoxycarbonyl)amino]ethyl}-3-{2-[4- (hydroxymethyl)phenyl]pyridin-4-yl}-1H- pyrazole-5-carboxylate | 200 |
| 160 | 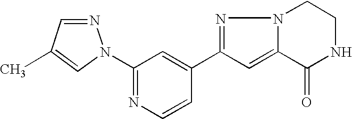 | 2-[2-(4-methyl-1H-pyrazol-1-yl)pyridin-4-yl]- 6,7-dihydropyrazolo[1,5-a]pyrazin-4(5H)- one | 200 |
| 161 | 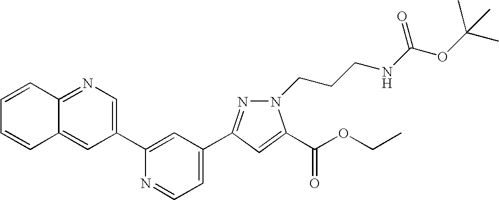 | 2-{2-[4-(trifluoromethoxy)phenyl]pyridin-4- yl}-6,7-dihydropyrazolo[1,5-a]pyrazin-4(5H)- one trifluoroacetate | 200 |
| 162 | 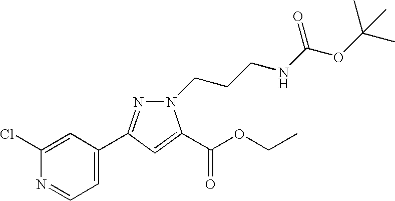 | ethyl 1-{3-[(tert- butoxycarbonyl)amino]propyl}-3-(2- chloropyridin-4-yl)-1H-pyrazole-5- carboxylate | 200 |
| 163 | 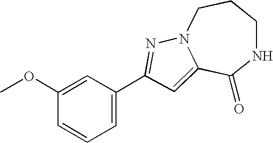 | 2-(3-methoxyphenyl)-5,6,7,8-tetrahydro-4H- pyrazolo[1,5-a][1,4]diazepin-4-one | 200 |
| 164 | 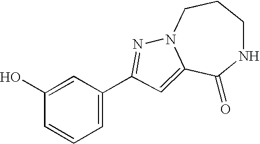 | 2-(3-hydroxyphenyl)-5,6,7,8-tetrahydro-4H- pyrazolo[1,5-a][1,4]diazepin-4-one | 200 |
| 165 | 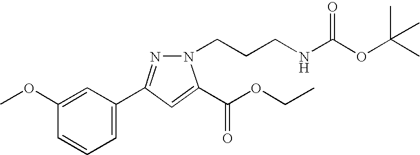 | ethyl 1-{3-[(tert- butoxycarbonyl)amino]propyl}-3-(3- methoxyphenyl)-1H-pyrazole-5-carboxylate | 200 |
| 166 | 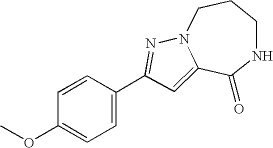 | 2-(4-methoxyphenyl)-5,6,7,8-tetrahydro-4H- pyrazolo[1,5-a][1,4]diazepin-4-one | 200 |
| 167 | 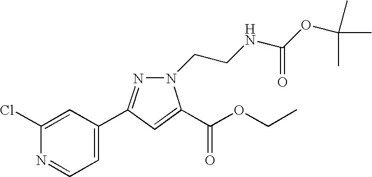 | ethyl 1-{2-[(tert- butoxycarbonyl)amino]ethyl}-3-(2- chloropyridin-4-yl)-1H-pyrazole-5- carboxylate | |
| 168 | 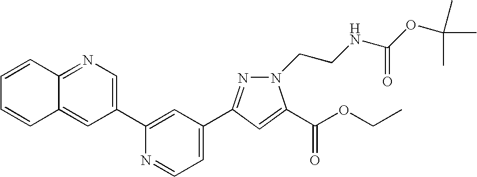 | ethyl 1-{2-[(tert- butoxycarbonyl)amino]ethyl}-3-(2-quinolin- butoxycarbonyl)amino]ethyl}-3-(2-quinolin- 3-ylpyridin-4-yl)-1H-pyrazole-5-carboxylate | |
| 169 | 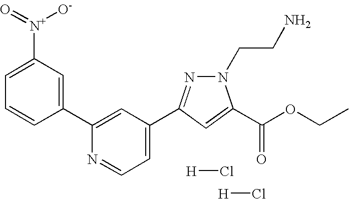 | ethyl 1-(2-aminoethyl)-3-[2-(3- nitrophenyl)pyridin-4-yl]-1H-pyrazole-5- carboxylate dihydrochloride | |
| 170 | 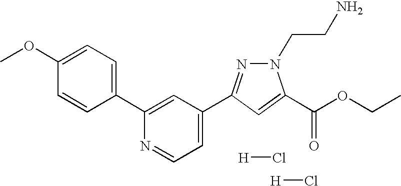 | ethyl 1-(2-aminoethyl)-3-[2-(4- methoxyphenyl)pyridin-4-yl]-1H-pyrazole-5- carboxylate dihydrochloride | |
| 171 | 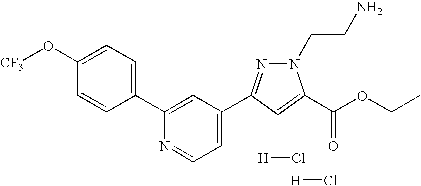 | ethyl 1-(2-aminoethyl)-3-{2-[4- (trifluoromethoxy)phenyl]pyridin-4-yl}-1H- pyrazole-5-carboxylate dihydrochloride | |
| 172 | 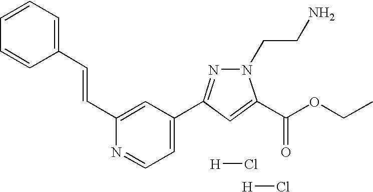 | ethyl 1-(2-aminoethyl)-3-{2-[(E)-2- phenylethenyl]pyridin-4-yl}-1H-pyrazole-5- carboxylate dihydrochloride | |
| 173 | 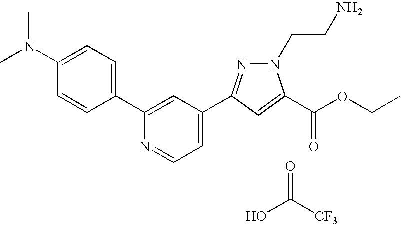 | ethyl 1-(2-aminoethyl)-3-{2-[4- (dimethylamino)phenyl]pyridin-4-yl}-1H- pyrazole-5-carboxylate trifluoroacetate | |
| 174 | 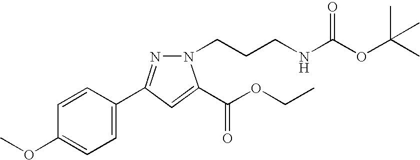 | ethyl 1-{3-[(tert- butoxycarbonyl)amino]propyl}-3-(4- methoxyphenyl)-1H-pyrazole-5-carboxylate | |
| 175 | 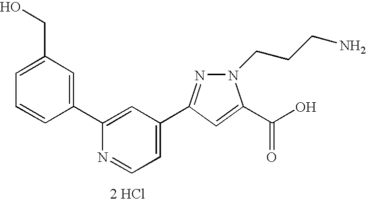 | 1-(3-aminopropyl)-3-{2-[3- (hydroxymethyl)phenyl]pyridin-4-yl}-1H- pyrazole-5-carboxylic acid hydrochloride | |
| 176 | 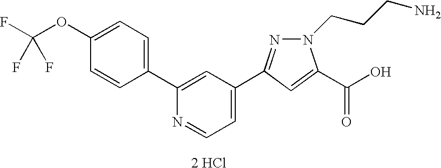 | 1-(3-aminopropyl)-3-{2-[4- (trifluoromethoxy)phenyl]pyridin-4-yl]-1H- pyrazole-5-carboxylic acid hydrochloride | |
| 177 | 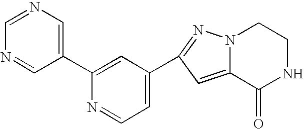 | 2-(2-pyrimidin-5-ylpyridin-4-yl)-6,7- dihydropyrazolo[1,5-a]pyrazin-4(5H)-one | |
| 178 | 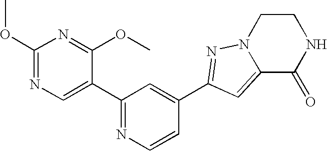 | 2-[2-(2,4-dimethoxypyrimidin-5-yl)pyridin-4- yl]-6,7-dihydropyrazolo[1,5-a]pyrazin-4(5H)- one | |
| 179 | 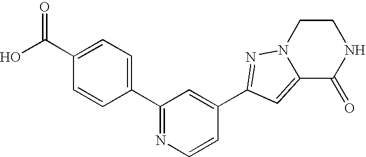 | 4-[4-(4-oxo-4,5,6,7-tetrahydropyrazolo[1,5- a]pyrazin-2-yl)pyridin-2-yl]benzoic acid | |
| 180 | 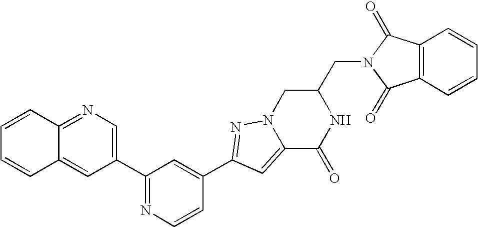 | 2-{[4-oxo-2-(2-quinolin-3-ylpyridin-4-yl)- 4,5,6,7-tetrahydropyrazolo[1,5-a]pyrazin-6- yl]methyl}-1H-isoindole-1,3(2H)-dione | |

TABLE II
Additional MK-2 Inhibiting Compounds

| | | | MK-2 Avg. |
|---|---|---|---|
| No. | Structure^{a} | Compound Name(s)^{b} | IC₅₀ (uM) |
| 181 | 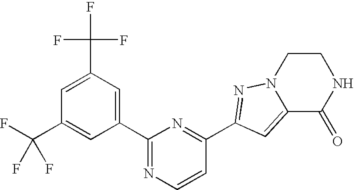 | 2-{2-[3,5- bis(trifluoromethyl)phenyl]pyrimidin-4- yl}-6,7-dihydropyrazolo[1,5-a]pyrazin- 4(5H)-one | |
| 182 | 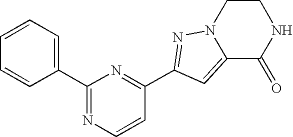 | 2-(2-phenylpyrimidin-4-yl)-6,7- dihydropyrazolo[1,5-a]pyrazin-4(5H)- one | |
| 183 | 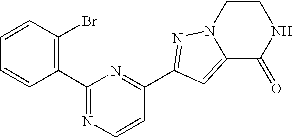 | 2-[2-(2-bromophenyl)pyrimidin- 4-yl]-6,7-dihydropyrazolo[1,5-a]pyrazin-4(5H)-one | |
| 184 | 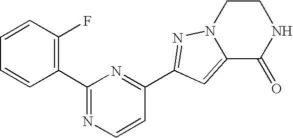 | 2-[2-(2-fluorophenyl)pyrimidin-4- yl]-6,7-dihydropyrazolo[1,5-a]pyrazin- 4(5H)-one | |
| 185 | 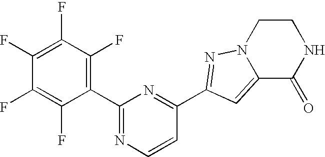 | 2-[2-(pentafluorophenyl)pyrimidin- 4-yl]-6,7-dihydropyrazoio[1,5-a]pyrazin-4(5H)-one | |
| 186 | 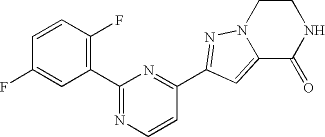 | 2-[2-(2,5-difluorophenyl)pyrimidin- 4-yl]-6,7-dihydropyrazolo[1,5-a]pyrazin-4(5H)-one | |
| 187 | 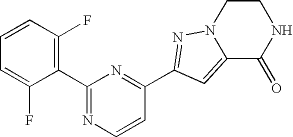 | 2-[2-(2,6-difluorophenyl)pyrimidin- 4-yl]-6,7-dihydropyrazolo[1,5-a]pyrazin-4(5H)-one | |
| 188 | 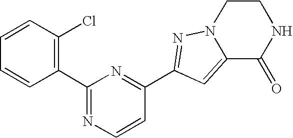 | 2-[2-(2-chlorophenyl)pyrimidin-4-yl]- 6,7-dihydropyrazolo[1,5-a]pyrazin- 4(5H)-one | |
| 189 | 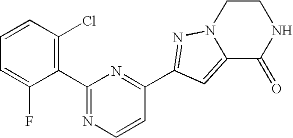 | 2-[2-(2-chloro-6-fluorophenyl) pyrimidin-4-yl]-6,7-dihydropyrazolo[1, 5-a]pyrazin-4(5H)-one | |
| 190 | 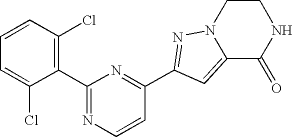 | 2-[2-(2,6-dichlorophenyl)pyrimidin- 4-yl]-6,7-dihydropyrazolo[1,5-a]pyrazin-4(5H)-one | |
| 191 | 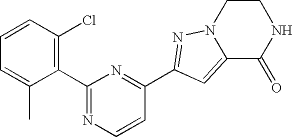 | 2-[2-(2-chloro-6-methylphenyl) pyrimidin-4-yl]-6,7-dihydrapyrazolo[1, 5-a]pyrazin-4(5H)-one | |
| 192 | 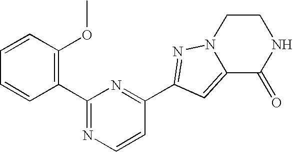 | 2-[2-(2-methoxyphenyl)pyrimidin-4- yl]-6,7-dihydropyrazolo[1 5-a]pyrazin-4(5H)-one | |
| 193 | 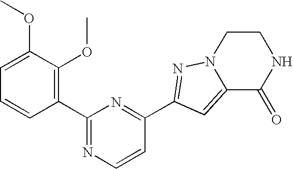 | 2-[2-(2,3-dimethoxyphenyl)pyrimidin-4- yl]-6,7-dihydropyrazolo[1,5-a]pyrazin- 4(5H)-one | |
| 194 | 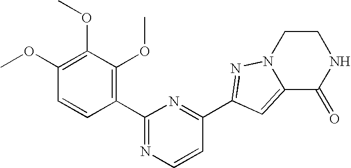 | 2-[2-(2,3,4-trimethoxyphenyl) pyrimidin-4-yl]-6,7-dihydropyrazolo[1, 5-a]pyrazin-4(5H)-one | |
| 195 | 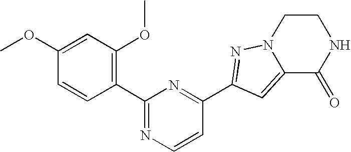 | 2-[2-(2,4-dimethoxyphenyl)pyrimidin- 4-yl]-6,7-dihydropyrazolo[1,5-a]pyrazin-4(5H)-one | |
| 196 | 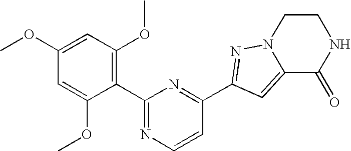 | 2-[2-(2,4,6-trimethoxyphenyl) pyrimidin-4-yl]-6,7-dihydropyrazolo[1, 5-a]pyrazin-4(5H)-one | |
| 197 | 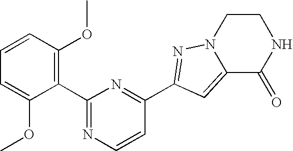 | 2-[2-(2,6-dimethoxyphenyl)pyrimidin-4- yl]-6,7-dihydropyrazolo[1,5-a]pyrazin- 4(5H)-one | |
| 198 | 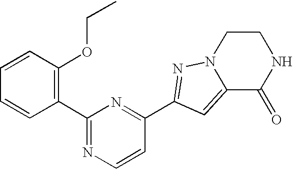 | 2-[2-(2-ethoxyphenyl)pyrimidin-4-yl]- 6,7-dihydropyrazolo[1,5-a]pyrazin- 4(5H)-one | |
| 199 | 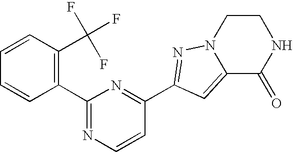 | 2-{2-[2-(trifluoromethyl)phenyl]pyrimidin-4-yl]-6,7-dihydropyrazolo [1,5-a]pyrazin-4(5H)-one | |
| 200 | 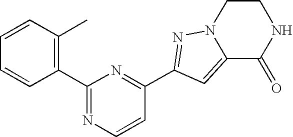 | 2-[2-(2-methylphenyl)pyrimidin-4-yl]- 6,7-dihydropyrazolo[1,5-a]pyrazin- 4(5H)-one | |
| 201 | 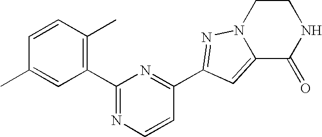 | 2-[2-(2,5-dimethylphenyl)pyrimidin-4- yl]-6,7-dihydropyrazolo[1,5-a]pyrazin-4(5H)-one | |
| 202 | 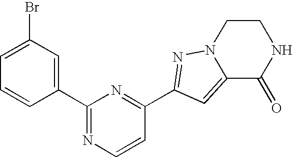 | 2-[2-(3-bromophenyl)pyrimidin-4-yl]- 6,7-dihydropyrazolo[1,5-a]pyrazin- 4(5H)-one | |
| 203 | 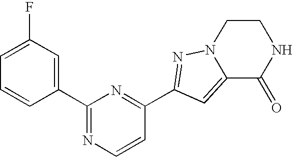 | 2-(2-(3-fluorophenyl)pyrimidin-4-yl]-6, 7-dihydropyrazolo[1,5-a]pyrazin- 4(5H)-one | |
| 204 | 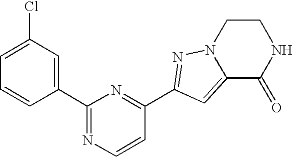 | 2-[2-(3-chlorophenyl)pyrimidin-4-yl]- 6,7-dihydropyrazolo[1,5-a]pyrazin- 4(5H)-one | |
| 205 | 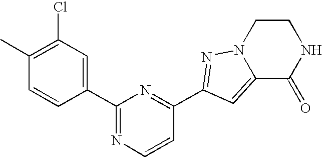 | 2-[2-(3-chloro-4-methylphenyl) pyrimidin-4-yl]-6,7-dihydropyrazolo[1, 5-a]pyrazin-4(5H)-one | |
| 206 | 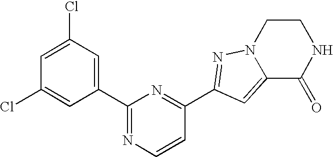 | 2-(2-(3,5-dichlorophenyl)pyrimidin-4- yl]-6,7-dihydropyrazolo[1,5-a]pyrazin- 4(5H)-one | |
| 207 | 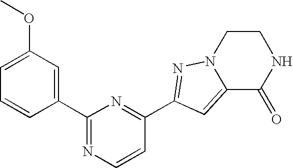 | 2-(2-(3-methoxyphenyl)pyrimidin-4-yl]- 6,7-dihydropyrazolo[1,5-a]pyrazin- 4(5H)-one | |
| 208 | 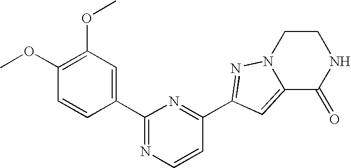 | 2-[2-(3,4-dimethoxyphenyl)pyrimidin- 4-yl]-6,7-dihydropyrazolo[1,5-a]pyrazin-4(5H)-one | |
| 209 | 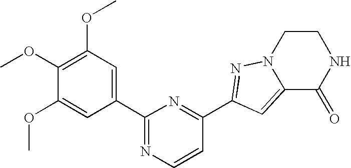 | 2-[2-(3,4,5-trimethoxyphenyl) pyrimidin-4-yl]- 6,7-dihydropyrazolo[1,5-a]pyrazin- 4(5H)-one | |
| 210 | 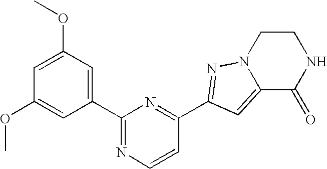 | 2-[2-(3,5-dimethoxyphenyl)pyrimidin- 4-yl]-6,7-dihydropyrazolo[1,5-a]pyrazin-4(5H)-one | |
| 211 | 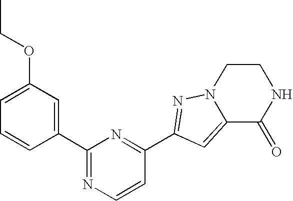 | 2-[2-(3-ethoxyphenyl)pyrimidin-4-yl]- 6,7-dihydropyrazolo[1,5-a]pyrazin-4(5H)-one | |
| 212 | 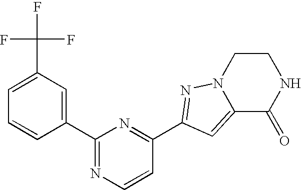 | 2-{2-(3-(trifluoromethyl)phenyl]pyrimidin-4-yl}-6,7- dihydropyrazolo[1,5-a]pyrazin- 4(5H)-one | |
| 213 | 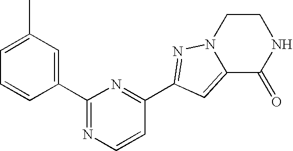 | 2-[2-(3-methylphenyl)pyrimidin-4-yl]- 6,7-dihydropyrazolo[1,5-a]pyrazin-4(5H)-one | |
| 214 | 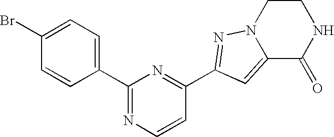 | 2-[2-(4-bromophenyl)pyrimidin-4-yl]- 6,7-dihydropyrazolo[1,5-a]pyrazin-4(5H)-one | |
| 215 | 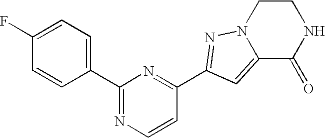 | 2-[2-(4-fluorophenyl)pyrimidin-4-yl]- 6,7-dihydropyrazolo[1,5-a]pyrazin-4(5H)-one | |
| 216 | 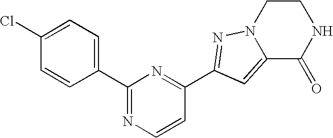 | 2-(2-(4-chlorophenyl)pyrimidin-4-yl]- 6,7-dihydropyrazoio[1,5-a]pyrazin-4(5H)-one | |
| 217 | 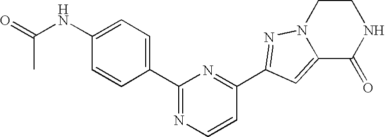 | N-{4-[4-(4-oxo-4,5,6,7- tetrahydropyrazolo[1,5-a]pyrazin-2- yl)pyrimidin-2-yl]phenyl}acetamide | |
| 218 | 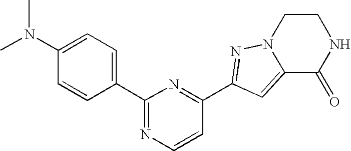 | 2-{2-[4-(dimethylamino)phenyl]pyrimidin-4-yl}-6,7- dihydropyrazolo[1,5-a]pyrazin-4(5H)-one | |
| 219 | 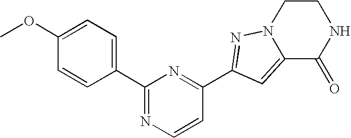 | 2-[2-(4-methoxyphenyl)pyrimidin-4- yl]-6,7-dihydropyrazolo[1,5- a]pyrazin-4(5H)-one | |
| 220 | 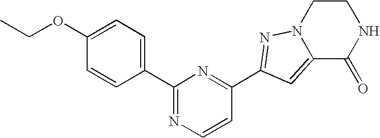 | 2-[2-(4-ethoxyphenyl)pyrimidin-4-yl]- 6,7-dihydropyrazolo[1,5-a]pyrazin-4(5H)-one | |
| 221 | 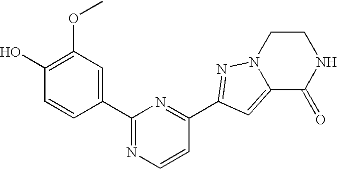 | 2-[2-(4-hydroxy-3-methoxyphenyl) pyrimidin-4-yl]-6,7- dihydropyrazolo[1,5-a]pyrazin- 4(5H)-one | |
| 222 | 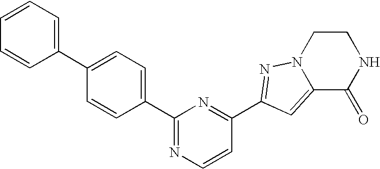 | 2-[2-(1,1′-biphenyl-4-yl)pyrimidin-4- yl]-6,7-dihydropyrazolo[1,5- a]pyrazin-4(5H)-one | |
| 223 | 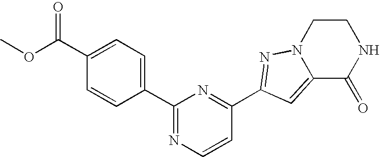 | methyl 4-[4-(4-oxo-4,5,6,7- tetrahydropyrazolo[1,5-a]pyrazin-2- yl)pyrimidin-2-yl]benzoate | |
| 224 | 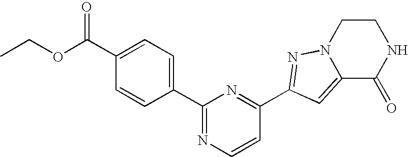 | ethyl 4-[4-(4-oxo-4,5,6,7- tetrahydropyrazolo[1,5-a]pyrazin-2- yl)pyrimidin-2-yl]benzoate | |
| 225 | 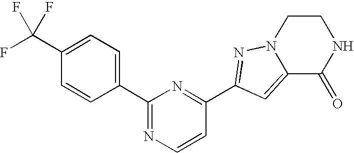 | 2-{2-[4-(trifluoromethyl)phenyl]pyrimidin-4-yl}-6,7- dihydropyrazolo[1,5-a]pyrazin- 4(5H)-one | |
| 226 | 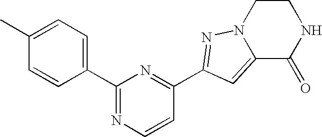 | 2-[2-(4-methylphenyl)pyrimidin-4-yl]- 6,7-dihydropyrazolo[1,5-a]pyrazin- 4(5H)-one | |
| 227 | 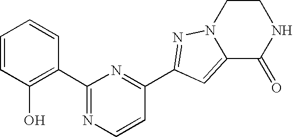 | 2-[2-(2-hydroxyphenyl)pyrimidin-4-yl]- 6,7-dihydropyrazolo[1,5-a]pyrazin- 4(5H)-one | |
| 228 | 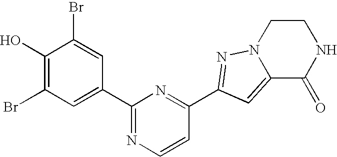 | 2-[2-(3,5-dibromo-4- hydroxyphenyl)pyrimidin-4-yl]-6,7- dihydropyrazolo[1,5-a]pyrazin- 4(5H)-one | |
| 229 | 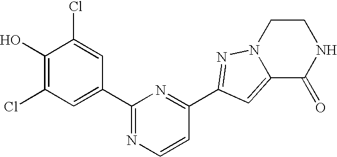 | 2-[2-(3,5-dichloro-4- hydroxyphenyl)pyrimidin-4-yl]-6,7- dihydropyrazolo[1,5-a]pyrazin- 4(5H)-one | |
| 230 | 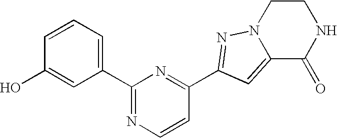 | 2-[2-(3-hydroxyphenyl)pyrimidin-4-yl]- 6,7-dihydropyrazolo[1,5-a]pyrazin-4(5H)-one | |
| 231 | 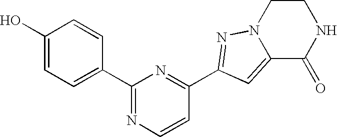 | 2-[2-(4-hydroxyphenyl)pyrimidin-4-yl]- 6,7-dihydropyrazolo[1,5-a]pyrazin-4(5H)-one | |
| 232 | 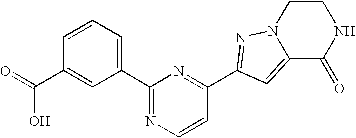 | 3-[4-(4-oxo-4,5,6,7-tetrahydropyrazolo [1,5-a]pyrazin-2-yl)pyrimidin- 2-yl]benzoic acid | |
| 233 | 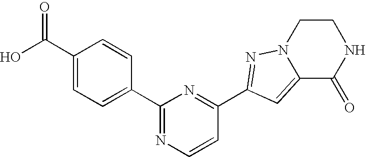 | 4-[4-(4-oxo-4,5,6,7-tetrahydropyrazolo [1,5-a]pyrazin-2-yl)pyrimidin- 2-yl]benzoic acid | |
| 234 | 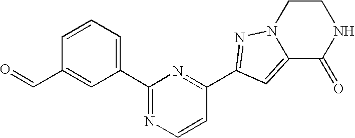 | 3-[4-(4-oxo-4,5,6,7-tetrahydropyrazolo [1,5-a]pyrazin-2-yl)pyrimidin-2- yl]benzaldehyde | |
| 235 | 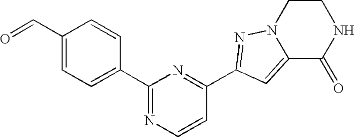 | 4-[4-(4-oxo-4,5,6,7-tetrahydropyrazolo [1,5-a]pyrazin-2-yl)pyrimidin-2- yl]benzaldehyde | |
| 236 | 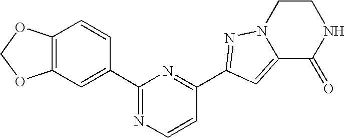 | 2-[2-(1,3-benzodioxol-5-yl)pyrimidin- 4-yl]-6,7-dihydropyrazolo[1,5-a]pyrazin-4(5H)-one | |
| 237 | 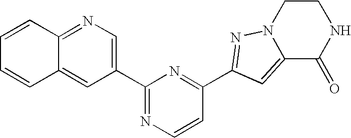 | 2-(2-quinolin-3-ylpyrimidin-4-yl)-6,7- dihydropyrazolo[1,5-a]pyrazin- 4(5H)-one | |
| 238 | 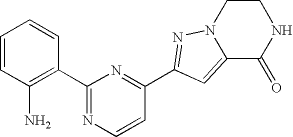 | 2-[2-(2-aminophenyl)pyrimidin-4-yl]- 6,7-dihydropyrazolo[1,5-a]pyrazin-4(5H)-one | |
| 239 | 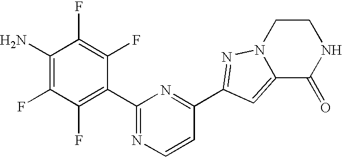 | 2-[2-(4-amino-2,3,5,6- tetrafluorophenyl)pyrimidin-4-yl]- 6,7-dihydropyrazolo[1,5- a]pyrazin-4(5H)-one | |
| 240 | 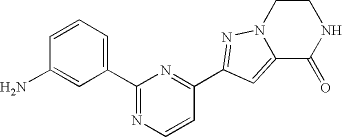 | 2-[2-(3-aminophenyl)pyrimidin-4-yl]- 6,7-dihydropyrazolo[1,5-a]pyrazin-4(5H)-one | |
| 241 | 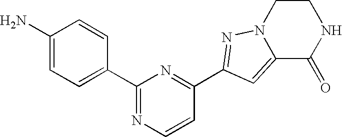 | 2-[2-(4-aminophenyl)pyrimidin-4-yl]- 6,7-dihydropyrazolo[1,5-a]pyrazin-4(5H)-one | |
| 242 | 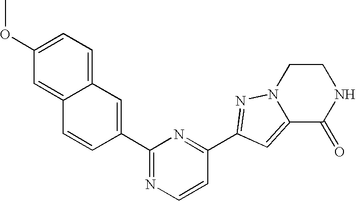 | 2-[2-(6-methoxy-2-naphthyl)pyrimidin- 4-yl]-6,7-dihydropyrazolo[1,5- a]pyrazin-4(5H)-one | |
| 243 | 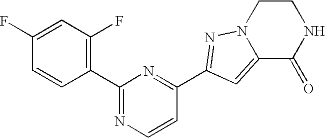 | 2-[2-(2,4-difluorophenyl)pyrimidin- 4-yl]-6,7-dihydropyrazolo[1,5- a]pyrazin-4(5H)-one | |
| 244 | 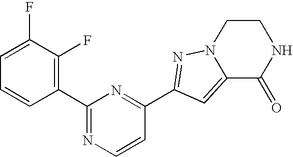 | 2-[2-(2,3-difluorophenyl)pyrimidin- 4-yl]-6,7-dihydropyrazolo[1,5- a]pyrazin-4(5H)-one | |
| 245 | 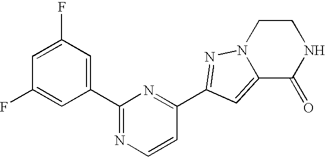 | 2-[2-(3,5-difluorophenyl)pyrimidin- 4-yl]-6,7-dihydropyrazolo[1,5- a]pyrazin-4(5H)-one | |
| 246 | 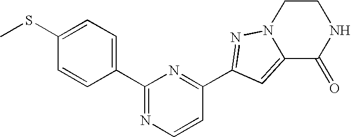 | 2-{2-[4-(methylthio)phenyl]pyrimidin- 4-yl}-6,7-dihydropyrazolo [1,5-a]pyrazin-4(5H)-one | |
| 247 | 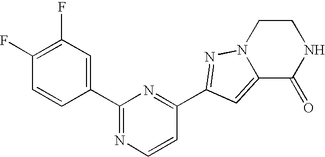 | 2-[2-(3,4-difluorophenyl)pyrimidin- 4-yl]-6,7-dihydropyrazolo[1,5- a]pyrazin-4(5H)-one | |
| 248 | 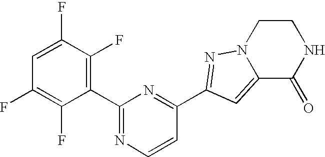 | 2-[2-(2,3,5,6-tetrafluorophenyl) pyrimidin-4-yl]-6,7- dihydropyrazolo[1,5-a]pyrazin- 4(5H)-one | |
| 249 | 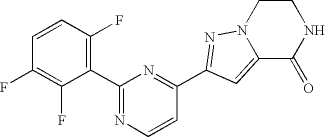 | 2-[2-(2,3,6-trifluorophenyl)pyrimidin- 4-yl]-6,7-dihydropyrazolo [1,5-a]pyrazin-4(5H)-one | |
| 250 | 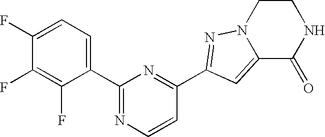 | 2-[2-(2,3,4-trifluorophenyl)pyrimidin- 4-yl]-6,7-dihydropyrazolo [1,5-a]pyrazin-4(5H)-one | |
| 251 | 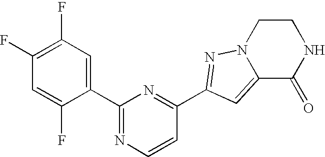 | 2-[2-(2,4,5-trifluorophenyl)pyrimidin- 4-yl]-6,7-dihydropyrazolo [1,5-a]pyrazin-4(5H)-one | |
| 252 | 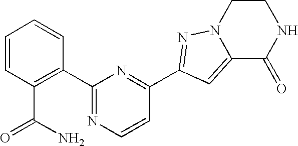 | 2-[4-(4-oxo-4,5,6,7-tetrahydropyrazolo [1,5-a]pyrazin-2-yl)pyrimidin- 2-yl]benzamide | |
| 253 | 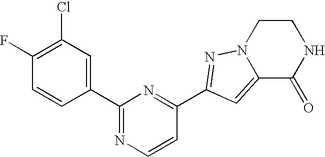 | 2-[2-(3-chloro-4-fluorophenyl) pyrimidin-4-yl]-6,7- dihydropyrazolo[1,5-a]pyrazin- 4(5H)-one | |
| 254 | 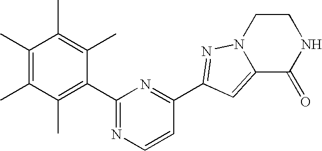 | 2-[2-(pentamethylphenyl)pyrimidin-4- yl]-6,7-dihydropyrazolo[1,5-a]pyrazin-4(5H)-one | |
| 255 | 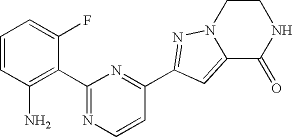 | 2-[2-(2-amino-6-fluorophenyl) pyrimidin-4-yl]-6,7- dihydropyrazolo[1,5-a]pyrazin- 4(5H)-one | |
| 256 | 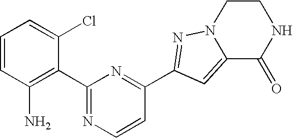 | 2-[2-(2-amino-6-chlorophenyl) pyrimidin-4-yl]-6,7- dihydropyrazolo[1,5-a]pyrazin- 4(5H)-one | |
| 257 | 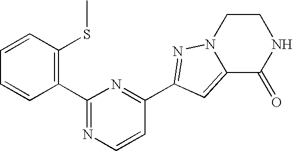 | 2-{2-[2-(methylthio)phenyl]pyrimidin- 4-yl}-6,7-dihydropyrazolo[1,5-a]pyrazin-4(5H)-one | |
| 258 | 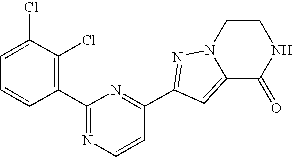 | 2-[2-(2,3-dichlorophenyl)pyrimidin- 4-yl]-6,7-dihydropyrazolo [1,5-a]pyrazin-4(5H)-one | |
| 259 | 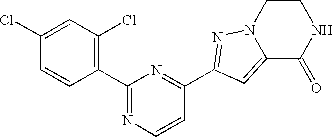 | 2-[2-(2,4-dichlorophenyl)pyrimidin- 4-yl]-6,7-dihydropyrazolo [1,5-a]pyrazin-4(5H)-one | |
| 260 | 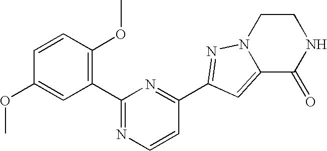 | 2-[2-(2,5-dimethoxyphenyl)pyrimidin- 4-yl]-6,7-dihydropyrazolo [1,5-a]pyrazin-4(5H)-one | |
| 261 | 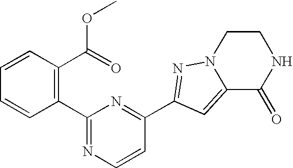 | methyl 2-[4-(4-oxo-4,5,6,7- tetrahydropyrazolo[1,5-a]pyrazin-2-yl)pyrimidin- 2-yl]benzoate | |
| 262 | 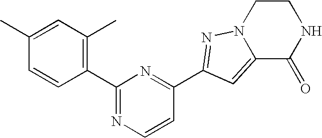 | 2-[2-(2,4-dimethylphenyl)pyrimidin-4- yl]-6,7-dihydropyrazolo[1,5-a]pyrazin-4(5H)-one | |
| 263 | 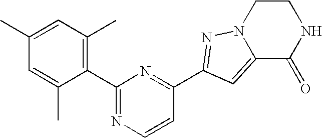 | 2-(2-mesitylpyrimidin-4-yl)-6,7- dihydropyrazolo[1,5-a]pyrazin- 4(5H)-one | |
| 264 | 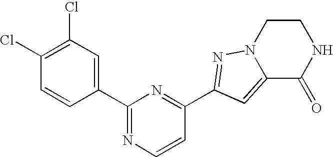 | 2-[2-(3,4-dichlorophenyl)pyrimidin- 4-yl]-6,7-dihydropyrazolo[1, 5-a]pyrazin-4(5H)-one | |
| 265 | 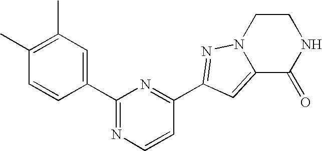 | 2-[2-(3,4-dimethylphenyl)pyrimidin- 4-yl]-6,7-dihydropyrazolo[1, 5-a]pyrazin-4(5H)-one | |
| 266 | 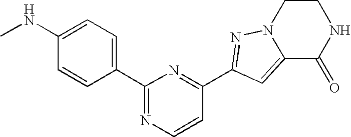 | 2-[2-[4-(methylamino)phenyl]pyrimidin-4-yl}-6,7- dihydropyrazolo[1,5-a]pyrazin- 4(5H)-one | |
| 267 | 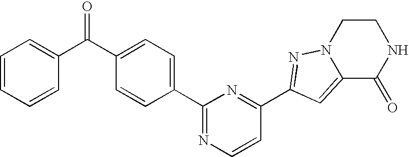 | 2-[2-(4-benzoylphenyl)pyrimidin- 4-yl]-6,7-dihydropyrazolo[1,5- a]pyrazin-4(5H)-one | |
| 268 | 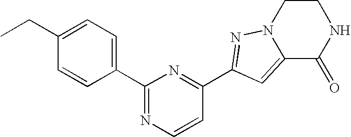 | 2-[2-(4-ethylphenyl)pyrimidin-4- yl]-6,7-dihydrapyrazolo[1 5- a]pyrazin-4(5H)-one | |
| 269 | 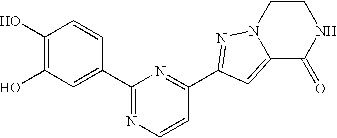 | 2-[2-(3,4-dihydroxyphenyl)pyrimidin- 4-yl]-6,7-dihydropyrazolo [1,5-a]pyrazin-4(5H)-one | |
| 270 | 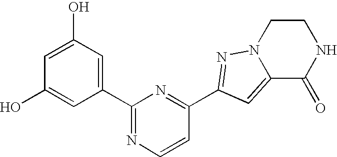 | 2-[2-(3,5-dihydroxyphenyl)pyrimidin- 4-yl]-6,7-dihydropyrazolo [1,5-a]pyrazin-4(5H)-one | |
| 271 | 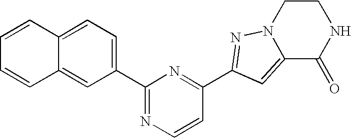 | 2-[2-(2-naphthyl)pyrimidin-4-yl]- 6,7-dihydropyrazolo[1,5-a]pyrazin-4(5H)-one | |
| 272 | 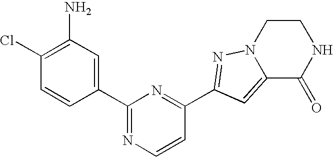 | 2-[2-(3-amino-4-chlorophenyl) pyrimidin-4-yl]-6,7- dihydropyrazolo[1,5-a]pyrazin- 4(5H)-one | |
| 273 | 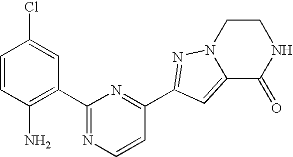 | 2-[2-(2-amino-5-chlorophenyl) pyrimidin-4-yl]-6,7- dihydropyrazolo[1,5-a]pyrazin- 4(5H)-one | |
| 274 | 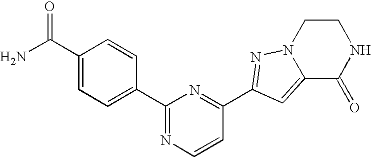 | 4-[4-(4-oxo-4,5,6,7-tetrahydropyrazolo [1,5-a]pyrazin-2-yl)pyrimidin-2- yl]benzamide | |
| 275 | 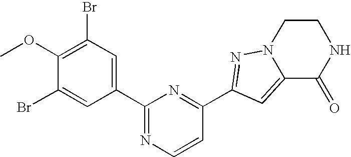 | 2-[2-(3,5-dibromo-4-methoxyphenyl) pyrimidin-4-yl]-6,7- dihydropyrazolo[1,5-a]pyrazin-4(5H)-one | |
| 276 | 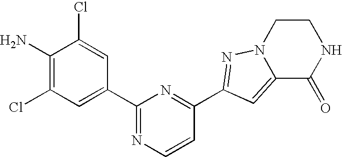 | 2-[2-(4-amino-3,5-dichlorophenyl) pyrimidin-4-yl]-6,7- dihydropyrazolo[1,5-a]pyrazin- 4(5H)-one | |
| 277 | 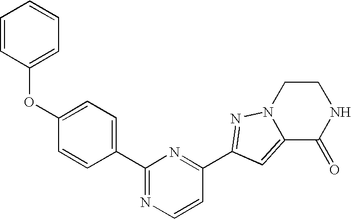 | 2-[2-(4-phenoxyphenyl)pyrimidin-4- yl]-6,7-dihydropyrazolo[1,5-a]pyrazin-4(5H)-one | |
| 278 | 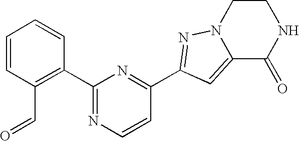 | 2-[4-(4-oxo-4,5,6,7-tetrahydropyrazolo [1,5-a]pyrazin-2-yl)pyrimidin- 2-yl]benzaldehyde | |
| 279 | 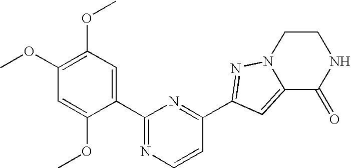 | 2-[2-(2,4,5-trimethoxyphenyl) pyrimidin-4-yl]-6,7- dihydropyrazolo[1,5-a]pyrazin- 4(5H)-one | |
| 280 | 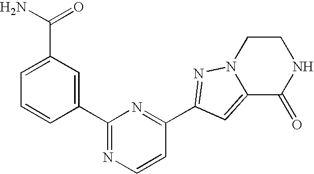 | 3-[4-(4-oxo-4,5,6,7- tetrahydropyrazolo[1,5-a]pyrazin-2-yl)pyrimidin-2- yl]benzamide | |
| 281 | 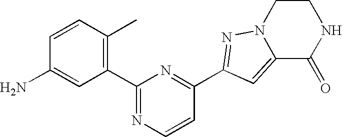 | 2-[2-(5-amino-2-methylphenyl) pyrimidin-4-yl]-6,7- dihydropyrazolo[1,5-a]pyrazin-4(5H)-one | |
| 282 | 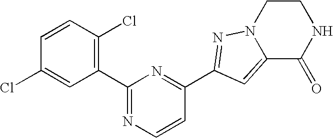 | 2-[2-(2,5-dichlorophenyl) pyrimidin-4-yl]-6,7- dihydropyrazolo[1,5-a]pyrazin-4(5H)-one | |
| 283 | 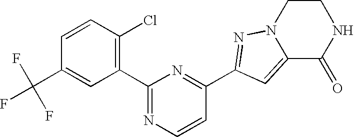 | 2-{2-(2-chloro-5- (trifluoromethyl)phenyl]pyrimidin-4- yl}-6,7-dihydropyrazolo [1,5-a]pyrazin-4(5H)-one | |
| 284 | 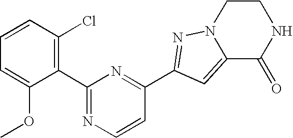 | 2-[2-(2-chloro-6-methoxyphenyl) pyrimidin-4-yl]-6,7- dihydrapyrazolo[1,5-a]pyrazin- 4(5H)-one | |
| 285 | 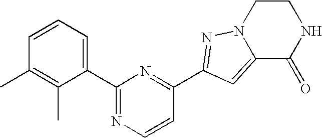 | 2-[2-(2,3-dimethylphenyl)pyrimidin-4- yl]-6,7-dihydropyrazolo [1,5-a]pyrazin-4(5H)-one | |
| 286 | 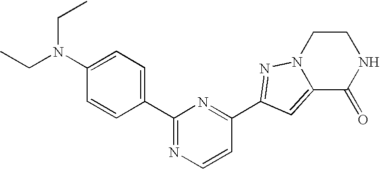 | 2-{2-[4-(diethylamino)phenyl]pyrimidin-4-yl}-6,7- dihydropyrazolo[1,5-a]pyrazin- 4(5H)-one | |
| 287 | 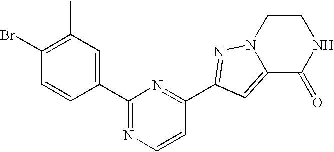 | 2-[2-(4-bromo-3-methylphenyl) pyrimidin-4-yl]-6,7- dihydropyrazolo[1,5-a]pyrazin- 4(5H)-one | |
| 288 | 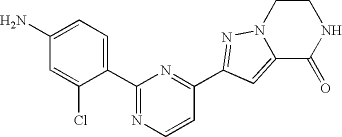 | 2-[2-(4-amino-2-chlorophenyl) pyrimidin-4-yl]-6,7- dihydropyrazolo[1,5-a]pyrazin- 4(5H)-one | |
| 289 | 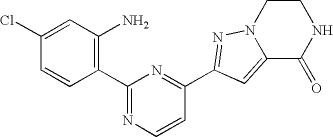 | 2-[2-(2-amino-4-chlorophenyl) pyrimidin-4-yl]-6,7- dihydropyrazolo[1,5-a]pyrazin- 4(5H)-one | |
| 290 | 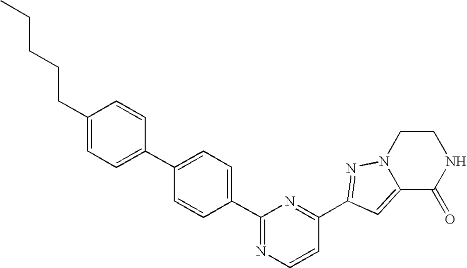 | 2-[2-(4′-pentyl-1,1′-biphenyl-4-yl) pyrimidin-4-yl]-6,7- dihydropyrazolo[1,5-a]pyrazin- 4(5H)-one | |
| 291 | 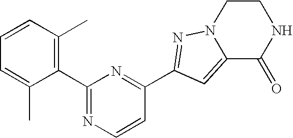 | 2-[2-(2,6-dimethylphenyl)pyrimidin- 4-yl]-6,7- dihydropyrazolo[1,5-a]pyrazin-4(5H)-one | |
| 292 | 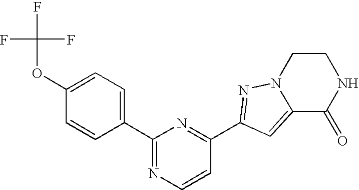 | 2-{2-[4-(trifluoromethoxy)phenyl]pyrimidin-4-yl}-6,7- dihydropyrazolo[1,5-a]pyrazin-4(5H)-one | |
| 293 | 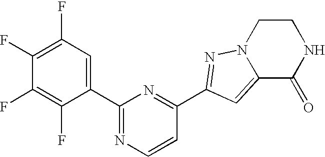 | 2-[2-(2,3,4,5-tetrafluorophenyl) pyrimidin-4-yl]-6,7- dihydropyrazolo[1,5-a]pyrazin- 4(5H)-one | |
| 294 | 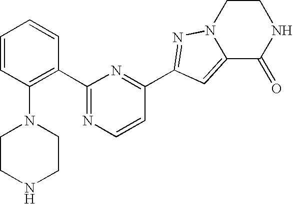 | 2-[2-(2-piperazin-1-ylphenyl)pyrimidin- 4-yl]-6,7-dihydropyrazolo [1,5-a]pyrazin-4(5H)-one | |
| 295 | 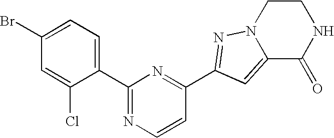 | 2-[2-(4-bromo-2-chlorophenyl)pyrimidin- 4-yl]-6,7-dihydropyrazolo [1,5-a]pyrazin-4(5H)-one | |
| 296 | 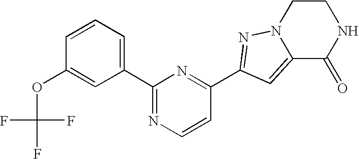 | 2-[2-[3-(trifluoromethoxy)phenyl]pyrimidin-4-yl]-6,7- dihydropyrazolo[1,5-a]pyrazin-4(5H)-one | |
| 297 | 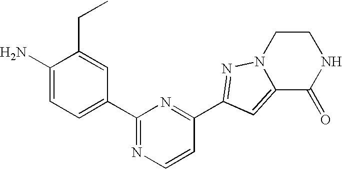 | 2-[2-(4-amino-3-ethylphenyl)pyrimidin- 4-yl]-6,7-dihydropyrazolo [1,5-a]pyrazin-4(5H)-one | |
| 298 | 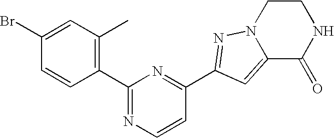 | 2-[2-(4-bromo-2-methylphenyl) pyrimidin-4-yl]-6,7- dihydropyrazolo[1,5-a]pyrazin- 4(5H)-one | |
| 299 | 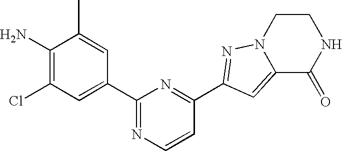 | 2-[2-(4-amino-3-chloro-5- methylphenyl)pyrimidin-4-yl]-6,7- dihydropyrazolo[1,5-a]pyrazin- 4(5H)-one | |
| 300 | 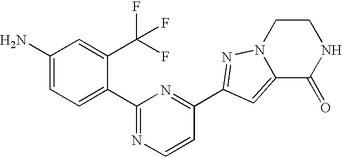 | 2-{2-(4-amino-2- (trifluoromethyl)phenyl]pyrimidin- 4-yl}-6,7-dihydropyrazolo [1,5-a]pyrazin-4(5H)-one | |
| 301 | 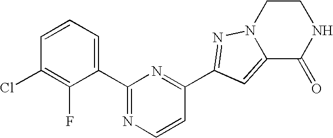 | 2-[2-(3-chloro-2-fluorophenyl) pyrimidin-4-yl]-6,7- dihydropyrazolo[1,5-a]pyrazin- 4(5H)-one | |
| 302 | 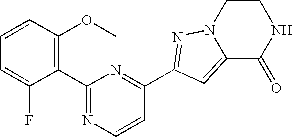 | 2-[2-(2-fluoro-6-methoxyphenyl) pyrimidin-4-yl]-6,7- dihydropyrazolo[1,5-a]pyrazin- 4(5H)-one | |
| 303 | 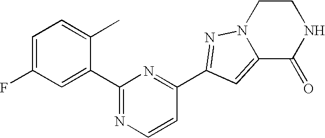 | 2-[2-(5-fluoro-2-methylphenyl) pyrimidin-4-yl]-6,7- dihydropyrazolo[1,5-a]pyrazin- 4(5H)-one | |
| 304 | 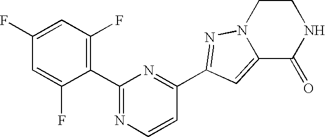 | 2-[2-(2,4,6-trifluorophenyl)pyrimidin- 4-yl]-6,7-dihydropyrazolo [1,5-a]pyrazin-4(5H)-one | |
| 305 | 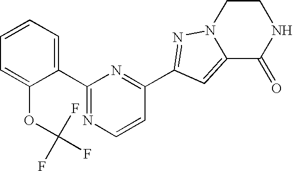 | 2-{2-[2-(trifluoromethoxy)phenyl]pyrimidin-4-yl}-6,7- dihydropyrazolo[1,5-a]pyrazin- 4(5H)-one | |
| 306 | 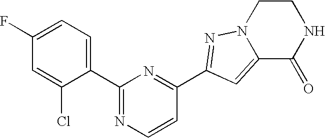 | 2-[2-(2-chloro-4-fluorophenyl) pyrimidin-4-yl]-6,7- dihydropyrazolo[1,5-a]pyrazin- 4(5H)-one | |
| 307 | 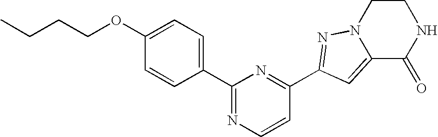 | 2-[2-(4-butoxyphenyl)pyrimidin- 4-yl]-6,7-dihydropyrazolo [1,5-a]pyrazin-4(5H)-one | |
| 308 | 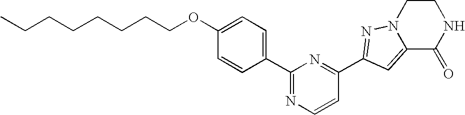 | 2-{2-[4-(octyloxy)phenyl]pyrimidin-4-yl}-6,7- dihydropyrazolo[1,5-a]pyrazin-4(5H)-one | |
| 309 | 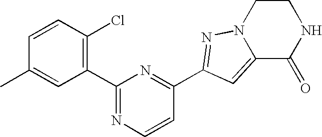 | 2-[2-(2-chloro-5-methylphenyl) pyrimidin-4-yl]-6,7- dihydropyrazolo[1,5-a]pyrazin-4(5H)-one | |
| 310 | 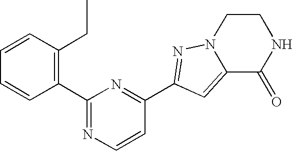 | 2-[2-(2-ethylphenyl)pyrimidin-4- yl]-6,7-dihydropyrazolo [1,5-a]pyrazin-4(5H)-one | |
| 311 | 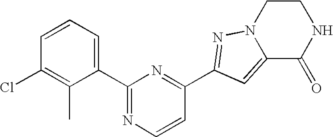 | 2-[2-(3-chloro-2-methylphenyl) pyrimidin-4-yl]-6,7- dihydropyrazolo[1,5-a]pyrazin- 4(5H)-one | |
| 312 | 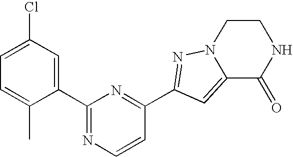 | 2-[2-(5-chloro-2-methylphenyl) pyrimidin-4-yl]-6,7- dihydropyrazolo[1,5-a]pyrazin- 4(5H)-one | |
| 313 | 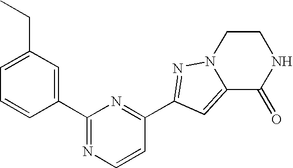 | 2-[2-(3-ethylphenyl)pyrimidin- 4-yl]-6,7-dihydropyrazolo [1,5-a]pyrazin-4(5H)-one | |
| 314 | 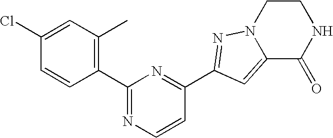 | 2-[2-(4-chloro-2-methylphenyl) pyrimidin-4-yl]-6,7- dihydropyrazolo[1,5-a]pyrazin- 4(5H)-one | |
| 315 | 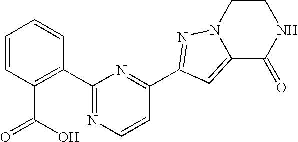 | 2-[4-(4-oxo-4,5,6,7-tetrahydropyrazolo [1,5-a]pyrazin-2-yl)pyrimidin- 2-yl]benzoic acid | |
| 316 | 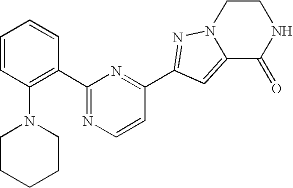 | 2-[2-(2-piperidin-1-ylphenyl)pyrimidin- 4-yl]-6,7-dihydropyrazolo[1,5- a]pyrazin-4(5H)-one | |
| 317 | 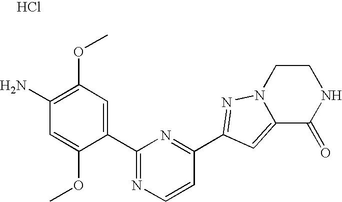 | 2-[2-(4-amino-2,5-dimethoxyphenyl) pyrimidin-4-yl]-6,7- dihydropyrazolo[1,5-a]pyrazin- 4(5H)-one hydrochloride | |
| 318 | 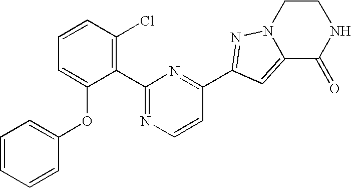 | 2-[2-(2-chloro-6-phenoxyphenyl) pyrimidin-4-yl]-6,7- dihydropyrazolo[1,5-a]pyrazin- 4(5H)-one | |
| 319 | 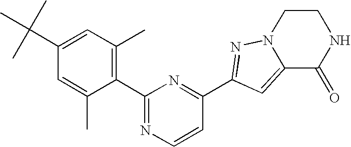 | 2-[2-(4-tert-butyl-2,6- dimethylphenyl)pyrimidin-4-yl]- 6,7-dihydropyrazolo[1,5-a]pyrazin- 4(5H)-one | |
| 320 | 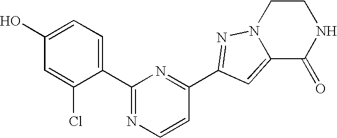 | 2-[2-(2-chloro-4-hydroxyphenyl) pyrimidin-4-yl]-6,7- dihydropyrazolo[1,5-a]pyrazin- 4(5H)-one | |
| 321 | 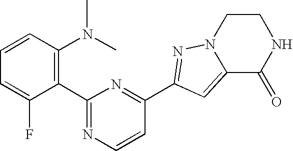 | 2-{2-[2-(dimethylamino)-6- fluorophenyl]pyrimidin-4-yl}-6,7- dihydropyrazolo[1,5-a]pyrazin- 4(5H)-one | |
| 322 | 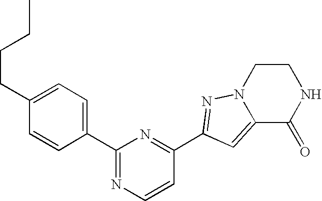 | 2-[2-(4-butylphenyl)pyrimidin-4- yl]-6,7-dihydropyrazolo[1,5-a]pyrazin-4(5H)-one | |
| 323 | 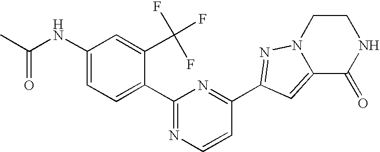 | N-[4-[4-(4-oxo-4,5,6,7- tetrahydropyrazolo[1,5-a]pyrazin-2- yl)pyrimidin-2-yl]-3- (trifluoromethyl)phenyl]acetamide | |
| 324 | 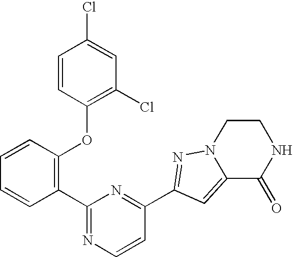 | 2-{2-[2-(2,4- dichlorophenoxy)phenyl]pyrimidin-4-yl}-6,7- dihydropyrazolo[1,5-a]pyrazin- 4(5H)-one | |
| 325 | 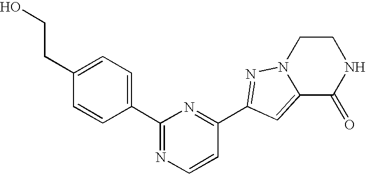 | 2-{2-[4-(2-hydroxyethyl)phenyl]pyrimidin-4-yl}-6,7- dihydropyrazolo[1,5-a]pyrazin- 4(5H)-one | |
| 326 | 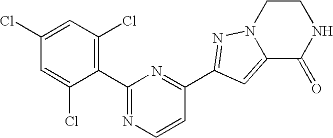 | 2-[2-(2,4,6-trichlorophenyl)pyrimidin- 4-yl]-6,7-dihydropyrazolo[1, 5-a]pyrazin-4(5H)-one | |
| 327 | 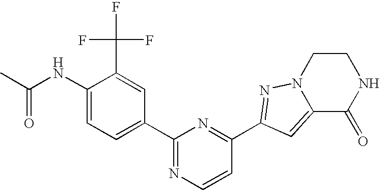 | N-[4-[4-(4-oxo-4,5,6,7- tetrahydropyrazolo[1,5-a]pyrazin-2- yl)pyrimidin-2-yl]-2- (trifluoromethyl)phenyl]acetamide | |
| 328 | 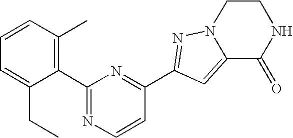 | 2-[2-(2-ethyl-6-methylphenyl) pyrimidin-4-yl]-6,7- dihydropyrazolo[1,5-a]pyrazin- 4(5H)-one | |
| 329 | 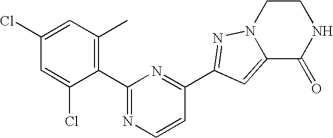 | 2-[2-(2,4-dichloro-6-methylphenyl) pyrimidin-4-yl]-6,7- dihydropyrazolo[1,5-a]pyrazin- 4(5H)-one | |
| 330 | 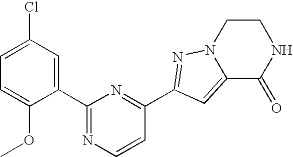 | 2-[2-(5-chloro-2-methoxyphenyl) pyrimidin-4-yl]-6,7- dihydropyrazolo[1,5-a]pyrazin- 4(5H)-one | |
| 331 | 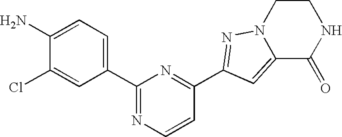 | 2-[2-(4-amino-3-chlorophenyl) pyrimidin-4-yl]-6,7- dihydropyrazolo[1,5-a]pyrazin- 4(5H)-one | |
| 332 | 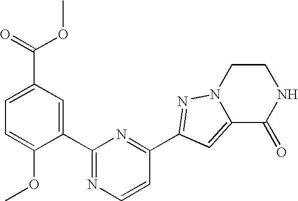 | methyl 4-methoxy-3-[4-(4-oxo-4,5,6,7- tetrahydropyrazolo[1,5-a]pyrazin-2-yl) pyrimidin-2-yl]benzoate | |
| 333 | 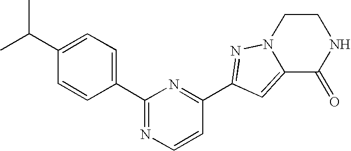 | 2-[2-(4-isopropylphenyl)pyrimidin-4- yl]-6,7-dihydropyrazolo [1,5-a]pyrazin-4(5H)-one | |
| 334 | 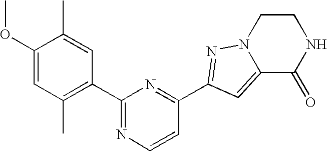 | 2-[2-(4-methoxy-2,5- dimethylphenyl)pyrimidin-4-yl]-6,7- dihydropyrazolo[1,5-a]pyrazin-4(5H)-one | |
| 335 | 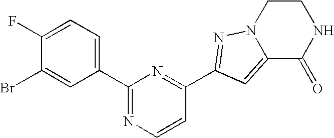 | 2-[2-(3-bromo-4-fluorophenyl) pyrimidin-4-yl]-6,7- dihydropyrazolo[1,5-a]pyrazin-4(5H)-one | |
| 336 | 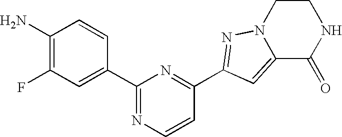 | 2-[2-(4-amino-3-fluorophenyl) pyrimidin-4-yl]-6,7- dihydropyrazolo[1,5-a]pyrazin-4(5H)-one | |
| 337 | 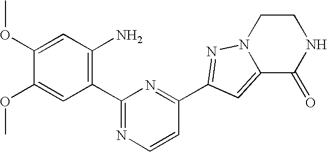 | 2-[2-(2-amino-4,5- dimethoxyphenyl)pyrimidin-4-yl]-6,7- dihydropyrazolo[1,5-a]pyrazin-4(5H)-one | |
| 338 | 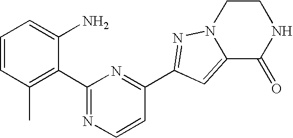 | 2-[2-(2-amino-6-methylphenyl) pyrimidin-4-yl]-6,7- dihydropyrazolo[1,5-a]pyrazin- 4(5H)-one | |
| 339 | 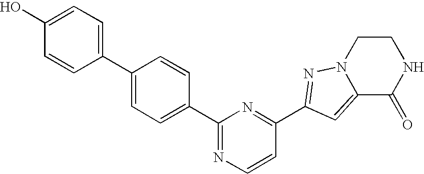 | 2-[2-(4′-hydroxy-1,1′-biphenyl-4-yl) pyrimidin 4-yl]-6,7- dihydropyrazolo[1,5-a]pyrazin- 4(5H)-one | |
| 340 | 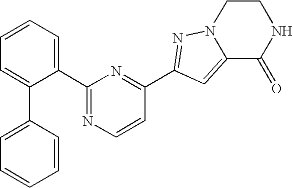 | 2-[2-(1,1′-biphenyl-2-yl)pyrimidin-4- yl]-6,7-dihydropyrazolo[1,5-a]pyrazin-4(5H)-one | |
| 341 | 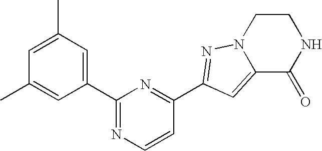 | 2-[2-(3,5-dimethylphenyl)pyrimidin-4- yl]-6,7-dihydropyrazolo [1,5-a]pyrazin-4(5H)-one | |
| 342 | 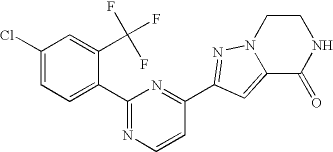 | 2-{2-(4-chloro-2- (trifluoromethyl)phenyl]pyrimidin- 4-yl}-6,7- dihydropyrazolo[1,5-a]pyrazin4(5H)-one | |
| 343 | 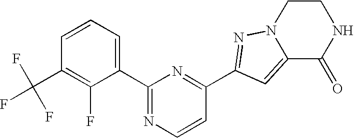 | 2-{2-[2-fluoro-3- (trifluoromethyl)phenyl]pyrimidin- 4-yl}-6,7- dihydropyrazolo[1,5-a]pyrazin- 4(5H)-one | |
| 344 | 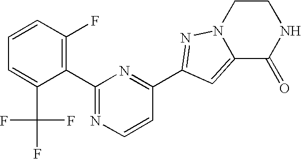 | 2-{2-[2-fluoro-6- (trifluoromethyl)phenyl]pyrimidin- 4-yl}-6,7- dihydropyrazolo[1,5-a]pyrazin- 4(5H)-one | |
| 345 | 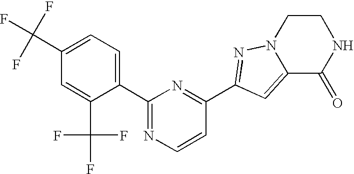 | 2-{2-[2,4- bis(trifluoromethyl)phenyl]pyrimidin- 4-yl}-6,7- dihydropyrazolo[1,5-a]pyrazin- 4(5H)-one | |
| 346 | 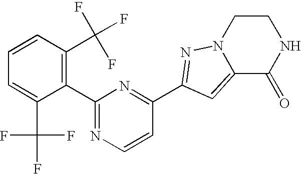 | 2-{2-[2,6- bis(trifluoromethyl)phenyl]pyrimidin- 4-yl}-6,7- dihydropyrazolo[1,5-a]pyrazin- 4(5H)-one | |
| 347 | 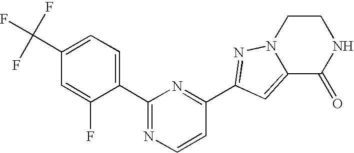 | 2-{2-[2-fluoro-4- (trifluoromethyl)phenyl]pyrimidin- 4-yl}-6,7-dihydropyrazolo [1,5-a]pyrazin-4(5H)-one | |
| 348 | 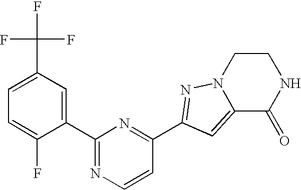 | 2-{2-[2-fluoro-5- (trifluoromethyl)phenyl]pyrimidin- 4-yl}-6,7- dihydropyrazolo[1,5-a]pyrazin- 4(5H)-one | |
| 349 | 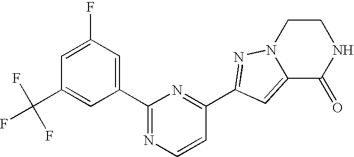 | 2-{2-[3-fluoro-5- (trifluoromethyl)phenyl]pyrimidin- 4-yl}-6,7- dihydropyrazolo[1,5-a]pyrazin- 4(5H)-one | |
| 350 | 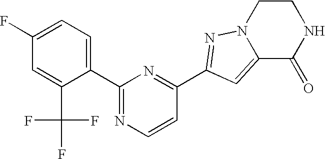 | 2-{2-(4-fluoro-2- (trifluoromethyl)phenyl]pyrimidin- 4-yl}-6,7-dihydropyrazolo [1,5-a]pyrazin-4(5H)-one | |
| 351 | 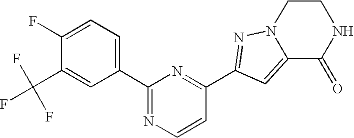 | 2-{2-[4-fluoro-3- (trifluoromethyl)phenyl]pyrimidin- 4-yl}-6,7-dihydropyrazolo [1,5-a]pyrazin-4(5H)-one | |
| 352 | 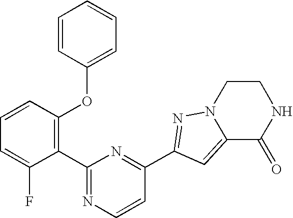 | 2-[2-(2-fluoro-6-phenoxyphenyl) pyrimidin-4-yl]-6,7- dihydropyrazolo[1,5-a]pyrazin-4(5H)-one | |
| 353 | 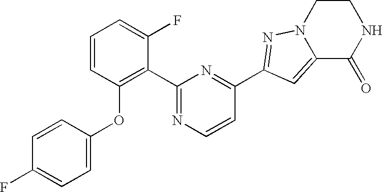 | 2-{2-[2-fluoro-6-(4- fluorophenoxy)phenyl]pyrimidin- 4-yl}-6,7- dihydropyrazolo[1,5-a]pyrazin-4(5H)-one | |
| 354 | 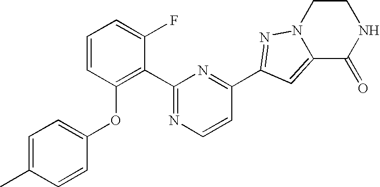 | 2-{2-[2-fluoro-6-(4- methylphenoxy)phenyl]pyrimidin- 4-yl}-6,7- dihydropyrazolo[1,5-a]pyrazin-4(5H)-one | |
| 355 | 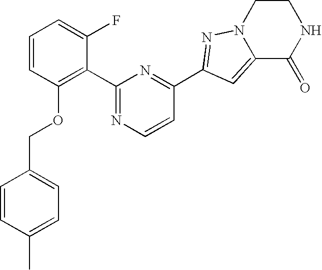 | 2-(2-{2-fluoro-6-[(4- methylbenzyl)oxy]phenyl}pyrimidin-4-yl)- 6,7-dihydropyrazolo[1,5-a]pyrazin-4(5H)-one | |
| 356 | 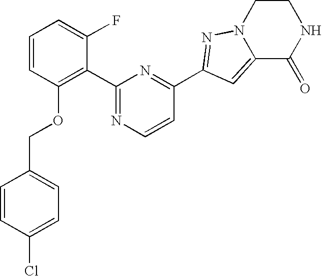 | 2-(2-{2-[(4-chlorobenzyl)oxy]-6- fluorophenyl}pyrimidin-4-yl)-6,7- dihydropyrazolo[1,5-a]pyrazin-4(5H)-one | |
| 357 | 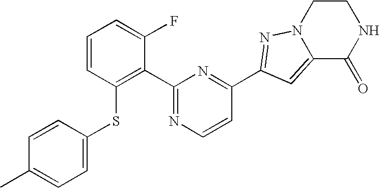 | 2-(2-{2-fluoro-6-[(4- methylphenyl)thio]phenyl}pyrimidin-4-yl)- 6,7-dihydropyrazolo[1,5-a]pyrazin-4(5H)-one | |
| 358 | 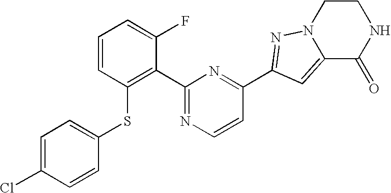 | 2-(2-{2-[(4-chlorophenyl)thio]-6- fluorophenyl}pyrimidin-4-yl)-6,7- dihydropyrazolo[1,5-a]pyrazin- 4(5H)-one | |
| 359 | 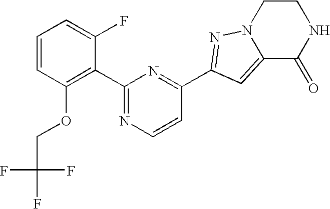 | 2-{2-[2-fluoro-6-(2,2,2- trifluoroethoxy)phenyl]pyrimidin- 4-yl}-6,7- dihydropyrazolo[1,5-a]pyrazin- 4(5H)-one | |
| 360 | 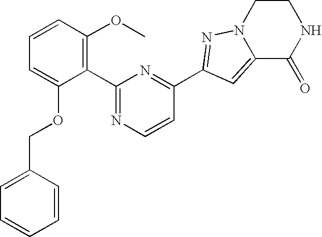 | 2-{2-[2-(benzyloxy)-6- methoxyphenyl]pyrimidin-4-yl}- 6,7-dihydropyrazolo[1,5-a]pyrazin-4(5H)-one | |
| 361 | 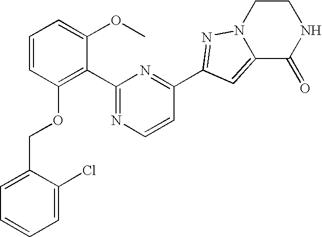 | 2-(2-{2-[(2-chlorobenzyl)oxy]-6- methoxyphenyl}pyrimidin-4-yl)-6,7- dihydropyrazolo[1,5-a]pyrazin- 4(5H)-one | |
| 362 | 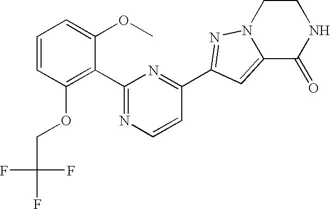 | 2-{2-[2-methoxy-6-(2,2,2- trifluoroethoxy)phenyl]pyrimidin- 4-yl}-6,7-dihydropyrazolo [1,5-a]pyrazin-4(5H)-one | |
| 363 | 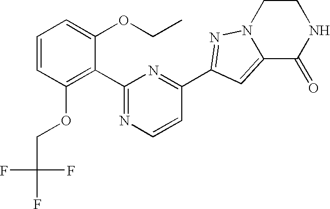 | 2-{2-[2-ethoxy-6-(2,2,2- trifluoroethoxy)phenyl]pyrimidin- 4-yl}-6,7-dihydropyrazolo [1,5-a]pyrazin-4(5H)-one | |
| 364 | 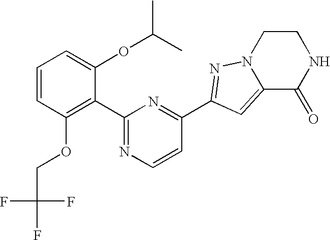 | 2-{2-[2-isopropoxy-6-(2,2,2- trifluoroethoxy)phenyl]pyrimidin- 4-yl}-6,7-dihydropyrazolo [1,5-a]pyrazin-4(5H)-one | |
| 365 | 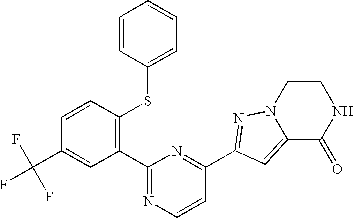 | 2-{2-[2-(phenylthio)-5- (trifluoromethyl)phenyl]pyrimidin- 4-yl}-6,7-dihydropyrazolo[1,5-a]pyrazin-4(5H)-one | |
| 366 | 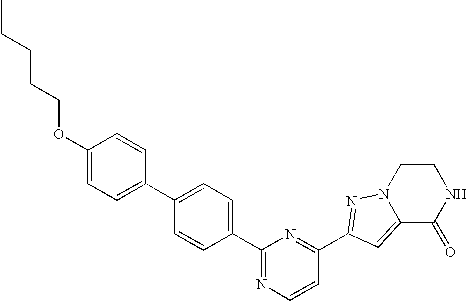 | 2-{2-[4′-(pentyloxy)-1,1′-biphenyl-4- yl]pyrimidin-4-yl}-6,7- dihydropyrazolo[1,5-a]pyrazin-4(5H)-one | |
| 367 | 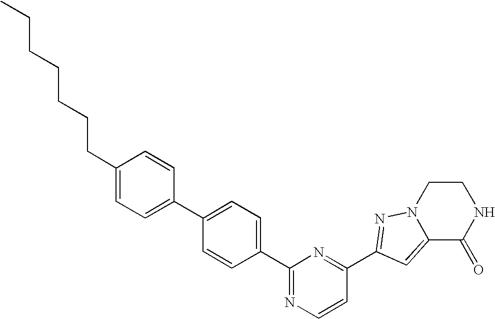 | 2-[2-(4′-heptyl-1,1′-biphenyl-4-yl) pyrimidin-4-yl]-6,7- dihydropyrazolo[1,5-a]pyrazin- 4(5H)-one | |
| 368 | 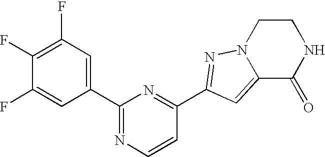 | 2-[2-(3,4,5-trifluorophenyl)pyrimidin-4- yl]-6,7-dihydropyrazolo[1,5-a]pyrazin-4(5H)-one | |
| 369 | 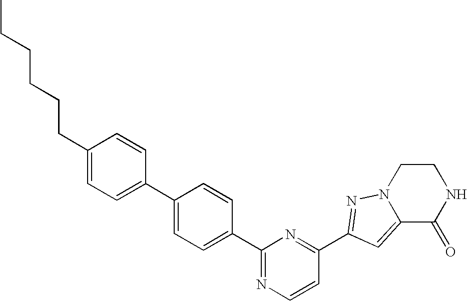 | 2-[2-(4′-hexyl-1,1′-biphenyl-4-yl) pyrimidin-4-yl]-6,7- dihydropyrazolo[1,5-a]pyrazin- 4(5H)-one | |
| 370 | 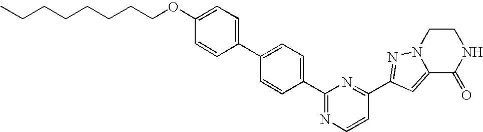 | 2-{2-[4′-(octyloxy)-1,1′-biphenyl-4- yl]pyrimidin-4-yl}-6,7- dihydropyrazolo[1,5-a]pyrazin-4(5H)-one | |
| 371 | 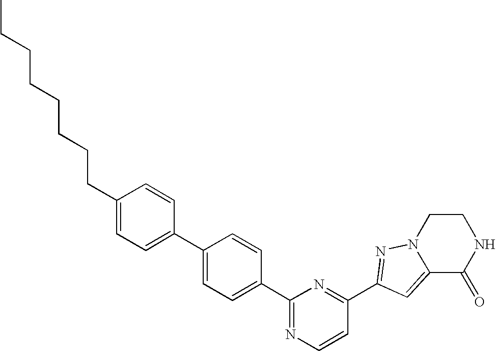 | 2-[2-(4′-octyl-1,1′biphenyl-4-yl) pyrimidin-4-yl]-6,7- dihydropyrazolo[1,5-a]pyrazin- 4(5H)-one | |
| 372 | 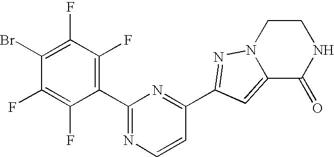 | 2-[2-(4-bromo-2,3,5,6- tetrafluorophenyl)pyrimidin-4-yl]-6,7- dihydropyrazolo[1,5-a]pyrazin- 4(5H)-one | |
| 373 | 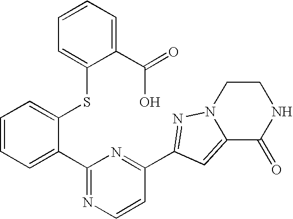 | 2-({2-[4-(4-oxo-4,5,6,7- tetrahydropyrazolo[1,5-a]pyrazin- 2-yl)pyrimidin-2-yl]phenyl}thio)benzoic acid | |
| 374 | 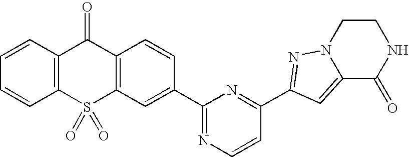 | 2-[2-(10,10-dioxido-9-oxo-9H- thioxanthen-3-yl)pyrimidin-4-yl]- 6,7-dihydropyrazolo[1,5- a]pyrazin-4(5H)-one | |
| 375 | 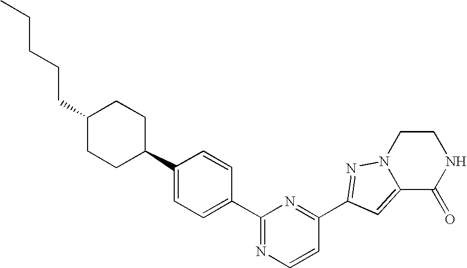 | 2-{2-[4-(4-pentylcyclohexyl) phenyl]pyrimidin-4-yl}-6,7- dihydropyrazolo[1,5-a]pyrazin- 4(5H)-one | |
| 376 | 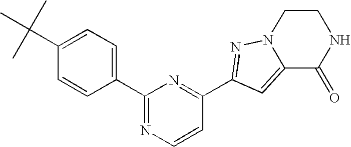 | 2-[2-(4-tert-butylphenyl)pyrimidin- 4-yl]-6,7-dihydropyrazolo[1,5-a]pyrazin-4(5H)-one | |
| 377 | 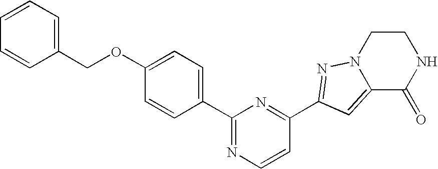 | 2-{2-[4-(benzyloxy)phenyl]pyrimidin-4-yl}-6,7- dihydropyrazolo[1,5-a]pyrazin- 4(5H)-one | |
| 378 | 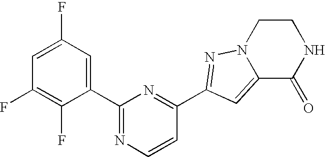 | 2-[2-(2,3,5-trifluorophenyl)pyrimidin-4- yl]-6,7-dihydropyrazolo [1,5-a]pyrazin-4(5H)-one | |
| 379 | 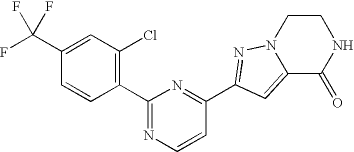 | 2-{2-[2-chloro-4- (trifluoromethyl)phenyl]pyrimidin- 4-yl}-6,7-dihydropyrazolo [1,5-a]pyrazin-4(5H)-one | |
| 380 | 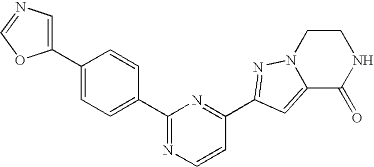 | 2-({2-[4-(1,3-oxazol-5-yl)phenyl]pyrimidin-4-yl}-6,7- dihydropyrazolo[1,5-a]pyrazin-4(5H)-one | |
| 381 | 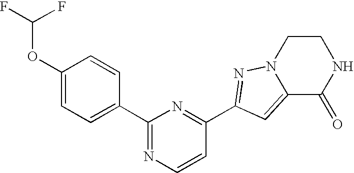 | 2-{2-[4-(difluoromethoxy)phenyl]pyrimidin-4-yl}-6,7- dihydropyrazolo[1,5-a]pyrazin-4(5H)-one | |
| 382 | 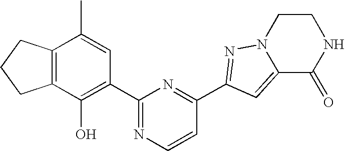 | 2-[2-(4-hydroxy-7-methyl-2,3-dihydro- 1H-inden-5-yl)pyrimidin-4- yl]-6,7-dihydropyrazolo[1,5-a]pyrazin-4(5H)-one | |
| 383 | 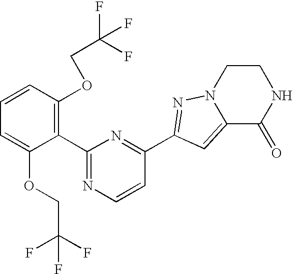 | 2-{2-[2,6-bis(2,2,2- trifluoroethoxy)phenyl]pyrimidin-4- yl}-6,7-dihydropyrazolo [1,5-a]pyrazin-4(5H)-one | |
| 384 | 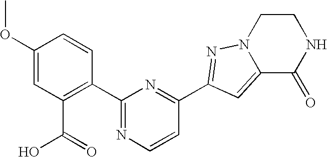 | 5-methoxy-2-[4-(4-oxo-4,5,6,7- tetrahydropyrazolo[1,5-a]pyrazin-2- yl)pyrimidin-2-yl]benzoic acid | |
| 385 | 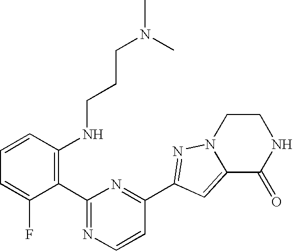 | 2-[2-(2-{[3-(dimethylamino)propyl]amino}-6-fluorophenyl) pyrimidin-4-yl]-6,7- dihydropyrazolo[1,5-a]pyrazin- 4(5H)-one | |
| 386 | 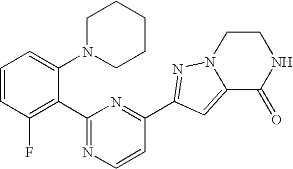 | 2-[2-(2-fluoro-6-piperidin-1- ylphenyl)pyrimidin-4-yl]-6,7- dihydropyrazolo[1,5-a]pyrazin-4(5H)- one | |
| 387 | 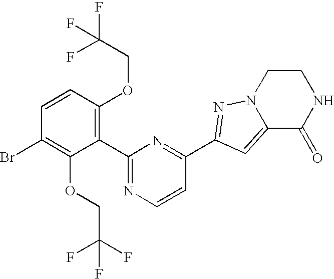 | 2-{2-[3-bromo-2,6-bis(2,2,2- trifluoroethoxy)phenyl]pyrimidin-4- yl}-6,7-dihydropyrazolo [1,5-a]pyrazin-4(5H)-one | |
| 388 | 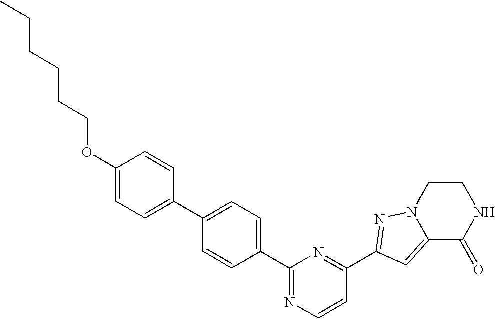 | 2-{2-[4′-(hexyloxy)-1,1′-biphenyl-4- yl]pyrimidin-4-yl}-6,7- dihydropyrazolo[1,5-a]pyrazin-4(5H)-one | |
| 389 | 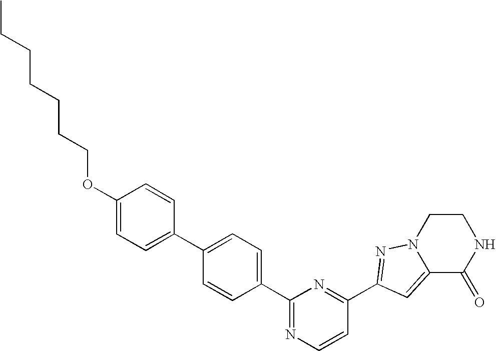 | 2-{2-[4′-(heptyloxy)-1,1′-biphenyl-4- yl]pyrimidin-4-yl}-6,7- dihydropyrazolo[1,5-a]pyrazin-4(5H)-one | |
| 390 | 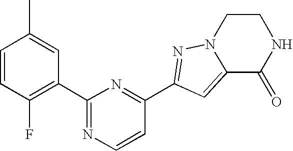 | 2-[2-(2-fluoro-5-methylphenyl)pyrimidin- 4-yl]-6,7-dihydropyrazolo[1,5- a]pyrazin-4(5H)-one | |
| 391 | 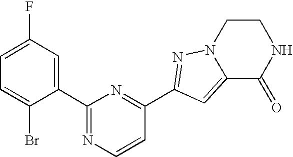 | 2-[2-(2-bromo-5-fluorophenyl)pyrimidin- 4-yl]-6,7-dihydropyrazolo[1,5- a]pyrazin-4(5H)-one | |
| 392 | 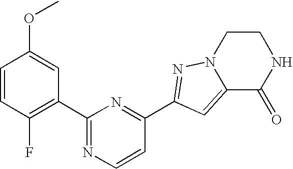 | 2-[2-(2-fluoro-5-methoxyphenyl)pyrimidin- 4-yl]-6,7-dihydropyrazolo[1,5- a]pyrazin-4(5H)-one | |
| 393 | 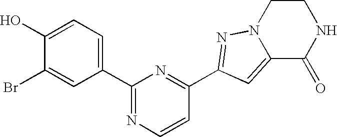 | 2-[2-(3-bromo-4-hydroxyphenyl)pyrimidin-4- yl]-6,7-dihydropyrazolo[1,5- a]pyrazin-4(5H)-one | |
| 394 | 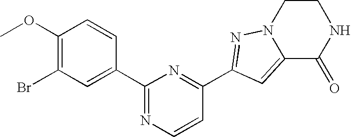 | 2-[2-(3-bromo-4-methoxyphenyl)pyrimidin-4- yl]-6,7-dihydropyrazolo[1,5- a]pyrazin-4(5H)-one | |
| 395 | 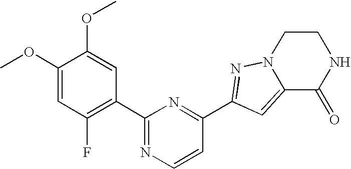 | 2-[2-(2-fluoro-4,5-dimethoxyphenyl) pyrimidin-4-yl]-6,7- dihydropyrazolo[1,5-a]pyrazin-4(5H)-one | |
| 396 | 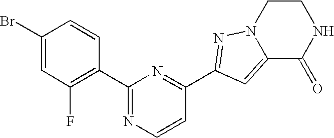 | 2-[2-(4-bromo-2-fluorophenyl) pyrimidin-4-yl]-6,7- dihydropyrazolo[1,5-a]pyrazin- 4(5H)-one | |
| 397 | 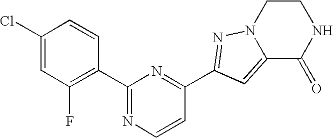 | 2-[2-(4-chloro-2-fluorophenyl) pyrimidin-4-yl]-6,7- dihydropyrazolo[1,5-a]pyrazin- 4(5H)-one | |
| 398 | 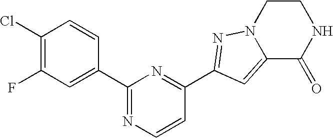 | 2-[2-(4-chloro-3-fluorophenyl) pyrimidin-4-yl]-6,7- dihydropyrazolo[1,5-a]pyrazin- 4(5H)-one | |
| 399 | 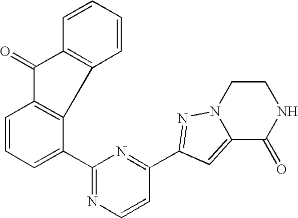 | 2-[2-(9-oxo-9H-fluoren-4-yl) pyrimidin-4-yl]-6,7- dihydropyrazolo[1,5-a]pyrazin- 4(5H)-one | |
| 400 | 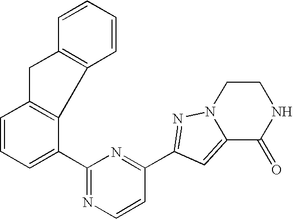 | 2-[2-(9H-fluoren-4-yl)pyrimidin-4- yl]-6,7-dihydropyrazolo[1,5-a]pyrazin-4(5H)-one | |
| 401 | 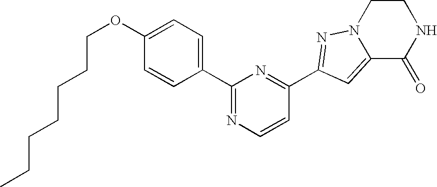 | 2-{2-[4-(heptyloxy)phenyl]pyrimidin- 4-yl}-6,7-dihydropyrazolo [1,5-a]pyrazin-4(5H)-one | |
| 402 | 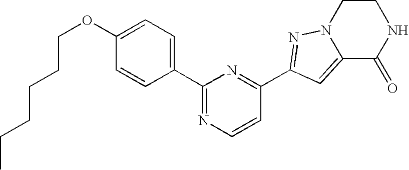 | 2-{2-[4-(hexyloxy)phenyl]pyrimidin- 4-yl}-6,7-dihydropyrazolo [1,5-a]pyrazin-4(5H)-one | |
| 403 | 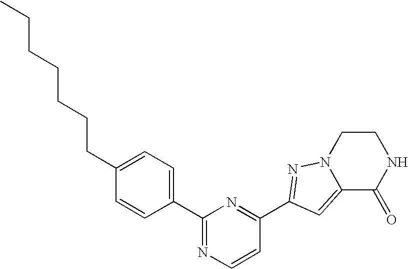 | 2-[2-(4-heptylphenyl)pyrimidin-4-yl]- 6,7-dihydropyrazolo[1,5-a]pyrazin-4(5H)-one | |
| 404 | 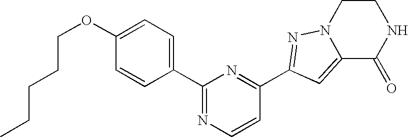 | 2-{2-[4-(pentyloxy)phenyl]pyrimidin- 4-yl}-6,7-dihydropyrazolo [1,5-a]pyrazin-4(5H)-one | |
| 405 | 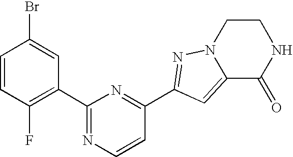 | 2-[2-(5-bromo-2-fluorophenyl) pyrimidin-4-yl]-6,7- dihydropyrazolo[1,5-a]pyrazin- 4(5H)-one | |
| 406 | 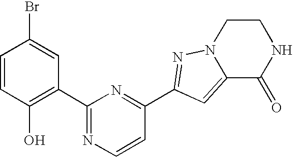 | 2-[2-(5-bromo-2-hydroxyphenyl) pyrimdin-4-yl]-6,7- dihydropyrazolo[1,5-a]pyrazin- 4(5H)-one | |
| 407 | 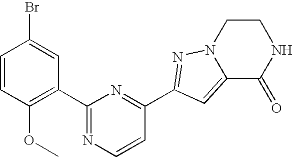 | 2-[2-(5-bromo-2-methoxyphenyl) pyrimidin-4-yl]-6,7- dihydropyrazolo[1,5-a]pyrazin- 4(5H)-one | |
| 408 | 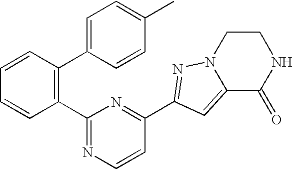 | 2-[2-(4′-methyl-1,1′-biphenyl-2- yl)pyrimidin-4-yl]-6,7-dihydropyrazolo [1,5-a]pyrazin-4(5H)-one | |
| 409 | 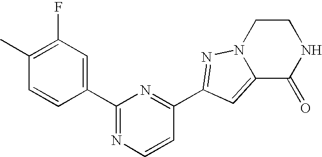 | 2-[2-(3-fluoro-4-methylphenyl) pyrimidin-4-yl]-6,7- dihydropyrazolo[1,5-a]pyrazin- 4(5H)-one | |
| 410 | 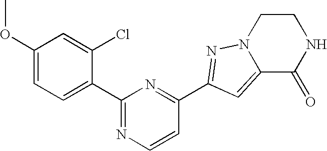 | 2-[2-(2-chloro-4-methoxyphenyl) pyrimidin-4-yl]-6,7- dihydropyrazolo[1,5-a]pyrazin- 4(5H)-one | |
| 411 | 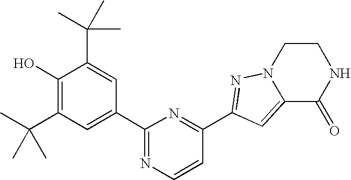 | 2-[2-(3,5-di-tert-butyl-4- hydroxyphenyl)pyrimidin-4-yl]-6,7- dihydropyrazolo[1,5-a]pyrazin-4(5H)-one | |
| 412 | 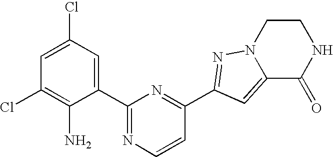 | 2-[2-(2-amino-3,5-dichlorophenyl) pyrimidin-4-yl]-6,7- dihydropyrazolo[1,5-a]pyrazin- 4(5H)-one | |
| 413 | 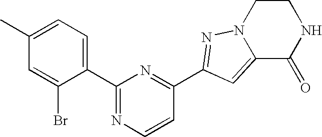 | 2-[2-(2-bromo-4-methylphenyl) pyrimidin-4-yl]-6,7- dihydropyrazolo[1,5-a]pyrazin- 4(5H)-one | |
| 414 | 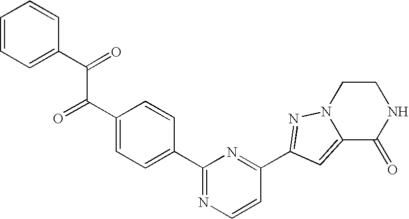 | 1-{4-[4-(4-oxo-4,5,6,7- tetrahydropyrazolo[1,5-a]pyrazin-2-yl)pyrimidin-2- yl]phenyl}-2-phenylethane- 1,2-dione | |
| 415 | 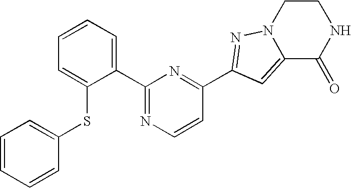 | 2-{2-[2-(phenylthio)phenyl]pyrimidin- 4-yl}-6,7-dihydropyrazolo [1,5-a]pyrazin-4(5H)-one | |
| 416 | 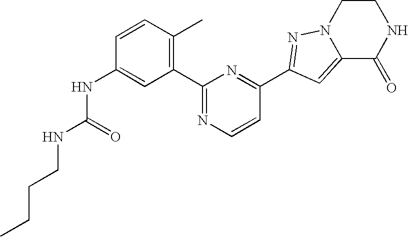 | N-butyl-N[40 -{4-methyl-3-[4-(4- oxo-4,5,6,7-tetrahydropyrazolo[1,5- a]pyrazin-2-yl)pyrimidin- 2-yl]phenyl}urea | |
| 417 | 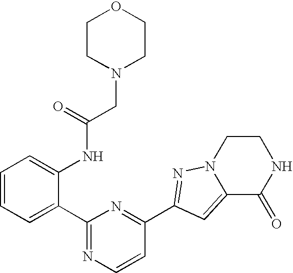 | 2-morpholin-4-yl-N-{2-[4-(4-oxo- 4,5,6,7-tetrahydropyrazolo[1,5-a]pyrazin-2-yl)pyrimidin-2- yl]phenyl}acetamide | |
| 418 | 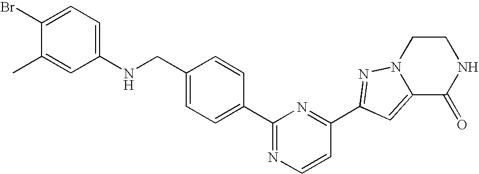 | 2-[2-(4-{[(4-bromo-3- methylphenyl)amino]methyl}phenyl)pyrimidin-4-yl]-6,7- dihydropyrazolo[1,5-a]pyrazin- 4(5H)-one | |
| 419 | 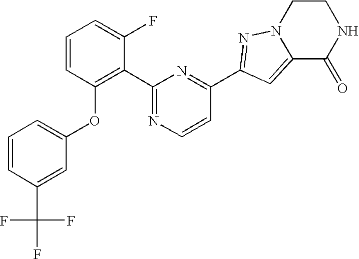 | 2-(2-{2-fluoro-6-[3- (trifluoromethyl)phenoxy]phenyl}pyrimidin-4-yl)-6,7- dihydropyrazolo[1,5-a]pyrazin-4(5H)-one | |
| 420 | 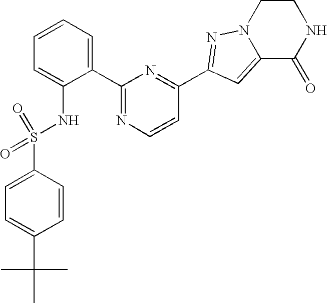 | 4-tert-butyl-N-{2-[4-(4-oxo-4,5,6,7- tetrahydropyrazolo[1,5-a]pyrazin-2- yl)pyrimidin-2- yl]phenyl}benzenesulfonamide | |
| 421 | 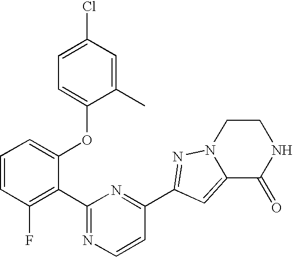 | 2-{2-[2-(4-chloro-2-methylphenoxy)-6- fluorophenyl]pyrimidin-4-yl}-6,7- dihydropyrazolo[1,5-a]pyrazin- 4(5H)-one | |
| 422 | 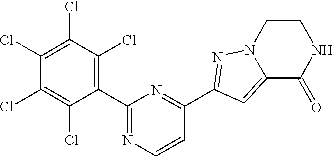 | 2-[2-(pentachlorophenyl)pyrimidin-4- yl]-6,7-dihydropyrazolo [1,5-a]pyrazin-4(5H)-one | |
| 423 | 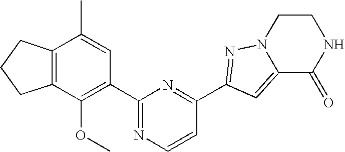 | 2-[2-(4-methoxy-7-methyl-2,3-dihydro- 1H-inden-5-yl)pyrimidin-4-yl]-6,7- dihydropyrazolo[1,5-a]pyrazin- 4(5H)-one | |
| 424 | 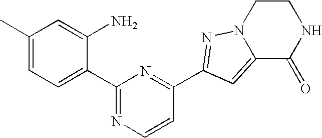 | 2-[2-(2-amino-4-methylphenyl) pyrimidin-4-yl]-6,7- dihydropyrazolo[1,5-a]pyrazin-4(5H)-one | |
| 425 | 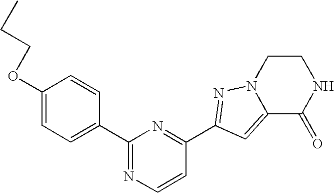 | 2-[2-(4-propoxyphenyl)pyrimidin- 4-yl]-6,7-dihydropyrazolo[1,5- a]pyrazin-4(5H)-one | |
| 426 | 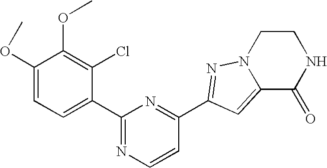 | 2-[2-(2-chloro-3,4- dimethoxyphenyl)pyrimidin-4- yl]-6,7-dihydropyrazolo [1,5-a]pyrazin-4(5H)-one | |
| 427 | 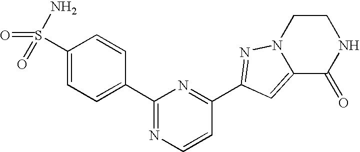 | 4-[4-(4-oxo-4,5,6,7- tetrahydropyrazolo[1,5-a]pyrazin-2-yl)pyrimidin-2- yl]benzenesulfonamide | |
| 428 | 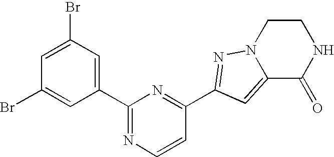 | 2-[2-(3,5-dibromophenyl)pyrimidin-4- yl]-6,7-dihydropyrazolo[1, 5-a]pyrazin-4(5H)-one | |
| 429 | 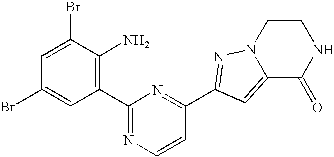 | 2-[2-(2-amino-3,5-dibromophenyl) pyrimidin-4-yl]-6,7- dihydropyrazolo[1,5-a]pyrazin- 4(5H)-one | |
| 430 | 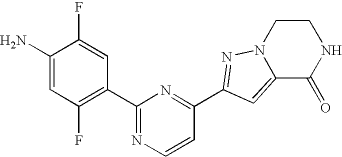 | 2-[2-(4-amino-2,5-difluorophenyl) pyrimidin-4-yl]-6,7- dihydropyrazolo[1,5-a]pyrazin- 4(5H)-one | |
| 431 | 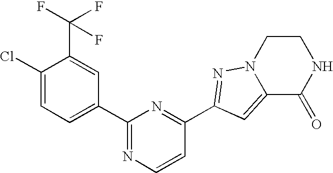 | 2-{2-[4-chloro-3- (trifluoromethyl)phenyl]pyrimidin- 4-yl}-6,7-dihydropyrazolo [1,5-a]pyrazin-4(5H)-one | |
| 432 | 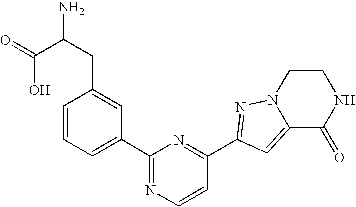 | 3-[4-(4-oxo-4,5,6,7- tetrahydropyrazolo[1,5-a]pyrazin-2-yl)pyrimidin-2- yl]phenylalanine | |
| 433 | 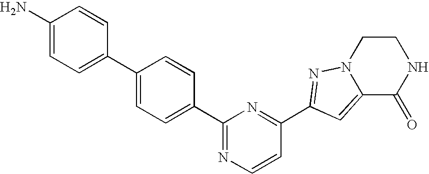 | 2-[2-(4′-amino-1,1-biphenyl-4- yl)pyrimidin-4-yl]-6,7- dihydropyrazolo[1,5-a]pyrazin- 4(5H)-one | |
| 434 | 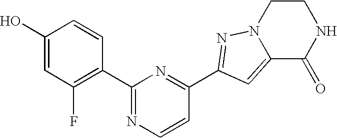 | 2-[2-(2-fluoro-4-hydroxyphenyl) pyrimidin-4-yl]-6,7- dihydropyrazolo[1,5-a]pyrazin-4(5H)-one | |
| 435 | 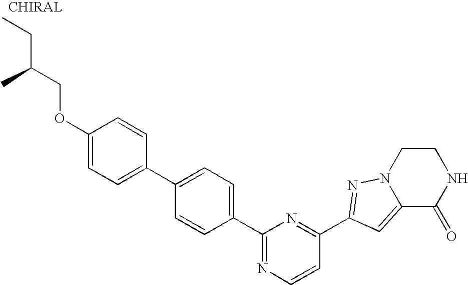 | 2-[2-(4′-{[(2S)-2-methylbutyl]oxy}-1,1′-biphenyl-4-yl)pyrimidin- 4-yl]-6,7-dihydropyrazolo[1,5-a]pyrazin-4(5H)-one | |
| 436 | 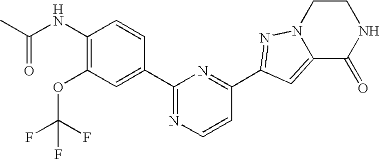 | N-[4-[4-(4-oxo-4,5,6,7- tetrahydropyrazolo[1,5-a]pyrazin-2- yl)pyrimidin-2-yl]-2- (trifluoromethoxy)phenyl]acetamide | |
| 437 | 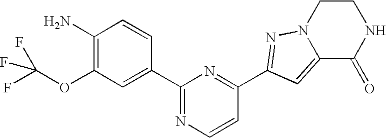 | 2-{2-[4-amino-3- (trifluoromethoxy)phenyl]pyrimidin- 4-yl}-6,7- dihydropyrazolo[1,5-a]pyrazin-4(5H)-one | |
| 438 | 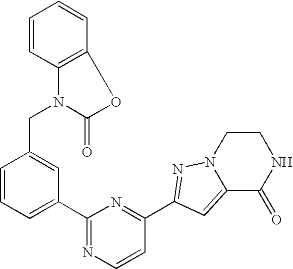 | 2-(2-{3-[(2-oxo-1,3-benzoxazol-3(2H)- yl)methyl]phenyl}pyrimidin- 4-yl)-6,7-dihydropyrazolo [1,5-a]pyrazin-4(5H)-one | |
| 439 | 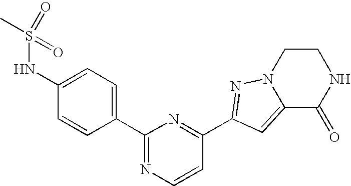 | N-{4-[4-(4-oxo-4,5,6,7- tetrahydropyrazolo[1,5-a]pyrazin-2- yl)pyrimidin-2- yl]phenyl}methanesulfonamide | |
| 440 | 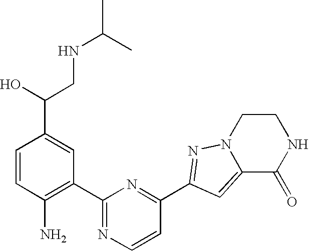 | 2-(2-{2-amino-5-[1-hydroxy-2- (isopropylamino)ethyl]phenyl}pyrimidin-4-yl)-6,7- dihydropyrazolo[1,5-a]pyrazin- 4(5H)-one | |
| 441 | 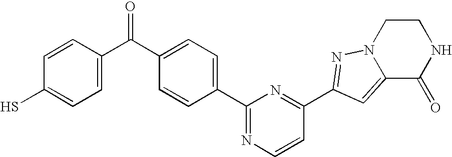 | 2-{2-[4-(4- mercaptobenzoyl)phenyl]pyrimidin- 4-yl}-6,7- dihydropyrazolo[1,5-a]pyrazin- 4(5H)-one | |
| 442 | 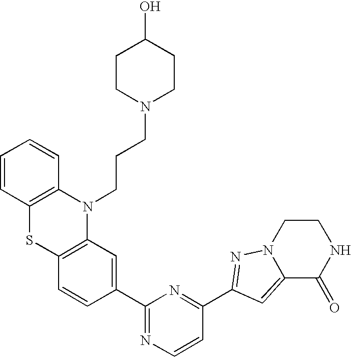 | 2-(2-{10-[3-(4-hydroxypiperidin-1-yl) propyl]-10H-phenothiazin- 2-yl}pyrimidin-4-yl)- 6,7-dihydropyrazolo [1,5-a]pyrazin-4(5H)-one | |
| 443 | 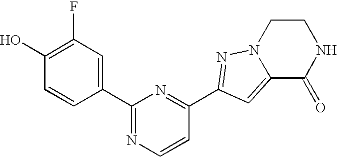 | 2-[2-(3-fluoro-4-hydroxyphenyl) pyrimidin-4-yl]-6,7- dihydropyrazolo[1,5-a]pyrazin- 4(5H)-one | |
| 444 | 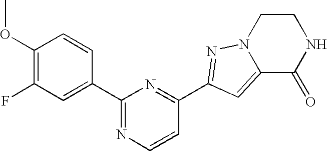 | 2-[2-(3-fluoro-4-methoxyphenyl) pyrimidin-4-yl]-6,7- dihydropyrazolo[1,5-a]pyrazin- 4(5H)-one | |
| 445 | 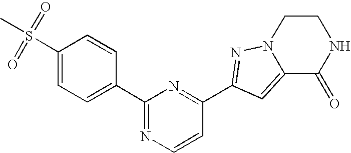 | 2-{2-[4-(methylsulfonyl) phenyl]pyrimidin-4-yl}-6,7- dihydropyrazolo[1,5-a]pyrazin- 4(5H)-one | |
| 446 | 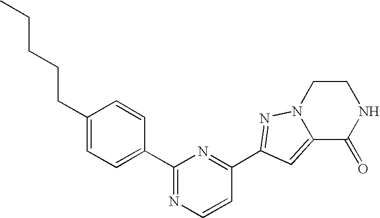 | 2-[2-(4-pentylphenyl)pyrimidin-4- yl]-6,7-dihydropyrazolo [1,5-a]pyrazin-4(5H)-one | |
| 447 | 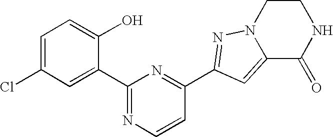 | 2-[2-(5-chloro-2-hydroxyphenyl) pyrimidin-4-yl]-6,7- dihydropyrazolo[1,5-a]pyrazin- 4(5H)-one | |
| 448 | 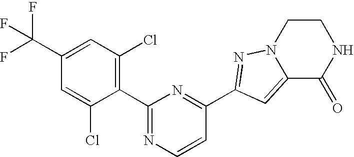 | 2-{2-[2,6-dichloro-4- (trifluoromethyl)phenyl]pyrimidin- 4-yl}-6,7- dihydropyrazolo[1,5-a]pyrazin-4(5H)-one | |
| 449 | 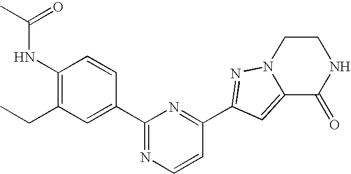 | N-{2-ethyl-4-[4-(4-oxo-4,5,6,7- tetrahydropyrazolo[1,5-a]pyrazin-2-yl)pyrimidin- 2-yl]phenyl}acetamide | |
| 450 | 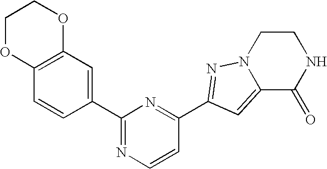 | 2-[2-(2,3-dihydro-1,4-benzodioxin-6- yl)pyrimidin-4-yl]-6,7- dihydropyrazolo[1,5-a]pyrazin-4(5H)-one | |
| 451 | 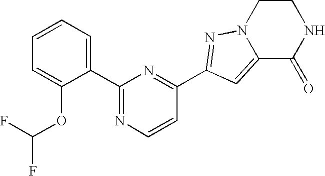 | 2-{2-[2-(difluoromethoxy)phenyl]pyrimidin-4-yl}-6,7- dihydropyrazolo[1,5-a]pyrazin-4(5H)-one | |
| 452 | 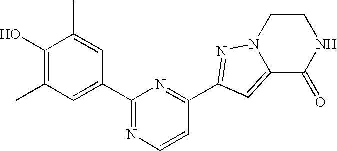 | 2-[2-(4-hydroxy-3,5- dimethylphenyl)pyrimidin-4-yl]-6,7- dihydropyrazolo[1,5-a]pyrazin- 4(5H)-one | |
| 453 | 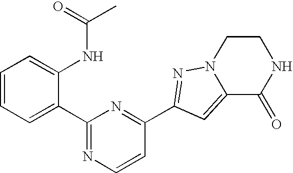 | N-{2-[4-(4-oxo-4,5,6,7- tetrahydropyrazolo[1,5-a]pyrazin-2- yl)pyrimidin-2-yl]phenyl}acetamide | |
| 454 | 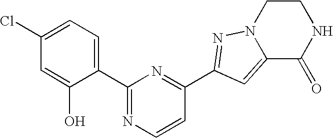 | 2-[2-(4-chloro-2-hydroxyphenyl) pyrimidin-4-yl]-6,7- dihydropyrazolo[1,5-a]pyrazin-4(5H)-one | |
| 455 | 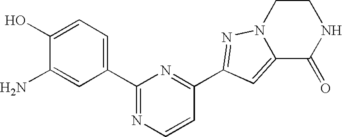 | 2-[2-(3-amino-4-hydroxyphenyl) pyrimidin-4-yl]-6,7- dihydropyrazolo[1,5-a]pyrazin-4(5H)-one | |
| 456 | 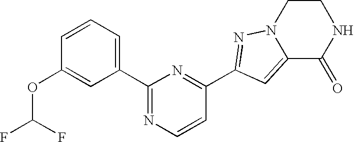 | 2-{2-[3-(difluoromethoxy)phenyl]pyrimidin-4-yl}-6,7- dihydropyrazolo[1,5-a]pyrazin- 4(5H)-one | |
| 457 | 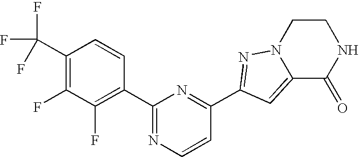 | 2-{2-(2,3-difluoro-4- (trifluoromethyl)phenyl]pyrimidin- 4-yl}-6,7-dihydropyrazolo [1,5-a]pyrazin-4(5H)-one | |
| 458 | 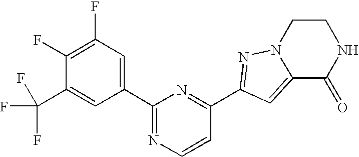 | 2-{2-[3,4-difluoro-5- (trifluoromethyl)phenyl]pyrimidin- 4-yl}-6,7-dihydropyrazolo [1,5-a]pyrazin-4(5H)-one | |
| 459 | 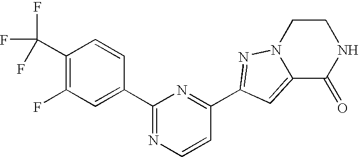 | 2-{2-[3-fluoro-4- (trifluoromethyl)phenyl]pyrimidin- 4-yl}-6,7-dihydropyrazolo [1,5-a]pyrazin-4(5H)-one | |
| 460 | 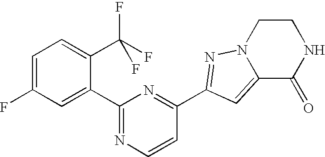 | 2-{2-[5-fluoro-2- (trifluoromethyl)phenyl]pyrimidin- 4-yl}-6,7-dihydropyrazolo [1,5-a]pyrazin-4(5H)-one | |
| 461 | 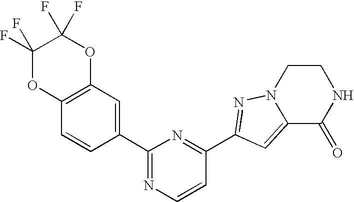 | 2-[2-(2,2,3,3-tetrafluoro-2,3- dihydro-1,4-benzodioxin-6-yl) pyrimidin-4-yl]-6,7- dihydropyrazolo[1,5-a]pyrazin-4(5H)-one | |
| 462 | 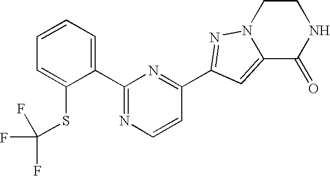 | 2-(2-{2- [(trifluoromethyl)thio]phenyl}pyrimidin-4-yl)-6,7- dihydropyrazolo[1,5-a]pyrazin-4(5H)-one | |
| 463 | 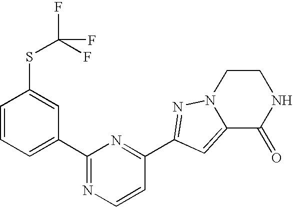 | 2-(2-{3- [(trifluoromethyl)thio]phenyl}pyrimidin-4-yl)-6,7- dihydropyrazolo[1,5-a]pyrazin-4(5H)-one | |
| 464 | 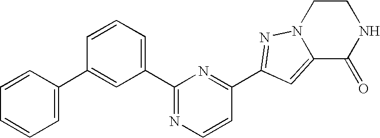 | 2-[2-(1,1′-biphenyl-3-yl)pyrimidin- 4-yl]-6,7-dihydropyrazolo[1,5-a]pyrazin-4(5H)-one | |
| 465 | 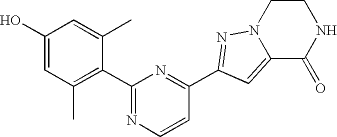 | 2-[2-(4-hydroxy-2,6- dimethylphenyl)pyrimidin-4-yl]- 6,7-dihydropyrazolo[1,5-a]pyrazin- 4(5H)-one | |
| 466 | 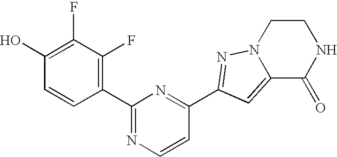 | 2-[2-(2,3-difluoro-4- hydroxyphenyl)pyrimidin-4-yl]- 6,7-dihydropyrazolo[1,5-a]pyrazin- 4(5H)-one | |
| 467 | 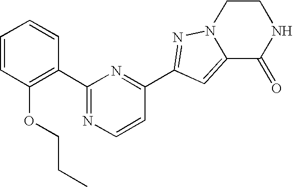 | 2-[2-(2-propoxyphenyl)pyrimidin-4- yl]-6,7-dihydropyrazolo[1,5-a]pyrazin-4(5H)-one | |
| 468 | 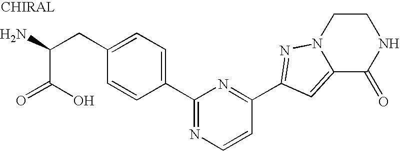 | 4-[4-(4-oxo-4,5,6,7- tetrahydropyrazolo[1,5-a]pyrazin-2-yl)pyrimidin-2- yl]-L-phenylalanine | |
| 469 | 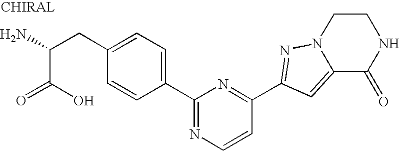 | 4-[4-(4-oxo-4,5,6,7- tetrahydropyrazolo[1,5-a]pyrazin-2-yl)pyrimidin-2- yl]-D-phenylalanine | |
| 470 | 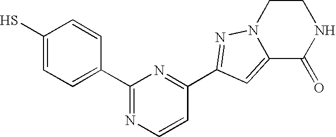 | 2-[2-(4-mercaptophenyl)pyrimidin- 4-yl]-6,7-dihydropyrazolo [1,5-a]pyrazin-4(5H)-one | |
| 471 | 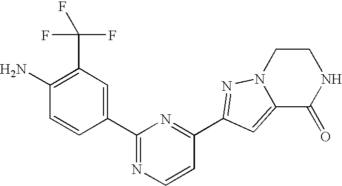 | 2-{2-(4-amino-3- (trifluoromethyl)phenyl]pyrimidin- 4-yl}-6,7-dihydropyrazolo [1,5-a]pyrazin-4(5H)-one | |
| 472 | 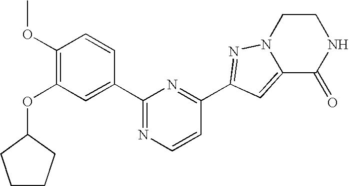 | 2-{2-[3-(cyclopentyloxy)-4- methoxyphenyl]pyrimidin-4-yl}-6,7- dihydropyrazolo[1,5-a]pyrazin-4(5H)-one | |
| 473 | 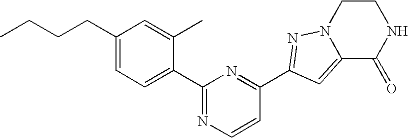 | 2-[2-(4-butyl-2-methylphenyl) pyrimidin-4-yl]-6,7- dihydropyrazolo[1,5-a]pyrazin- 4(5H)-one | |
| 474 | 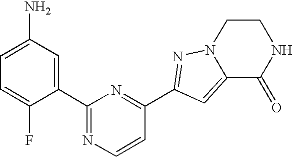 | 2-[2-(5-amino-2-fluorophenyl) pyrimidin-4-yl]-6,7- dihydropyrazolo[1,5-a]pyrazin- 4(5H)-one | |
| 475 | 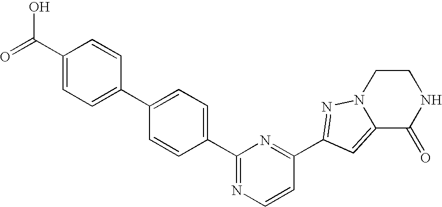 | 4′-(4-(4-oxo-4,5,6,7- tetrahydropyrazolo[1,5-a]pyrazin-2-yl]-pyrimidin-2-yl]- 1,1′-biphenyl-4- carboxylic acid | |
| 476 | 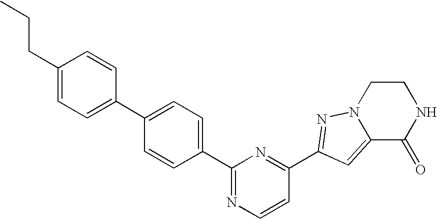 | 2-[2-(4′-propyl-1,1′-biphenyl-4- yl)pyrimidin-4-yl]-6,7- dihydropyrazolo[1,5-a]pyrazin-4(5H)-one | |
| 477 | 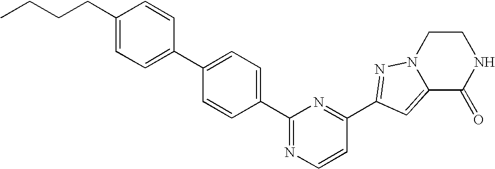 | 2-[2-(4′-butyl-1,1′-biphenyl-4- yl)pyrimidin-4-yl]-6,7- dihydropyrazolo[1,5-a]pyrazin-4(5H)-one | |
| 478 | 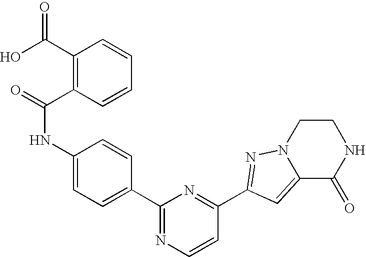 | 2-[({4-[4-(4-oxo-4,5,6,7- tetrahydropyrazolo[1,5-a]pyrazin-2- yl)pyrimidin-2-yl]phenyl}amino)carbonyl]benzoic acid | |
| 479 | 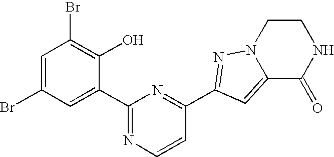 | 2-[2-(3,5-dibromo-2- hydroxyphenyl)pyrimidin-4-yl]-6,7- dihydropyrazolo[1,5-a]pyrazin-4(5H)-one | |
| 480 | 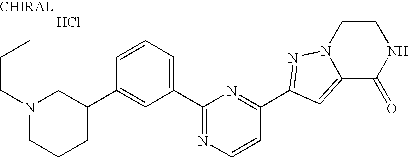 | 2-(2-{3-[(3S)-1-propylpiperidin-3- yl]phenyl}pyrimidin-4-yl)-6,7- dihydropyrazolo[1,5-a]pyrazin-4(5H)-one hydrochloride | |
| 481 | 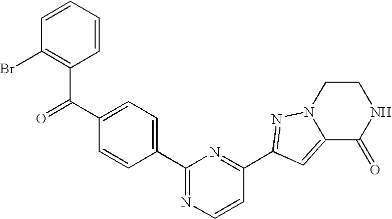 | 2-{2-[4-(2-bromobenzoyl)phenyl]pyrimidin-4-yl}-6,7- dihydropyrazolo[1,5-a]pyrazin-4(5H)-one | |
| 482 | 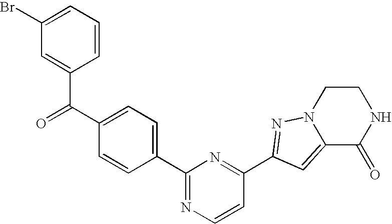 | 2-{2-[4-(3-bromobenzoyl)phenyl]pyrimidin-4-yl}-6,7- dihydropyrazolo[1,5-a]pyrazin-4(5H)-one | |
| 483 | 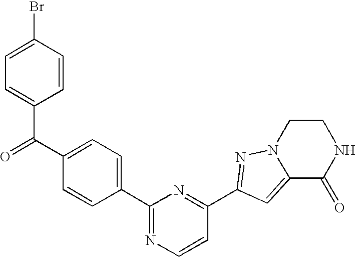 | 2-{2-[4-(4-bromobenzoyl)phenyl]pyrimidin-4-yl}-6,7- dihydropyrazolo[1,5-a]pyrazin-4(5H)-one | |
| 484 | 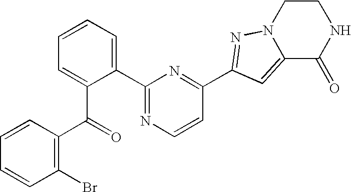 | 2-{2-[2-(2-bromobenzoyl)phenyl]pyrimidin-4-yl}-6,7- dihydropyrazolo[1,5-a]pyrazin-4(5H)-one | |
| 485 | 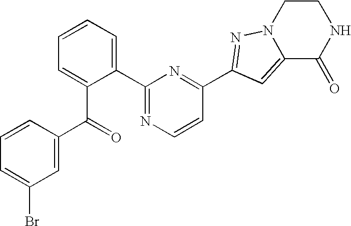 | 2-{2-[2-(3-bromobenzoyl)phenyl]pyrimidin-4-yl}-6,7- dihydropyrazolo[1,5-a]pyrazin-4(5H)-one | |
| 486 | 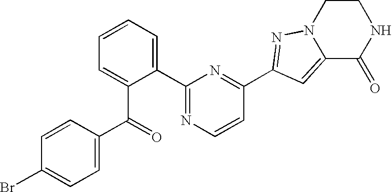 | 2-{2-[2-(4-bromobenzoyl)phenyl]pyrimidin-4-yl}-6,7- dihydropyrazolo[1,5-a]pyrazin-4(5H)-one | |
| 487 | 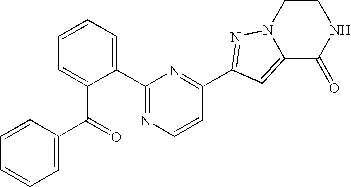 | 2-[2-(2-benzoylphenyl)pyrimidin-4-yl]- 6,7-dihydropyrazolo[1,5-a]pyrazin-4(5H)-one | |
| 488 | 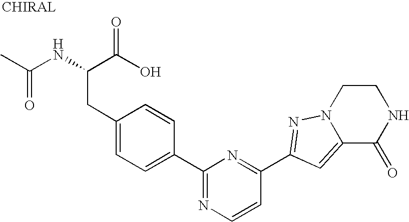 | N-acetyl-4-[4-(4-oxo-4,5,6,7- tetrahydropyrazolo[1,5-a]pyrazin-2- yl)pyrimidin-2-yl]-L-phenylalanine | |
| 489 | 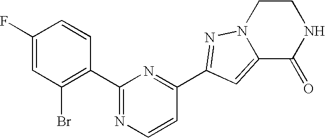 | 2-[2-(2-bromo-4-fluorophenyl) pyrimidin-4-yl]-6,7- dihydropyrazolo[1,5-a]pyrazin- 4(5H)-one | |
| 490 | 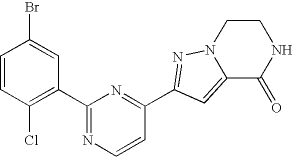 | 2-[2-(5-bromo-2-chlorophenyl) pyrimidin-4-yl]-6,7- dihydropyrazolo[1,5-a]pyrazin- 4(5H)-one | |
| 491 | 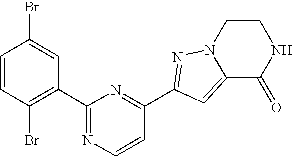 | 2-[2-(2,5-dibromophenyl)pyrimidin-4- yl]-6,7-dihydropyrazolo[1,5-a]pyrazin-4(5H)-one | |
| 492 | 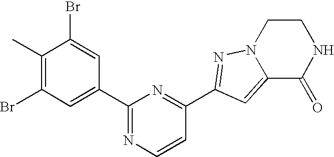 | 2-[2-(3,5-dibromo-4-methylphenyl) pyrimidin-4-yl]-6,7- dihydropyrazolo[1,5-a]pyrazin- 4(5H)-one | |
| 493 | 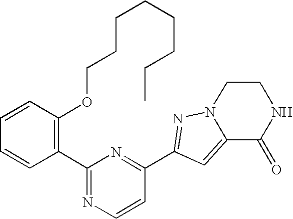 | 2-{2-[2-(octyloxy)phenyl]pyrimidin- 4-yl}-6,7-dihydropyrazolo [1,5-a]pyrazin-4(5H)-one | |
| 494 | 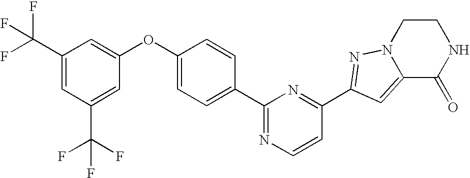 | 2-(2-{4-(3,5-bis(trifluoromethyl) phenoxy]phenyl}pyrimidin- 4-yl)-6,7-dihydropyrazolo[1,5-a]pyrazin-4(5H)-one | |
| 495 | 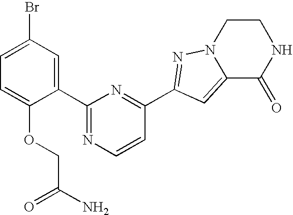 | 2-{4-bromo-2-[4-(4-oxo-4,5,6,7- tetrahydropyrazolo[1,5-a]pyrazin-2- yl)pyrimidin-2-yl]phenoxy}acetamide | |
| 496 | 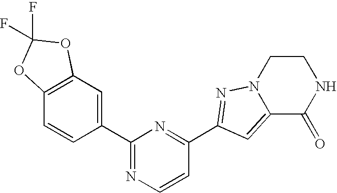 | 2-[2-(2,2-difluoro-1,3-benzodioxol-5- yl)pyrimidin-4-yl]-6,7- dihydropyrazolo[1,5-a]pyrazin-4(5H)-one | |
| 497 | 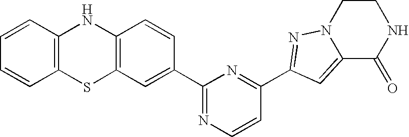 | 2-[2-(10H-phenothiazin-3-yl)pyrimidin- 4-yl]-6,7-dihydropyrazolo[1,5-a]pyrazin-4(5H)-one | |
| 498 | 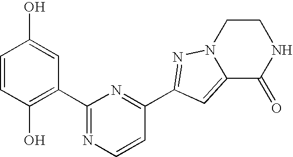 | 2-[2-(2,5-dihydroxyphenyl)pyrimidin-4- yl]-6,7-dihydropyrazolo [1,5-a]pyrazin-4(5H)- one | |
| 499 | 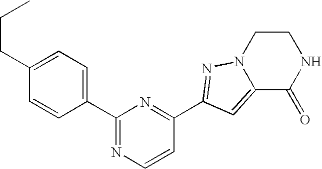 | 2-[2-(4-propylphenyl)pyrimidin-4-yl]-6, 7-dihydropyrazolo[1,5-a]pyrazin-4(5H)- one | |
| 500 | 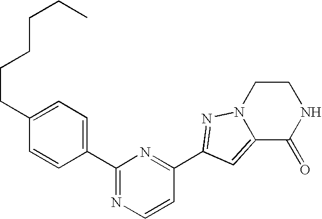 | 2-[2-(4-hexylphenyl)pyrimidin-4-yl]-6, 7-dihydropyrazolo[1,5-a]pyrazin-4(5H)- one | |
| 501 | 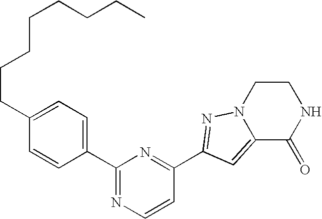 | 2-[2-(4-octylphenyl)pyrimidin-4-yl]-6,7- dihydropyrazolo[1,5-a]pyrazin-4(5H)- one | |
| 502 | 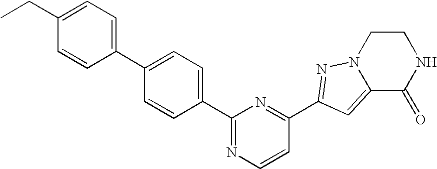 | 2-[2-(4′-ethyl-1,1′-biphenyl-4-yl) pyrimidin-4-yl]-6,7-dihydropyrazolo[1, 5-a]pyrazin-4(5H)-one | |
| 503 | 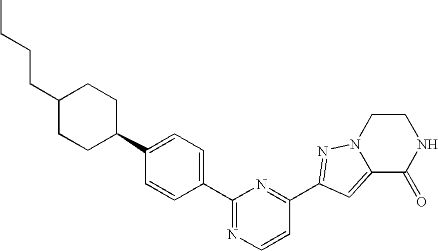 | 2-[2-[4-(4-butylcyclohexyl)phenyl]pyrimidin-4-yl}-6,7-dihydropyrazolo[1, 5-a]pyrazin-4(5H)-one | |
| 504 | 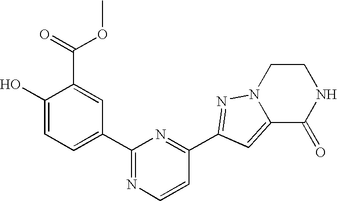 | methyl 2-hydroxy-5-[4-(4-oxo-4,5,6,7- tetrahydropyrazolo[1,5-a]pyrazin-2- yl)pyrimidin-2-yl]benzoate | |
| 505 | 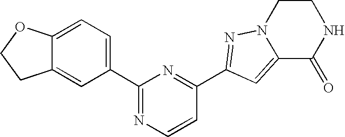 | 2-[2-(2,3-dihydro-1-benzofuran-5- yl)pyrimidin-4-yl]-6,7-dihydropyrazolo [1,5-a]pyrazin-4(5H)-one | |
| 506 | 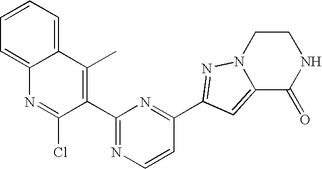 | 2-[2-(2-chloro-4-methylquinolin-3- yl)pyrimidin-4-yl]-6,7-dihydropyrazolo [1,5-a]pyrazin-4(5H)-one | |
| 507 | 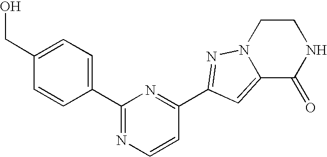 | 2-{2-[4-(hydroxymethyl)phenyl]pyrimidin-4-yl}-6,7- dihydropyrazolo[1,5-a]pyrazin-4(5H)-one | |
| 508 | 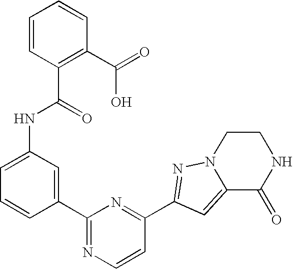 | 2-[({3-[4-(4-oxo-4,5,6,7- tetrahydropyrazolo[1,5-a]pyrazin-2- yl)pyrimidin-2-yl]phenyl}amino)carbonyl]benzoic acid | |
| 509 | 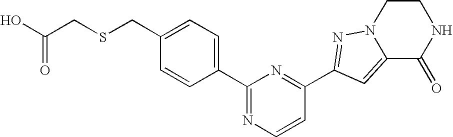 | ({4-[4-(4-oxo-4,5,6,7- tetrahydropyrazolo[1,5-a]pyrazin- 2-yl)pyrimidin-2-yl]benzyl}thio)acetic acid | |
| 510 | 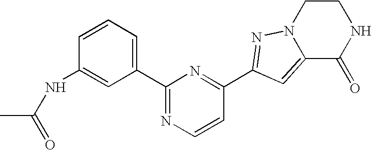 | N-{3-[4-(4-oxo-4,5,6,7- tetrahydropyrazolo[1,5-a]pyrazin-2- yl)pyrimidin-2-yl]phenyl}acetamide | |
| 511 | 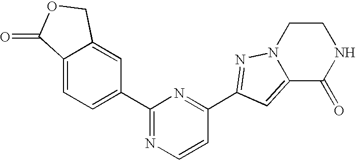 | 2-[2-(1-oxo-1,3-dihydro-2-benzofuran- 5-yl)pyrimidin-4-yl]-6,7- dihydropyrazolo[1,5-a]pyrazin-4(5H)-one | |
| 512 | 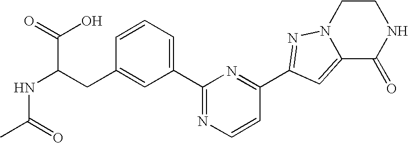 | N-acetyl-3-[4-(4-oxo-4,5,6,7- tetrahydropyrazolo[1,5-a]pyrazin-2- yl)pyrimidin-2-yl]phenylalanine | |
| 513 | 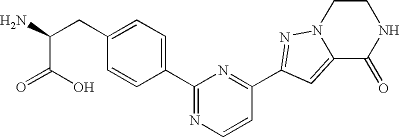 | 4-[4-(4-oxo-4,5,6,7-tetrahydropyrazolo [1,5-a]pyrazin-2-yl)pyrimidin- 2-yl]-L-phenylalanine | |
| 514 | 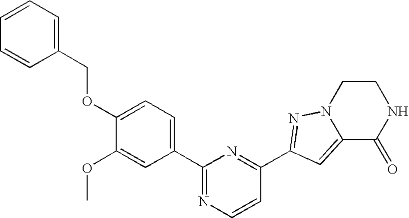 | 2-{2-[4-(benzyloxy)-3- methoxyphenyl]pyrimidin-4-yl}-6,7- dihydropyrazolo[1,5-a]pyrazin- 4(5H)-one | |
| 515 | 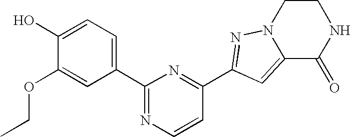 | 2-[2-(3-ethoxy-4-hydroxyphenyl) pyrimidin-4-yl]-6,7- dihydropyrazolo[1,5-a]pyrazin- 4(5H)-one | |
| 516 | 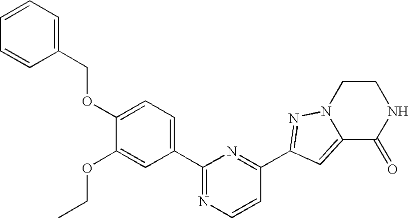 | 2-{2-[4-(benzyloxy)-3- ethoxyphenyl]pyrimidin-4-yl}-6,7- dihydropyrazolo[1,5-a]pyrazin- 4(5H)-one | |
| 517 | 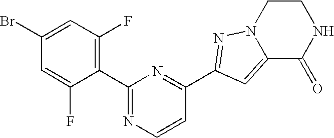 | 2-[2-(4-bromo-2,6-difluorophenyl) pyrimidin-4-yl]-6,7- dihydropyrazolo[1,5-a]pyrazin- 4(5H)-one | |
| 518 | 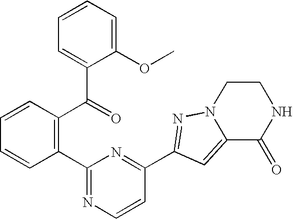 | 2-{2-[2-(2-methoxybenzoyl)phenyl]pyrimidin-4-yl}-6,7- dihydropyrazolo[1,5-a]pyrazin- 4(5H)-one | |
| 519 | 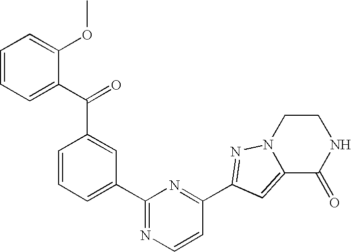 | 2-{2-[3-(2-methoxybenzoyl)phenyl]pyrimidin-4-yl}-6,7- dihydropyrazolo[1,5-a]pyrazin- 4(5H)-one | |
| 520 | 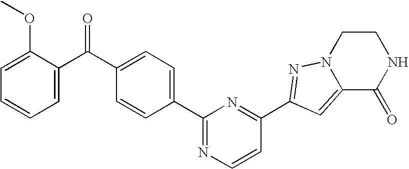 | 2-{2-[4-(2-methoxybenzoyl)phenyl]pyrimidin-4-yl}-6,7- dihydropyrazolo[1,5-a]pyrazin- 4(5H)-one | |
| 521 | 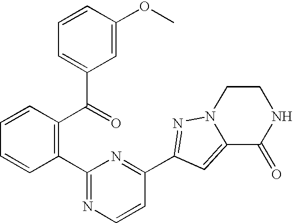 | 2-{2-[2-(3-methoxybenzoyl)phenyl]pyrimidin-4-yl}-6,7- dihydropyrazolo[1,5-a]pyrazin- 4(5H)-one | |
| 522 | 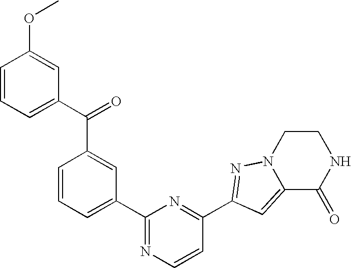 | 2-{2-[3-(3-methoxybenzoyl)phenyl]pyrimidin-4-yl}-6,7- dihydropyrazolo[1,5-a]pyrazin- 4(5H)-one | |
| 523 | 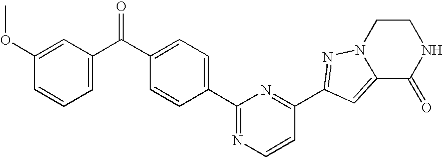 | 2-{2-[4-(3-methoxybenzoyl)phenyl]pyrimidin-4-yl}-6,7- dihydropyrazolo[1,5-a]pyrazln- 4(5H)-one | |
| 524 | 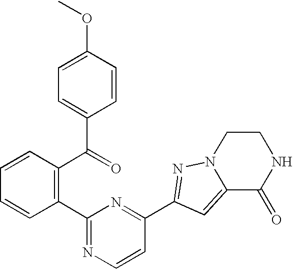 | 2-{2-[2-(4-methoxybenzoyl)phenyl]pyrimidin-4-yl}-6,7- dihydropyrazolo[1,5-a]pyrazin- 4(5H)-one | |
| 525 | 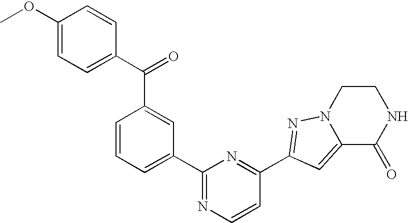 | 2-{2-[3-(4-methoxybenzoyl)phenyl]pyrimidin-4-yl}-6,7- dihydropyrazolo[1,5-a]pyrazin- 4(5H)-one | |
| 526 | 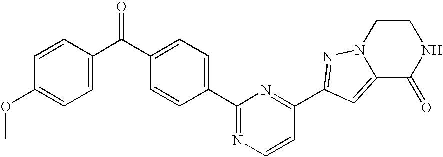 | 2-{2-[4-(4-methoxybenzoyl)phenyl]pyrimidin-4-yl}-6,7- dihydropyrazolo[1,5-a]pyrazin- 4(5H)-one | |
| 527 | 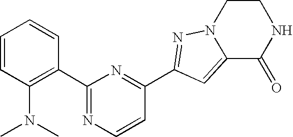 | 2-{2-[2-(dimethylamino)phenyl]pyrimidin-4-yl}-6,7- dihydropyrazolo[1,5-a]pyrazin- 4(5H)-one | |
| 528 | 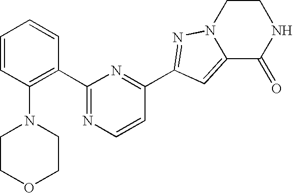 | 2-[2-(2-morpholin-4-ylphenyl)pyrimidin- 4-yl]-6,7-dihydropyrazolo[1,5-a]pyrazin-4(5H)-one | |
| 529 | 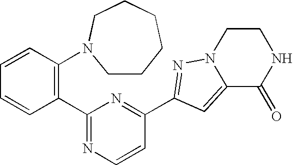 | 2-[2-(2-azepan-1-ylphenyl)pyrimidin-4- yl]-6,7-dihydropyrazolo [1,5-a]pyrazin-4(5H)-one | |
| 530 | 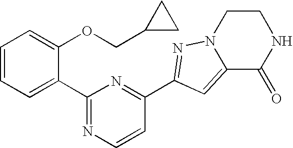 | 2-{2-[2-(cyclopropylmethoxy)phenyl]pyrimidin-4-yl}-6,7- dihydropyrazolo[1,5-a]pyrazin- 4(5H)-one | |
| 531 | 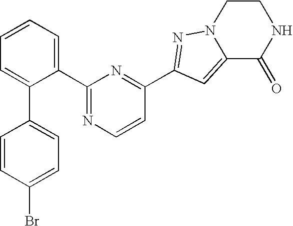 | 2-[2-(4′-bromo-1,1′-biphenyl-2-yl) pyrimidin-4-yl]-6,7-dihydropyrazolo [1,5-a]pyrazin-4(5H)-one | |
| 532 | 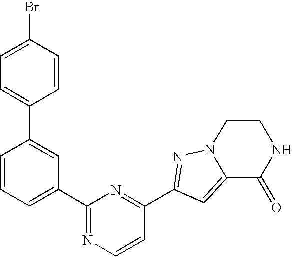 | 2-[2-(4′-bromo-1,1′-biphenyl-3-yl) pyrimidin-4-yl]-6,7-dihydropyrazolo [1,5-a]pyrazin-4(5H)-one | |
| 533 | 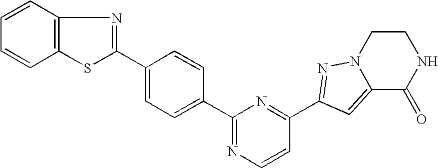 | 2-{2-[4-(1,3-benzothiazol-2- yl)phenyl]pyrimidin-4-yl}-6,7- dihydropyrazolo[1,5-a]pyrazin- 4(5H)-one | |
| 534 | 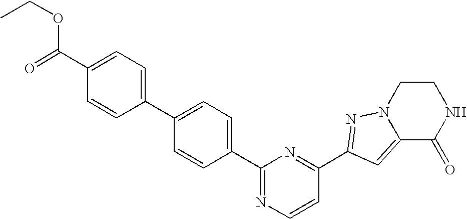 | ethyl 4′-[4-(4-oxo-4,5,6,7- tetrahydropyrazolo[1,5-a]pyrazin-2- yl)pyrimidin-2-yl]-1,1′-biphenyl-4- carboxylate | |
| 535 | 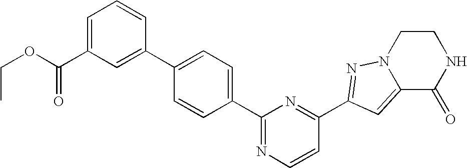 | ethyl 4′-[4-(4-oxo-4,5,6,7- tetrahydropyrazolo[1,5-a]pyrazin-2- yl)pyrimidin-2-yl]-1,1′-biphenyl-3- carboxylate | |
| 536 | 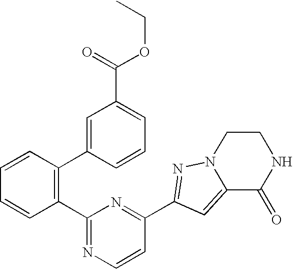 | ethyl 2′-[4-(4-oxo-4,5,6,7- tetrahydropyrazolo[1,5-a]pyrazin-2- yl)pyrimidin-2-yl]-1,1′-biphenyl-3- carboxylate | |
| 537 | 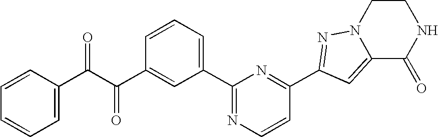 | 1-{3-[4-(4-oxo-4,5,6,7- tetrahydropyrazolo[1,5-a]pyrazin-2- yl)pyrimidin-2-yl]phenyl}-2- phenylethane-1,2-dione | |
| 538 | 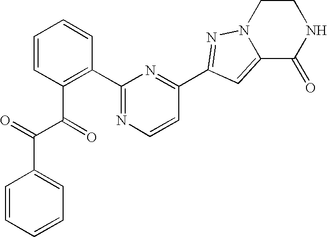 | 1-{2-[4-(4-oxo-4,5,6,7- tetrahydropyrazolo[1,5-a]pyrazin-2- yl)pyrimidin-2-yl]phenyl}-2- phenylethane-1,2-dione | |
| 539 | 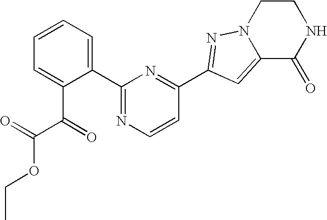 | ethyl oxo{2-[4-(4-oxo-4,5,6,7- tetrahydropyrazolo[1,5-a]pyrazin-2- yl)pyrimidin-2-yl]phenyl}acetate | |
| 540 | 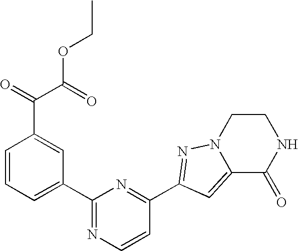 | ethyl oxo{3-[4-(4-oxo-4,5,6,7- tetrahydropyrazolo[1,5-a]pyrazin-2- yl)pyrimidin-2-yl]phenyl}acetate | |
| 541 | 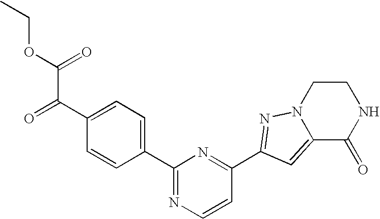 | ethyl oxo{4-[4-(4-oxo-4,5,6,7- tetrahydropyrazolo[1,5-a]pyrazin-2- yl)pyrimidin-2-yl]phenyl}acetate | |
| 542 | 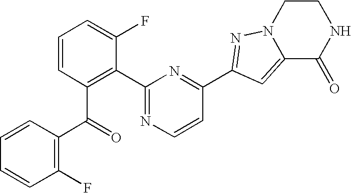 | 2-{2-[2-fluoro-6-(2-fluorobenzoyl) phenyl]pyrimidin-4-yl}-6,7- dihydropyrazolo[1,5-a]pyrazin- 4(5H)-one | |
| 543 | 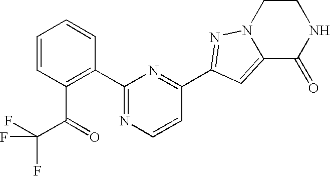 | 2-{2-[2-(trifluoroacetyl)phenyl]pyrimidin-4-yl}-6,7- dihydropyrazolo[1,5-a]pyrazin- 4(5H)-one | |
| 544 | 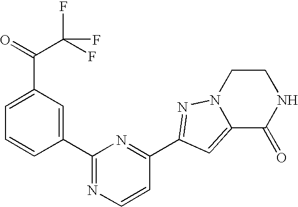 | 2-{2-[3-(trifluoroacetyl)phenyl]pyrimidin-4-yl}-6,7- dihydropyrazolo[1,5-a]pyrazin- 4(5H)-one | |
| 545 | 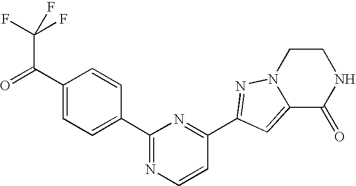 | 2-{2-[4-(trifluoroacetyl)phenyl]pyrimidin-4-yl}-6,7- dihydropyrazolo[1,5-a]pyrazin- 4(5H)-one | |
| 546 | 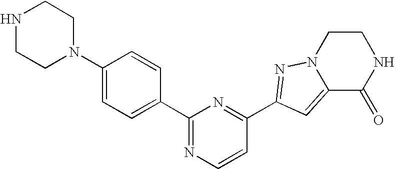 | 2-[2-(4-piperazin-1-ylphenyl)pyrimidin- 4-yl]-6,7-dihydropyrazolo[1,5-a]pyrazin-4(5H)-one | |
| 547 | 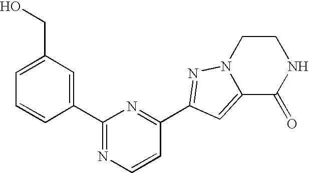 | 2-{2-[3-(hydroxymethyl)phenyl]pyrimidin-4-yl}-6,7- dihydropyrazolo[1,5-a]pyrazin- 4(5H)-one | |
| 548 | 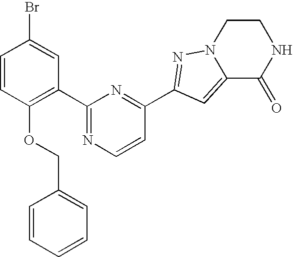 | 2-{2-[2-(benzyloxy)-5-bromophenyl]pyrimidin-4-yl}-6,7- dihydropyrazolo[1,5-a]pyrazin- 4(5H)-one | |
| 549 | 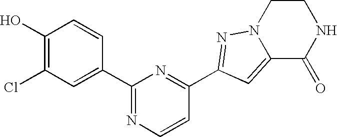 | 2-[2-(3-chloro-4-hydroxyphenyl) pyrimidin-4-yl]-6,7- dihydropyrazolo[1,5-a]pyrazin- 4(5H)-one | |
| 550 | 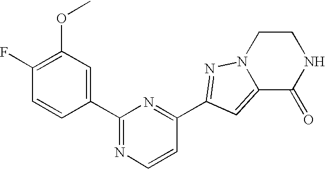 | 2-[2-(4-fluoro-3-methoxyphenyl) pyrimidin-4-yl]-6,7- dihydropyrazolo[1,5-a]pyrazin- 4(5H)-one | |
| 551 | 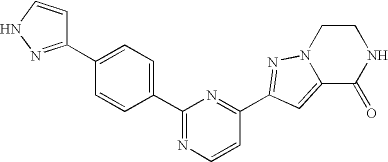 | 2-{2-[4-(1H-pyrazol-3-yl)phenyl]pyrimidin-4-yl}-6,7- dihydropyrazolo[1,5-a]pyrazin- 4(5H)-one | |
| 552 | 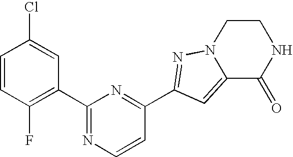 | 2-[2-(5-chloro-2-fluorophenyl) pyrimidin-4-yl]-6,7- dihydropyrazolo[1,5-a]pyrazin- 4(5H)-one | |
| 553 | 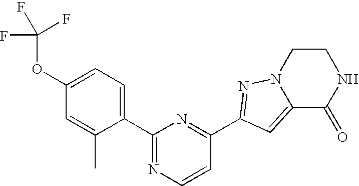 | 2-{2-[2-methyl-4-(trifluoromethoxy) phenyl]pyrimidin-4-yl}-6,7- dihydropyrazolo[1,5-a]pyrazin- 4(5H)-one | |
| 554 | 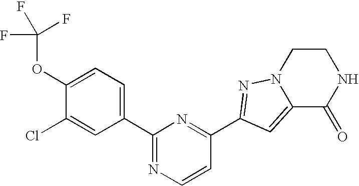 | 2-{2-[3-chloro-4-(trifluoromethoxy) phenyl]pyrimidin-4-yl}-6,7- dihydropyrazolo[1,5-a]pyrazin- 4(5H)-one | |
| 555 | 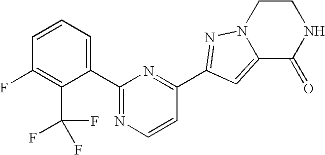 | 2-{2-[3-fluoro-2-(trifluoromethyl) phenyl]pyrimidin-4-yl}-6,7- dihydropyrazolo[1,5-a]pyrazin- 4(5H)-one | |
| 556 | 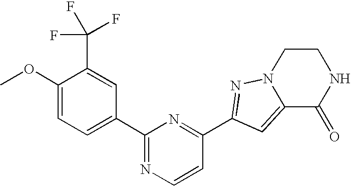 | 2-{2-[4-methoxy-3-(trifluoromethyl) phenyl]pyrimidin-4-yl}-6,7- dihydropyrazolo[1,5-a]pyrazin- 4(5H)-one | |
| 557 | 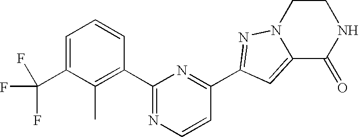 | 2-{2-[2-methyl-3-(trifluoromethyl) phenyl]pyrimidin-4-yl}-6,7- dihydropyrazolo[1,5-a]pyrazin- 4(5H)-one | |
| 558 | 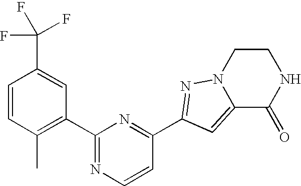 | 2-{2-[2-methyl-5-(trifluoromethyl) phenyl]pyrimidin-4-yl}-6,7- dihydropyrazolo[1,5-a]pyrazin- 4(5H)-one | |
| 559 | 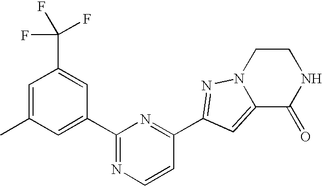 | 2-{2-[3-methyl-5-(trifluoromethyl) phenyl]pyrimidin-4-yl}-6,7- dihydropyrazolo[1,5-a]pyrazin- 4(5H)-one | |
| 560 | 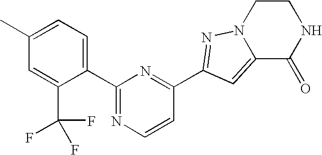 | 2-{2-[4-methyl-2-(trifluoromethyl) phenyl]pyrimidin-4-yl}-6,7- dihydropyrazolo[1,5-a]pyrazin- 4(5H)-one | |
| 561 | 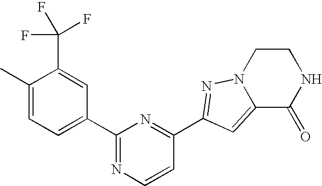 | 2-{2-[4-methyl-3-(trifluoromethyl) phenyl]pyrimidin-4-yl}-6,7- dihydropyrazolo[1,5-a]pyrazin- 4(5H)-one | |
| 562 | 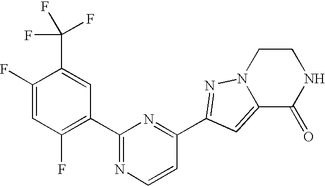 | 2-{2-[2,4-difluoro-5-(trifluoromethyl) phenyl]pyrimidin-4-yl}-6,7- dihydropyrazolo[1,5-a]pyrazin- 4(5H)-one | |
| 563 | 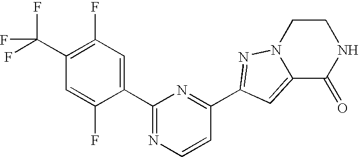 | 2-{2-[2,5-difluoro-4-(trifluoromethyl) phenyl]pyrimidin-4-yl}-6,7- dihydropyrazolo[1,5-a]pyrazin- 4(5H)-one | |
| 564 | 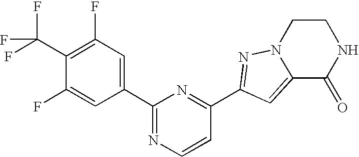 | 2-{2-[3,5-difluoro-4-(trifluoromethyl) phenyl]pyrimidin-4-yl}-6,7- dihydropyrazolo[1,5-a]pyrazin- 4(5H)-one | |
| 565 | 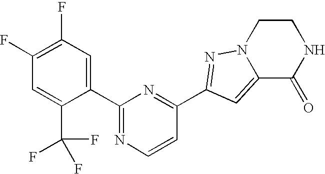 | 2-{2-[4,5-difluoro-2-(trifluoromethyl) phenyl]pyrimidin-4-yl}-6,7- dihydropyrazolo[1,5-a]pyrazin- 4(5H)-one | |
| 566 | 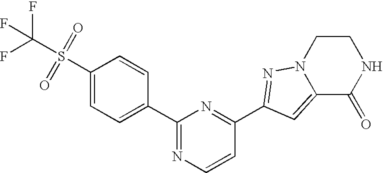 | 2-(2-{4-(trifluoromethyl)sulfonyl]phenyl}pyrimidin-4-yl)-6,7- dihydropyrazolo[1,5-a]pyrazin- 4(5H)-one | |
| 567 | 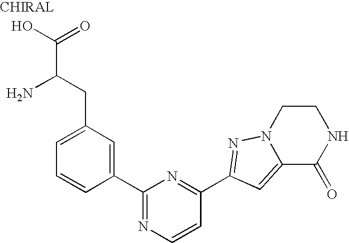 | 3-[4-(4-oxo-4,5,6,7-tetrahydropyrazolo [1,5-a]pyrazin-2-yl) pyrimidin-2-yl]-D- phenylalanine | |
| 568 | 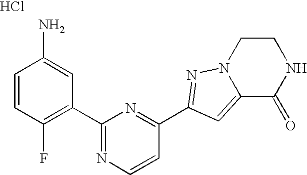 | 2-[2-(5-amino-2-fluorophenyl) pyrimidin-4-yl]-6,7- dihydropyrazolo[1,5-a]pyrazin- 4(5H)-one hydrochloride | |
| 569 | 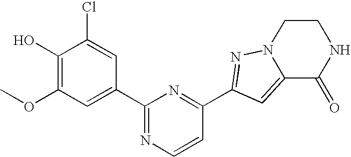 | 2-[2-(3-chloro-4-hydroxy-5- methoxyphenyl)pyrimidin-4-yl]-6,7- dihydropyrazolo[1,5-a]pyrazin- 4(5H)-one | |
| 570 | 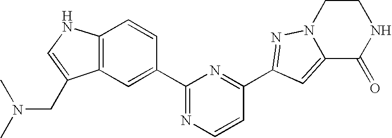 | 2-(2-{3-[(dimethylamino)methyl]- 1H-indol-5-yl}pyrimidin-4-yl)- 6,7-dihydropyrazolo[1,5-a]pyrazin-4(5H)-one | |
| 571 | 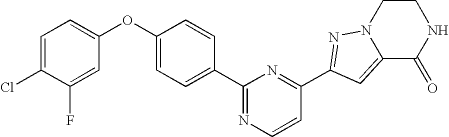 | 2-{2-[4-(4-chloro-3- fluorophenoxy)phenyl]pyrimidin- 4-yl}-6,7-dihydropyrazolo [1,5-a]pyrazin-4(5H)-one | |
| 572 | 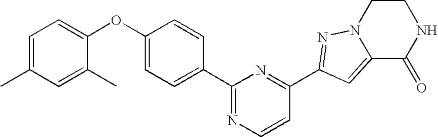 | 2-{2-(4-(2,4-dimethylphenoxy) phenyl]pyrimidin-4-yl}-6,7- dihydropyrazolo[1,5-a]pyrazin- 4(5H)-one | |
| 573 | 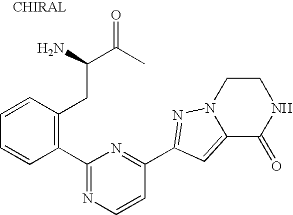 | 2-[4-(4-oxo-4,5,6,7-tetrahydropyrazolo [1,5-a]pyrazin-2-yl)pyrimidin-2- yl]-D-phenylalanine | |
| 574 | 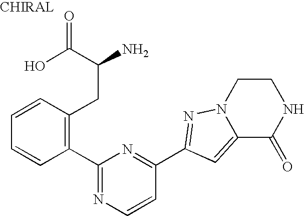 | 2-[4-(4-oxo-4,5,6,7-tetrahydropyrazolo [1,5-a]pyrazin-2-yl)pyrimidin-2- yl]-L-phenylalanine | |
| 575 | 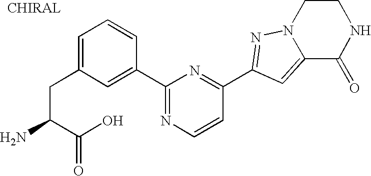 | 3-[4-(4-oxo-4,5,6,7-tetrahydropyrazolo [1,5-a]pyrazin-2-yl)pyrimidin-2- yl]-L-phenylalanine | |
| 576 | 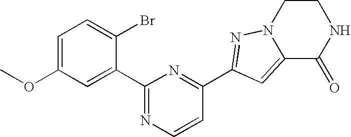 | 2-[2-(2-bromo-5-methoxyphenyl) pyrimidin-4-yl]-6,7- dihydropyrazolo[1,5-a]pyrazin- 4(5H)-one | |
| 577 | 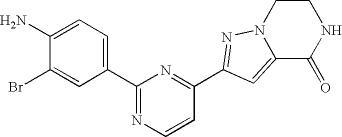 | 2-[2-(4-amino-3-bromophenyl) pyrimidin-4-yl]-6,7- dihydropyrazolo[1,5-a]pyrazin- 4(5H)-one | |
| 578 | 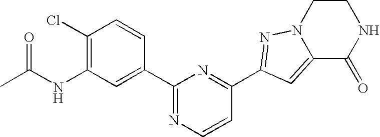 | N-{2-chloro-5-[4-(4-oxo-4,5,6,7- tetrahydropyrazolo[1,5-a]pyrazin-2- yl)pyrimidin-2-yl]phenyl}acetamide | |
| 579 | 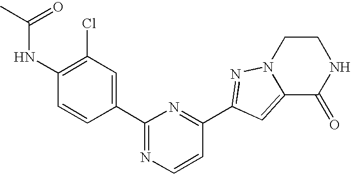 | N-{2-chloro-4-[4-(4-oxo-4,5,6,7- tetrahydropyrazolo[1,5-a]pyrazin-2- yl)pyrimidin-2-yl]phenyl}acetamide | |
| 580 | 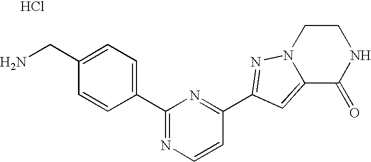 | 2-{2-[4-(aminomethyl)phenyl]pyrimidin-4-yl}-6,7- dihydropyrazolo[1,5-a]pyrazin- 4(5H)-one hydrochloride | |
| 581 | 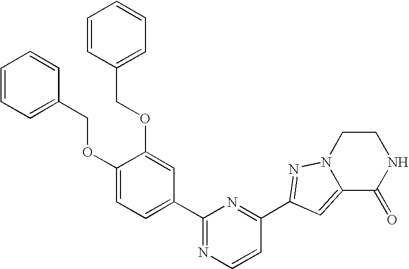 | 2-{2-[3,4-bis(benzyloxy)phenyl]pyrimidin-4-yl}-6,7- dihydropyrazolo[1,5-a]pyrazin- 4(5H)-one | |
| 582 | 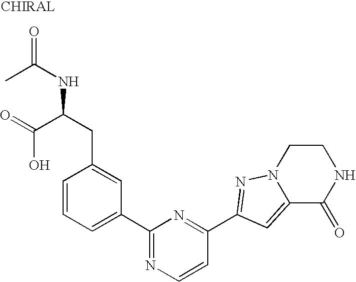 | N-acetyl-3-[4-(4-oxo-4,5,6,7- tetrahydropyrazolo[1,5-a]pyrazin-2- yl)pyrimidin-2-yl]-L-phenylalanine | |
| 583 | 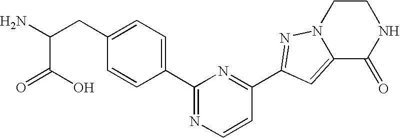 | 4-[4-(4-oxo-4,5,6,7-tetrahydropyrazolo [1,5-a]pyrazin-2-yl)pyrimidin- 2-yl]phenylalanine | |
| 584 | 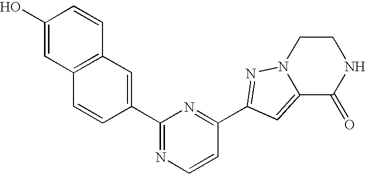 | 2-[2-(6-hydroxy-2-naphthyl) pyrimidin-4-yl]-6,7- dihydropyrazolo[1,5-a]pyrazin- 4(5H)-one | |
| 585 | 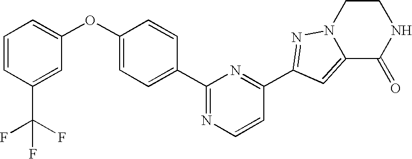 | 2-(2-{4-[3-(trifluoromethyl) phenoxy]phenyl}pyrimidin-4- yl)-6,7-dihydropyrazolo[1,5-a]pyrazin-4(5H)-one | |
| 586 | 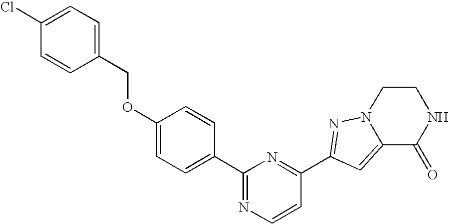 | 2-(2-{4-[(4-chlorobenzyl)oxy]phenyl}pyrimidin-4-yl)- 6,7-dihydropyrazolo[1,5-a]pyrazin-4(5H)-one | |
| 587 | 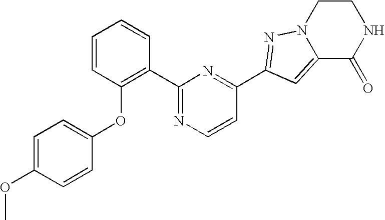 | 2-{2-[2-(4-methoxyphenoxy)phenyl]pyrimidin-4-yl}-6,7- dihydropyrazolo[1,5-a]pyrazin-4(5H)-one | |
| 588 | 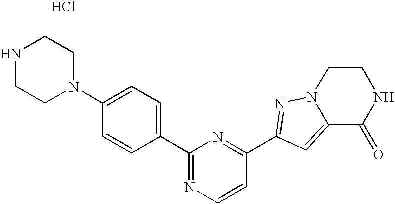 | 2-[2-(4-piperazin-1-ylphenyl)pyrimidin- 4-yl]-6,7-dihydropyrazolo [1,5-a]pyrazin-4(5H)- one hydrochloride | |
| 589 | 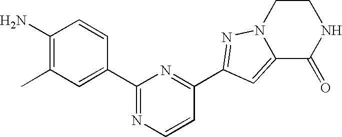 | 2-[2-(4-amino-3-methylphenyl) pyrimidin-4-yl]-6,7- dihydropyrazolo[1,5-a]pyrazin- 4(5H)-one | |
| 590 | 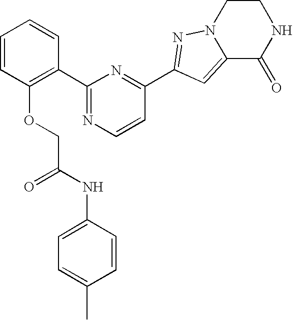 | N-(4-methylphenyl)-2-{2-[4-(4-oxo- 4,5,6,7-tetrahydropyrazolo [1,5-a]pyrazin-2-yl) pyrimidin-2-yl]phenoxy}acetamide | |
| 591 | 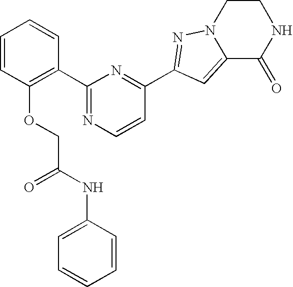 | 2-{2-[4-(4-oxo-4,5,6,7- tetrahydropyrazolo[1,5-a]pyrazin-2- yl)pyrimidin-2-yl]phenoxy}-N- phenylacetamide | |
| 592 | 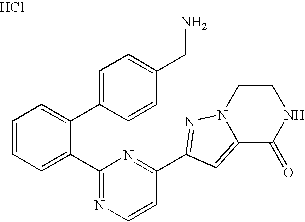 | 2-{2-[4′-(aminomethyl)-1,1′- biphenyl-2-yl]pyrimidin-4-yl}- 6,7-dihydropyrazolo[1,5- a]pyrazin-4(5H)-one hydrochloride | |
| 593 | 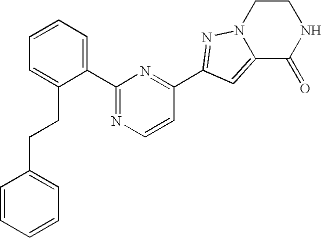 | 2-{2-[2-(2-phenylethyl)phenyl]pyrimidin-4-yl}-6,7- dihydropyrazolo[1,5-a]pyrazin- 4(5H)-one | |
| 594 | 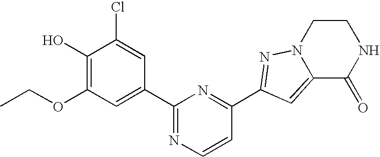 | 2-[2-(3-chloro-5-ethoxy-4- hydroxyphenyl)pyrimidin-4-yl]- 6,7-dihydropyrazolo[1,5-a]pyrazin-4(5H)-one | |
| 595 | 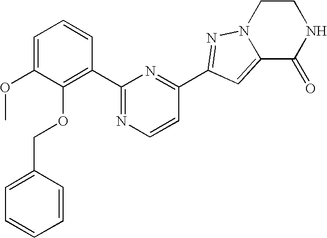 | 2-{2-[2-(benzyloxy)-3- methoxyphenyl]pyrimidin-4-yl}-6,7- dihydropyrazolo[1,5-a]pyrazin- 4(5H)-one | |
| 596 | 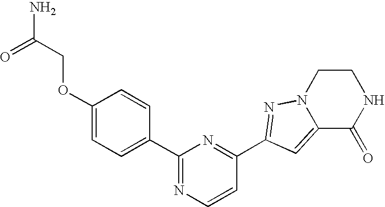 | 2-{4-[4-(4-oxo-4,5,6,7- tetrahydropyrazolo[1,5-a]pyrazin- 2-yl)pyrimidin-2-yl]phenoxy}acetamide | |
| 597 | 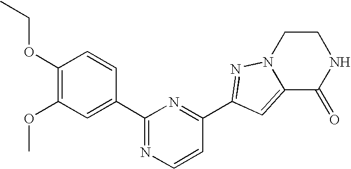 | 2-[2-(4-ethoxy-3-methoxyphenyl) pyrimidin-4-yl]-6,7- dihydropyrazolo[1,5-a]pyrazln- 4(5H)-one | |
| 598 | 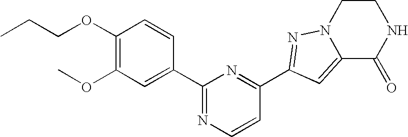 | 2-[2-(3-methoxy-4-propoxyphenyl) pyrimidin-4-yl]-6,7- dihydropyrazolo[1,5-a]pyrazin- 4(5H)-one | |
| 599 | 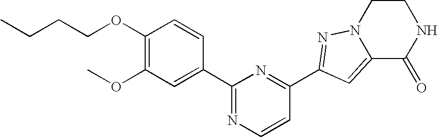 | 2-[2-(4-butoxy-3-methoxyphenyl) pyrimidin-4-yl]-6,7- dihydropyrazolo[1,5-a]pyrazin- 4(5H)-one | |
| 600 | 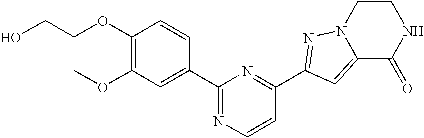 | 2-{2-[4-(2-hydroxyethoxy)-3- methoxyphenyl]pyrimidin-4-yl}-6,7- dihydropyrazolo[1,5-a]pyrazin- 4(5H)-one | |
| 601 | 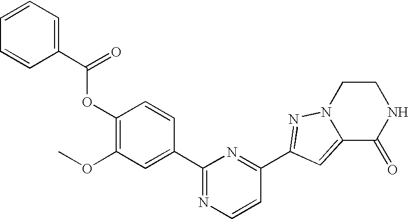 | 2-methoxy-4-[4-(4-oxo-4,5,6,7- tetrahydropyrazolo[1,5-a]pyrazin-2- yl)pyrimidin-2-yl]phenyl benzoate | |
| 602 | 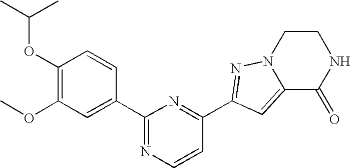 | 2-[2-(4-isopropoxy-3- methoxyphenyl)pyrimidin-4-yl]-6,7- dihydropyrazolo[1,5-a]pyrazin- 4(5H)-one | |
| 603 | 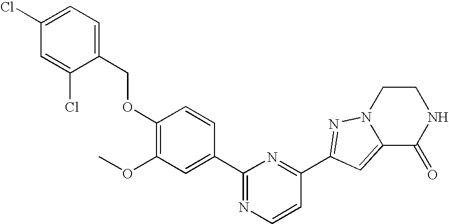 | 2-(2-{4-[(2,4-dichlorobenzyl)oxy]-3- methoxyphenyl}pyrimidin-4-yl)-6,7- dihydropyrazolo[1,5-a]pyrazin- 4(5H)-one | |
| 604 | 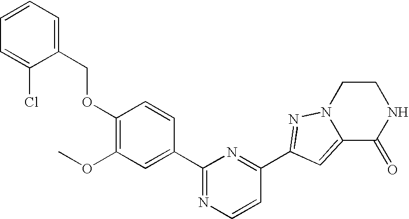 | 2-(2-{4-[(2-chlorobenzyl)oxy]-3- methoxyphenyl}pyrimidin-4-yl)-6,7- dihydropyrazolo[1,5-a]pyrazin- 4(5H)-one | |
| 605 | 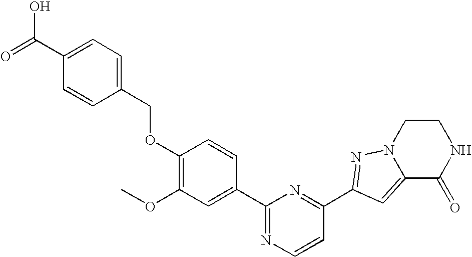 | 4-({2-methoxy-4-[4-(4-oxo-4,5,6,7- tetrahydropyrazolo[1,5-a]pyrazin-2-yl)pyrimidin-2-yl]phenoxy}methyl)benzoic acid | |
| 606 | 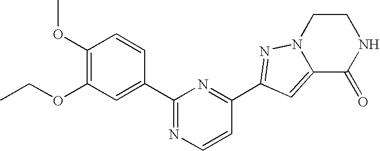 | 2-[2-(3-ethoxy-4-methoxyphenyl) pyrimidin-4-yl]-6,7- dihydropyrazolo[1,5-a]pyrazin- 4(5H)-one | |
| 607 | 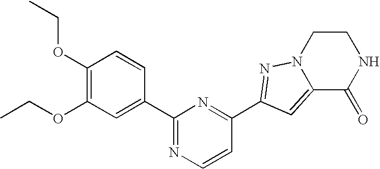 | 2-[2-(3,4-diethoxyphenyl)pyrimidin- 4-yl]-6,7-dihydropyrazolo [1,5-a]pyrazin-4(5H)-one | |
| 608 | 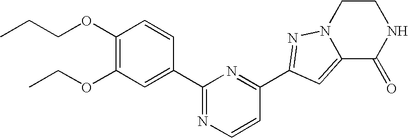 | 2-[2-(3-ethoxy-4-propoxyphenyl) pyrimidin-4-yl]-6,7- dihydropyrazolo[1,5-a]pyrazin- 4(5H)-one | |
| 609 | 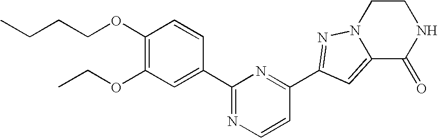 | 2-[2-(4-butoxy-3-ethoxyphenyl) pyrimidin-4-yl]-6,7- dihydropyrazolo[1,5-a]pyrazin- 4(5H)-one | |
| 610 | 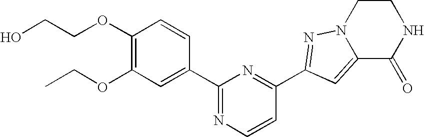 | 2-{2-[3-ethoxy-4-(2- hydroxyethoxy)phenyl]pyrimidin- 4-yl}-6,7-dihydropyrazolo [1,5-a]pyrazin-4(5H)-one | |
| 611 | 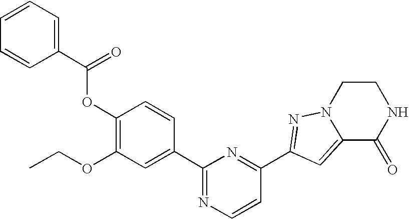 | 2-ethoxy-4-[4-(4-oxo-4,5,6,7- tetrahydropyrazolo[1,5-a]pyrazin-2-yl)pyrimidin-2-yl]phenyl benzoate | |
| 612 | 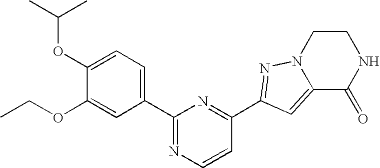 | 2-[2-(3-ethoxy-4-isopropoxyphenyl) pyrimidin-4-yl]-6,7- dihydropyrazolo[1,5-a]pyrazin- 4(5H)-one | |
| 613 | 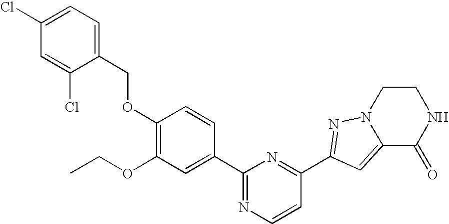 | 2-(2-{4-[(2,4-dichlorobenzyl)oxy]- 3-ethoxyphenyl)pyrimidin-4- yl)-6,7-dihydropyrazolo[1,5-a]pyrazin-4(5H)-one | |
| 614 | 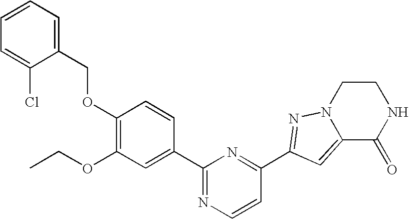 | 2-(2-{4-[(2-chlorobenzyl)oxy]-3- ethoxyphenyl}pyrimidin-4- yl)-6,7-dihydropyrazolo[1,5-a]pyrazin-4(5H)-one | |
| 615 | 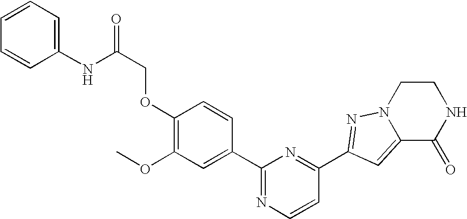 | 2-{2-methoxy-4-[4-(4-oxo- 4,5,6,7-tetrahydropyrazolo [1,5-a]pyrazin-2-yl) pyrimidin-2-yl]phenoxy}- N-phenylacetamide | |
| 616 | 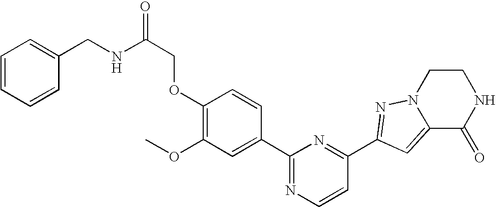 | N-benzyl-2-{2-methoxy-4-[4- (4-oxo-4,5,6,7-tetrahydropyrazolo [1,5-a]pyrazin-2-yl) pyrimidin-2-yl]phenoxy}acetamide | |
| 617 | 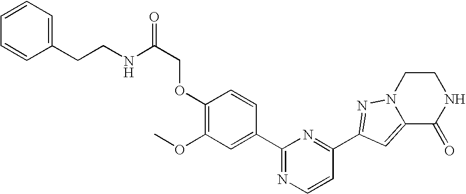 | 2-{2-methoxy-4-[4-(4-oxo- 4,5,6,7-tetrahydropyrazolo [1,5-a]pyrazin-2-yl) pyrimidin-2-yl]phenoxy}-N-(2- phenylethyl)acetamide | |
| 618 | 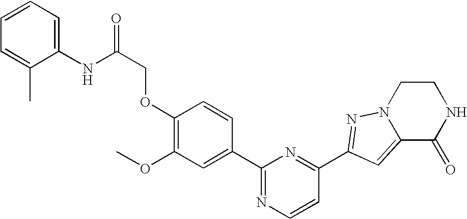 | 2-{2-methoxy-4-[4-(4-oxo- 4,5,6,7-tetrahydropyrazolo [1,5-a]pyrazin-2-yl) pyrimidin-2-yl]phenoxy}- N-(2-methylphenyl)acetamide | |
| 619 | 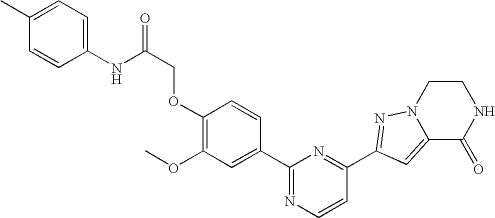 | 2-{2-methoxy-4-[4-(4-oxo-4,5,6,7- tetrahydropyrazolo[1,5-a]pyrazin-2- yl)pyrimidin-2-yl]phenoxy}-N-(4- methylphenyl)acetamide | |
| 620 | 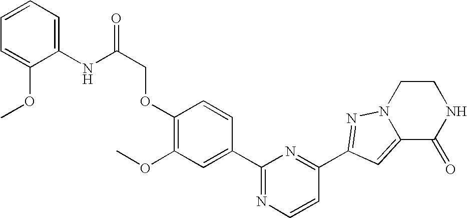 | 2-{2-methoxy-4-[4-(4-oxo-4,5,6,7- tetrahydropyrazolo[1,5-a]pyrazin-2- yl)pyrimidin-2-yl]phenoxy}-N-(2- methoxyphenyl)acetamide | |
| 621 | 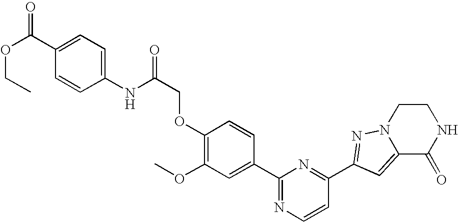 | ethyl 4-[({2-methoxy-4-[4-(4-oxo- 4,5,6,7-tetrahydropyrazolo[1,5-a]pyrazin-2-yl)pyrimidin-2- yl]phenoxy}acetyl) amino]benzoate | |
| 622 | 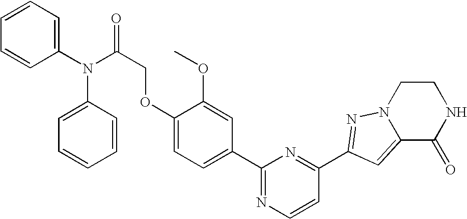 | 2-{2-methoxy-4-[4-(4-oxo-4,5,6,7- tetrahydropyrazolo[1,5-a]pyrazin- 2-yl)pyrimidin-2-yl]phenoxy}- N,N-diphenylacetamide | |
| 623 | 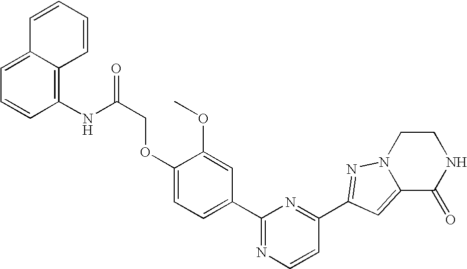 | 2-{2-methoxy-4-[4-(4-oxo-4,5,6,7- tetrahydropyrazolo[1,5-a]pyrazin- 2-yl)pyrimidin-2-yl]phenoxy}- N-1-naphthylacetamide | |
| 624 | 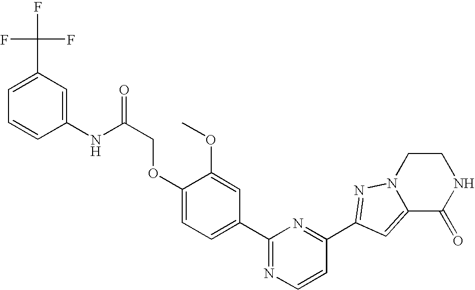 | 2-{2-methoxy-4-[4-(4-oxo-4,5,6,7- tetrahydroyrazolo[1,5-a]pyrazin- 2-yl)pyrimidin-2-yl]phenoxy}- N-[3-(trifluoromethyl) phenyl]acetamide | |
| 625 | 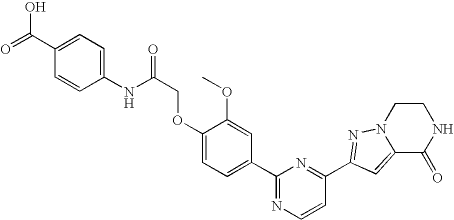 | 4-[({2-methoxy-4-[4-(4-oxo-4,5,6,7- tetrahydropyrazolo[1,5-a]pyrazin- 2-yl)pyrimidin-2-yl]phenoxy}acetyl)amino]benzoic acid | |
| 626 | 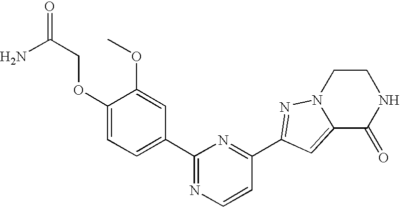 | 2-{2-methoxy-4-[4-(4-oxo-4,5,6,7- tetrahydropyrazolo[1,5-a]pyrazin-2- yl)pyrimidin-2-yl]phenoxy}acetamide | |
| 627 | 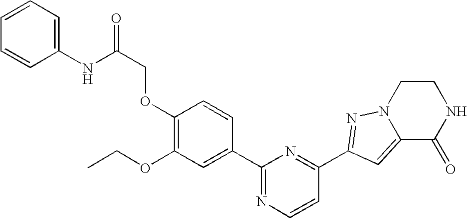 | 2-{2-ethoxy-4-(4-(4-oxo-4,5,6,7- tetrahydropyrazolo[1,5-a]pyrazin- 2-yl)pyrimidin-2-yl]phenoxy}- N-phenylacetamide | |
| 628 | 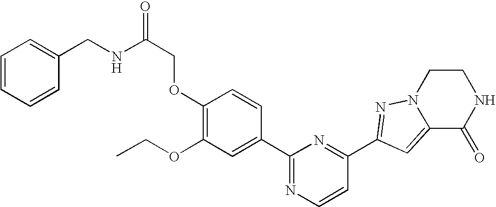 | N-benzyl-2-{2-ethoxy-4-[4- (4-oxo-4,5,6,7- tetrahydropyrazolo[1,5-a]pyrazin-2-yl)pyrimidin-2- yl]phenoxy}acetamide | |
| 629 | 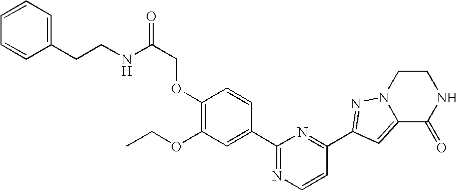 | 2-{2-ethoxy-4-[4-(4-oxo- 4,5,6,7-tetrahydropyrazolo [1,5-a]pyrazin-2-yl) pyrimidin-2-yl]phenoxy}- N-(2-phenylethyl)acetamide | |
| 630 | 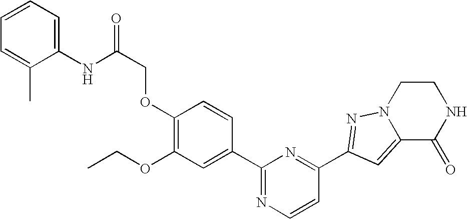 | 2-{2-ethoxy-4-[4-(4-oxo- 4,5,6,7-tetrahydropyrazolo [1,5-a]pyrazin-2-yl) pyrimidin-2-yl]phenoxy}- N-(2-methylphenyl)acetamide | |
| 631 | 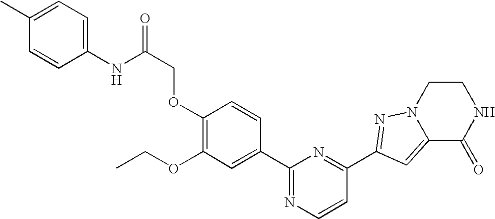 | 2-{2-ethoxy-4-[4-(4-oxo- 4,5,6,7-tetrahydropyrazolo [1,5-a]pyrazin-2-yl) pyrimidin-2-yl]phenoxy}- N-(2-methylphenyl)acetamide | |
| 632 | 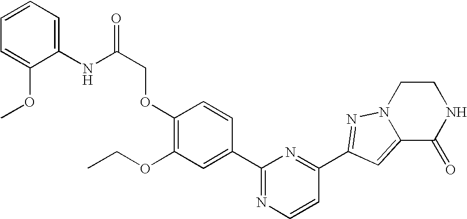 | 2-{2-ethoxy-4-[4-(4-oxo- 4,5,6,7-tetrahydropyrazolo [1,5-a]pyrazin-2-yl) pyrimidin-2-yl]phenoxy}- N-(2-methoxyphenyl)acetamide | |
| 633 | 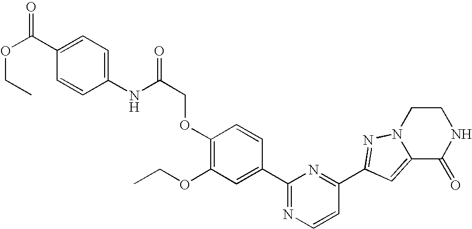 | ethyl 4-[({2-ethoxy-4-[4- (4-oxo-4,5,6,7- tetrahydropyrazolo[1,5-a]pyrazin-2-yl)pyrimidin- 2-yl]phenoxy}acetyl) amino]benzoate | |
| 634 | 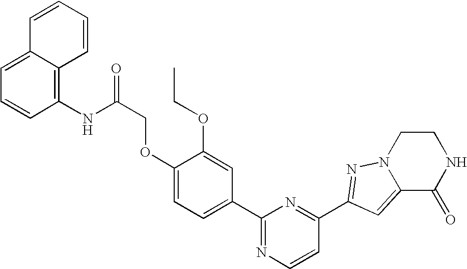 | 2-{2-ethoxy-4-[4-(4-oxo-4,5,6,7- tetrahydropyrazolo[1,5-a]pyrazin-2- yl)pyrimidin-2-yl]phenoxy}-N-1- naphthylacetamide | |
| 635 | 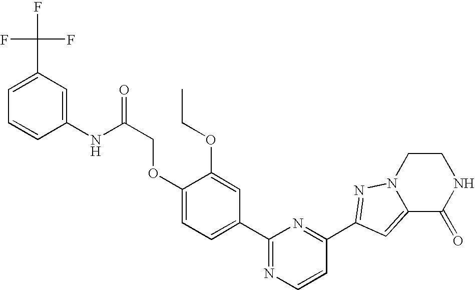 | 2-{2-ethoxy-4-[4-(4-oxo-4,5,6,7- tetrahydropyrazolo[1,5-a]pyrazin-2- yl)pyrimidin-2-yl]phenoxy}-N- [3-(trifluoromethyl) phenyl]acetamide | |
| 636 | 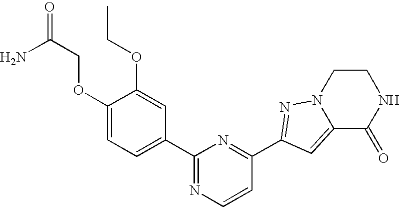 | 2-{2-ethoxy-4-[4-(4-oxo-4,5,6,7- tetrahydropyrazolo[1,5-a]pyrazin-2- yl)pyrimidin-2-yl]phenoxy}acetamide | |
| 637 | 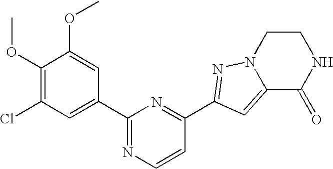 | 2-[2-(3-chloro-4,5- dimethoxyphenyl)pyrimidin-4-yl]-6,7- dihydropyrazolo[1,5-a]pyrazin- 4(5H)-one | |
| 638 | 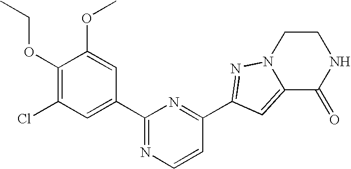 | 2-[2-(3-chloro-4-ethoxy-5- methoxyphenyl)pyrimidin-4-yl]-6,7- dihydropyrazolo[1,5-a]pyrazin- 4(5H)-one | |
| 639 | 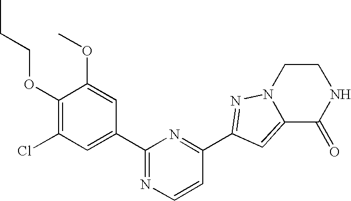 | 2-[2-(3-chloro-5-methoxy-4- propoxyphenyl)pyrimidin-4-yl]-6,7- dihydropyrazolo[1,5-a]pyrazin- 4(5H)-one | |
| 640 | 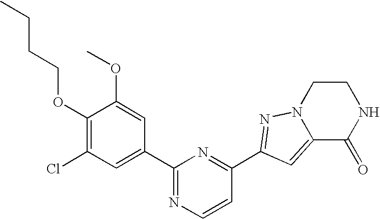 | 2-[2-(4-butoxy-3-chloro-5- methoxyphenyl)pyrimidin-4-yl]-6,7- dihydropyrazolo[1,5-a]pyrazin- 4(5H)-one | |
| 641 | 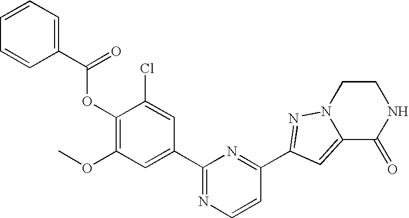 | 2-chloro-6-methoxy-4-[4-(4-oxo- 4,5,6,7-tetrahydropyrazolo [1,5-a]pyrazin-2-yl) pyrimidin-2-yl]phenyl benzoate | |
| 642 | 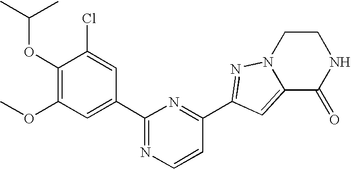 | 2-[2-(3-chloro-4-isopropoxy-5- methoxyphenyl)pyrimidin-4-yl]-6,7- dihydropyrazolo[1,5-a]pyrazin- 4(5H)-one | |
| 643 | 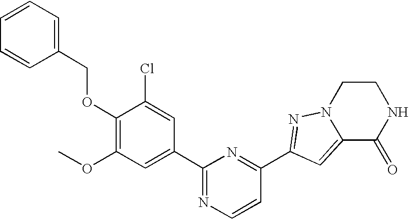 | 2-{2-[4-(benzyloxy)-3-chloro-5- methoxyphenyl]pyrimidin-4-yl}- 6,7-dihydropyrazolo[1,5-a]pyrazin-4(5H)-one | |
| 644 | 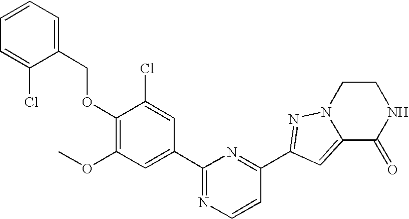 | 2-(2-{3-chloro-4-[(2- chlorobenzyl)oxy]-5- methoxyphenyl}pyrimidin-4-yl)- 6,7-dihydropyrazolo[1,5-a]pyrazin-4(5H)-one | |
| 645 | 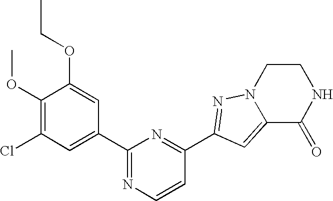 | 2-[2-(3-chloro-5-ethoxy-4- methoxyphenyl)pyrimidin-4- yl]-6,7-dihydropyrazolo [1,5-a]pyrazin-4(5H)-one | |
| 646 | 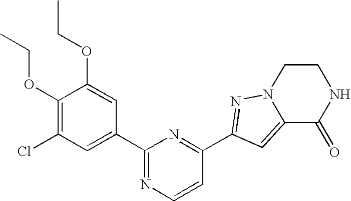 | 2-[2-(3-chloro-4,5-diethoxyphenyl) pyrimidin-4-yl]-6,7- dihydropyrazolo[1,5-a]pyrazin-4(5H)-one | |
| 647 | 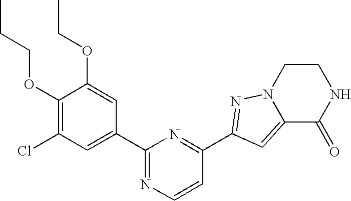 | 2-[2-(3-chloro-5-ethoxy-4- propoxyphenyl)pyrimidin-4-yl]- 6,7-dihydropyrazolo[1,5-a]pyrazin-4(5H)-one | |
| 648 | 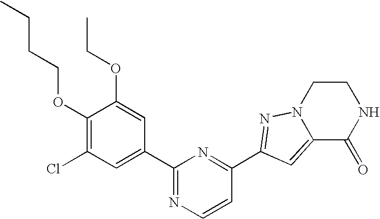 | 2-[2-(4-butoxy-3-chloro-5- ethoxyphenyl)pyrimidin-4-yl]- 6,7-dihydropyrazolo[1,5-a]pyrazin-4(5H)-one | |
| 649 | 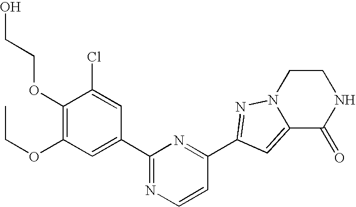 | 2-{2-[3-chloro-5-ethoxy-4-(2- hydroxyethoxy)phenyl]pyrimidin-4-yl}-6,7- dihydropyrazolo[1,5-a]pyrazin-4(5H)-one | |
| 650 | 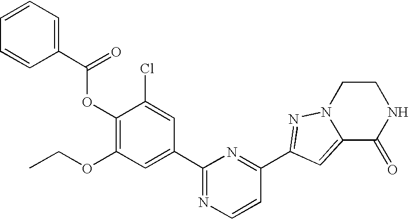 | 2-chloro-6-ethoxy-4-[4-(4-oxo- 4,5,6,7-tetrahydropyrazolo [1,5-a]pyrazin-2-yl) pyrimidin-2-yl]phenyl benzoate | |
| 651 | 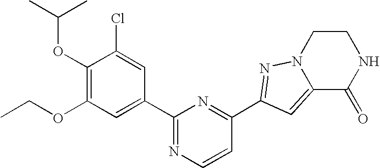 | 2-[2-(3-chloro-5-ethoxy-4- isopropoxyphenyl)pyrimidin-4-yl]-6,7- dihydropyrazolo[1,5-a]pyrazin- 4(5H)-one | |
| 652 | 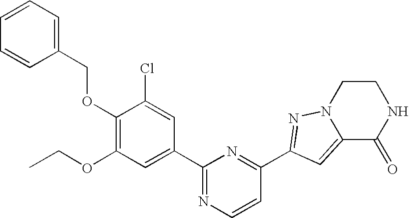 | 2-{2-[4-(benzyloxy)-3-chloro-5- ethoxyphenyl]pyrimidin-4-yl}-6,7- dihydropyrazolo[1,5-a]pyrazin- 4(5H)-one | |
| 653 | 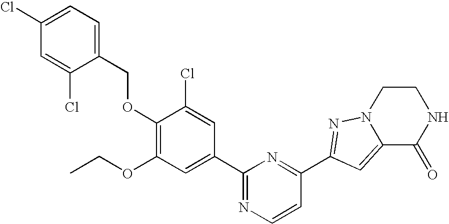 | 2-(2-[3-chloro-4-[(2,4-dichlorobenzyl) oxy]-5-ethoxyphenyl}pyrimidin-4-yl)-6,7- dihydropyrazolo[1,5-a]pyrazin-4(5H)-one | |
| 654 | 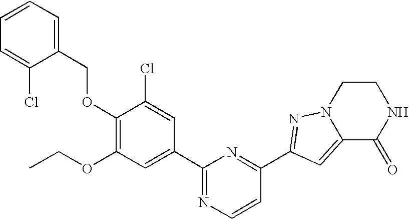 | 2-(2-{3-chloro-4-[(2- chlorobenzyl)oxy]-5- ethoxyphenyl}pyrimidin-4- yl)-6,7-dihydropyrazolo [1,5-a]pyrazin- 4(5H)-one | |
| 655 | 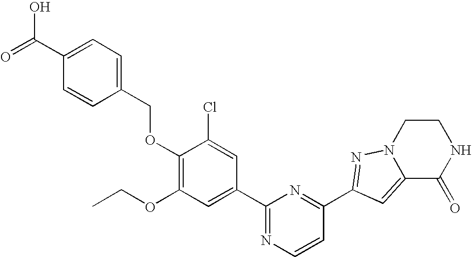 | 4-({2-chloro-6-ethoxy-4-[4-(4-oxo- 4,5,6,7-tetrahydropyrazolo [1,5-a]pyrazin-2-yl) pyrimidin-2-yl]phenoxy}methyl)benzoic acid | |
| 656 | 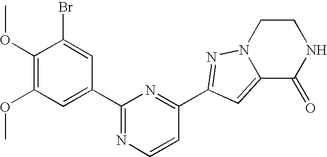 | 2-[2-(3-bromo-4,5- dimethoxyphenyl)pyrimidin-4- yl]-6,7-dihydropyrazolo [1,5-a]pyrazin-4(5H)-one | |
| 657 | 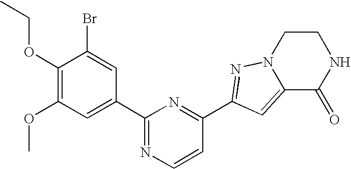 | 2-[2-(3-bromo-4-ethoxy-5- methoxyphenyl)pyrimidin-4- yl]-6,7-dihydropyrazolo [1,5-a]pyrazin-4(5H)-one | |
| 658 | 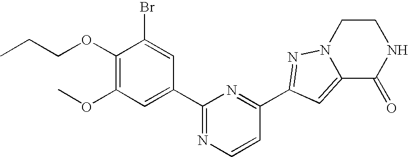 | 2-[2-(3-bromo-5-methoxy-4- propoxyphenyl)pyrimidin-4- yl]-6,7-dihydropyrazolo [1,5-a]pyrazin-4(5H)-one | |
| 659 | 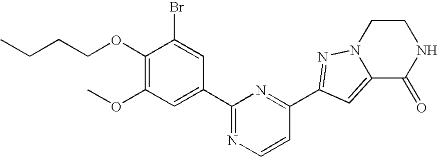 | 2-[2-(3-bromo-4-butoxy-5- methoxyphenyl)pyrimidin-4- yl]-6,7-dihydropyrazolo [1,5-a]pyrazin-4(5H)-one | |
| 660 | 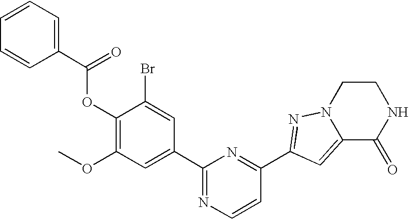 | 2-bromo-6-methoxy-4-[4-(4-oxo- 4,5,6,7-tetrahydropyrazolo [1,5-a]pyrazin-2-yl) pyrimidin-2-yl]phenyl benzoate | |
| 661 | 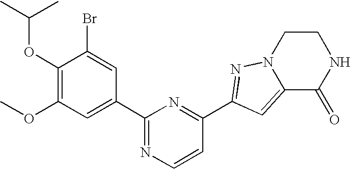 | 2-[2-(3-bromo-4-isopropoxy-5- methoxyphenyl)pyrimidin-4- yl]-6,7-dihydropyrazolo [1,5-a]pyrazin-4(5H)-one | |
| 662 | 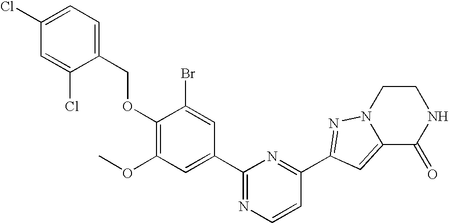 | 2-(2-{3-bromo-4-[(2,4- dichlorobenzyl)oxy]-5- methoxyphenyl}pyrimidin-4-yl)-6,7- dihydropyrazolo[1,5-a]pyrazin-4(5H)-one | |
| 663 | 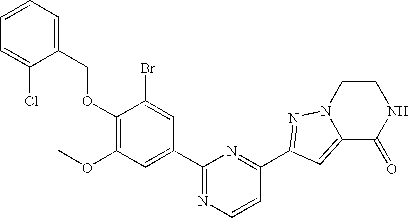 | 2-(2-{3-bromo-4-[(2- chlorobenzyl)oxy]-5- methoxyphenyl}pyrimidin-4-yl)-6,7- dihydropyrazolo[1,5-a]pyrazin- 4(5H)-one | |
| 664 | 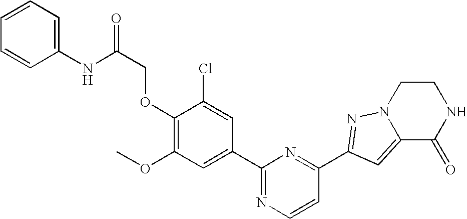 | 2-{2-chloro-6-methoxy-4- [4-(4-oxo-4,5,6,7- tetrahydroyrazolo[1,5-a]pyrazin-2-yl)pyrimidin- 2-yl]phenoxy}-N- phenylacetamide | |
| 665 | 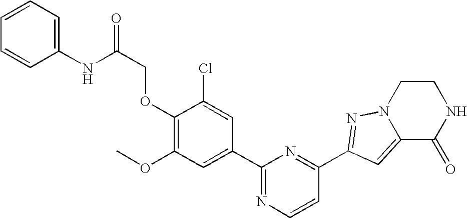 | 2-{2-chloro-6-methoxy-4-[4- (4-oxo-4,5,6,7-tetrahydropyrazolo [1,5-a]pyrazin-2-yl) pyrimidin-2-yl]phenoxy}acetamide | |
| 666 | 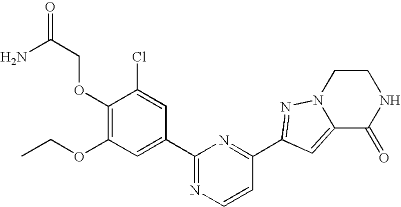 | 2-{2-chloro-6-ethoxy-4-[4-(4- oxo-4,5,6,7-tetrahydropyrazolo [1,5-a]pyrazin-2-yl) pyrimidin-2-yl]phenoxy}acetamide | |
| 667 | 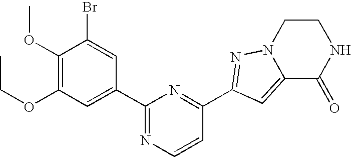 | 2-[2-(3-bromo-5-ethoxy-4- methoxyphenyl)pyrimidin-4- yl]-6,7-dihydropyrazolo [1,5-a]pyrazin-4(5H)-one | |
| 668 | 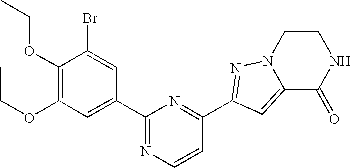 | 2-[2-(3-bromo-4,5-diethoxyphenyl) pyrimidin-4-yl]-6,7- dihydropyrazolo[1,5-a]pyrazin- 4(5H)-one | |
| 669 | 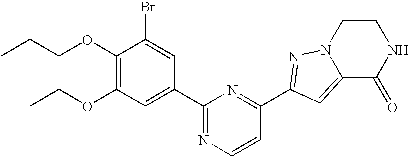 | 2-[2-(3-bromo-5-ethoxy-4- propoxyphenyl)pyrimidin-4-yl]-6,7- dihydropyrazolo[1,5-a]pyrazin- 4(5H)-one | |
| 670 | 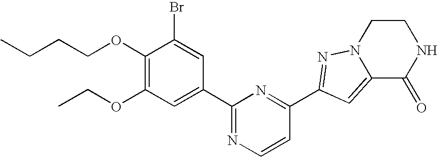 | 2-[2-(3-bromo-4-butoxy-5- ethoxyphenyl)pyrimidin-4-yl]-6,7- dihydropyrazolo[1,5-a]pyrazin- 4(5H)-one | |
| 671 | 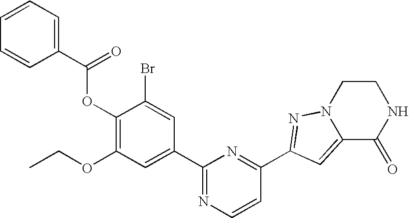 | 2-bromo-6-ethoxy-4-[4-(4-oxo- 4,5,6,7-tetrahydropyrazolo [1,5-a]pyrazin-2-yl) pyrimidin-2-yl]phenyl benzoate | |
| 672 | 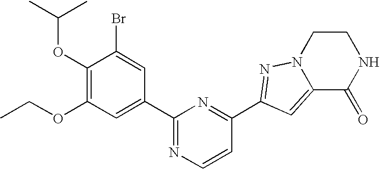 | 2-[2-(3-bromo-5-ethoxy-4- isopropoxyphenyl)pyrimidin-4-yl]-6,7- dihydropyrazolo[1,5-a]pyrazin- 4(5H)-one | |
| 673 | 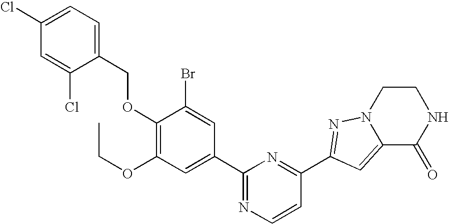 | 2-(2-{3-bromo-4-[(2,4- dichlorobenzyl)oxy]-5- ethoxyphenyl}pyrimidin-4- yl)-6,7-dihydropyrazolo [1,5-a]pyrazin-4(5H)-one | |
| 674 | 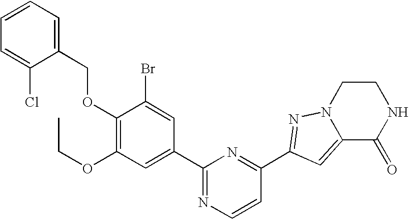 | 2-(2-{3-bromo-4-[(2-chlorobenzyl) oxy]-5-ethoxyphenyl}pyrimidin-4-yl)-6,7- dihydropyrazolo[1,5-a]pyrazin-4(5H)-one | |
| 675 | 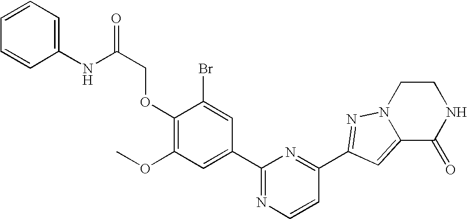 | 2-{2-bromo-6-methoxy-4-[4- (4-oxo-4,5,6,7- tetrahydropyrazolo[1,5-a]pyrazin-2- yl)pyrimidin-2-yl]phenoxy}-N- phenylacetamide | |
| 676 | 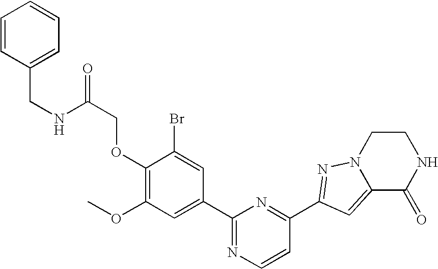 | N-benzyl-2-{2-bromo-6-methoxy- 4-[4-(4-oxo-4,5,6,7- tetrahydropyrazolo[1,5-a]pyrazin-2-yl)pyrimidin-2- yl]phenoxy}acetamide | |
| 677 | 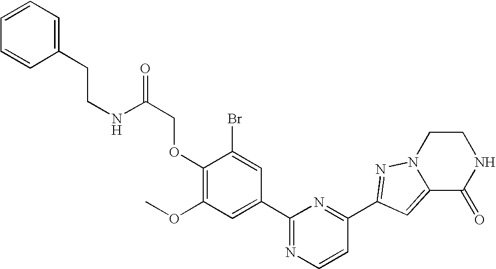 | 2-{2-bromo-6-methoxy-4-[4-(4- oxo-4,5,6,7-tetrahydropyrazolo[1,5-a]pyrazin-2-yl)pyrimidin-2- yl]phenoxy}-N-(2- phenylethyl)acetamide | |
| 678 | 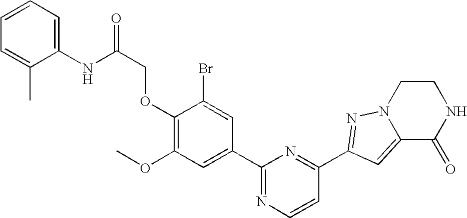 | 2-{2-bromo-6-methoxy-4-[4-(4- oxo-4,5,6,7-tetrahydropyrazolo[1,5-a]pyrazin-2-yl)pyrimidin-2- yl]phenoxy}-N-(2- methylphenyl)acetamide | |
| 679 | 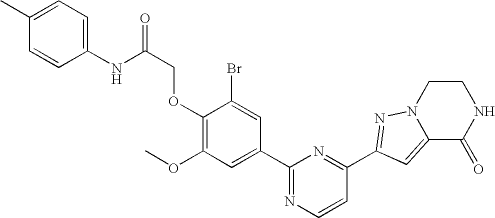 | 2-{2-bromo-6-methoxy-4-[4-(4- oxo-4,5,6,7-tetrahydropyrazolo[1,5-a]pyrazin-2-yl)pyrimidin-2- yl]phenoxy}-N-(4- methylphenyl)acetamide | |
| 680 | 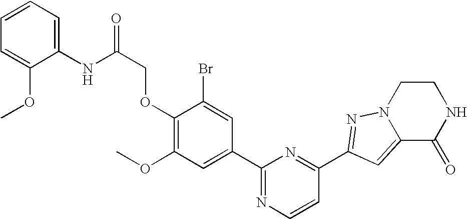 | 2-{2-bromo-6-methoxy-4-[4-(4- oxo-4,5,6,7-tetrahydropyrazolo[1,5-a]pyrazin-2-yl)pyrimidin-2- yl]phenoxy}-N-(2- methoxyphenyl)acetamide | |
| 681 | 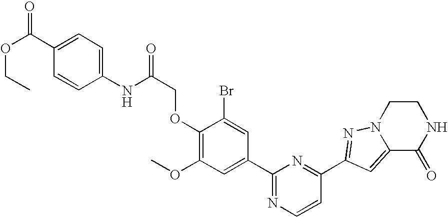 | ethyl 4-[({2-bromo-6-methoxy-4-[4- (4-oxo-4,5,6,7-tetrahydropyrazolo [1,5-a]pyrazin-2-yl) pyrimidin-2-yl]phenoxy}acetyl)amino]benzoate | |
| 682 | 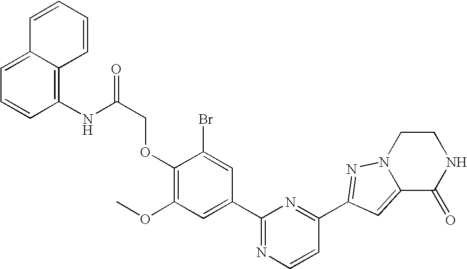 | 2-{2-bromo-6-methoxy-4-[4-(4- oxo-4,5,6,7-tetrahydropyrazolo[1,5-a]pyrazin-2-yl)pyrimidin-2-yl]phenoxy}-N-1- naphthylacetamide | |
| 683 | 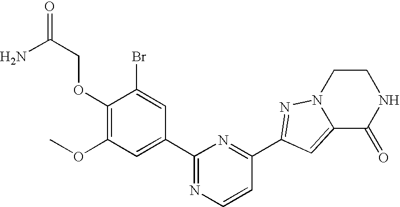 | 2-{2-bromo-6-methoxy-4-[4-(4- oxo-4,5,6,7-tetrahydropyrazolo[1,5-a]pyrazin-2-yl)pyrimidin-2-yl]phenoxy}acetamide | |
| 684 | 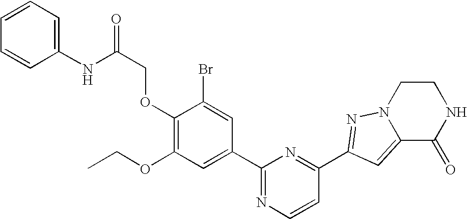 | 2-{2-bromo-6-ethoxy-4-[4-(4- oxo-4,5,6,7-tetrahydropyrazolo[1,5-a]pyrazin-2-yl)pyrimidin-2-yl]phenoxy}-N-phenylacetamide | |
| 685 | 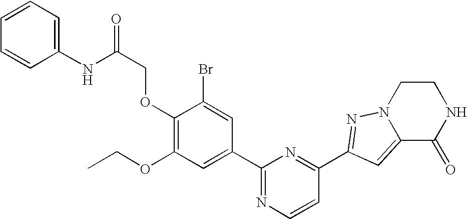 | N-benzyl-2-{2-bromo-6-ethoxy-4-[4- (4-oxo-4,5,6,7-tetrahydropyrazolo [1,5-a]pyrazin-2-yl) pyrimidin-2-yl]phenoxy}acetamide | |
| 686 | 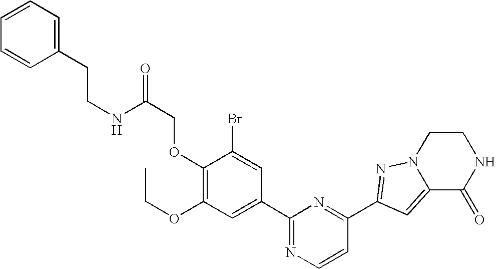 | 2-{2-bromo-6-ethoxy-4-[4-(4-oxo- 4,5,6,7-tetrahydropyrazolo[1,5-a]pyrazin-2-yl)pyrimidin-2- yl]phenoxy}-N-(2- phenylethyl)acetamide | |
| 687 | 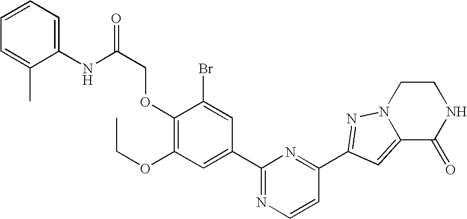 | 2-{2-bromo-6-ethoxy-4-[4-(4-oxo- 4,5,6,7-tetrahydropyrazolo[1,5-a]pyrazin-2-yl)pyrimidin-2- yl]phenoxy}-N-(2- methylphenyl)acetamide | |
| 688 | 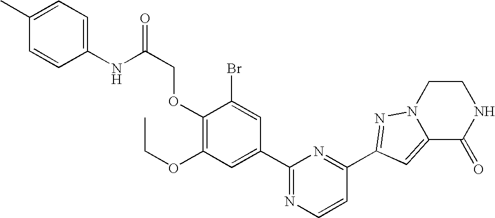 | 2-{2-bromo-6-ethoxy-4-[4-(4-oxo- 4,5,6,7-tetrahydropyrazolo[1,5-a]pyrazin-2-yl)pyrimidin-2- yl]phenoxy}-N-(4- methylphenyl)acetamide | |
| 689 | 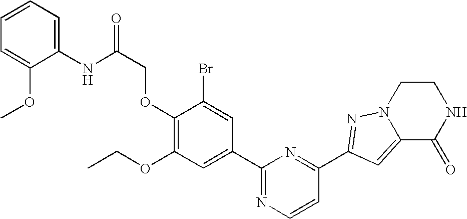 | 2-{2-bromo-6-ethoxy-4-[4-(4-oxo- 4,5,6,7-tetrahydropyrazolo[1,5-a]pyrazin-2-yl)pyrimidin-2- yl]phenoxy}-N-(2- methoxyphenyl)acetamide | |
| 690 | 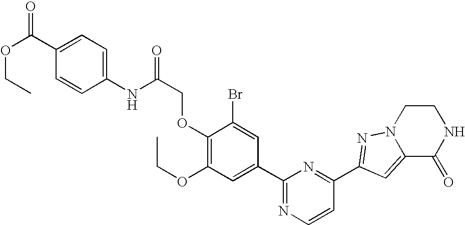 | ethyl 4-[({2-bromo-6-ethoxy-4- [4-(4-oxo-4,5,6,7- tetrahydropyrazolo[1.5-a]pyrazin- 2-yl)pyrimidin-2- yl]phenoxy}acetyl) amino]benzoate | |
| 691 | 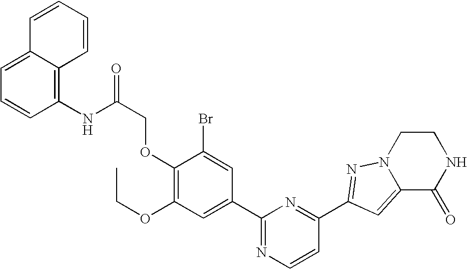 | 2-{2-bromo-6-ethoxy-4-[4-(4- oxo-4,5,6,7-tetrahydropyrazolo [1,5-a]pyrazin-2-yl) pyrimidin-2-yl]phenoxy}-N-1- naphthylacetamide | |
| 692 | 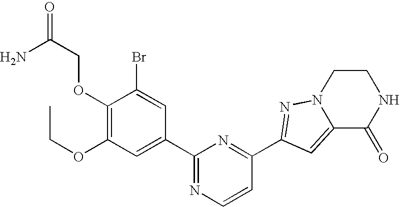 | 2-{2-bromo-6-ethoxy-4-[4-(4- oxo-4,5,6,7-tetrahydropyrazolo [1,5-a]pyrazin-2-yl) pyrimidin-2-yl]phenoxy}acetamide | |
| 693 | 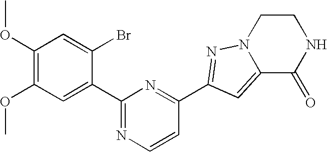 | 2-[2-(2-bromo-4,5-dimethoxyphenyl) pyrimidin-4-yl]-6,7- dihydropyrazolo[1,5-a]pyrazin-4(5H)-one | |
| 694 | 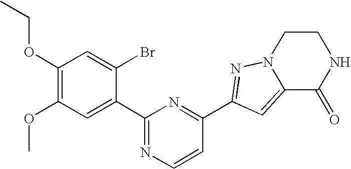 | 2-[2-(2-bromo-4-ethoxy-5- methoxyphenyl)pyrimidin-4-yl]- 6,7-dihydropyrazolo[1,5-a]pyrazin-4(5H)-one | |
| 695 | 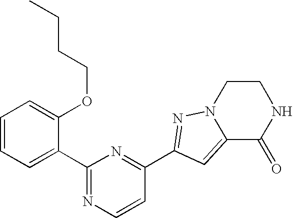 | 2-[2-(2-butoxyphenyl)pyrimidin-4- yl]-6,7-dihydropyrazolo [1,5-a]pyrazin-4(5H)-one | |
| 696 | 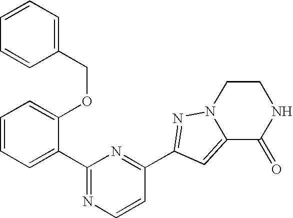 | 2-{2-[2-(benzyloxy)phenyl]pyrimidin-4-yl}-6,7- dihydropyrazolo[1,5-a]pyrazin- 4(5H)-one | |
| 697 | 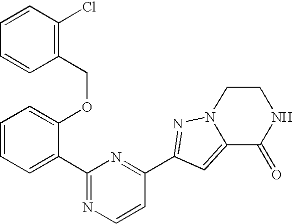 | 2-(2-{2-[(2-chlorobenzyl)oxy]phenyl}pyrimidin-4-yl)-6,7- dihydropyrazolo[1,5-a]pyrazin- 4(5H)-one | |
| 698 | 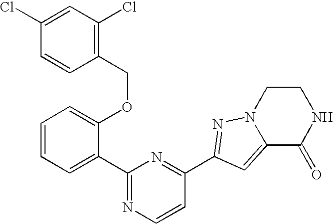 | 2-(2-{2-[(2,4-dichlorobenzyl) oxy]phenyl}pyrimidin-4-yl)- 6,7-dihydropyrazolo[1,5-a]pyrazin-4(5H)-one | |
| 699 | 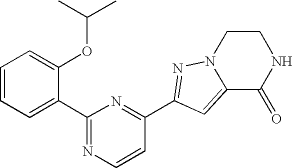 | 2-[2-(2-isopropoxyphenyl)pyrimidin- 4-yl]-6,7-dihydropyrazolo[1,5-a]pyrazin-4(5H)-one | |
| 700 | 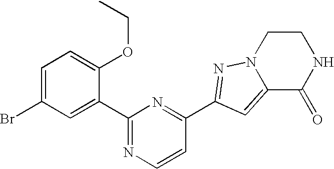 | 2-[2-(5-bromo-2-ethoxyphenyl) pyrimidin-4-yl]-6,7- dihydropyrazolo[1,5-a]pyrazin-4(5H)-one | |
| 701 | 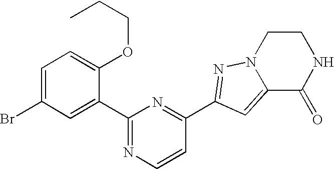 | 2-[2-(5-bromo-2-propoxyphenyl) pyrimidin-4-yl]-6,7- dihydropyrazolo[1,5-a]pyrazin-4(5H)-one | |
| 702 | 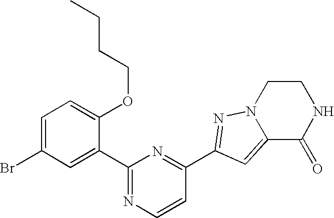 | 2-[2-(5-bromo-2-butoxyphenyl) pyrimidin-4-yl]-6,7- dihydropyrazolo[1,5-a]pyrazin-4(5H)-one | |
| 703 | 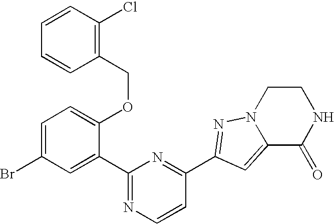 | 2-(2-[5-bromo-2-[(2-chlorobenzyl)oxy]phenyl}pyrimidin-4-yl)-6,7- dihydropyrazolo[1,5-a]pyrazin-4(5H)-one | |
| 704 | 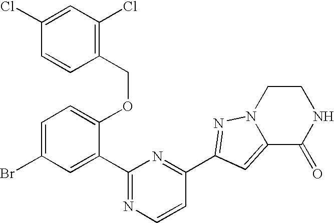 | 2-(2-{5-bromo-2-[(2,4- dichlorobenzyl)oxy]phenyl}pyrimidin-4-yl)-6,7- dihydropyrazolo[1,5-a]pyrazin- 4(5H)-one | |
| 705 | 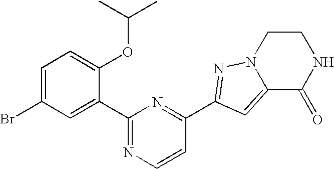 | 2-[2-(5-bromo-2- isopropoxyphenyl)pyrimidin-4- yl]-6,7-dihydropyrazolo[1,5-a]pyrazin-4(5H)-one | |
| 706 | 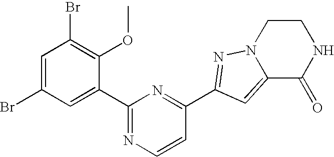 | 2-[2-(3,5-dibromo-2- methoxyphenyl)pyrimidin-4-yl]-6,7- dihydropyrazolo[1,5-a]pyrazin- 4(5H)-one | |
| 707 | 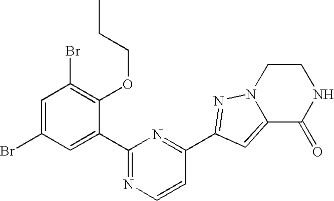 | 2-[2-(3,5-dibromo-2-propoxyphenyl) pyrimidin-4-yl]-6,7- dihydropyrazolo[1,5-a]pyrazin- 4(5H)-one | |
| 708 | 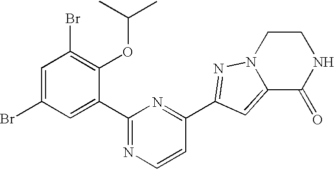 | 2-[2-(3,5-dibromo-2-isopropoxyphenyl) pyrimidin-4-yl]-6,7- dihydropyrazolo[1,5-a]pyrazin- 4(5H)-one | |
| 709 | 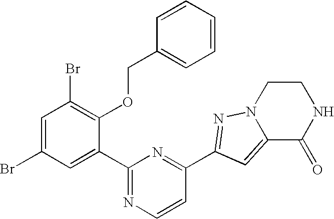 | 2-{2-[2-(benzyloxy)-3,5- dibromophenyl]pyrimidin-4-yl}- 6,7-dihydropyrazolo[1,5-a]pyrazin-4(5H)-one | |
| 710 | 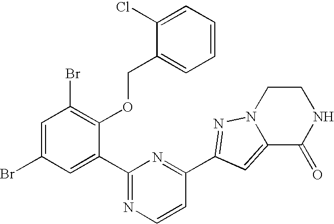 | 2-(2-{3,5-dibromo-2-[(2- chlorobenzyl)oxy]phenyl}pyrimidin-4- yl)-6,7-dihydropyrazolo[1,5-a]pyrazin-4(5H)-one | |
| 711 | 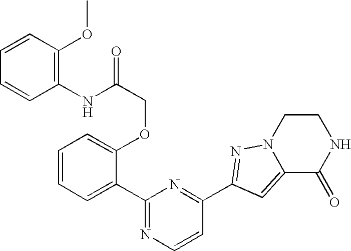 | N-(2-methoxyphenyl)-2-{2-[4-(4- oxo-4,5,6,7-tetrahydropyrazolo[1,5-a]pyrazin-2-yl)pyrimidin-2- yl]phenoxy}acetamide | |
| 712 | 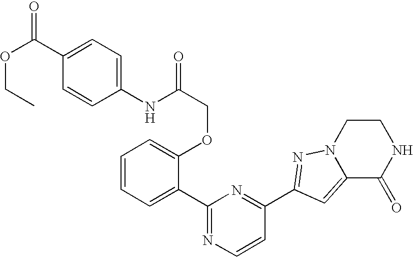 | ethyl 4-[({2-[4-(4-oxo-4,5,6,7- tetrahydropyrazolo[1,5-a]pyrazin-2-yl)pyrimidin-2- yl]phenoxy}acetyl) amino]benzoate | |
| 713 | 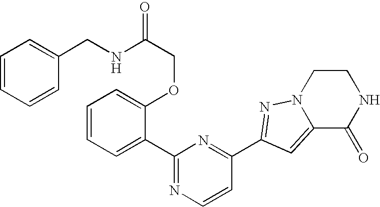 | N-benzyl-2-{2-[4-(4-oxo-4,5,6,7- tetrahydropyrazolo[1,5-a]pyrazin-2- yl)pyrimidin-2-yl]phenoxy}acetamide | |
| 714 | 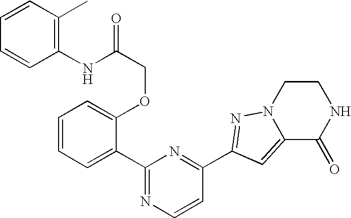 | N-(2-methylphenyl)-2-{2-[4-(4-oxo- 4,5,6,7-tetrahydropyrazolo [1,5-a]pyrazin-2-yl) pyrimidin-2-yl]phenoxy}acetamide | |
| 715 | 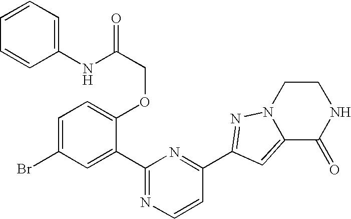 | 2-{4-bromo-2-[4-(4-oxo-4,5,6,7- tetrahydropyrazolo[1,5-a]pyrazin-2-yl)pyrimidin-2-yl]phenoxy}-N-phenylacetamide | |
| 716 | 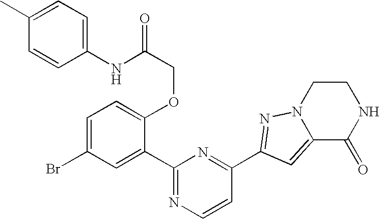 | 2-{4-bromo-2-[4-(4-oxo-4,5,6,7- tetrahydropyrazolo[1,5-a]pyrazin-2-yl)pyrimidin-2-yl]phenoxy}-N-(4- methylphenyl)acetamide | |
| 717 | 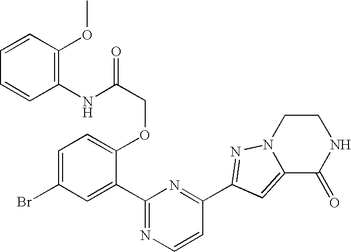 | 2-{4-bromo-2-[4-(4-oxo-4,5,6,7- tetrahydropyrazolo[1,5-a]pyrazin-2-yl)pyrimidin-2-yl]phenoxy}-N-(2- methoxyphenyl)acetamide | |
| 718 | 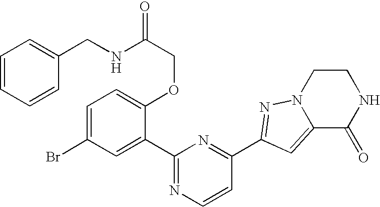 | N-benzyl-2-{4-bromo-2-[4-(4-oxo- 4,5,6,7-tetrahydropyrazolo [1,5-a]pyrazin-2-yl) pyrimidin-2-yl]phenoxy}acetamide | |
| 719 | 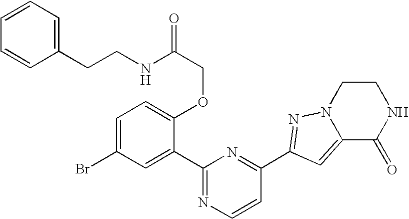 | 2-{4-bromo-2-[4-(4-oxo-4,5,6,7- tetrahydropyrazolo[1,5-a]pyrazin-2-yl)pyrimidin- 2-yl]phenoxy}-N-(2- phenylethyl)acetamide | |
| 720 | 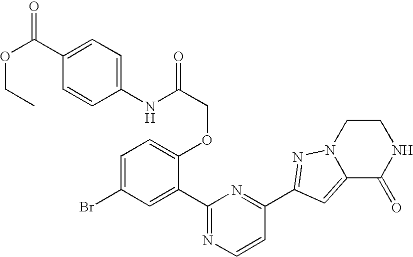 | ethyl 4-[({4-bromo-2-[4-(4-oxo- 4,5,6,7-tetrahydropyrazolo [1,5-a]pyrazin-2-yl) pyrimidin-2-yl]phenoxy}acetyl)amino]benzoate | |
| 721 | 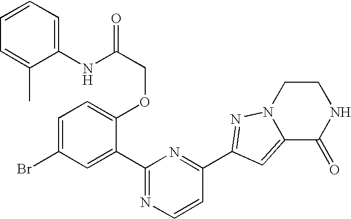 | 2-{4-bromo-2-[4-(4-oxo-4,5,6,7- tetrahydropyrazolo[1,5-a]pyrazin-2-yl)pyrimidin- 2-yl]phenoxy}-N-(2- methylphenyl)acetamide | |
| 722 | 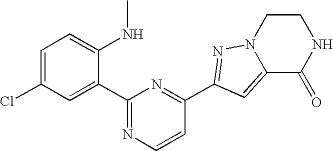 | 2-{2-[5-chloro-2- (methylamino)phenyl]pyrimidin- 4-yl}-6,7-dihydropyrazolo [1,5-a]pyrazin-4(5H)-one | |
| 723 | 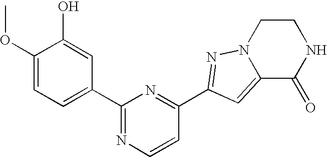 | 2-[2-(3-hydroxy-4-methoxyphenyl) pyrimidin-4-yl]-6,7- dihydropyrazolo[1,5-a]pyrazin- 4(5H)-one | |
| 724 | 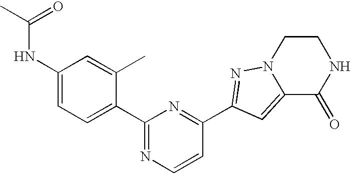 | N-{3-methyl-4-[4-(4-oxo-4,5,6,7- tetrahydropyrazolo[1,5-a]pyrazin-2- yl)pyrimidin-2-yl]phenyl}acetamide | |
| 725 | 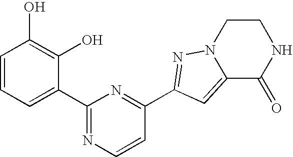 | 2-[2-(2,3-dihydroxyphenyl)pyrimidin-4- yl]-6,7-dihydropyrazolo[1,5-a]pyrazin-4(5H)-one | |
| 726 | 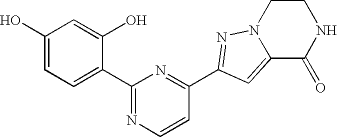 | 2-[2-(2,4-dihydroxyphenyl)pyrimidin-4- yl]-6,7-dihydropyrazolo[1,5-a]pyrazin-4(5H)-one | |
| 727 | 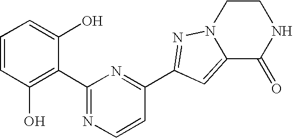 | 2-[2-(2,6-dihydroxyphenyl)pyrimidin-4- yl]-6,7-dihydropyrazolo[1,5-a]pyrazin-4(5H)-one | |
| 728 | 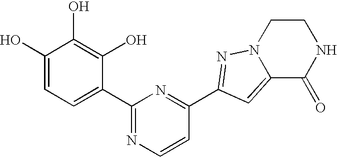 | 2-[2-(2,3,4-trihydroxyphenyl)pyrimidin- 4-yl]-6,7-dihydropyrazolo [1,5-a]pyrazin-4(5H)-one | |
| 729 | 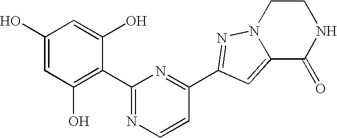 | 2-[2-(2,4,6-trihydroxyphenyl)pyrimidin- 4-yl]-6,7-dihydropyrazolo [1,5-a]pyrazin-4(5H)-one | |
| 730 | 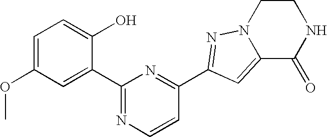 | 2-[2-(2-hydroxy-5-methoxyphenyl) pyrimidin-4-yl]-6,7- dihydropyrazolo[1,5-a]pyrazin- 4(5H)-one | |
| 731 | 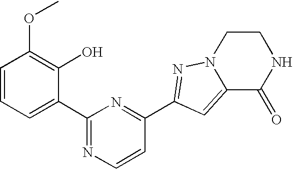 | 2-[2-(2-hydroxy-3-methoxyphenyl) pyrimidin-4-yl]-6,7- dihydropyrazolo[1,5-a]pyrazin- 4(5H)-one | |
| 732 | 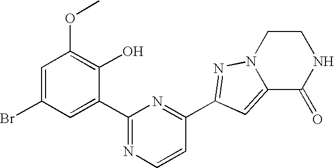 | 2-[2-(5-bromo-2-hydroxy-3- methoxyphenyl)pyrimidin-4-yl]-6,7- dihydropyrazolo[1,5-a]pyrazin-4(5H)-one | |
| 733 | 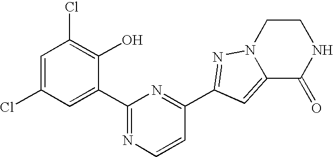 | 2-[2-(3,5-dichloro-2- hydroxyphenyl)pyrimidin-4- yl]-6,7-dihydropyrazolo [1,5-a]pyrazin-4(5H)-one | |
| 734 | 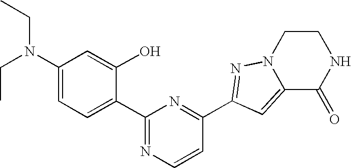 | 2-{2-(4-(diethylamino)-2- hydroxyphenyl]pyrimidin-4- yl}-6,7-dihydropyrazolo [1,5-a]pyrazin-4(5H)-one | |
| 735 | 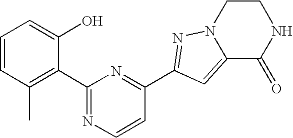 | 2-[2-(2-hydroxy-6-methylphenyl) pyrimidin-4-yl]-6,7- dihydropyrazolo[1,5-a]pyrazin-4(5H)-one | |
| 736 | 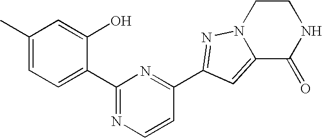 | 2-[2-(2-hydroxy-4-methylphenyl) pyrimidin-4-yl]-6,7- dihydropyrazolo[1,5-a]pyrazin-4(5H)-one | |
| 737 | 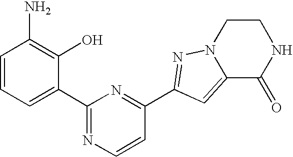 | 2-[2-(3-amino-2-hydroxyphenyl) pyrimidin-4-yl]-6,7- dihydropyrazolo[1,5-a]pyrazin-4(5H)-one | |
| 738 | 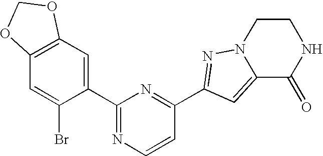 | 2-[2-(6-bromo-1,3-benzodioxol-5- yl)pyrimidin-4-yl]-6,7- dihydropyrazolo[1,5-a]pyrazin-4(5H)-one | |
| 739 | 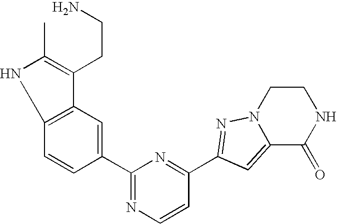 | 2-{2-[3-(2-aminoethyl)-2- methyl-1H-indol-5-yl]pyrimidin-4-yl}-6,7- dihydropyrazolo[1,5-a]pyrazin-4(5H)-one | |
| 740 | 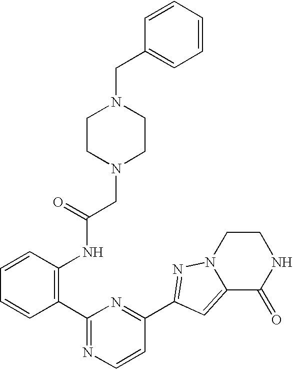 | 2-(4-benzylpiperazin-1-yl)-N-{2-[4- (4-oxo-4,5,6,7-tetrahydropyrazolo [1,5-a]pyrazin-2-yl) pyrimidin-2-yl]phenyl}acetamide | |
| 741 | 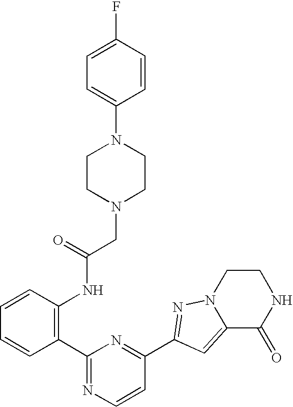 | 2-[4-(4-fluorophenyl)piperazin-1-yl]N- {2-[4-(4-oxo-4,5,6,7- tetrahydropyrazolo[1,5-a]pyrazin-2-yl)pyrimidin-2- yl]phenyl}acetamide | |
| 742 | 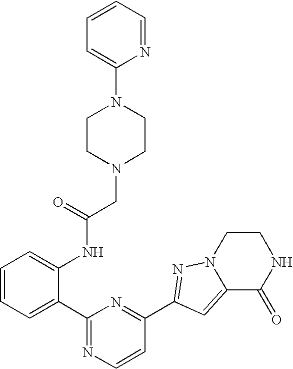 | N-{2-[4-(4-oxo-4,5,6,7- tetrahydropyrazolo[1,5-a]pyrazin-2- yl)pyrimidin-2-yl]phenyl}-2- (4-pyridin-2-ylpiperazin- 1-yl)acetamide | |
| 743 | 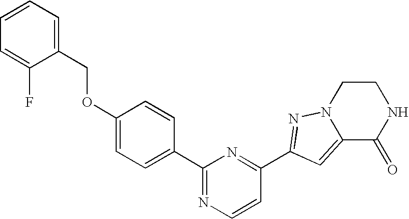 | 2-(2-{4-[(2-fluorobenzyl) oxy]phenyl}pyrimidin-4-yl)- 6,7-dihydropyrazolo [1,5-a]pyrazin-4(5H)-one | |
| 744 | 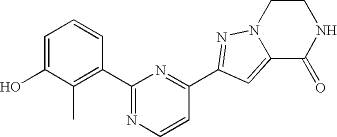 | 2-[2-(3-hydroxy-2-methylphenyl) pyrimidin-4-yl]-6,7- dihydropyrazolo[1,5-a]pyrazin- 4(5H)-one | |
| 745 | 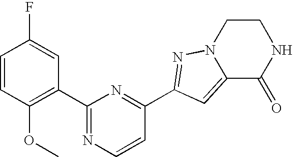 | 2-[2-(5-fluoro-2-methoxyphenyl) pyrimidin-4-yl]-6,7- dihydropyrazolo[1,5-a]pyrazin- 4(5H)-one | |
| 746 | 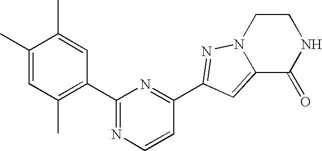 | 2-[2-(2,4,5-trimethylphenyl) pyrimidin-4-yl]-6,7- dihydropyrazolo[1,5-a]pyrazin-4(5H)-one | |
| 747 | 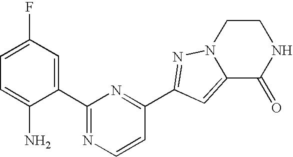 | 2-[2-(2-amino-5-fluorophenyl) pyrimidin-4-yl]-6,7- dihydropyrazolo[1,5-a]pyrazin-4(5H)-one | |
| 748 | 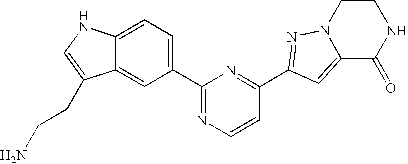 | 2-{2-[3-(2-aminoethyl)-1H- indol-5-yl]pyrimidin- 4-yl}-6,7-dihydropyrazolo [1,5-a]pyrazin-4(5H)-one | |
| 749 | 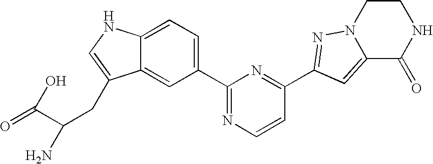 | 5-[4-(4-oxo-4,5,6,7-tetrahydropyrazolo [1,5-a]pyrazin-2-yl) pyrimidin-2-yl]tryptophan | |
| 750 | 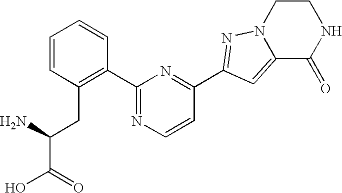 | 2-[4-(4-oxo-4,5,6,7-tetrahydropyrazolo [1,5-a]pyrazin-2-yl) pyrimidin-2-yl]-L- phenylalanine | |
| 751 | 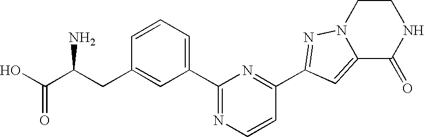 | 3-[4-(4-oxo-4,5,6,7-tetrahydropyrazolo [1,5-a]pyrazin-2-yl) pyrimidin-2-yl]-L- phenylalanine | |
| 752 | 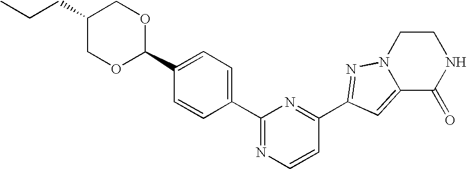 | 2-{2-[4-(5-propyl-1,3-dioxan-2- yl)phenyl]pyrimidin-4-yl}-6,7- dihydropyrazolo[1,5-a]pyrazin-4(5H)-one | |
| 753 | 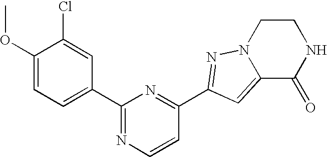 | 2-[2-(3-chloro-4-methoxyphenyl) pyrimidin-4-yl]-6,7- dihydropyrazolo[1,5-a]pyrazin-4(5H)-one | |
| 754 | 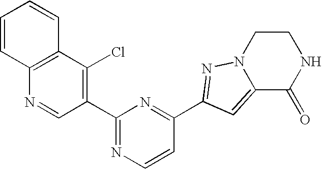 | 2-[2-(4-chloroquinolin-3-yl)pyrimidin- 4-yl]-6,7- dihydropyrazolo[1,5-a]pyrazin-4(5H)-one | |
| 755 | 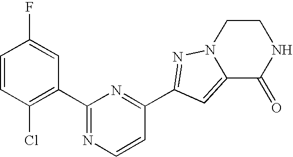 | 2-[2-(2-chloro-5-fluorophenyl) pyrimidin-4-yl]-6,7- dihydropyrazolo[1,5-a]pyrazin-4(5H)-one | |
| 756 | 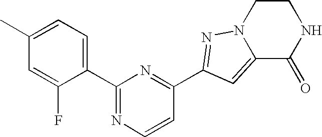 | 2-[2-(2-fluoro-4-methylphenyl) pyrimidin-4-yl]-6,7- dihydropyrazolo[1,5-a]pyrazin-4(5H)-one | |
| 757 | 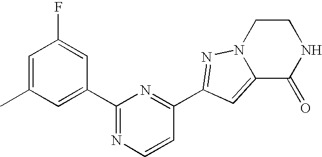 | 2-[2-(3-fluoro-5-methylphenyl) pyrimidin-4-yl]-6,7- dihydropyrazolo[1,5-a]pyrazin-4(5H)-one | |
| 758 | 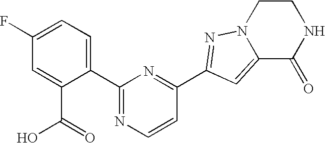 | 5-fluoro-2-[4-(4-oxo-4,5,6,7- tetrahydropyrazolo[1,5-a]pyrazin-2- yl)pyrimidin-2-yl]benzoic acid | |
| 759 | 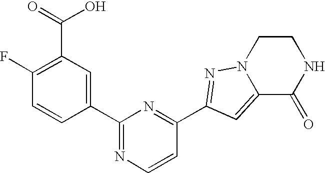 | 2-fluoro-5-[4-(4-oxo-4,5,6,7- tetrahydropyrazolo[1,5-a]pyrazin-2- yl)pyrimidin-2-yl]benzoic acid | |
| 760 | 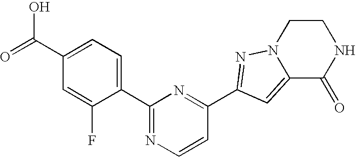 | 3-fluoro-4-[4-(4-oxo-4,5,6,7- tetrahydropyrazolo[1,5-a]pyrazin-2- yl)pyrimidin-2-yl]benzoic acid | |
| 761 | 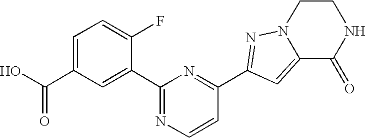 | 4-fluoro-3-[4-(4-oxo-4,5,6,7- tetrahydropyrazolo[1,5-a]pyrazin-2- yl)pyrimidin-2-yl]benzoic acid | |
| 762 | 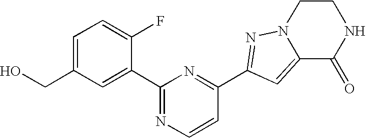 | 2-(2-[2-fluoro-5- (hydroxymethyl)phenyl]pyrimidin- 4-yl}-6,7- dihydropyrazolo[1,5-a]pyrazin-4(5H)-one | |
| 763 | 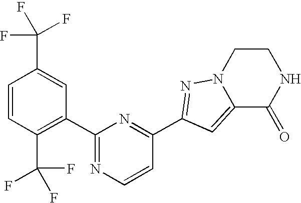 | 2-{2-[2,5-bis(trifluoromethyl) phenyl]pyrimidin-4-yl}- 6,7-dihydropyrazolo[1,5-a]pyrazin-4(5H)-one | |
| 764 | 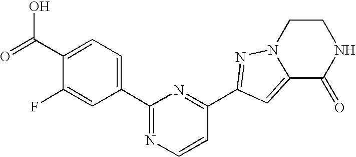 | 2-fluoro-4-[4-(4-oxo-4,5,6,7- tetrahydropyrazolo[1,5-a]pyrazin-2- yl)pyrimidin-2-yl]benzoic acid | |
| 765 | 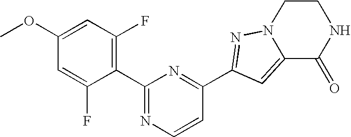 | 2-[2-(2,6-difluoro-4- methoxyphenyl)pyrimidin-4-yl]- 6,7-dihydropyrazolo[1,5-a]pyrazin-4(5H)-one | |
| 766 | 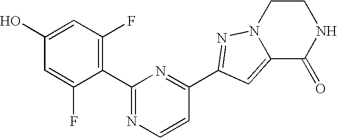 | 2-[2-(2,6-difluoro-4- hydroxyphenyl)pyrimidin-4-yl]- 6,7-dihydropyrazolo[1,5-a]pyrazin-4(5H)-one | |
| 767 | 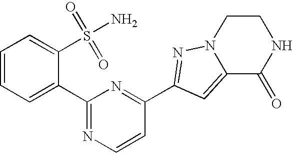 | 2-[4-(4-oxo-4,5,6,7-tetrahydropyrazolo [1,5-a]pyrazin-2-yl) pyrimidin-2-yl]benzenesulfonamide | |
| 768 | 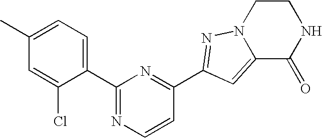 | 2-[2-(2-chloro-4-methylphenyl) pyrimidin-4-yl]-6,7- dihydropyrazolo[1,5-a]pyrazin-4(5H)-one | |
| 769 | 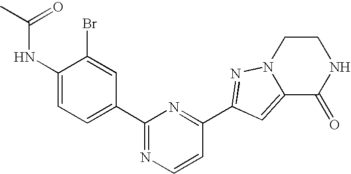 | N-{2-bromo-4-[4-(4-oxo-4,5,6,7- tetrahydropyrazolo[1,5-a]pyrazin-2- yl)pyrimidin-2-yl]phenyl}acetamide | |
| 770 | 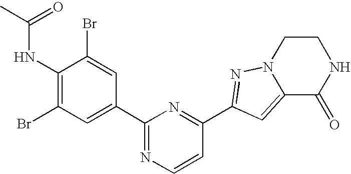 | N-{2,6-dibromo-4-[4-(4-oxo- 4,5,6,7-tetrahydropyrazolo[1,5-a]pyrazin-2-yl)pyrimidin-2- yl]phenyl}acetamide | |
| 771 | 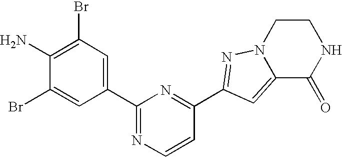 | 2-[2-(4-amino-3,5-dibromophenyl) pyrimidin-4-yl]-6,7- dihydropyrazolo[1,5-a]pyrazin- 4(5H)-one | |
| 772 | 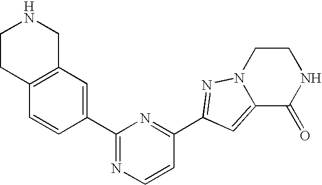 | 2-[2-(1,2,3,4-tetrahydroisoquinolin-7- yl)pyrimidin-4-yl]-6,7- dihydropyrazolo[1,5-a]pyrazin-4(5H)-one | |
| 773 | 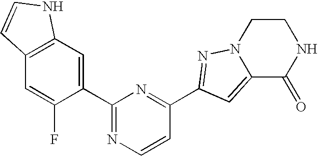 | 2-[2-(5-fluoro-1H-indol-6-yl)pyrimidin- 4-yl]-6,7-dihydropyrazolo [1,5-a]pyrazin-4(5H)-one | |
| 774 | 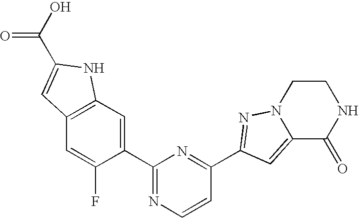 | 5-fluoro-6-[4-(4-oxo-4,5,6,7- tetrahydropyrazolo[1,5-a]pyrazin-2- yl)pyrimidin-2-yl]-1H- indole-2-carboxylic acid | |
| 775 | 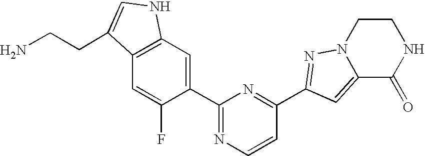 | 2-{2-[3-(2-aminoethyl)-5-fluoro-1H- indol-6-yl]pyrimidin-4-yl}-6,7- dihydropyrazolo[1,5-a]pyrazin-4(5H)-one | |
| 776 | 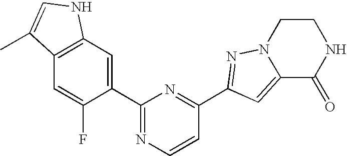 | 2-[2-(5-fluoro-3-methyl-1H-indol-6- yl)pyrimidin-4-yl]-6,7- dihydropyrazolo[1,5-a]pyrazin-4(5H)-one | |
| 777 | 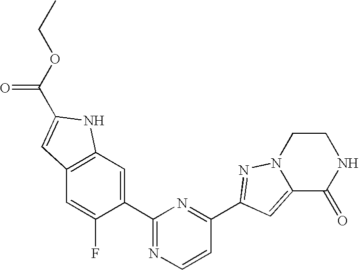 | ethyl 5-fluoro-6-[4-(4-oxo-4,5,6,7- tetrahydropyrazolo[1,5-a]pyrazin-2- yl)pyrimidin-2-yl]-1H- indole-2-carboxylate | |
| 778 | 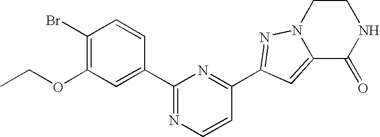 | 2-[2-(4-bromo-3-ethoxyphenyl) pyrimidin-4-yl]-6,7- dihydropyrazolo[1,5-a]pyrazin-4(5H)-one | |
| 779 | 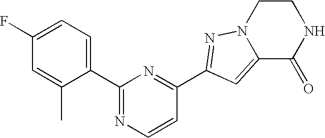 | 2-[2-(4-fluoro-2-methylphenyl) pyrimidin-4-yl]-6,7- dihydropyrazolo[1,5-a]pyrazin-4(5H)-one | |
| 780 | 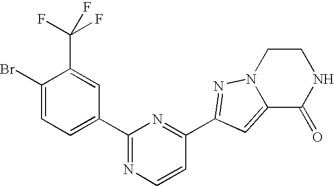 | 2-{2-[4-bromo-3-(trifluoromethyl) phenyl]pyrimidin-4-yl}-6,7- dihydropyrazolo[1,5-a]pyrazin-4(5H)-one | |
| 781 | 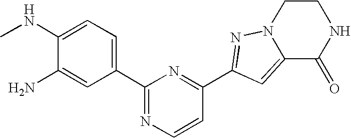 | 2-{2-[3-amino-4-(methylamino) phenyl]pyrimidin-4-yl}-6,7- dihydropyrazolo[1,5-a]pyrazin-4(5H)-one | |
| 782 | 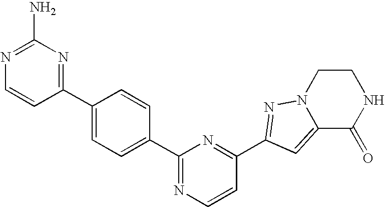 | 2-{2-[4-(2-aminopyrimidin-4- yl)phenyl]pyrimidin-4-yl}-6,7- dihydropyrazolo[1,5- a]pyrazin-4(5H)-one | |
| 783 | 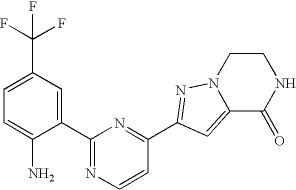 | 2-{2-[2-amino-5-(trifluoromethyl) phenyl]pyrimidin-4-yl}-6,7- dihydropyrazolo[1,5-a]pyrazin-4(5H)-one | |
| 784 | 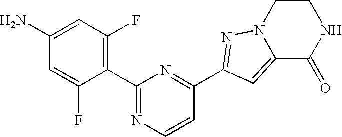 | 2-{2-(4-amino-2,6-difluorophenyl) pyrimidin-4-yl]-6,7- dihydropyrazolo[1,5-a]pyrazin- 4(5H)-one | |
| 785 | 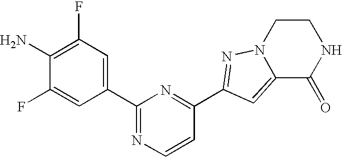 | 2-[2-(4-amino-3,5-difluorophenyl) pyrimidin-4-yl]-6,7- dihydropyrazolo[1,5-a]pyrazin- 4(5H)-one | |
| 786 | 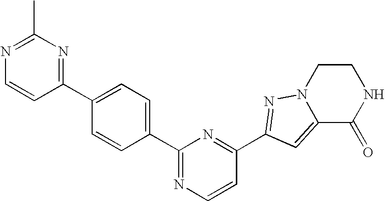 | 2-{2-[4-(2-methylpyrimidin-4- yl)phenyl]pyrimidin-4-yl}-6,7- dihydropyrazolo[1,5-a]pyrazin-4(5H)-one | |
| 787 | 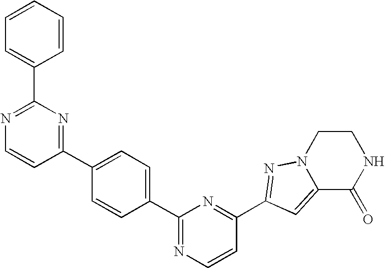 | 2-{2-[4-(2-phenylpyrimidin-4-yl) phenyl]pyrimidin-4-yl}-6,7- dihydropyrazolo[1,5-a]pyrazin-4(5H)-one | |
| 788 | 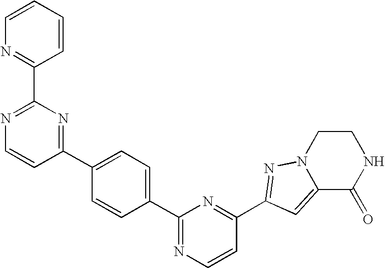 | 2-{2-[4-(2-pyridin-2-ylpyrimidin-4- yl)phenyl]pyrimidin-4-yl}-6,7- dihydropyrazolo[1,5-a]pyrazin- 4(5H)-one | |
| 789 | 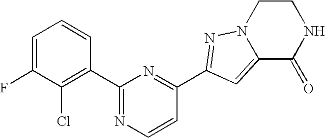 | 2-[2-(2-chloro-3-fluorophenyl) pyrimidin-4-yl]-6,7- dihydropyrazolo[1,5-a]pyrazin-4(5H)-one | |
| 790 | 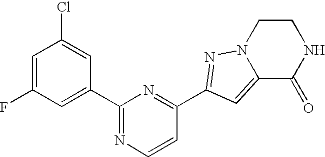 | 2-[2-(3-chloro-5-fluorophenyl) pyrimidin-4-yl]-6,7- dihydropyrazolo[1,5-a]pyrazin-4(5H)-one | |
| 791 | 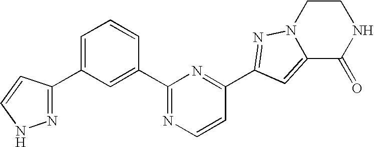 | 2-{2-[3-(1H-pyrazol-3-yl)phenyl]pyrimidin-4-yl}-6,7- dihydropyrazolo[1,5-a]pyrazin-4(5H)-one | |
| 792 | 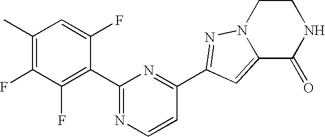 | 2-[2-(2,3,6-trifluoro-4- methylphenyl)pyrimidin-4-yl]-6,7- dihydropyrazolo[1,5-a]pyrazin- 4(5H)-one | |
| 793 | 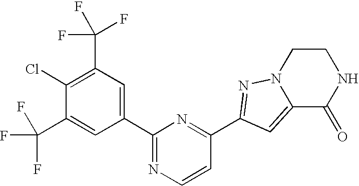 | 2-[2-[4-chloro-3,5- bis(trifluoromethyl)phenyl]pyrimidin-4-yl}-6,7- dihydropyrazolo[1,5-a]pyrazin-4(5H)-one | |
| 794 | 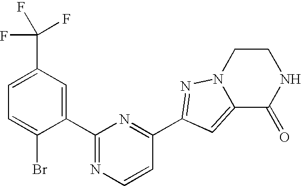 | 2-{2-[2-bromo-5- (trifluoromethyl)phenyl]pyrimidin-4-yl}-6,7- dihydropyrazolo[1,5-a]pyrazin-4(5H)-one | |
| 795 | 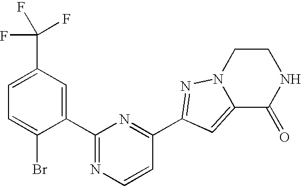 | 2-{2-[2-chloro-6- (trifluoromethyl)phenyl]pyrimidin-4-yl}-6,7- dihydropyrazolo[1,5-a]pyrazin-4(5H)-one | |
| 796 | 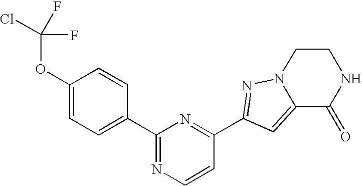 | 2-(2-{4-[chloro(difluoro)methoxy]phenyl}pyrimidin-4-yl)- 6,7-dihydropyrazolo[1,5-a]pyrazin-4(5H)-one | |
| 797 | 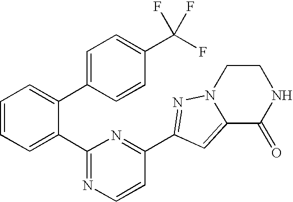 | 2-{2-[4′-(trifluoromethyl)-1,1′- biphenyl-2-yl]pyrimidin-4-yl}-6,7- dihydropyrazolo[1,5-a]pyrazin-4(5H)-one | |
| 798 | 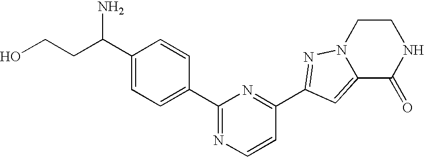 | 2-{2-[4-(1-amino-3- hydroxypropyl)phenyl]pyrimidin-4- yl}-6,7-dihydropyrazolo [1,5-a]pyrazin-4(5H)-one | |
| 799 | 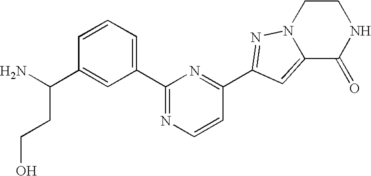 | 2-{2-[3-(1-amino-3- hydroxypropyl)phenyl]pyrimidin-4- yl)-6,7-dihydropyrazolo [1,5-a]pyrazin-4(5H)-one | |
| 800 | 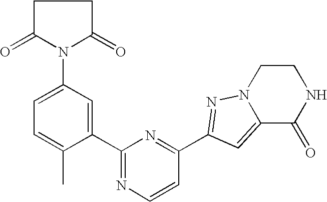 | 1-{4-methyl-3-[4-(4-oxo-4,5,6,7- tetrahydropyrazolo[1,5-a]pyrazin-2-yl)pyrimidin- 2-yl]phenyl}pyrrolidine- 2,5-dione | |
| 801 | 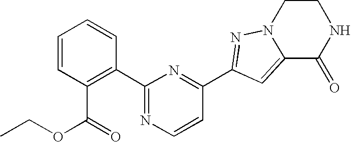 | ethyl 2-[4-(4-oxo-4,5,6,7- tetrahydropyrazolo[1,5-a]pyrazin-2-yl) pyrimidin-2-yl]benzoate | |
| 802 | 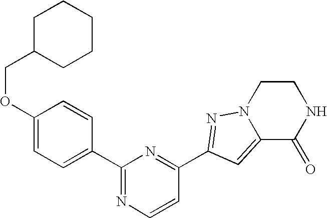 | 2-{2-[4-(cyclohexylmethoxy) phenyl]pyrimidin-4-yl}-6,7- dihydropyrazolo[1,5-a]pyrazin- 4(5H)-one | |
| 803 | 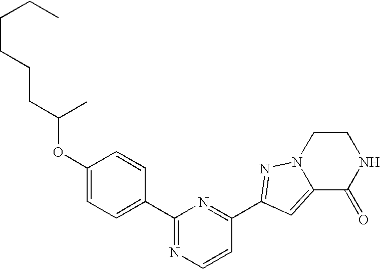 | 2-(2-{4-[(1-methylheptyl) oxy]phenyl}pyrimidin-4- yl)-6,7-dihydropyrazolo [1,5-a]pyrazin-4(5H)-one | |
| 804 | 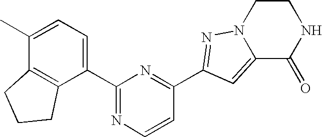 | 2-[2-(7-methyl-2,3-dihydro-1H-inden-4- yl)pyrimidin-4-yl]-6,7- dihydropyrazolo[1,5-a]pyrazin-4(5H)-one | |
| 805 | 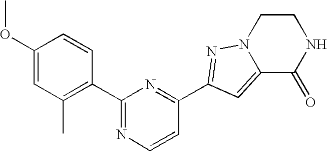 | 2-[2-(4-methoxy-2-methylphenyl) pyrimidin-4-yl]-6,7- dihydropyrazolo[1,5-a]pyrazin-4(5H)-one | |
| 806 | 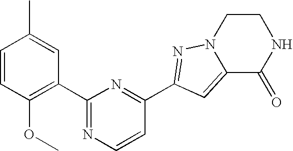 | 2-[2-(2-methoxy-5-methylphenyl) pyrimidin-4-yl]-6,7- dihydropyrazolo[1,5-a]pyrazin-4(5H)-one | |
| 807 | 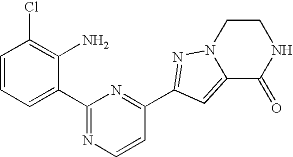 | 2-[2-(2-amino-3-chlorophenyl) pyrimidin-4-yl]-6,7- dihydropyrazolo[1,5-a]pyrazin-4(5H)-one | |
| 808 | 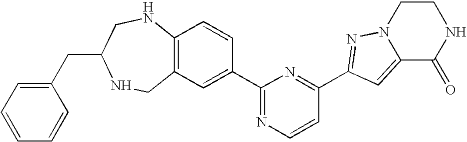 | 2-[2-(3-benzyl-2,3,4,5-tetrahydro- 1H-1,4-benzodiazepin-7-yl) pyrimidin-4-yl]-6,7- dihydropyrazolo[1,5-a]pyrazin-4(5H)-one | |
| 809 | 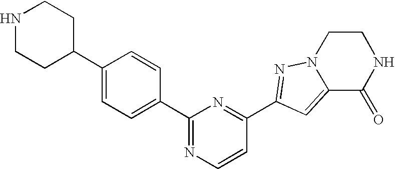 | 2-[2-(4-piperidin-4-ylphenyl) pyrimidin-4-yl]-6,7- dihydropyrazolo[1,5-a]pyrazin-4(5H)-one | |
| 810 | 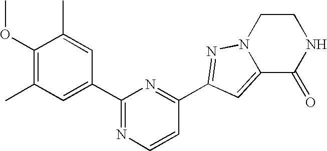 | 2-[2-(4-methoxy-3,5-dimethylphenyl) pyrimidin-4-yl]-6,7- dihydropyrazolo[1,5-a]pyrazin-4(5H)-one | |
| 811 | 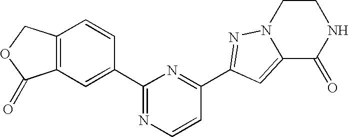 | 2-[2-(3-oxo-1,3-dihydro-2-benzofuran- 5-yl)pyrimidin-4-yl]-6,7- dihydropyrazolo[1,5-a]pyrazin-4(5H)-one | |
| 812 | 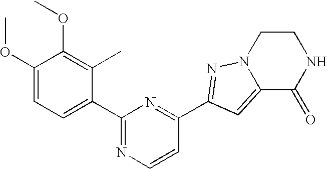 | 2-[2-(3,4-dimethoxy-2-methylphenyl) pyrimidin-4-yl]-6,7- dihydropyrazolo[1,5-a]pyrazin-4(5H)-one | |
| 813 | 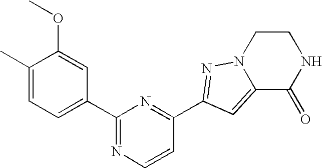 | 2-[2-(3-methoxy-4-methylphenyl) pyrimidin-4-yl]-6,7- dihydropyrazolo[1,5-a]pyrazin-4(5H)-one | |
| 814 | 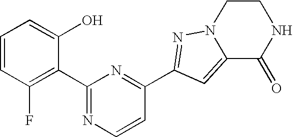 | 2-[2-(2-fluoro-6-hydroxyphenyl) pyrimidin-4-yl]-6,7- dihydropyrazolo[1,5-a]pyrazin-4(5H)-one | |
| 815 | 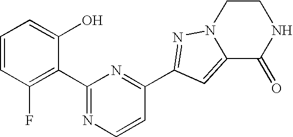 | 2-[2-(3,4-difluoro-2- methoxyphenyl)pyrimidin-4-yl]-6,7- dihydropyrazolo[1,5-a]pyrazin-4(5H)-one | |
| 816 | 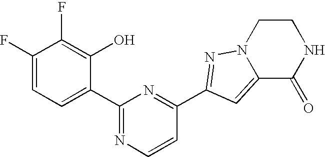 | 2-[2-(3,4-difluoro-2- hydroxyphenyl)pyrimidin-4-yl]-6,7- dihydrapyrazolo[1,5-a]pyrazin-4(5H)-one | |
| 817 | 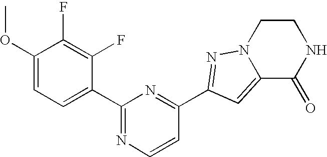 | 2-[2-(2,3-difluoro-4- methoxyphenyl)pyrimidin-4-yl]-6,7- dihydropyrazolo[1,5-a]pyrazin-4(5H)-one | |
| 818 | 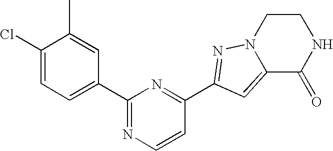 | 2-[2-(4-chloro-3-methylphenyl) pyrimidin-4-yl]-6,7- dihydrapyrazalo[1,5-a]pyrazin- 4(5H)-one | |
| 819 | 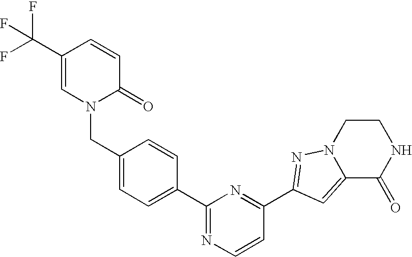 | 2-[2-(4-{[2-oxo-5-(trifluoramethyl) pyridin-1(2H)-yl]methyl}phenyl)pyrimidin-4-yl]-6,7- dihydropyrazolo[1,5-a]pyrazin-4(5H)-one | |
| 820 | 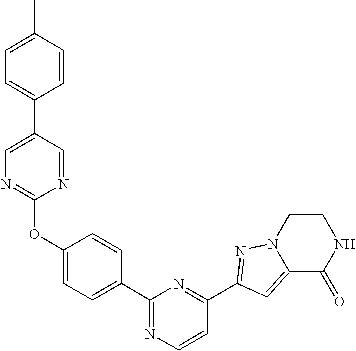 | 2-[2-(4-{[5-(4-methylphenyl)pyrimidin- 2-yl]oxy}phenyl)pyrimidin-4- yl]-6,7-dihydropyrazolo [1,5-a]pyrazin-4(5H)-one | |
| 821 | 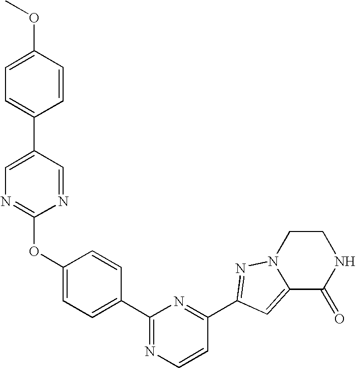 | 2-[2-(4-{[5-(4-methoxyphenyl) pyrimidin-2-yl]oxy}phenyl)pyrimidin-4-yl]-6,7- dihydropyrazolo[1,5-a]pyrazin- 4(5H)-one | |
| 822 | 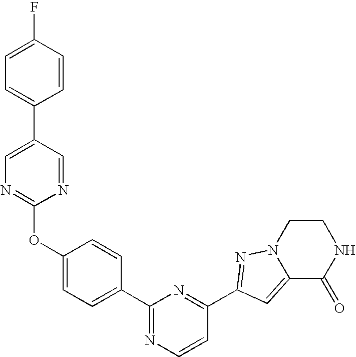 | 2-[2-(4-{[5-(4-fluorophenyl) pyrimidin-2-yl]oxy}phenyl)pyrimidin-4-yl]-6,7- dihydropyrazolo[1,5-a]pyrazin- 4(5H)-one | |
| 823 | 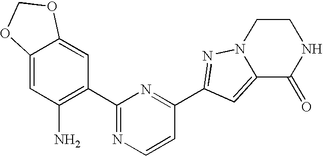 | 2-[2-(6-amino-1,3-benzodioxol-5- yl)pyrimidin-4-yl]-6,7- dihydropyrazolo[1,5-a]pyrazin-4(5H)-one | |
| 824 | 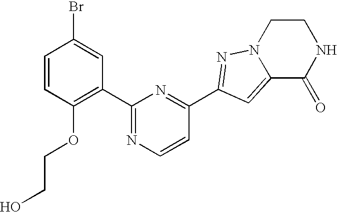 | 2-{2-[5-bromo-2-(2- hydroxyethoxy)phenyl]pyrimidin- 4-yl}-6,7-dihydropyrazolo[1,5-a]pyrazin-4(5H)-one | |
| 825 | 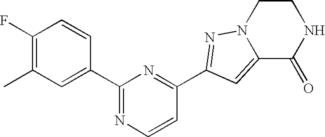 | 2-[2-(4-fluoro-3-methylphenyl) pyrimidin-4-yl]-6,7- dihydropyrazolo[1,5-a]pyrazin-4(5H)-one | |
| 826 | 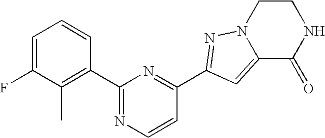 | 2-[2-(3-fluoro-2-methylphenyl) pyrimidin-4-yl]-6,7- dihydropyrazolo[1,5-a]pyrazin-4(5H)-one | |
| 827 | 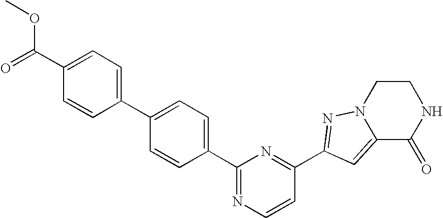 | methyl 4′-[4-(4-oxo-4,5,6,7- tetrahydropyrazolo[1,5-a]pyrazin-2- yl)pyrimidin-2-yl]- 1,1′-biphenyl-4- carboxylate | |
| 828 | 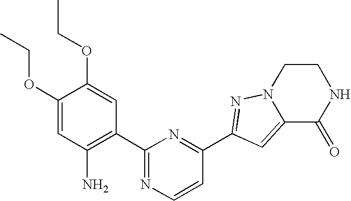 | 2-[2-(2-amino-4,5-diethoxyphenyl) pyrimidin-4-yl]-6,7- dihydropyrazolo[1,5-a]pyrazin- 4(5H)-one | |

Notes:
^{a}Chemical names were generated by ACD/Name software.
^{b}The MK-2 inhibiting compound may be shown with a solvent, such as, for example, trifluoroacetate, with which it can form a salt. Both the salt and base forms of each compound are included in the present invention.

In one embodiment of the present invention, the MK-2 inhibiting compound is one that is listed in Table I.

In another embodiment of the present invention, the MK-2 inhibiting compound is one that is listed in Table 1 or in Table 2.

In yet another embodiment of the present invention, the MK-2 inhibiting compound is one that is listed in Table 2.

It is preferred that the MK-2 inhibiting compound is one that has an IC₅₀ value for the inhibition of MK-2 that is lower than 1. By way of example, this would include the compounds in Table I numbered 1-56. An MK-2 IC₅₀ value that is lower than 0.5 is still more preferred (examples of these compounds include the compounds in Table I numbered 1-32), lower than 0.1 is even more preferred yet (examples of these compound include the compounds in Table I numbered 1-7).

In one embodiment of the present invention, the MK-2 inhibiting compound is one having the structure of formula I, except when Z² and Z³ are both nitrogen, R⁴ is other than pyrrole, or optionally when Z⁴ and Z⁵ are both nitrogen and R^{a} is ring Q, Q² is other than nitrogen.

In another embodiment of the present invention, the MK-2 inhibiting compound is one having the structure of formula I, except that R^{a} is selected from an M-ring or a Q-ring.

In another embodiment of the present invention, the MK-2-inhibiting compound is one having the structure of formula I, except that R^{a} is an M-ring.

In another embodiment of the present invention, the MK-2 inhibiting compound is one having the structure of formula 1, except that R^{a} is an M-ring wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if M² and/or M⁶ is carbon, the carbon is substituted with (L)ₙR¹.

In another embodiment of the present invention, the MK-2 inhibiting compound is one having the structure of formula I, except that R^{a} is an M-ring wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if M² and/or M⁶ is carbon, the carbon is substituted with (L)ₙR¹; and

R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

In another embodiment of the present invention, the MK-2 inhibiting compound is one having the structure of formula I, except that R^{a} is an M-ring wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if carbon, the carbon is substituted with (L)ₙR¹; and

R³ and R⁴ optionally join to form a ring of 6 or 7 atoms, where the atoms in the ring are independently selected from Z³, Z⁴, C═O, C that is mono or di-substituted with an R¹ group, and N that is unsubstituted or substituted with an R¹ group.

In another embodiment of the present invention, the MK-2 inhibiting compound is one having the structure of formula I, except that R^{a} is an M-ring, wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if carbon, the carbon is substituted with (L)ₙR¹; and

R³ and R⁴ optionally join to form a ring of 6 atoms, where the atoms in the ring are independently selected from Z³, Z⁴, C═O, C that is mono or di-substituted with an R¹ group, and N that is unsubstituted or substituted with an R¹ group.

In another embodiment of the present invention, the MK-2 inhibiting compound is one having the structure of formula I, except that R¹ is an M-ring wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if carbon, the carbon is substituted with (L)ₙR¹; and

R³ and R⁴ optionally join to form a ring that is selected from:

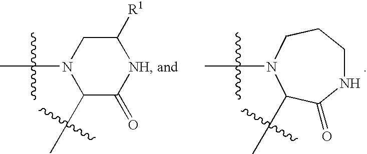

In another embodiment of the present invention, the MK-2 inhibiting compound is one having the structure of formula I, except that R^{a} is an M-ring, wherein ring M is an aromatic pyridine ring, wherein M¹, M³, M⁴ and M⁶ are carbon and are substituted with (L)ₙR¹, M⁵ is carbon, M² is nitrogen.

In another embodiment of the present invention, the MK-2 inhibiting compound is one having the structure of formula I, except that R^{a} is an M-ring, wherein ring M is an aromatic pyrimidine ring, wherein M¹, M³ and M⁴ are carbon and are substituted with (L)ₙR¹, M⁵ is carbon, M² and M⁶ are nitrogen.

In another embodiment of the present invention, the MK-2 inhibiting compound is one having the structure of formula I, except that R^{a} is an M-ring wherein ring M is an aromatic pyridine ring, wherein:

M¹, M³, M⁴ and M⁶ are carbon and are substituted with (L)ₙR¹;

M⁵ is carbon;

M² is nitrogen;

R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR¹⁷, COR₆, CO₂R₆, CONHR₆, N(R⁸)₂, amino C₁-C₄ alkyl, hydroxy C₁-C₄ alkyl, amino, amino C₁-C₄ alkyl-R⁷, C₁-C₆ alkyl-NHR⁷, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R⁷, R⁸, are each independently selected from —H, C₁-C₆ alkyl, C₁-C₄ alkyl-R¹¹, C₁-C₆ alkyl-N(R³)₂, CO₂R¹⁶, COR¹⁷, aryl, and arylalkyl, wherein aryl and arylalkyl, are optionally substituted with one or more of the groups defined by R¹⁸;

R⁹, R¹⁰ are each independently selected from —H, hydroxyl, C₁-C₆ alkyl, C₁-C₆ alkyl-R¹⁷, C₁-C₆ alkyl-NH₂R¹³, CO₂R¹⁶, COR⁷, C₁-C₆ alkyl-CO₂R¹⁶, C₁-C₆ alkyl-CONH—R¹⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;

R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₁-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;

R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R⁸)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, COR²³, and aryl;

R¹⁵, R¹⁶ are each independently selected from —H, aryl, arylalkyl, wherein aryl, arylalkyl, are optionally substituted with one or more of the groups defined by R²⁴;

R¹⁷ is selected from —H, C₁-C₆ alkyl, C₁-C₆ alkyl-R¹⁹, NHR¹⁹, aryl, heteroarylalkyl, and heterocyclylalkyl, wherein aryl is optionally substituted with one or more of the groups defined by R²⁴;

R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;

R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;

R²¹ and R²² are each independently selected from —H and C₁-C₆ alkyl;

R²³ is selected from —H and C₁-C₆ alkyl;

R²⁴ is selected from —H, C₁-C₆ alkyl, C₁-C₆ alkoxy, CO₂R²⁹, halo, and halo C₁-C₄ alkyl;

R²⁹ is selected from —H, and C₁-C₆ alkyl;

R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;

R³⁶ is selected from —H and halo; and

R², R³, R⁴, R³⁷ and R³⁸ are each independently selected from an R¹ group.

In an embodiment of the present invention, the MK-2 inhibiting compound is one having the structure of formula I, except that R^{a} is an M-ring wherein ring M is an aromatic pyrimidine ring, wherein:

M¹, M³ and M⁴ are carbon and are substituted with (L)ₙR¹;

M⁵ is carbon;

M² and M⁶ are nitrogen;

R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR⁷, COR₆, CO₂R₆, CONHR₆, N(R⁸)₂, amino C₁-C₄ alkyl, hydroxy C₁-C₄ alkyl, amino, amino C₁-C₄ alkyl-R⁷, halo C₁-C₄ alkyl, C₁-C₆ alkyl-NHR⁷, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R⁷, R⁸, are each independently selected from —H, C₁-C₆ alkyl, C₁-C₄ alkyl-R¹¹, C₁-C₆ alkyl-N(R³)₂, CO₂R¹⁶, COR⁷, aryl, and arylalkyl, wherein aryl and arylalkyl, are optionally substituted with one or more of the groups defined by R¹⁸;

R⁹, R¹⁰ are each independently selected from —H, hydroxyl, C₁-C₆ alkyl, C₁-C₆ alkyl-R¹⁷, C₁-C₆ alkyl-NH₂R¹³, CO₂R¹⁶, COR¹⁷, C₁-C₆ alkyl-CO₂R¹⁶, C₁-C₆ alkyl-CONH—R¹⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;

R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₁-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;

R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R⁸)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, COR²³, and aryl;

R¹⁵, R¹⁶ are each independently selected from —H, aryl, arylalkyl, wherein aryl, arylalkyl, are optionally substituted with one or more of the groups defined by R²⁴;

R¹⁷ is selected from —H, C₁-C₆ alkyl, C₁-C₆ alkyl-R¹⁹, NHR¹⁹, aryl; heteroarylalkyl, and heterocyclylalkyl, wherein aryl is optionally substituted with one or more of the groups defined by R²⁴.

R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;

R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;

R²¹ and R²² are each independently selected from —H and C₁-C₆ alkyl;

R²³ is selected from —H and C₁-C₆ alkyl;

R²⁴ is selected from —H, C₁-C₆ alkyl, C₁-C₆ alkoxy, CO₂R²⁹, halo, and halo C₁-C₄ alkyl;

R²⁹ is selected from —H, and C₁-C₆ alkyl;

R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;

R³⁶ is selected from —H and halo; and

R², R³, R⁴, R³⁷ and R³⁸ are each independently selected from an R¹ group.

In another embodiment, the present MK-2 inhibiting compound has the structure shown in formula III:

Formula III:

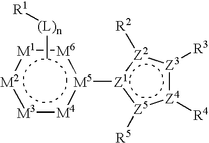

wherein:

dashed lines indicate optional single or double bonds;

Z² and Z³ are nitrogen, Z¹, Z⁴ and Z⁵ are carbon, and join with Z² and Z³ to form a pyrazole ring;

where dashed lines indicate optional single or double bonds;

M¹, M³ and M⁴ is carbon and is substituted with (L)ₙR¹, M⁵ is carbon, and each of M² and M⁶ is independently selected from nitrogen and carbon, and if carbon, it is unsubstituted or substituted with (L)ₙR¹;

R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR¹⁷, CO₂R⁷, CONHR⁷, N(R⁸)₂, amino C₁-C₄ alkyl, hydroxy C₁-C₄ alkyl, amino, amino C₁-C₄ alkyl-R⁷, halo C₁-C₄ alkyl, C₁-C₆ alkyl-NHR⁷, C₁-C₆ alkyl-N(R⁷)₂, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R⁷ and R⁸ are each independently selected from —H, C₁-C₆ alkyl, C₁-C₄ alkyl-R¹¹, C₁-C₆ alkyl-N(R³)₂, CO₂R¹⁶, COR¹⁷, aryl, and arylalkyl, wherein aryl and arylalkyl, are optionally substituted with one or more of the groups defined by R¹⁸;

R⁹ and R¹⁰ are each independently selected from —H, hydroxyl, C₁-C₆ alkyl, C₁-C₆ alkyl-R¹⁷, C₁-C₆ alkyl-NH₂R¹³, CO₂R¹⁶, COR¹⁷, C₁-C₆ alkyl-CO₂R¹⁶, C₁-C₆ alkyl-CONH—R¹⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;

R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₁-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;

R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R⁸)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;

R¹³ and R¹⁴ are each independently selected from —H, oxo, C₁-C₆ alkyl, COR²³, and aryl;

R¹⁵ and R¹⁶ are each independently selected from —H, aryl, arylalkyl, wherein aryl, arylalkyl, are optionally substituted with one or more of the groups defined by R²⁴;

R¹⁷ is selected from —H, C₁-C₆ alkyl, C₁-C₆ alkyl-R¹⁹, NHR¹⁹, aryl, heteroarylalkyl, and heterocyclylalkyl, wherein aryl is optionally substituted with one or more of the groups defined by R²⁴;

R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;

R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;

R²¹ and R²² are each independently selected from —H and C₁-C₆ alkyl;

R²³ is selected from —H and C₁-C₆ alkyl;

R²⁴ is selected from —H, C₁-C₆ alkyl, C₁-C₆ alkoxy, CO₂R²⁹, halo, and halo C₁-C₄ alkyl;

R²⁹ is selected from —H, and C₁-C₆ alkyl;

R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;

R³⁶ is selected from —H and halo; R², R³, R⁴, R³⁷ and R³⁸ are each independently selected from an R¹ group;

n is 0; and

R³ and R⁴ optionally join to form a ring structure that is selected from:

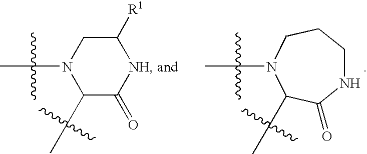

The MK-2 inhibiting compounds that are described in formulas I-III, and in Tables I and II can be made by the methods that are described in the Examples below. Compounds that are not described specifically in the Examples can be made by reference to the methods used in the Examples, but with the substitution of starting compounds that are suitable for the compound that is desired.

The present invention also includes a method of inhibiting mitogen activated protein kinase-activated protein kinase-2, the method comprising contacting a mitogen activated protein kinase-activated protein kinase-2 with one or more of any of the MK-2 inhibiting compounds described herein. In one embodiment, the contacting of MK-2 with an MK-2 inhibitory compound takes place inside a cell. The cell can be one of any type of organism, but is preferably an animal cell. Contacting can occur in vitro or in vivo, and the cell can be a living cell, or it can be non-living. When the contacting is carried out in vitro, the cell can be attached to other cells, or it can be a single cell, or clump of cells in suspension or on a solid medium. When the contacting is carried out in vivo, the MK-2 inhibitory compound can be administered as described below.

In one embodiment, the present invention provides a method for treating or preventing an MK-2 modulated disease or disorder in a subject, the method comprises contacting a mitogen activated protein kinase-activated protein kinase-2 in a subject with one or more of the MK-2 inhibiting compounds that are described herein. A preferred MK-2 inhibiting compound for the present method is one having the structure described by formula I. In another perferred embodiment, the MK-2 inhibiting compound is one having the structure described by formula II.

The present invention also includes a method of inhibiting mitogen activated protein kinase-activated protein kinase-2 in a subject in need of such inhibition, the method comprising administering to the subject one or more of the MK-2 inhibiting compounds described herein.

The present invention also includes a method of preventing or treating a TNFα mediated disease or disorder in a subject, the method comprising administering to the subject an effective amount of one or more of the MK-2 inhibiting compounds described herein. In a preferred embodiment, the subject is one that is in need of such prevention or treatment.

The present methods can be practiced by the administration of any one or more of the present MK-2 inhibiting compounds. It is preferred tht the MK-2 inhibiting compound is one having an MK-2 IC₅₀ of less than about 10 μM, in an in vitro assay of MK-2 inhibitory activity, more preferred is a compound having an MK-2 IC₅₀ of less than about 1.0 μM, yet more preferred is a compound having an MK-2 IC₅₀ of less than about 0.5 μM.

It should be understood that the base forms, salts, pharmaceutically acceptable salts, and prodrugs of the compounds that are described herein, as well as isomeric forms, tautomers, racemic mixtures of the compounds, and the like, which have the same or similar activity as the compounds that are described, are to be considered to be included within the description of the compound.

The MK-2 inhibiting activity of any of the compounds described herein can be determined by any one of several methods that are well known to those having skill in the art of enzyme activity testing. One such method is described in detail in the general methods section of the examples. In addition, the efficacy of any one of the present MK-2 inhibiting compounds in therapeutic applications can be determined by testing for inhibition of TNFα production in cell culture and in animal model assays. In general, it is preferred that the MK-2 inhibiting compounds of the present invention be capable of inhibiting the production and/or the release of TNFα in cell cultures and in animal models.

In the present method, the MK-2 inhibiting compounds that are described herein can be used as inhibitors of MAPKAP kinase-2. When this inhibition is for a therapeutic purpose, one or more of the present MK-2 inhibitory compounds can be administered to a subject that is in need of MK-2 inhibition. As used herein, a “subject in need of MK-2 inhibition” is a subject who has, or who is at risk of contracting a TNFα mediated disease or disorder. TNFα mediated diseases and disorders are described in more detail below.

As described above, in an embodiment of the present method, a subject in need of prevention or treatment of a TNFα mediated disease or disorder is treated with one or more of the present MK-2 inhibiting compounds. In one embodiment, the subject is treated with an effective amount of the MK-2 inhibiting compound. The effective amount can be an amount that is sufficient for preventing or treating the TNFα mediated disease or disorder.

The MK-2 inhibiting compound that is used in the subject method can be any MK-2 inhibiting compound that is described herein.

In the subject method, the MK-2 inhibiting compound can be used in any amount that is an effective amount. It is preferred, however, that the amount of the MK-2 inhibiting compound that is administered is within a range of about 0.1 mg/day per kilogram (kg) of the subject to about 1500 mg/day/kg. It is more preferred that the amount of the compound is within a range of about 1 mg/day/kg to about 500 mg/day/kg. An amount that is within a range of about 10 mg/day/kg to about 400 mg/day/kg, is even more preferred.

When the term “about” is used herein in relation to a dosage amount of the MK-2 inhibiting compound, it is to be understood to mean an amount that is within ±10% by weight of the amount or range that is described. By way of example, “about 0.1-10 mg/day” includes all dosages within 0.9 to 11 mg/day.

In an embodiment of the present invention, a therapeutic composition is provided that contains at least one of the MK-2 inhibiting compounds that are described herein. A preferred therapeutic composition contains a therapeutically effective amount of a compound that is described by formula I. In another embodiment, a preferred therapeutic composition is one having an MK-2 inhibiting compound that is described by formula II.

In another embodiment of the present invention, a pharmaceutical composition that contains one or more of the present MK-2 inhibitors can be administered to a subject for the prevention or treatment of a TNFα mediated disease or disorder. The pharmaceutical composition includes an MK-2 inhibitor of the present invention and a pharmaceutically acceptable carrier. A preferred MK-2 inhibitor for use in the pharmaceutical composition is described by formula I. In another embodiment, a preferred pharmaceutical composition is one having an MK-2 inhibiting compound that is described by formula II.

In another embodiment, a kit can be produced that is suitable for use in the prevention or treatment of a TNFα mediated disease or disorder. The kit comprises a dosage form comprising at least one of the MK-2 inhibitors that is described herein in an amount which comprises a therapeutically effective amount.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used, the nature and severity of the illness to be treated, as well as the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of the MK-2 inhibitory compound for use in therapy which will achieve the goal of improvement in the severity of pain and inflammation and the frequency of incidence over treatment, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

The frequency of dose will depend upon the half-life of the active components of the composition. If the active molecules have a short half life (e.g. from about 2 to 10 hours) it may be necessary to give one or more doses per day. Alternatively, if the active molecules have a long half-life (e.g. from about 2 to about 15 days) it may only be necessary to give a dosage once per day, per week, or even once every 1 or 2 months. A preferred dosage rate is to administer the dosage amounts described above to a subject once per day.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

For the purposes of calculating and expressing a dosage rate, all dosages that are expressed herein are calculated on an average amount-per-day basis irrespective of the dosage rate. For example, one 100 mg dosage of an MK-2 inhibitor taken once every two days would be expressed as a dosage rate of 50 mg/day. Similarly, the dosage rate of an ingredient where 50 mg is taken twice per day would be expressed as a dosage rate of 100 mg/day.

For purposes of calculation of dosage amounts, the weight of a normal adult human will be assumed to be 70 kg.

When the MK-2 inhibitor is supplied along with a pharmaceutically acceptable carrier, the pharmaceutical compositions that are described above can be formed. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is-being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the compounds and compositions of the invention are the isomeric forms and tautomers and the pharmaceutically-acceptable salts of the present MK-2 inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group IA) salts, alkaline earth metal (Group IIA) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trifluoroacetate, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The method of the present invention is useful for, but not limited to, the prevention and/or treatment of diseases and disorders that are mediated by TNFα and/or mediated by MK-2, including pain, inflammation and/or arthritis. For example, the compounds described herein would be useful for the treatment of any inflammation-related disorder described below, such as an analgesic in the treatment of pain and headaches, or as an antipyretic for the treatment of fever. The compounds described herein would also be useful for the treatment of an inflammation-related disorder in a subject suffering from such an inflammation-associated disorder.

As used herein, the terms “treating”, “treatment”, “treated”, or “to treat,” mean to alleviate symptoms, eliminate the causation either on a temporary or permanent basis. The term “treatment” includes alleviation, elimination of causation of pain and/or inflammation associated with, but not limited to, any of the diseases or disorders described herein. The terms “prevent”, “prevention”, “prevented”, or “to prevent,” mean to prevent or to slow the appearance of symptoms associated with, but not limited to, any of the diseases or disorders described herein.

In preferred embodiments, the methods and compositions of the present invention encompass the prevention and/or treatment of pain, inflammation and inflammation-related disorders.

In other preferred embodiments, the methods and compositions of the present invention encompass the treatment of any one or more of the disorders selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

In still other preferred embodiments, the methods and compositions of the present invention encompass the prevention and treatment of the connective tissue and joint disorders selected from the group consisting of arthritis, rheumatoid arthritis, spondyloarthopathies, gouty arthritis, lumbar spondylarthrosis, carpal tunnel syndrome, canine hip dysplasia, systemic lupus erythematosus, juvenile arthritis, osteoarthritis, tendonitis and bursitis.

In other preferred embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, skin cancer, brain cancer, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar-cancer, Waldenstrom's macroglobulinemia, well differentiated carcinoma, and Wilm's tumor.

In other preferred embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis, arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, vascular diseases, migraine headaches, aplastic anemia, cardiac damage, diabetic cardiac myopathy, renal insufficiency, renal injury, renal arteriopathy, peripheral vascular disease, left ventricular hypertrophy, cognitive dysfunction, stroke, and headache.

In other preferred embodiments, the methods and compositions of the present invention encompass the prevention and treatment of the metabolic disorders selected from the group consisting of obesity, overweight, type I and type II diabetes, hypothyroidism, and hyperthyroidism.

In other preferred embodiments, the methods and compositions of the present invention encompass the prevention and treatment of the respiratory disorders selected from the group consisting of asthma, bronchitis, chronic obstructive pulmonary disease (COPD), cystic fibrosis, pulmonary edema, pulmonary embolism, pneumonia, pulmonary sarcoisosis, silicosis, pulmonary fibrosis, respiratory failure, acute respiratory distress syndrome and emphysema.

In other preferred embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In other preferred embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In still further embodiments, the methods and compositions of the present invention encompass the prevention and treatment of the infectious diseases and disorders selected from the group consisting of hepatitis, HIV (AIDS), small pox, chicken pox, common cold, bacterial influenza, viral influenza, warts, oral herpes, genital herpes, herpes simplex infections, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

In other preferred embodiments, the methods and compositions of the present invention encompass the prevention and treatment of the neurological and neurodegenerative disorders selected from the group consisting of headaches, migraine headaches, Alzheimer's disease, Parkinson's disease, dementia, memory loss, senility, amyotrophy, ALS, amnesia, seizures, multiple sclerosis, muscular dystrophies, epilepsy, schizophrenia, depression, anxiety, attention deficit disorder, hyperactivity, bulimia, anorexia nervosa, anxiety, autism, phobias, spongiform encephalopathies, Creutzfeldt-Jakob disease, Huntington's Chorea, ischemia, obsessive-compulsive disorder, manic depression, bipolar disorders, drug addiction, alcoholism and smoking addiction.

In other preferred embodiments, the methods and compositions of the present invention encompass the prevention and treatment of the dermatological disorders selected from the group consisting of acne, psoriasis, eczema, burns, poison ivy, poison oak and dermatitis.

In other preferred embodiments, the methods and compositions of the present invention encompass the prevention and treatment of the surgical disorders selected from the group consisting of pain and swelling following surgery, infection following surgery and inflammation following surgery.

In other preferred embodiments, the methods and compositions of the present invention encompass the prevention and treatment of the gastrointestinal disorders selected from the group consisting of inflammatory bowel disease, irritable bowel syndrome, Crohn's disease, gastritis, irritable bowel syndrome, diarrhea, constipation, dysentery, ulcerative colitis, gastric esophageal reflux, gastric ulcers, gastric varices, ulcers, and heartburn.

In other preferred embodiments, the methods and compositions of the present invention encompass the prevention and treatment of the otic disorders selected from the group consisting of otic pain, inflammation, otorrhea, otalgia, fever, otic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, otic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, otic neoplasm, otomycosis, trauma, acute barotitis media, acute eustachian tube obstruction, post-otic surgery, postsurgical otalgia, cholesteatoma, conductive and sensorineural hearing loss, epidural abscess, lateral sinus thrombosis, subdural empyema and otitic hydrocephalus.

In other preferred embodiments, the methods and compositions of the present invention encompass the prevention and treatment of the ophthalmic disorders selected from the group consisting of retinopathies, uveitis, ocular photophobia, acute injury to the eye tissue, conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocorneal endothelial syndrome, keratoconus, lattice dystrophy, map-dot-fingerprint dystrophy, ocular herpes, pterygium, myopia, hyperopia, and cataracts.

In other preferred embodiments, the methods and compositions of the present invention encompass the prevention and treatment of menstrual cramps, kidney stones, minor injuries, wound healing, vaginitis, candidiasis, sinus headaches, tension headaches, dental pain, periarteritis nodosa, thyroiditis, myasthenia gravis, multiple sclerosis, sarcoidosis, nephrotic syndrome, Bahcet's syndrome, polymyositis, gingivitis, hypersensitivity, swelling occurring after injury, closed head injury, liver disease, and endometriosis.

As used herein, the terms “TNFα mediated disease or disorder” are meant to include, without limitation, each of the symptoms or diseases that are mentioned above.

The term “subject” for purposes of treatment includes any human or animal subject who is in need of the prevention of or treatment of any one of the TNFα mediated diseases or disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any human or animal subject, and preferably is a subject that is in need of prevention and/or treatment of a TNFα mediated diseases or disorders. The subject may be a human subject who is at risk of obtaining a TNFα mediated disease or disorder, such as those described above. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

In particular, the pharmaceutical compositions of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the MK-2 inhibitors in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel MK-2 inhibitory compounds may be formulated with sweetening agents, for example glycerol, sorbitol or sucrose. Such formulations may also contain a demulcent, a preservative and flavoring and coloring agents.

The subject compositions can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-, or di-, glycerides. In addition, n-3 polyunsaturated fatty acids may find use in the preparation of injectables.

The subject compositions can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Various delivery systems include capsules, tablets, and gelatin capsules, for example.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### General Information for Preparation Methods

Unless otherwise noted, reagents and solvents were used as received from commercial suppliers.

NMR Analysis:

Proton nuclear magnetic resonance spectra were obtained on a Varian Unity Innova 400, a Varian Unity Innova 300 a Varian Unity 300, a Bruker AMX 500 or a Bruker AV-300 spectrometer. Chemical shifts are given in ppm (δ) and coupling constants, J, are reported in Hertz. Tetramethylsilane was used as an internal standard for proton spectra and the solvent peak was used as the reference peak for carbon spectra. Mass spectra were obtained on a Perkin Elmer Sciex 100 atmospheric pressure ionization (APCI) mass spectrometer, a Finnigan LCQ Duo LCMS ion trap electrospray ionization (ESI) mass spectrometer, a PerSeptive Biosystems Mariner TOF HPLC-MS (ESI), or a Waters ZQ mass spectrometer (ESI).

Determination of MK-2 IC₅₀:

Recombinant MAPKAPK2 was phosphorylated at a concentration of 42-78 μM by incubation with 0.23 μM of active p38α in 50 mM HEPES, 0.1 mM EDTA, 10 mM magnesium acetate, and 0.25 mM ATP, pH 7.5 for one hour at 30° C.

The phosphorylation of HSP-peptide (KKKALSRQLSVAA) by MAPKAPK2 was measured using an anion exchange resin capture assay method. The reaction was carried out in 50 mM P-glycerolphosphate, 0.04% BSA, 10 mM magnesium acetate, 2% DMSO and 0.8 mM dithiotheritol, pH 7.5 in the presence of the HSP-peptide with 0.2 μCi [γ³³P]ATP and 0.03 mM ATP. The reaction was initiated by the addition of 15 nM MAPKAPK2 and was allowed to incubate at 30° C. for 30 min. The reaction was terminated and [γ³³P]ATP was removed from solution by the addition of 150 μl of AG 1×8 ion exchange resin in 900 mM sodium formate pH 3.0. A 50 μl aliquot of head volume was removed from the quenched reaction mixture and added to a 96-well plate, 150 μl of Microscint-40 (Packard) was added and the amount of phosphorylated-peptide was determined. Allow the Microscint to sit in the plates for 60 minutes prior to counting.

Compounds are evaluated as potential inhibitors of the MK2 kinase by measuring their effects on MK2 phosphorylation of the peptide substrate. Compounds may be screened initially at two concentrations prior to determination of IC₅₀ values. Screening results are expressed as percent inhibition at the concentrations of compound tested. For IC₅₀ value determinations, compounds are tested at six concentrations in ten-fold serial dilutions with each concentration tested in triplicate. Results are expressed as IC₅₀ values in micromolar. The assay is performed at a final concentration of 2% DMSO.

U937 Cell TNFα Release Assay

The human monocyte-like cell line, U937 (ATCC #CRL-1593.2), is cultured in RPMI1640 media with 10% heat-inactivated fetal calf serum (GIBCO), glutamine and pen/strep at 37° C. and 5% CO₂. Differentiation of U937 to monocytic/macrophage-like cells is induced by the addition of phorbol12-myristate 13-acetate (Sigma) at final concentration of 20 ng/ml to a culture of U937 cells at ˜0.5 million cells/ml and incubated for 24 hrs. The cells are centrifuged, washed with PBS and resuspended in fresh media without PMA and incubated for 24 hrs. Cells adherent to the culture flask are harvested by scraping, centrifugation, and resuspended in fresh media to 2 million cells/ml, and 0.2 ml is aliquoted to each of 96 wells in flat-bottom plate. Cells are then incubated for an additional 24 hrs to allow for recovery. The media is removed from the cells, and 0.1 ml of fresh media is added per well. 0.05 ml of serially diluted compound or control vehicle (Media with DMSO) is added to the cells. The final DMSO concentration does not exceed 1%. After 1 hr incubation, 0.05 ml of 400 ng/ml LPS (E Coli serotype 0111:B4, Sigma) in media is added for final concentration of 100 ng/ml. Cells are incubated at 37° C. for 4 hrs. After 4 hrs incubation, supernatants are harvest and assayed by ELISA for the presence of TNFα.

U937 cell TNFα ELISA

ELISA plates (NUNC-Immuno™ Plate Maxisorb™ Surface) were coated with purified mouse monoclonal IgG1 anti-human TNFα antibody (R&D Systems #MAB610; 1.25 ug/ml in sodium bicarbonate pH 8.0, 0.1 ml/well) and incubated at 4° C. Coating solution was aspirated the following day and wells were blocked with 1 mg/ml gelatin in PBS (plus 1× thimerasol) for 2 days at 4° C. Prior to using, wells were washed 3× with wash buffer (PBS with 0.05% Tween). Cultured media samples were diluted in EIA buffer (5 mg/ml boviney-globulin, 1 mg/ml gelatin, 1 ml/l Tween-20, 1 mg/ml thimerasol in PBS), added to wells (0.1 ml/well) in triplicate and allowed to incubate for 1.5 hr at 37° C. in a humidified chamber. Plates were again washed and 0.1 ml/well of a mixture of rabbit anti-human TNFα polyclonal antibodies in EIA buffer (1:400 dilution of Sigma #T8300, and 1:400 dilution of Calbiochem #654250) was added for 1 hr at 37° C. Plates were washed as before and peroxidase-conjugated goat anti-rabbit IgG (H+L) antibody (Jackson ImmunoResearch #111-035-144,1 ug/ml in EIA buffer, 0.1 ml/well) was added for 45 min. After final washing, plates were developed with peroxidase-ABTS solution (Kirkegaard/Perry #50-66-01, 0.1 ml/well). Enzymatic conversion of ABTS to colored product was measured after 5-30 minutes using a SpectroMax 340 spectrophotometer (Molecular Devices) at 405 nm. TNF levels were quantitated from a recombinant human TNFα (R&D Systems #210-TA-010) standard curve using a quadratic parameter fit generated by SoftMaxPRO software. ELISA sensitivity was approximately 30 pg TNF/ml. IC₅₀ values for compounds were generated using BioAssay Solver.

Lipopolysaccharide (LPS)-Induced TNFα Production.

Adult male 225-250 gram Lewis rats (Harlan Sprague-Dawley) were used. Rats were fasted 18 hr prior to oral dosing, and allowed free access to water throughout the experiment. Each treatment group consisted of 5 animals.

Compounds were prepared as a suspension in a vehicle consisting of 0.5% methylcellulose, 0.025% Tween-20 in PBS. Compounds or vehicle were orally administered in a volume of 1 ml using an 18 gauge gavage needle. LPS (E. coli serotype 0111:B4, Lot #39H4103, Cat. # L-2630, Sigma) was administered 1-4 hr later by injection into the penile vein at a dose of 1 mg/kg in 0.5 ml sterile saline. Blood was collected in serum separator tubes via cardiac puncture 1.5 hr after LPS injection, a time point corresponding to maximal TNFα production. After clotting, serum was withdrawn and stored at −20° C. until assay by ELISA (described below).

Rat LPS TNFα ELISA

ELISA plates (NUNC-Immuno™ Plate Maxisorb™ Surface) were coated with 0.1 ml per well of a Protein G purified fraction of a 2.5 ug/ml of hamster anti-mouse/rat TNFα monoclonal antibody TN19.12 (2.5 ug/ml in PBS, 0.1 ml/well). The hybridoma cell line was kindly provided by Dr. Robert Schreiber, Washington University. Wells were blocked the following day with 1 mg/ml gelatin in PBS. Serum samples were diluted in a buffer consisting of 5 mg/ml bovine y-globulin, 1 mg/ml gelatin, 1 ml/l Tween-20, 1 mg/ml thimerasol in PBS, and 0.1 ml of diluted serum was added wells in duplicate and allowed to incubate for 2 hr at 37° C. Plates were washed with PBS-Tween, and 0.1 ml per well of a 1:300 dilution of rabbit anti-mouse/rat TNFα antibody (BioSource International, Cat. #AMC3012) was added for 1.5 hr at 37° C. Plates were washed, and a 1:1000 fold dilution of peroxidase-conjugated donkey anti-rabbit IgG antibody (Jackson ImmunoResearch, Cat. #711-035-152) was added for 45 min. After washing, plates were developed with 0.1 ml of ABTS-peroxide solution (Kirkegaard/Perry, Cat. #50-66-01). Enzymatic conversion of ABTS to colored product was measured after ˜30 minutes using a SpectroMax 340 spectrophotometer (Molecular Devices Corp.) at 405 nm. TNF levels in serum were quantitated from a recombinant rat TNFα (BioSource International, Cat. #PRC3014) standard curve using a quadratic parameter fit generated by SoftMaxPRO software. ELISA sensitivity was approximately 30 pg TNF/ml. Results are expressed in percent inhibition of the production of TNFα as compared to blood collected from control animals dosed only with vehicle.

NMR Analysis:

Proton nuclear magnetic resonance spectra were obtained on a Varian Unity Innova 400, a Varian Unity Innova 300 a Varian Unity 300, a Bruker AMX 500 or a Bruker AV-300 spectrometer. Chemical shifts are given in ppm (δ) and coupling constants, J, are reported in Hertz. Tetramethylsilane was used as an internal standard for proton spectra and the solvent peak was used as the reference peak for carbon spectra. Mass spectra were obtained on a Perkin Elmer Sciex 100 atmospheric pressure ionization (APCI) mass spectrometer, a Finnigan LCQ Duo LCMS ion trap electrospray ionization (ESI) mass spectrometer, a PerSeptive Biosystems Mariner TOF HPLC-MS (ESI), or a Waters ZQ mass spectrometer (ESI).

Determination of MK-2 IC₅₀:

Recombinant MAPKAPK2 was phosphorylated at a concentration of 42-78 μM by incubation with 0.23 μM of active p38α in 50 mM HEPES, 0.1 mM EDTA, 10 mM magnesium acetate, and 0.25 mM ATP, pH 7.5 for one hour at 30° C.

The phosphorylation of HSP-peptide (KKKALSRQLSVAA) by MAPKAPK2 was measured using an anion exchange resin capture assay method. The reaction was carried out in 50 mM P-glycerolphosphate, 0.04% BSA, 10 mM magnesium acetate, 2% DMSO and 0.8 mM dithiotheritol, pH 7.5 in the presence of the HSP-peptide with 0.2 μCi [γ³³P]ATP and 0.03 mM ATP. The reaction was initiated by the addition of 15 nM MAPKAPK2 and was allowed to incubate at 30° C. for 30 min. The reaction was terminated and [γ³³P]ATP was removed from solution by the addition of 150 μl of AG 1×8 ion exchange resin in 900 mM sodium formate pH 3.0. A 50 μl aliquot of head volume was removed from the quenched reaction mixture and added to a 96-well plate, 150 μl of Microscint-40 (Packard) was added and the amount of phosphorylated-peptide was determined. Allow the Microscint to sit in the plates for 60 minutes prior to counting.

Compounds are evaluated as potential inhibitors of the MK-2 kinase by measuring their effects on MK-2 phosphorylation of the peptide substrate. Compounds may be screened initially at two concentrations prior to determination of IC₅₀ values. Screening results are expressed as percent inhibition at the concentrations of compound tested. For IC₅₀ value determinations, compounds are tested at six concentrations in ten-fold serial dilutions with each concentration tested in triplicate. Results are expressed as IC₅₀ values in micromolar. The assay is performed at a final concentration of 2% DMSO.

U937 Cell TNFα Release Assay

The human monocyte-like cell line, U937 (ATCC #CRL-1593.2), is cultured in RPMI1640 media with 10% heat-inactivated fetal calf serum (GIBCO), glutamine and pen/strep at 37° C. and 5% CO₂. Differentiation of U937 to monocytic/macrophage-like cells is induced by the addition of phorbol12-myristate 13-acetate (Sigma) at final concentration of 20 ng/ml to a culture of U937 cells at ˜0.5 million cells/ml and incubated for 24 hrs. The cells are centrifuged, washed with PBS and resuspended in fresh media without PMA and incubated for 24 hrs. Cells adherent to the culture flask are harvested by scraping, centrifugation, and resuspended in fresh media to 2 million cells/ml, and 0.2 ml is aliquoted to each of 96 wells in flat-bottom plate. Cells are then incubated for an additional 24 hrs to allow for recovery. The media is removed from the cells, and 0.1 ml of fresh media is added per well. 0.05 ml of serially diluted compound or control vehicle (Media with DMSO) is added to the cells. The final DMSO concentration does not exceed 1%. After 1 hr incubation, 0.05 ml of 400 ng/ml LPS (E Coli serotype 0111:B4, Sigma) in media is added for final concentration of 100 ng/ml. Cells are incubated at 37° C. for 4 hrs. After 4 hrs incubation, supernatants are harvest and assayed by ELISA for the presence of TNFα.

U937 cell TNFα ELISA

ELISA plates (NUNC-Immuno™ Plate Maxisorb™ Surface) were coated with purified mouse monoclonal IgG1 anti-human TNFα antibody (R&D Systems #MAB610; 1.25 μg/ml in sodium bicarbonate pH 8.0, 0.1 ml/well) and incubated at 4° C. Coating solution was aspirated the following day and wells were blocked with 1 mg/ml gelatin in PBS (plus 1× thimerasol) for 2 days at 4° C. Prior to using, wells were washed 3× with wash buffer (PBS with 0.05% Tween). Cultured media samples were diluted in EIA buffer (5 mg/ml bovine gamma-globulin, 1 mg/ml gelatin, 1 ml/l Tween-20, 1 mg/ml thimerasol in PBS), added to wells (0.1 ml/well) in triplicate and allowed to incubate for 1.5 hr at 37° C. in a humidified chamber. Plates were again washed and 0.1 ml/well of a mixture of rabbit anti-human TNFα polyclonal antibodies in EIA buffer (1:400 dilution of Sigma #T8300, and 1:400 dilution of Calbiochem #654250) was added for 1 hr at 37° C. Plates were washed as before and peroxidase-conjugated goat anti-rabbit IgG (H+L) antibody (Jackson ImmunoResearch #111-035-144, 1 ug/ml in EIA buffer, 0.1 ml/well) was added for 45 min. After final washing, plates were developed with peroxidase-ABTS solution (Kirkegaard/Perry #50-66-01, 0.1 ml/well). Enzymatic conversion of ABTS to colored product was measured after 5-30 minutes using a SpectroMax 340 spectrophotometer (Molecular Devices) at 405 nm. TNF levels were quantitated from a recombinant human TNFα (R&D Systems #210-TA-010) standard curve using a quadratic parameter fit generated by SoftMaxPRO software. ELISA sensitivity was approximately 30 pg TNF/ml. IC₅₀ values for compounds were generated using BioAssay Solver.

Lipopolysaccharide (LPS)-Induced TNFα Production.

Adult male 225-250 gram Lewis rats (Harlan Sprague-Dawley) were used. Rats were fasted 18 hr prior to oral dosing, and allowed free access to water throughout the experiment. Each treatment group consisted of 5 animals.

Compounds were prepared as a suspension in a vehicle consisting of 0.5% methylcellulose, 0.025% Tween-20 in PBS. Compounds or vehicle were orally administered in a volume of 1 ml using an 18 gauge gavage needle. LPS (E. coli serotype 0111:B4, Lot #39H4103, Cat. # L-2630, Sigma) was administered 1-4 hr later by injection into the penile vein at a dose of 1 mg/kg in 0.5 ml sterile saline. Blood was collected in serum separator tubes via cardiac puncture 1.5 hr after LPS injection, a time point corresponding to maximal TNFα production. After clotting, serum was withdrawn and stored at −20° C. until assay by ELISA (described below).

Rat LPS TNFα ELISA

ELISA plates (NUNC-Immuno™ Plate Maxisorb™ Surface) were coated with 0.1 ml per well of a Protein G purified fraction of a 2.5 ug/ml of hamster anti-mouse/rat TNFα monoclonal antibody TN19.12 (2.5 ug/ml in PBS, 0.1 ml/well). The hybridoma cell line was provided by Dr. Robert Schreiber, Washington University. Wells were blocked the following day with 1 mg/ml gelatin in PBS. Serum samples were diluted in a buffer consisting of 5 mg/ml bovine gamma-globulin, 1 mg/ml gelatin, 1 ml/l Tween-20, 1 mg/ml thimerasol in PBS, and 0.1 ml of diluted serum was added wells in duplicate and allowed to incubate for 2 hr at 37° C. Plates were washed with PBS-Tween, and 0.1 ml per well of a 1:300 dilution of rabbit anti-mouse/rat TNFα antibody (BioSource International, Cat. #AMC3012) was added for 1.5 hr at 37° C. Plates were washed, and a 1:1000 fold dilution of peroxidase-conjugated donkey anti-rabbit IgG antibody (Jackson ImmunoResearch, Cat. #711-035-152) was added for 45 min. After washing, plates were developed with 0.1 ml of ABTS-peroxide solution (Kirkegaard/Perry, Cat. #50-66-01). Enzymatic conversion of ABTS to colored product was measured after ˜30 minutes using a SpectroMax 340 spectrophotometer (Molecular Devices Corp.) at 405 nm. TNF levels in serum were quantitated from a recombinant rat TNFα (BioSource International, Cat. #PRC3014.) standard curve using a quadratic parameter fit generated by SoftMaxPRO software. ELISA sensitivity was approximately 30 pg TNF/ml. Results are expressed in percent inhibition of the production of TNFα as compared to blood collected from control animals dosed only with vehicle.

Synthesis of MK-2 Inhibiting Compounds of the Present Invention:

Unless otherwise noted, reagents and solvents were used as received from commercial suppliers. Proton nuclear magnetic resonance spectra were obtained on a Varian-300, Bruker AMX 500 or a Bruker AV-300 spectrometer. Spectra are given in ppm (δ) and coupling constants, J, are reported in Hertz. Tetramethylsilane was used as an internal standard for proton spectra and the solvent peak was used as the reference peak for carbon spectra. 3-Bromobenzotrifluoride was used as an internal standard in ¹⁹F nuclear magnetic resonance spectra to calibrate the amount of TFA for TFA salts. Mass spectra were obtained on a Mariner electrospray ionization (ESI), Perkin Elmer Sciex 100 atmospheric pressure ionization (APCI) mass spectrometer, Hewlett Packard G1947A LCMS ion trap ionization (ESI), or a Finnigan LCQ Duo LCMS ion trap electrospray ionization (ESI) mass spectrometer. Thin-layer chromatography (TLC) was performed using Analtech silica gel plates and visualized by ultraviolet (UV) light. HPLC analyses were obtained using a YMC CombiScreen ODS-A (50×4.6 mm) with UV with diode array detection, using aqueous TFA in acetonitrile as the eluent on a Hewlett Packard 1100, or a Phenomenex C₁₈ Luna column (150×4.6 mm) with UV detection at 254 nm, using aqueous TFA in acetonitrile as the eluent on a Varian Prostar. Purification using preparative HPLC was performed using a Phenomenex C₁₈ Luna column (250×22 mm) with UV detection at 254 nm, using aqueous TFA in acetonitrile as the eluent on a Varian Prostar, or using a Waters Deltapack C 18 column (47×300 mm) with UV detection at 254 nm, using aqueous TFA in acetonitrile as the eluent on a Gilson. All reactions were carried out under nitrogen unless specified.

### EXAMPLE 1

This example illustrates the production of ethyl 1-(2-aminoethyl)-3-pyridin-4-yl-1H-pyrazole-5-carboxylate dihydrochloride.

Step 1. The preparation of lithium (1Z)-4-ethoxy-3,4-dioxo-1-pyridin-4-ylbut-1-en-1-olate.

4-Acetylpyridine (960.4 g, 7.93 mole) and diethyl oxylate (1170.8 g, 8.08 mole) were dissolved in 8 L toluene in a 22 L reactor. The reaction was cooled to −78° C. under N₂, and a 1 M solution of Lithium bis(trimethylsilyl)amide in THF (8.08 L, 8.08 mole) was added in a medium stream over 45 minutes, keeping the temperature near 10° C. When the addition was complete, the reaction was allowed to warm to room temperature with stirring for 18 hours. The reaction mixture was filtered, and the solid washed with toluene followed by diethyl ether. The product was air dried and desiccated to afford 1562.5 g of the lithium salt of the diketoester (87% yield).

Step 2. Lithium (1Z)-4-ethoxy-3,4-dioxo-1-pyridin-4-ylbut-1-en-1-olate (1562.5 g, 6.88 mole) was placed in a 22 L reactor and slurried in 7 L of ethanol. The slurry was heated to 63° C. under N₂ with mixing. The heating mantle was removed, and the temperature stabilized at about 63° C. To this mixture hydrazine monohydrochloride (476.3 g, 6.95) was added. After a few minutes a slow exotherm started. An ice/water bath was applied when the reaction reached 80° C. The ice bath was removed, and the temperature held steady at 80° C. The heating mantle was re-installed, and the reaction was maintained at 80.5° C. (reflux) for one hour. The reaction was cooled and stirred at room temperature for 18 hours. The mixture was filtered, and the solids washed with ethanol followed by diethyl ether. The product was air dried and desiccated to afford 1365.6 g (91% yield) of the desired pyrazole as a tan solid. LCMS showed a single peak with m/z 218 (M+H). ¹H NMR (DMSO-d₆/300 MHz) δ 8.61 (d, 2H), 7.83 (d, 2H), 7.44 (s, 1H), 4.31 (q, 2H), 1.30 (t, 3H).

Step 3. To a cooled (0° C.) solution of ethyl 3-pyridin-4-yl-1H-pyrazole-5-carboxylate (5.57 g, 25.6 mmol) in anhydrous DMF (140 mL) was added lithium t-butoxide (1 M in THF, 38.5 mL) dropwise. The reaction stirred for 30 min, then a solution of tert-butyl 2-bromoethylcarbamate (8.62 g, 38.5 mmol) and sodium iodide (5.77 g, 38.5 mmol) in anhydrous DMF (25 mL) was added dropwise. The reaction was allowed to stir and warm to room temperature for 20 h. The reaction was poured into water and brine, extracted with ethyl acetate, dried over MgSO₄, and concentrated to an orange solid. The solid was rinsed with diethyl ether to afford an off-white solid (5.49 g, 59.5% yield): ¹H NMR (DMSO-d₆/300 MHz) δ 8.59 (d, 2H), 7.82 (d, 2H), 7.51 (s, 1H), 6.90 (t, 1H), 4.58 (t, 2H), 4.33 (q, 2H), 3.38-3.33 (m, 2H), 1.34 (t, 3H), 1.27 (s, 9H); HRMS calculated for (M+H) 361.1870, found 361.1890.

### EXAMPLE 2

This example illustrates the production of ethyl 1-(2-amino-ethyl)-3-pyridin-4-yl-1H-pyrazole-5-carboxylate dihydrochloride.

To a flask charged with ethyl 1-{2-[(tert-butoxycarbonyl)amino]ethyl}-3-pyridin-4-yl-1H-pyrazole-5-carboxylate (5.39 g, 15.0 mmol) was added 4N HCl/dioxane (20 mL). After 1 h the reaction mixture was filtered and rinsed with diethyl ether to afford an off-white solid (5.02 g, 100% yield): ¹H NMR (DMSO-d₆/300 MHz) δ 8.93 (d, 2H), 8.48-8.43 (m, 5H), 7.95 (s, 1H), 4.89 (t, 2H), 4.37 (q, 2H), 3.41-.3.38 (m, 2H), 1.35 (t, 3H); HRMS calculated for (M+H) 261.1346, found 261.1317.

### EXAMPLE 3

This example illustrates the production of 2-pyridin-4-yl-6,7-dihydro-pyrazolo[1,5-a]pyrazin-4(5H)-one trifluoroacetate.

A flask was charged with ethyl 1-(2-aminoethyl)-3-pyridin-4-yl-1H-pyrazole-5-carboxylate dihydrochloride (1.13 g, 3.39 mmol), NH₄OH (30 mL), and ethanol (15 mL). After stirring for 1 h, the reaction mixture was purified by Gilson RP HPLC (5-95% acetonitrile/water). The appropriate fractions were concentrated to a pale yellow solid (0.725 g, 65.4% yield): ¹H NMR (DMSO-d₆/300 MHz) δ 8.81 (d, 2H), 8.40 (br s, 1H), 8.22 (d, 2H), 7.67 (s, 1H), 4.43 (t, 2H), 3.70-3.64 (m, 2H); HRMS calculated for (M+H) 215.0927, found 215.0885.

### EXAMPLE 4

This example illustrates the production of 1-(2-aminoethyl)-3-pyridin-4-yl-1H-pyrazole-5-carboxylic acid trifluoroacetate.

A solution of ethyl 1-(2-aminoethyl)-3-pyridin-4-yl-1H-pyrazole-5-carboxylate dihydrochloride (0.794 g, 2.38 mmol) and LiOH.H₂O (0.310 g, 7.40 mmol) in 30 mL of THF/H₂O (1:1) was heated to 80° C. with stirring. After 2 h the reaction mixture was purified by Gilson RP HPLC (5-95% acetonitrile/water). The appropriate fractions were concentrated to a white solid (0.442 g, 53.7% yield): ¹H NMR (DMSO-d₆/300 MHz) δ 8.96 (br s, 2H), 8.48 (br s, 2H), 8.07-7.91 (m, 4H), 4.87 (br s, 2H), 3.41 (br s, 2H);

HRMS calculated for (M+H) 233.1033, found 233.1025.

### EXAMPLE 5

This example illustrates the production of 1-(2-{[3-(5-methyl-2-furyl)butyl]amino}ethyl)-3-pyridin-4-yl-1H-pyrazole-5-carboxylic acid trifluoroacetate.

Ethyl 1-(2-aminoethyl)-3-pyridin-4-yl-1H-pyrazole-5-carboxylate dihydrochloride (0.806 g, 2.42 mmol) was neutralized by stirring with morpholino-methylpolystyrene resin (4.15 g, ˜3.5 mmol base/g resin) in dichloromethane/methanol (20:1) for 1 h. The resin was filtered, rinsed with methanol, and the filtrates concentrated. The resulting white residue was dissolved in dichloromethane/methanol (20:1). Six drops of glacial acetic acid were added with stirring, followed by 3-(5-methyl-2-furyl)butyraldehyde (0.422 g, 6.60 mmol. After 5 min, sodium triacetoxyborohydride (1.04 g, 4.90 mmol) was added. The reaction stirred for 1 h, both the mono- and dialkylated products formed. The reaction was quenched with water and extracted with dichloromethane. The organic layers were concentrated to an oil, which was subjected to hydrolysis conditions [3 eq LiOH.H₂O, THF/H₂O (1:1)] for 3 h. The resulting product mixture was purified by Gilson RP HPLC (5-95% acetonitrile/water) and the fractions corresponding to the monoalkylated product were concentrated to a mauve solid (0.106 g, 9.0% yield): ¹H NMR (DMSO-d₆/300 MHz) δ 8.77-8.75 (m, 4H), 8.08 (d, 2H), 7.72 (s, 1H), 5.97-5.93 (m, 2H), 4.87 (t, 2H), 3.51-3.47 (m, 2H), 2.96 (br s, 2H), 2.82 (q, 1H), 2.19 (s, 3H), 1.92-1.72 (m, 2H), 1.16 (d, 3H); HRMS calculated for (M+H) 233.1033, found 233.1025.

### EXAMPLE 6

This example illustrates the production of ethyl 3-(1-oxidopyridin-4-yl)-1H-pyrazole-5-carboxylate.

A flask was charged with ethyl 3-pyridin-4-yl-1H-pyrazole-5-carboxylate (5.04 g, 23.2 mmol) and 3-chloroperoxybenzoic acid (7.06 g, 27.8 mmol) in 70 mL of dichloromethane. After 2 h the reaction was concentrated in vacuo to remove most of the dichloromethane. To the residue was added 5% ethyl acetate/hexanes and the suspension filtered. The solid was slurried in NaHCO₃ (sat.), filtered, and rinsed with NaHCO₃ (sat.). The solid was then slurried in NaS₂O₃, filtered, and then rinsed with water. The solid was slurried up in ethanol and concentrated to afford an orange-tan solid (4.25 g, 78.0% yield): ¹H NMR (DMSO-d₆/300 MHz) δ 8.24 (d, 2H), 7.86 (d, 2H), 7.42 (s, 1H), 4.31 (q, 2H), 1.31 (t, 3H); HRMS calculated for (M+H) 234.0873, found 234.0877.

### EXAMPLE 7

This example illustrates the production of ethyl 3-(2-chloropyridin-4-yl)-1H-pyrazole-5-carboxylate.

A suspension of ethyl 3-(1-oxidopyridin-4-yl)-1H-pyrazole-5-carboxylate (11.16 g, 14.9 mmol) and phosphorus oxychloride (110 mL, 1.2 mol) was heated to 106° C. for 48 h. The reaction mixture was concentrated, and then dissolved in chloroform (300 mL) and ice water (300 mL). Solid NaHCO₃ was added until foaming was no longer observed, then the heterogeneous solution was extracted multiple times with chloroform. The organic layers were dried over MgSO₄, filtered, and concentrated. The residue was triturated with dichloromethane. The solid was filtered and rinsed with ethyl acetate, and then with diethyl ether to afford a pale yellow solid (3.59 g, 29.8% yield): ¹H NMR (DMSO-d₆/300 MHz) δ 14.41 (br s, 1H), 8.44 (d, 1H), 7.98 (s, 1H), 7.88 (d, 1H), 7.61 (s, 1H), 4.33 (q, 2H), 1.32 (t, 3H); HRMS calculated for (M+H) 252.0534, found 252.0508.

### EXAMPLE 8

This example illustrates the production of ethyl 1-{3-[(tert-butoxycarbonyl)-amino]propyl}-3-(2-chloropyridin-4-yl)-1H-pyrazole-5-carboxylate.

Synthesis conducted as in the preparation of ethyl 1-{2-[(tert-butoxycarbonyl)amino]ethyl}-3-pyridin-4-yl-1H-pyrazole-5-carboxylate using ethyl 3-(2-chloropyridin-4-yl)-1H-pyrazole-5-carboxylate (3.36 g, 13.3 mmol), lithium t-butoxide (1 M in THF, 17.0 mL), tert-butyl 3-bromopropylcarbamate (3.81 g, 16.0 mmol) and sodium iodide (2.39 g, 16.0 mmol). Chromatographic purification (25% ethyl acetate/hexane) afforded a white solid (3.29 g, 60.5% yield): ¹H NMR (DMSO-d₆/300 MHz) δ 8.42 (d, 1H), 7.95 (s, 1H), 7.86 (dd, 1H), 7.67 (s, 1H), 6.85 (dd, 1H), 4.54 (t, 2H), 4.35 (q, 2H), 2.98-2.92 (m, 2H), 1.98-1.88 (m, 2H), 1.36-1.31 (m, 12H); HRMS calculated for (M+H) 409.1637, found 409.1634.

### EXAMPLE 9

This example illustrates the production of ethyl 1-{2-[(tert-butoxycarbonyl)-amino]ethyl}-3-(2-chloropyrid in-4-yl)-1H-pyrazole-5-carboxylate.

Synthesis was conducted as in the preparation of ethyl 1-{2-[(tert-butoxycarbonyl)amino]ethyl}-3-pyridin-4-yl-1H-pyrazole-5-carboxylate, using ethyl 3-(2-chloropyridin-4-yl)-1H-pyrazole-5-carboxylate (6.00 g, 23.8 mmol), lithium t-butoxide (1 M in THF, 31.0 mL), tert-butyl 2-bromoethylcarbamate (6.59 g, 29.4 mmol), and sodium iodide (4.41 g, 29.4 mmol). Flash chromatography (25% ethyl acetate/hexane) afforded a white solid (6.07 g, 64.6% yield): ¹H NMR (DMSO-d₆/300 MHz) δ 8.43 (d, 1H), 7.94 (s, 1H), 7.85 (dd, 1H), 7.66 (s, 1H), 6.89 (t, 1H), 4.59 (t, 2H), 4.33 (q, 2H), 3.39-3.32 (m, 2H), 1.36-1.23 (m, 12H); HRMS calculated for (M+H) 395.1481, found 395.1512.

### EXAMPLE 10

This example illustrates the production of 2-(2-chloropyridin-4-yl)-5,6,7,8-tetrahydro-4H-pyrazolo[1,5-a][1,4]diazepin-4-one.

A flask was charged with ethyl 1-(3-[(tert-butoxycarbonyl)amino]propyl}-3-(2-chloropyridin-4-yl)-1H-pyrazole-5-carboxylate (6.50 g, 15.9 mmol) and 4N HCl/dioxane (45 mL). The reaction mixture stirred for 1 h, then the suspension was filtered and the solid rinsed with diethyl ether. This solid was taken up in ethanol (20 mL) and NH₄OH (40 mL). After stirring for 20 h, the suspension was filtered and rinsed with ethanol and diethyl ether to afford a white solid (3.64 g, 87.4% yield): ¹H NMR (DMSO-d₆/300 MHz) δ 8.41 (d, 1H), 8.35 (t, 1H), 7.90 (s, 1H), 7.83 (d, 1H), 7.48 (s, 1H), 4.49 (t, 2H), 3.24-3.18 (m, 2H), 2.20-2.14 (m, 2H), HRMS calculated for (M+H) 263.0694, found 263.0689.

### EXAMPLE 11

This example illustrates the production of ethyl 1-{3-[(tert-butoxycarbonyl)amino]propyl}-3-[2-(3-nitrophenyl)pyridin-4-yl]-1H-pyrazole-5-carboxylate.

A flask was charged with ethyl 1-{3-[(tert-butoxycarbonyl)amino]propyl}-3-(2-chloropyridin-4-yl)-1H-pyrazole-5-carboxylate (0.875 g, 2.14 mmol), 3-nitrophenylboronic acid (0.536 g, 3.21 mmol), 2M Na₂CO₃ (9 mL), toluene (25 mL), and [1,1′ bis(diphenylphosphino) ferrocene]dichloropalladium(II), 1:1 complex with dichloromethane [Pd[dppf]Cl₂.CH₂Cl₂ (0.140 g, 0.170 mmol)], then purged with N₂ and heated at 90° C. for 20 h. The reaction mixture was diluted with ethyl acetate and washed with water and brine. The organic layer was dried over MgSO₄, filtered, and concentrated. Chromatographic purification (30% ethyl acetate/hexane) afforded a white solid (0.767 g, 72.4% yield): ¹H NMR (DMSO-d₆/300 MHz) δ 9.00 (dd, 1H), 8.75 (d, 1H), 8.68 (d, 1H), 8.54 (s, 1H), 8.30 (m, 1H), 7.91 (dd, 1H), 7.84-7.70 (m, 2H), 6.87 (dd, 1H), 4.58 (t, 2H), 4.36 (q, 2H), 3.00-2.95 (m, 2H), 1.98-1.92 (m, 2H), 1.38-1.31 (m, 12H); HRMS calculated for (M+H) 496.2191, found 496.2175.

### EXAMPLE 12

This example illustrates the production of 1-(3-aminopropyl)-3-[2-(3 nitrophenyl)pyridin-4-yl]-1H-pyrazole-5-carboxamide trifluoroacetate.

Ammonia gas was bubbled into a pressure tube charged with ethyl 1-{3-[(tert-butoxycarbonyl)amino]propyl}-3-[2-(3-nitrophenyl)pyridin-4-yl]-1H-pyrazole-5-carboxylate (0.084 g, 0.17 mmol) in 10 mL ethanol and cooled to −78° C. The tube was then sealed and heated to 70° C. for 6 days. The reaction mixture was concentrate in vacuo. To this residue was added 4N HCl/dioxane (6 mL). After 1.5 h the solution was purified by Gilson RP HPLC (5-95% acetonitrile/water). The appropriate fractions were concentrated to a light tan solid (0.048 g, 60% yield): ¹H NMR (DMSO-d₆/300 MHz) δ 8.94 (dd, 1H), 8.81 (d, 1H), 8.60 (d, 1H), 8.43 (s, 1H), 8.36 (dd, 1H), 8.20-8.09 (m, 4H), 7.87-7.74 (m, 3H), 4.66 (t, 2H), 2.86-2.79 (m, 2H), 2.20-2.15 (m, 2H); HRMS calculated for (M+H) 367.1513, found 367.1529.

### EXAMPLE 13

This example illustrates the production of 1-(3-aminopropyl)-3-(2-quinolin-3-ylpyridin-4-yl)-1H-pyrazole-5-carboxamide dihydrochloride.

Synthesis was conducted as for the production of 1-(3-aminopropyl)-3-[2-(3-nitrophenyl)pyridin-4-yl]-1H-pyrazole-5-carboxamide trifluoroacetate using ethyl 1-{3-[(tert-butoxycarbonyl)amino]propyl}-3-(2-quinolin-3-ylpyridin-4-yl)-1H-pyrazole-5-carboxylate (0.127 g, 0.254 mmol). The TFA salt residue was taken up in methanol and 4N HCl/dioxane. After 20 h the suspension was concentrated to an off-white solid (0.069 g, 0.16 mmol): ¹H NMR (DMSO-d₆/300 MHz) δ 9.90 (d, 1H), 9.78 (s, 1H), 8.87 (d, 1H), 8.69 (s, 1H), 8.46-8.39 (m, 2H), 8.22-8.08 (m, 5H), 7.96-7.89 (m, 2H), 7.82-7.78 (m, 2H), 4.67 (t, 2H), 2.86-2.77 (m, 2H), 2.25-2.16 (m, 2H); HRMS calculated for (M+H) 373.1771, found 373.1769.

### EXAMPLE 14

This example illustrates the production of ethyl 1-{2-[(tertbutoxy-carbonyl)amino]ethyl}-3-(2-quinolin-3-ylpyridin-4-yl)-1H-pyrazole-5-carboxylate.

Synthesis was conducted as in the preparation of ethyl 1-{3-[(tert-butoxycarbonyl)amino]propyl}-3-[2-(3-nitrophenyl)pyridin-4-yl]-1H-pyrazole-5-carboxylate using ethyl 1-{2-[(tert-butoxycarbonyl)amino]ethyl}-3-(2-chloropyridin-4-yl)-1H-pyrazole-5-carboxylate (0.732 g, 1.85 mmol), 3-quinolinylboronic acid (0.481 g, 2.78 mmol), 2M Na₂CO₃ (7 mL), toluene (20 mL), and Pd[dppf]Cl₂.CH₂Cl₂ (0.121 g, 0.148 mmol). The black residue obtained from the aqueous work-up was triturated with dichloromethane to afford an off-white solid (0.615 g, 68.2% yield): ¹H NMR (DMSO-d₆/300 MHz) δ 9.72 (s, 1H), 9.14 (s, 1H), 8.78 (d, 1H), 8.62 (s, 1H), 8.15-8.08 (m, 2H), 7.90-7.82 (m, 3H), 7.71-7.66 (m, 1H), 6.94 (t, 1H), 4.64 (t, 2H), 4.37 (q, 2H), 3.39-3.32 (m, 2H), 1.39-1.28 (m, 12H); HRMS calculated for (M+H) 488.2292, found 488.2296.

### EXAMPLE 15

This example illustrates the production of 2-{2-[4-(trifluoromethoxy)-phenyl]pyridin-4-yl}-6,7-dihydropyrazolo[1,5-a]pyrazin-4(5H)-one trifluoroacetate.

Synthesis was conducted as in the preparation of ethyl 1-{3-[(tert-butoxycarbonyl)amino]propyl}-3-[2-(3-nitrophenyl)pyridin-4-yl]-1H-pyrazole-5-carboxylate using ethyl 1-{3-[(tert-butoxycarbonyl)amino]propyl}-3-(2-chloropyridin-4-yl)-1H-pyrazole-5-carboxylate (0.954 g, 2.33 mmol), 3-quinolinylboronic acid (0.520 g, 3.00 mmol), 2M Na₂CO₃ (10 mL), toluene (25 mL), and Pd[dppf]Cl₂.CH₂Cl₂ (0.152 g, 0.186 mmol). Flash chromatography (25% ethyl acetate/hexane) afforded a beige solid (0.593 g, 50.7% yield): ¹H NMR (DMSO-d₆/300 MHz) δ 9.72 (d, 1H), 9.14 (d, 1H), 8.77 (d, 1H), 8.63 (s, 1H), 8.15-8.07 (m, 2H), 7.85-7.82 (m, 3H), 7.69 (dd, 1H), 6.88 (t, 1H), 4.60 (t, 2H), 4.37 (q, 2H), 3.02-2.96 (m, 2H), 2.01-1.93 (m, 2H), 1.39-1.34 (m, 12H); HRMS calculated for (M+H) 502.2449, found 502.2419.

### EXAMPLE 16

This example illustrates the production of ethyl 1-{2-[(tert-butoxycarbonyl)amino]ethyl}-3-{2-[4-(hydroxymethyl) phenyl]pyridin-4-yl}-1H-pyrazole-5-carboxylate.

Synthesis was conducted as in the preparation of ethyl 1-{3-[(tert-butoxycarbonyl)amino]propyl}-3-[2-(3-nitrophenyl)pyridin-4-yl]-1H-pyrazole-5-carboxylate using ethyl 1-{2-[(tert-butoxycarbonyl)amino]ethyl}-3-(2-chloropyridin-4-yl)-1H-pyrazole-5-carboxylate (1.100 g, 2.79 mmol), 4-hydroxymethyl-phenylboronic acid (0.550 g, 3.62 mmol), 2M Na₂CO₃ (12 mL), toluene (32 mL), and Pd[dppf]Cl₂.CH₂Cl₂ (0.182 g, 0.223 mmol). The black residue obtained from the aqueous work-up was triturated with dichloromethane to yield an off-white solid (0.567 g, 43.6% yield): ¹H NMR (DMSO-d₆/300 MHz) δ 8.67 (d, 1H), 8.32 (s, 1H), 8.15 (d, 2H), 7.78-7.76 (m, 2H), 7.45 (d, 2H), 6.92 (t, 1H), 5.26 (t, 1H), 4.62-4.56 (m, 4H), 4.35 (q, 2H), 3.39-3.35 (m, 2H), 1.36-1.27 (m, 12H); HRMS calculated for (M+H) 467.2289, found 467.2259.

### EXAMPLE 17

This example illustrates the production ethyl 1-(3-aminopropyl)-3-[2-(3-nitrophenyl)pyridin-4-yl]-1H-pyrazole-5-carboxylate dihydrochloride.

Synthesis was conducted as in the preparation of ethyl 1-(2-aminoethyl)-3-pyridin-4-yl-1H-pyrazole-5-carboxylate dihydrochloride using ethyl 1-{3-[(tert-butoxycarbonyl)amino]propyl}-3-[2-(3-nitrophenyl)pyridin-4-yl]-1H-pyrazole-5-carboxylate (0.763 g, 1.53 mmol) and 4N HCl/dioxane (10 mL). A light yellow solid was obtained (0.685 g, 95.4% yield): ¹H NMR (DMSO-d₆/300 MHz) δ 9.00 (dd, 1H), 8.80 (d, 1H), 8.68 (d, 1H), 8.63 (s, 1H), 8.35 (dd, 1H), 8.13 (brs, 3H), 8.02 (dd, 1H), 7.93 (s, 2H), 7.85 (dd, 1H), 4.68 (t, 2H), 4.37 (q, 2H), 2.87-2.80 (m, 2H), 2.21-2.16 (m, 2H), 1.36 (t, 3H); HRMS calculated for (M+H) 396.1666, found 396.1660.

### EXAMPLE 18

This example illustrates the production of ethyl 1-(2-aminoethyl)-3-(2-quinolin-3-ylpyridin-4-yl)-1H-pyrazole-5-carboxylate dihydrochloride.

Synthesis was conducted as in the preparation of ethyl 1-(2-aminoethyl)-3-pyridin-4-yl-1H-pyrazole-5-carboxylate dihydrochloride using ethyl 1-{2-[(tert-butoxycarbonyl)amino]ethyl}-3-(2-quinolin-3-ylpyridin-4-yl)-1H-pyrazole-5-carboxylate (0.588 g, 1.21 mmol) and 4N HCl/dioxane (12 mL). A white solid was obtained (0.557 g, 100% yield): ¹H NMR (DMSO-d₆/300 MHz) δ 9.97 (s, 1H), 9.86 (s, 1H), 8.87-8.85 (m, 2H), 8.62 (s, 1H), 8.48-8.41 (m, 5H), 8.15-8.08 (m, 2H), 7.98-7.90 (m, 2H), 4.89 (t, 2H), 4.39 (q, 2H), 3.43-3.36 (m, 2H), 1.37 (t, 3H); HRMS calculated for (M+H) 388.1768, found 388.1754.

### EXAMPLE 19

This example illustrates the production of ethyl 1-(3-aminopropyl)-3-(2-quinolin-3-ylpyridin-4-yl)-1H-pyrazole-5-carboxylate dihydrochloride.

Synthesis was conducted as for the preparation of ethyl 1-(2-aminoethyl)-3-pyridin-4-yl-1H-pyrazole-5-carboxylate dihydrochloride, using ethyl 1-{3-[(tert-butoxycarbonyl)amino]propyl}-3-(2-quinolin-3-ylpyridin-4-yl)-1H-pyrazole-5-carboxylate (0.404 g, 0.805 mmol) and 4N HCl/dioxane (10 mL). A yellow solid was obtained (0.357 g, 93.5% yield): ¹H NMR (DMSO-d₆/300 MHz) δ 9.92 (d, 1H), 9.72 (d, 1H), 8.84 (d, 1H), 8.79 (s, 1H), 8.40-8.36 (m, 2H), 8.16 (brs, 3H), 8.07-8.00 (m, 2H), 7.93-7.88 (m, 2H), 4.66 (t, 2H), 4.38 (q, 2H), 2.90-2.82 (m, 2H), 2.25-2.16 (m, 2H), 1.37 (t, 3H); HRMS calculated for (M+H) 402.1925, found 402.1937.

### EXAMPLE 20

This example illustrates the production of ethyl 1-(2-aminoethyl)-3-{2-[4-(hydroxymethyl)phenyl]pyridin-4-yl}-1H-pyrazole-5-carboxylate dihydrochloride.

Synthesis was conducted as for the preparation of ethyl 1-(2-aminoethyl)-3-pyridin-4-yl-1H-pyrazole-5-carboxylate dihydrochloride, using ethyl 1-{2-[(tert-butoxycarbonyl)amino]ethyl}-3-{2-[4-(hydroxymethyl)phenyl]pyridin-4-yl}-1H-pyrazole-5-carboxylate (0.498 g, 1.07 mmol) and 4N HCl/dioxane (10 mL). A white solid was obtained (0.474 g, 100% yield): ¹H NMR (DMSO-d₆/300 MHz) δ 8.83 (d, 1H), 8.74 (s, 1H), 8.38 (br s, 3H), 8.29-8.20 (m, 3H), 8.08 (s, 1H), 7.57 (d, 2H), 4.90 (t, 2H), 4.62 (m, 2H), 4.39 (q, 2H), 3.40-3.35 (m, 2H), 1.36 (t, 3H); HRMS calculated for (M+H) 367.1765, found 367.1751.

### EXAMPLE 21

This example illustrates the production of ethyl 1-(2-aminoethyl)-3-[2-(3-nitro-phenyl)pyridin-4-yl]-1H-pyrazole-5-carboxylate dihydrochloride.

A flask was charged with ethyl 1-{2-[(tert-butoxycarbonyl)amino] ethyl}-3-(2-chloropyridin-4-yl)-1H-pyrazole-5-carboxylate (0.700 g, 1.78 mmol), 3-nitrophenylboronic acid (0.446 g, 2.67 mmol), 2M Na₂CO₃ (7 mL), toluene (20 mL), and [1,1′ bis(diphenylphosphino) ferrocene]dichloropalladium (II), 1:1 complex with dichloromethane [Pd[dppf]Cl₂.CH₂Cl₂ (0.116 g, 0.142 mmol)], then purged with N₂ and heated at 90° C. for 20 h. The reaction mixture was filtered through Celite and the organic layer was concentrated. To this residue was added 4N HCl/dioxane (10.0 mL). After 2 h the reaction-mixture was purified by Gilson RP HPLC (5-95% acetonitrile/water). The appropriate fractions were concentrated to a tacky residue, which was converted to the HCl salt using 4N HCl/dioxane and methanol. After stirring 1 h the reaction was concentrated to a brown solid (0.688 g, 85.1% yield): ¹H NMR (DMSO-d₆/300 MHz) δ 9.01 (s, 1H), 8.80 (dd, 1H), 8.70-8.64 (m, 2H), 8.35-8.28 (m, 4H), 8.03 (d, 1H), 7.93 (s, 1H), 7.84 (dd, 1H), 4.87 (t, 2H), 4.38 (q, 2H), 3.39-3.33 (m, 2H), 1.36 (t, 3H); HRMS calculated for (M+H) 382.1510, found 382.1525.

### EXAMPLE 22

This example illustrates the production of ethyl 1-(2-aminoethyl)-3-[2-(4-methoxyphenyl)pyridin-4-yl]-1H-pyrazole-5-carboxylate dihydrochloride.

Synthesis was conducted as for the production of ethyl 1-(2-aminoethyl)-3-[2-(3-nitrophenyl)pyridin-4-yl]-1H-pyrazole-5-carboxylate dihydrochloride using ethyl 1-{2-[(tert-butoxycarbonyl)amino]ethyl}-3-(2-chloropyridin-4-yl)-1H-pyrazole-5-carboxylate (0.700 g, 1.78 mmol), 4-methoxyphenylboronic acid (0.406 g, 2.67 mmol), 2M Na₂CO₃ (7 mL), toluene (20 mL), Pd[dppf]Cl₂.CH₂Cl₂ (0.116 g, 0.142 mmol), and 4N HCl/dioxane (10.0 mL). The product was isolated as a yellow solid (0.709 g, 90.7% yield): ¹H NMR (DMSO-d₆/300 MHz) δ 8.77 (d, 1H), 8.65 (s, 1H), 8.35 (brs, 3H), 8.24 (d, 2H), 8.16 (d, 1H), 8.04 (s, 1H), 7.18 (d, 2H), 4.89 (t, 2H), 4.38 (q, 2H), 3.87 (s, 3H), 3.42-3.38 (m, 2H), 1.36 (t, 3H),

HRMS calculated for (M+H) 367.1765, found 367.1790.

### EXAMPLE 23

This example illustrates the production of Ethyl 1-(2-aminoethyl)-3-{2-[4-(trifluoromethoxy)phenyl]pyridin-4-yl}-1H-pyrazole-5-carboxylate dihydrochloride.

Synthesis was conducted as for the production of ethyl 1-(2-aminoethyl)-3-[2-(3-nitrophenyl)pyridin-4-yl]-1H-pyrazole-5-carboxylate dihydrochloride using ethyl 1-{2-[(tert-butoxycarbonyl)amino]ethyl}-3-(2-chloropyridin-4-yl)-1H-pyrazole-5-carboxylate (0.700 g, 1.78 mmol), 4-trifluoromethoxyphenylboronic acid (0.550 g, 2.67 mmol), 2M Na₂CO₃ (7 mL), toluene (20 mL), Pd[dppf]Cl₂.CH₂Cl₂ (0.116 g, 0.142 mmol), and 4N HCl/dioxane (10.0 mL). The product was isolated as a reddish-tan solid (0.821 g, 93.5% yield): ¹H NMR (DMSO-d₆/300 MHz) δ 8.79 (d, 1H), 8.59 (s, 1H), 8.36-8.33 (m, 5H), 8.07 (d, 1H), 7.94 (s, 1H), 7.57 (d, 2H), 4.87 (t, 2H), 4.38 (q, 2H), 3.42-3.38 (m, 2H), 1.36 (t, 3H); HRMS calculated for (M+H) 421.1482, found 421.1482.

### EXAMPLE 24

This example illustrates the production of ethyl 1-(2-aminoethyl)-3-{2-[(E)-2-phenylethenyl]pyridin-4-yl}-1H-pyrazole-5-carboxylate dihydrochloride.

Synthesis was conducted as for the production of ethyl 1-(2-aminoethyl)-3-[2-(3-nitrophenyl)pyridin-4-yl]-1H-pyrazole-5-carboxylate dihydrochloride in using ethyl 1-{2-[(tert-butoxycarbonyl)amino]ethyl}-3-(2-chloropyridin-4-yl)-1H-pyrazole-5-carboxylate (1.044 g, 2.64 mmol), trans-2-phenylvinylboronic acid (0.587 g, 3.97 mmol), 2M Na₂CO₃ (10 mL), toluene (30 mL), Pd[dppf]Cl₂.CH₂Cl₂ (0.172 g, 0.211 mmol), and 4N HCl/dioxane (10.0 mL). The product was isolated as a yellow solid (1.213 g, >100% yield, 73.0% pure): ¹H NMR (DMSO-d₆/300 MHz) δ 8.79-8.72 (m, 2H), 8.36 (brs, 3H), 8.24-8.19 (m, 2H), 7.96 (s, 1H), 7.71 (d, 2H), 7.62-7.45 (m, 5H), 4.89 (t, 2H), 4.39 (q, 2H), 3.41-3.39 (m, 2H), 1.37 (t, 3H); HRMS calculated for (M+H) 363.1816, found 363.1807.

### EXAMPLE 25

This example illustrates the production of ethyl 1-(2-aminoethyl)-3-{2-[4-(dimethylamino)phenyl]pyridin-4-yl}-1H-pyrazole-5-carboxylate trifluoroacetate.

Synthesis was conducted as for the production of ethyl 1-(2-aminoethyl)-3-[2-(3-nitrophenyl)pyridin-4-yl]-1H-pyrazole-5-carboxylate dihydrochloride in using ethyl 1-{2-[(tert-butoxycarbonyl)amino]ethyl}-3-(2-chloropyridin-4-yl)-1H-pyrazole-5-carboxylate (0.758 g, 1.92 mmol), 4-dimethylaminophenylboronic acid (0.475 g, 2.88 mmol), 2M Na₂CO₃ (8 mL), toluene (23 mL), Pd[dppf]Cl₂.CH₂Cl₂ (0.126 g, 0.154 mmol), and 4N HCl/dioxane (10.0 mL). The TFA salt was isolated as a yellow solid (0.666 g, 70.3% yield): ¹H NMR (DMSO-d₆/300 MHz) δ 8.64 (d, 1H), 8.46 (s, 1H), 8.07-8.04 (m, 5H), 7.95 (s, 1H), 7.88 (d, 1H), 6.86 (d, 2H), 4.85 (t, 2H), 4.38 (q, 2H), 3.47-3.37 (m, 2H), 3.03 (s, 6H), 1.36 (t, 3H); HRMS calculated for (M+H) 380.2081, found 380.2098.

### EXAMPLE 26

This example illustrates the production of ethyl 1-(2-aminoethyl)-3-[2-(3-methoxyphenyl)pyridin-4-yl]-1H-pyrazole-5-carboxylate dihydrochloride.

Synthesis was conducted as for the production of ethyl 1-(2-aminoethyl)-3-[2-(3-nitrophenyl)pyridin-4-yl]-1H-pyrazole-5-carboxylate dihydrochloride, using ethyl 1-{2-[(tert-butoxycarbonyl)amino]ethyl}-3-(2-chloropyridin-4-yl)-1H-pyrazole-5-carboxylate (0.752 g, 1.90 mmol), 3-methoxyphenylboronic acid (0.434 g, 2.86 mmol), 2M Na₂CO₃ (7 mL), toluene (20 mL), Pd[dppf]Cl₂.CH₂Cl₂ (0.124 g, 0.152 mmol), and 4N HCl/dioxane (10.0 mL). The product was isolated as an off-white solid (0.500 g, 60.0% yield): ¹H NMR (DMSO-d₆/300 MHz) δ 8.81 (d, 1H), 8.67 (s, 1H), 8.37 (br s, 3H), 8.22 (d, 1H), 8.04 (s, 1H), 7.82-7.79 (m, 2H), 7.53 (dd, 1H), 7.18 (d, 1H), 4.89 (t, 2H), 4.38 (q, 2H), 3.89 (s, 3H), 3.42-3.36 (m, 2H), 1.36 (t, 3H); HRMS calculated for (M+H) 367.1765, found 367.1755.

### EXAMPLE 27

This example illustrates the production of ethyl 1-(2-aminoethyl)-3-[2-(3-hydroxyphenyl)pyridin-4-yl]-1H-pyrazole-5-carboxylate dihydrochloride.

Synthesis was conducted as for the production of ethyl 1-(2-aminoethyl)-3-[2-(3-nitrophenyl)pyridin-4-yl]-1H-pyrazole-5-carboxylate dihydrochloride using ethyl 1-{2-[(tert-butoxycarbonyl)amino]ethyl}-3-(2-chloropyridin-4-yl)-1H-pyrazole-5-carboxylate (0.752 g, 1.90 mmol), 3-hydroxyphenylboronic acid (0.629 g, 2.86 mmol), 2M Na₂CO₃ (7 mL), toluene (20 mL), Pd[dppf]Cl₂.CH₂Cl₂ (0.124 g, 0.152 mmol), and 4N HCl/dioxane (10.0 mL). The product was isolated as an off-white solid (0.381 g, 46.8% yield): ¹H NMR (DMSO-d₆/300 MHz) δ 8.80 (d, 1H), 8.62 (s, 1H), 8.37 (br s, 3H), 8.22 (d, 1H), 8.04 (s, 1H), 7.62-7.58 (m, 2H), 7.41 (dd, 1H), 7.05 (dd, 1H), 4.89 (t, 2H), 4.38 (q, 2H), 3.89 (s, 3H), 3.42-3.36 (m, 2H), 1.36 (t, 3H); HRMS calculated for (M+H) 353.1608, found 353.1630.

### EXAMPLE 28

This example illustrates the production of 2-(2-quinolin-3-ylpyridin-4-yl)-6,7-dihydropyrazolo[1,5-a]pyrazin-4(5H)-one.

A flask was charged with ethyl 1-(2-aminoethyl)-3-(2-quinolin-3-ylpyridin-4-yl)-1H-pyrazole-5-carboxylate dihydrochloride (0.303 g, 0.659 mmol), NH₄OH (6 mL) and ethanol (3 mL). After stirring for 20 h, the reaction was filtered, and the solid rinsed with water, ethanol, and diethyl ether to obtain a white solid (0.178 g, 76.8% yield): ¹H NMR (DMSO-d₆/300 MHz) δ 9.73 (s, 1H), 9.16 (s, 1H), 8.79 (s, 1H), 8.64 (s, 1H), 8.35 (s, 1H), 8.16-8.08 (m, 2H), 7.90-7.69 (m, 4H), 4.45 (br s, 2H), 3.69 (br s, 2H); HRMS calculated for (M+H) 342.1349, found 342.1365.

### EXAMPLE 29

This example illustrates the production of 2-(2-quinolin-3-ylpyridin-4-yl)-5,6,7,8-tetrahydro-4H-pyrazolo[1,5-a][1,4]diazepin-4-one.

Synthesis was conducted as it was for the production of 2-(2-quinolin-3-ylpyridin-4-yl)-6,7-dihydropyrazolo[1,5-a]pyrazin-4(5H)-one using ethyl 1-(3-aminopropyl)-3-(2-quinolin-3-ylpyridin-4-yl)-1H-pyrazole-5-carboxylate dihydrochloride (0.144 g, 0.303 mmol), NH₄OH (6 mL), and ethanol (3 mL). A white solid was obtained (0.046 g, 43% yield): ¹H NMR (DMSO-d₆/300 MHz) δ 9.72 (d, 1H), 9.16 (d, 1H), 8.77 (d, 1H), 8.61 (s, 1H), 8.35 (brs, 1H), 8.15-8.07 (m, 2H), 7.88-7.79 (m, 2H), 7.71-7.66 (m, 2H), 4.55 (t, 2H), 3.29-3.23 (m, 2H), 2.22-2.18 (m, 2H); HRMS calculated for (M+H) 356.1506, found 356.1525.

### EXAMPLE 30

This example illustrates the production of 2-{2-[4-(hydroxymethyl)phenyl]pyridin-4-yl}-6,7-dihydropyrazolo[1,5-a]pyrazin-4(5H)-one.

Synthesis was conducted as it was for the production of 2-(2-quinolin-3-ylpyridin-4-yl)-6,7-dihydropyrazolo[1,5-a]pyrazin-4(5H)-one using ethyl 1-(2-aminoethyl)-3-{2-[4-(hydroxymethyl)phenyl]pyridin-4-yl}-1H-pyrazole-5-carboxylate dihydrochloride (0.202 g, 0.459 mmol), NH₄OH (8 mL), and ethanol (4 mL). A white solid was obtained (0.098 g, 66% yield): ¹H NMR (DMSO-d₆/300 MHz) δ 8.67 (d, 1H), 8.36-8.32 (m, 2H), 8.15 (d, 2H), 7.78 (d, 1H), 7.64 (s, 1H), 7.45 (d, 2H), 5.27 (t, 1H), 4.57 (d, 2H), 4.42 (t, 2H), 3.70-3.66 (m, 2H); HRMS calculated for (M+H) 321.1346, found 321.1333.

### EXAMPLE 31

This example illustrates the production of 2-[2-(3-methoxyphenyl)pyridin-4-yl]-6,7-dihydropyrazolo[1,5-a]pyrazin-4(5H)-one.

Synthesis was conducted as it was for the production of 2-(2-quinolin-3-ylpyridin-4-yl)-6,7-dihydropyrazolo[1,5-a]pyrazin-4(5H)-one using ethyl 1-(2-aminoethyl)-3-[2-(3-methoxyphenyl)pyridin-4-yl]-1H -pyrazole-5-carboxylate dihydrochloride (0.253 g, 0.575 mmol), NH₄OH (8 mL), and ethanol (4 mL). An off-white solid was obtained (0.160 g, 86.8% yield): ¹H NMR (DMSO-d₆/300 MHz) δ 8.69 (s, 1H), 8.37-8.32 (m, 2H), 7.80-7.68 (m, 4H), 7.42 (dd, 1H), 7.03 (d, 1H), 4.42 (t, 2H), 3.86 (s, 3H), 3.69-3.65 (m, 2H); HRMS calculated for (M+H) 321.1346, found 321.1344.

### EXAMPLE 32

This example illustrates the production of 2-[2-(3-hydroxyphenyl)pyridin-4-yl]-6,7-dihydropyrazolo[1,5-a]pyrazin-4(5H)-one.

Synthesis was conducted as it was for the production of 2-(2-quinolin-3-ylpyridin-4-yl)-6,7-dihydropyrazolo[1,5-a]pyrazin-4(5H)-one using ethyl 1-(2-aminoethyl)-3-[2-(3-hydroxyphenyl)pyridin-4-yl]-1H-pyrazole-5-carboxylate dihydrochloride (0.304 g, 0.715 mmol), NH₄OH (10 mL), and ethanol (5 mL). An off-white solid was obtained (0.178 g, 81.1% yield): ¹H NMR (DMSO-d₆/300 MHz) δ 9.56 (br s, 1H), 8.66 (d, 1H), 8.32-8.28 (m, 2H), 7.79 (s, 1H), 7.63-7.59 (m, 3H), 7.30 (dd, 1H), 6.85 (d, 1H), 4.42 (t, 2H), 3.69-3.64 (m, 2H); HRMS calculated for (M+H) 307.1190, found 307.1214.

### EXAMPLE 33

This example illustrates the production of 2-[2-(3-nitrophenyl)pyridin-4-yl]-5,6,7,8-tetrahydro-4H-pyrazolo[1,5-a][1,4]diazepin-4-one hydrochloride.

Synthesis was conducted as it was for the production of 2-pyridin-4-yl-6,7-dihydropyrazolo[1,5-a]pyrazin-4(5H)-one trifluoroacetate using ethyl 1-(3-aminopropyl)-3-[2-(3-nitrophenyl)pyridin-4-yl]-1H-pyrazole-5-carboxylate dihydrochloride (0.253 g, 0.540 mmol), NH₄OH (8 mL), and ethanol (4 mL). The isolated TFA salt was converted to the HCl salt using methanol and 4N HCl/dioxane. After 0.5 h, the suspension was concentrated and the solid rinsed with diethyl ether to afford a white solid (0.140 g, 65.2% yield): ¹H NMR (DMSO-d₆/300 MHz) δ 9.00 (dd, 1H), 8.78 (d, 1H), 8.65 (d, 1H), 8.60 (s, 1H), 8.39-8.33 (m, 2H), 8.00 (dd, 1H), 7.84 (dd, 1H), 7.74 (s, 1H), 4.54 (t, 2H), 3.27-3.21 (m, 2H), 2.20-2.15 (m, 2H); HRMS calculated for (M+H) 342.1349, found 342.1365.

### EXAMPLE 34

This example illustrates the production of 2-{2-[4-(trifluoromethoxy)-phenyl]pyridin-4-yl}-6,7-dihydropyrazolo[1,5-a]pyrazin-4(5H)-one trifluoroacetate.

Synthesis was conducted as it was for the production of 2-pyridin-4-yl-6,7-dihydropyrazolo[1,5-a]pyrazin-4(5H)-one trifluoroacetate using ethyl 1-(2-aminoethyl)-3-{2-[4-(trifluoromethoxy)phenyl]pyridin-4-yl}-1H-pyrazole-5-carboxylate dihydrochloride (0.439 g, 0.889 mmol), NH₄OH (8 mL), and ethanol (4 mL). An off-white solid was obtained (0.181 g, 43.0% yield): ¹H NMR (DMSO-d₆/300 MHz) δ 8.72 (d, 1H), 8.45 (s, 1H), 8.33-8.30 (m, 3H), 7.88 (d, 1H), 7.69 (s, 1H), 7.51 (d, 2H), 4.43 (q, 2H), 3.70-3.64 (m, 2H); HRMS calculated for (M+H) 375.1063, found 375.1068.

### EXAMPLE 35

This example illustrates the production of 2-{2-[(E)-2-phenylvinyl]pyridin-4-yl}-6,7-dihydropyrazolo[1,5-a]pyrazin-4(5H)-one trifluoroacetate.

Synthesis was conducted as it was for the production of 2-pyridin-4-yl-6,7-dihydropyrazolo[1,5-a]pyrazin-4(5H)-one trifluoroacetate using ethyl 1-(2-aminoethyl)-3-{2-[(E)-2-phenylvinyl]pyridin-4-yl}-1H-pyrazole-5-carboxylate dihydrochloride (0.610 g, 1.40 mmol), NH₄OH (10 mL), and ethanol (5 mL). A yellow solid was obtained (0.352 g, 58.4% yield): ¹H NMR (DMSO-d₆/300 MHz) δ 8.69 (d, 1H), 8.40 (br s, 2H), 7.97-7.92 (m, 2H), 7.71-7.68 (m, 3H), 7.49-7.37 (m, 4H), 4.45 (t, 2H), 3.70-3.66 (m, 2H); HRMS calculated for (M+H) 317.1397, found 317.1405.

### EXAMPLE 36

This example illustrates the production of 2-{2-[4-(dimethylamino)phenyl]-pyridin-4-yl}-6,7-dihydropyrazolo[1,5-a]pyrazin-4(5H)-one trifluoroacetate.

Synthesis was conducted as it was for the production of 2-pyridin-4-yl-6,7-dihydropyrazolo[1,5-a]pyrazin-4(5H)-one trifluoroacetate using ethyl 1-(2-aminoethyl)-3-{2-[4-(dimethylamino)phenyl]pyridin-4-yl}-1H-pyrazole-5-carboxylate trifluoroacetate (0.350 g, 708 mmol), NH₄OH (8 mL), and ethanol (4 mL). A yellow solid was obtained (0.204 g, 64.5% yield): ¹H NMR (DMSO-d₆/300 MHz) 88.61 (d, 1H), 8.51 (s, 1H), 8.40 (s, 1H), 8.03 (d, 2H), 7.96 (d, 1H), 7.86 (s, 1H), 6.88 (d, 2H), 4.46 (t, 2H), 3.71-3.66 (m, 2H), 3.05 (s, 6H); HRMS calculated for (M+H) 334.1662, found 334.1673.

### EXAMPLE 37

This example illustrates the production of 2-[2-(4-methoxyphenyl)-pyridin-4-yl]-6,7-dihydropyrazolo[1,5-a]pyrazin-4(5H)-one trifluoroacetate.

Synthesis was conducted as it was for the production of 2-pyridin-4-yl-6,7-dihydropyrazolo[1,5-a]pyrazin-4(5H)-one trifluoroacetate using ethyl 1-(2-aminoethyl)-3-[2-(4-methoxyphenyl)pyridin-4-yl]-1H-pyrazole-5-carboxylate dihydrochloride (0.368 g, 0.838 mmol), NH₄OH (8 mL), and ethanol (4 mL). A white solid was obtained (0.225 g, 61.8% yield): ¹H NMR (DMSO-d₆/300 MHz) 88.69 (d, 1H), 8.45 (s, 1H), 8.36 (s, 1H), 8.13 (d, 2H), 7.93 (d, 1H), 7.76 (s, 1H), 7.12 (d, 2H), 4.44 (t, 2H), 3.85 (s, 3H), 3.71-3.66 (m, 2H); HRMS calculated for (M+H) 321.1346, found 321.1359.

### EXAMPLE 38

This example illustrates the production of 2-[2-(3-nitrophenyl)pyridin-4-yl]-6,7-dihydropyrazolo[1,5-a]pyrazin-4(5H)-one trifluoroacetate.

Synthesis was conducted as it was for the production of 2-pyridin-4-yl-6,7-dihydropyrazolo[1,5-a]pyrazin-4(5H)-one trifluoroacetate using ethyl 1-(2-aminoethyl)-3-[2-(3-nitrophenyl)pyridin-4-yl]-1H-pyrazole-5-carboxylate dihydrochloride (0.350 g, 0.771 mmol), NH₄OH (8 mL), and ethanol (4 mL). A beige solid was obtained (0.152 g, 43.8% yield): ¹H NMR (DMSO-d₆/300 MHz) δ 9.00 (s, 1H), 8.75 (d, 1H), 8.66 (d, 1H), 8.32-8.28 (m, 2H), 7.89 (d, 1H), 7.81 (dd, 1H), 7.73 (s, 1H), 4.43 (t, 2H), 3.70-3.65 (m, 2H); HRMS calculated for (M+H) 336.1091, found 336.1068.

### EXAMPLE 39

This example illustrates the production of 2-[2-(3,4-difluorophenyl)pyridin-4-yl]-6,7-dihydropyrazolo[1,5-a]pyrazin-4(5H)-one trifluoroacetate.

Synthesis was conducted as for the production of ethyl 1-(2-aminoethyl)-3-[2-(3-nitrophenyl)pyridin-4-yl]-1H-pyrazole-5-carboxylate dihydrochloride using 2-(2-chloropyridin-4-yl)-6,7-dihydropyrazolo[1,5-a]pyrazin-4(5H)-one (0.175 g, 0.702 mmol), 3,4-difluorophenylboronic acid (0.167 g, 1.05 mmol), 2M Na₂CO₃ (2 mL), toluene (7 mL), and Pd[dppf]Cl₂.CH₂Cl₂ (0.046 g, 0.057 mmol). The TFA salt was isolated as a white solid (0.076 g, 25% yield): ¹H NMR (DMSO-d₆/300 MHz) δ 8.68 (d, 1H), 8.42 (s, 1H), 8.32-8.23 (m, 2H), 8.09 (d, 1H), 7.82 (s, 1H), 7.69 (s, 1H), 7.55 (dd, 1H), 4.41 (t, 2H), 3.69-3.64 (m, 2H); HRMS calculated for (M+H) 327.1052, found 327.1078.

### EXAMPLE 40

This example illustrates the production of 2-[2-(3,4-dichlorophenyl)pyridin-4-yl]-6,7-dihydropyrazolo[1,5-a]pyrazin-4(5H)-one trifluoroacetate.

Synthesis was conducted as for the production of ethyl 1-(2-aminoethyl)-3-[2-(3-nitrophenyl)pyridin-4-yl]-1H-pyrazole-5-carboxylate dihydrochloride using 2-(2-chloropyridin-4-yl)-6,7-dihydropyrazolo[1,5-a]pyrazin-4(5H)-one (0.175 g, 0.702 mmol), 3,4-dichlorophenylboronic acid (0.200 g, 1.05 mmol), 2M Na₂CO₃ (2 mL), toluene (7 mL), and Pd[dppf]Cl₂.CH₂Cl₂ (0.046 g, 0.057 mmol). The TFA salt was obtained as a gray solid (0.016 g, 4.9% yield): ¹H NMR (DMSO-d₆, TFA/300 MHz) δ 8.81 (d, 1H), 8.66 (s, 1H), 8.42-8.34 (m, 2H), 8.28-8.18 (m, 2H), 7.87-7.85 (m, 2H), 4.45 (t, 2H), 3.71-3.64 (m, 2H), HRMS calculated for (M+H) 359.0461, found 359.0476.

### EXAMPLE 41

This example illustrates the production of 1-(3-aminopropyl)-3-[2-(3-nitrophenyl)pyridin-4-yl]-1H-pyrazole-5-carboxylic acid dihydrochloride.

Synthesis was conducted as for the preparation of 1-(2-aminoethyl)-3-pyridin-4-yl-1H-pyrazole-5-carboxylic acid trifluoroacetate, using ethyl 1-{3-[(tert-butoxycarbonyl)amino]propyl}-3-[2-(3-nitrophenyl)pyridin-4-yl]-1H-pyrazole-5-carboxylate (0.253 g, 0.539 mmol) and LiOH.H₂O (0.0679 g, 1.62 mmol. The isolated TFA salt was a tacky solid, and thus converted to the HCl salt using methanol and 4N HCl/dioxane. After 0.5 h, the suspension was concentrated and the solid rinsed with diethyl ether to afford a white solid (0.223 g, 94.6% yield): ¹H NMR (DMSO-d₆/300 MHz) δ 9.00 (dd, 1H), 8.79 (d, 1H), 8.68 (d, 1H), 8.60 (s, 1H), 8.34 (dd, 1H), 8.11 (br s, 3H), 7.99 (dd, 1H), 7.88-7.81 (m, 2H), 4.68 (t, 2H), 2.86-2.79 (m, 2H), 2.20-2.15 (m, 2H); HRMS calculated for (M+H) 368.1353, found 368.1336.

### EXAMPLE 42

This example illustrates the production of 1-(2-aminoethyl)-3-(2-quinolin-3-ylpyridin-4-yl)-1H-pyrazole-5-carboxylic acid trifluoroacetate.

Synthesis was conducted as for the preparation of 1-(2-aminoethyl)-3-pyridin-4-yl-1H-pyrazole-5-carboxylic acid trifluoroacetate, using ethyl 1-(2-aminoethyl)-3-(2-quinolin-3-ylpyridin-4-yl)-1H-pyrazole-5-carboxylate dihydrochloride (0.211 g, 0.457 mmol) and LiOH.H₂O (0.077 g, 1.8 mmol). A pink solid was obtained (0.150 g, 69.4% yield): ¹H NMR (DMSO-d₆/300 MHz) δ 9.75 (s, 1H), 9.23 (d, 1H), 8.82 (d, 1H), 8.69 (s, 1H), 8.17-8.10 (m, 2H), 8.01-7.94 (m, 3H), 7.89-7.83 (m, 2H), 7.72 (dd, 1H), 4.86 (t, 2H), 3.44-3.38 (m, 2H); HRMS calculated for (M+H) 360.1455, found 360.1466.

### EXAMPLE 43

This example illustrates the production of 1-(2-aminoethyl)-3-[2-(3-nitrophenyl)pyridin-4-yl]-1H-pyrazole-5-carboxylic acid dihydrochloride.

Synthesis was conducted as for the preparation of 1-(2-aminoethyl)-3-pyridin-4-yl-1H-pyrazole-5-carboxylic acid trifluoroacetate, using ethyl 1-(2-aminoethyl)-3-[2-(3-nitrophenyl)pyridin-4-yl]-1H-pyrazole-5-carboxylate dihydrochloride (0.262 g, 0.516 mmol) and LiOH.H₂O (0.097 g, 2.3 mmol). The isolated TFA salt converted to the HCl salt using methanol and 4N HCl/dioxane. After 0.5 h, the suspension was concentrated and the solid rinsed with diethyl ether to afford a yellow solid (0.102 g, 46.5% yield): ¹H NMR (DMSO-d₆/300 MHz) δ 9.01 (s, 1H), 8.80 (d, 1H), 8.70-8.64 (m, 2H), 8.35-8.28 (m, 4H), 8.02 (d, 1H), 7.89-7.82 (m, 2H), 3.39-3.33 (m, 2H); HRMS calculated for (M+H) 354.1197, found 354.1176.

### EXAMPLE 44

This example illustrates the production of 1-(2-aminoethyl)-3-[2-(4-methoxyphenyl)pyridin-4-yl]-1H-pyrazole-5-carboxylic acid trifluoroacetate.

Synthesis was conducted as for the preparation of 1-(2-aminoethyl)-3-pyridin-4-yl-1H-pyrazole-5-carboxylic acid trifluoroacetate, using ethyl 1-(2-aminoethyl)-3-[2-(4-methoxyphenyl)pyridin-4-yl]-1H-pyrazole-5-carboxylate dihydrochloride (0.260 g, 0.591 mmol) and LiOH.H₂O (0.099 g, 2.4 mmol). A pale yellow solid was obtained (0.212 g, 79.4% yield): ¹H NMR (DMSO-d₆/300 MHz) δ 8.69 (d, 1H), 8.42 (s, 2H), 8.15 (d, 1H), 8.00 (br s, 3H), 7.88-7.84 (m, 2H), 7.09 (d, 2H), 4.84 (t, 2H), 3.84 (s, 3H), 3.38-3.42 (m, 2H); HRMS calcd for (M+H) 339.1452, found 339.1472.

### EXAMPLE 45

This example illustrates the production of 1-(2-aminoethyl)-3-{2-[4-(trifluoromethoxy)phenyl]pyridin-4-yl}-1H-pyrazole-5-carboxylic acid trifluoroacetate.

Synthesis was conducted as for the preparation of 1-(2-aminoethyl)-3-pyridin-4-yl-1H-pyrazole-5-carboxylic acid trifluoroacetate, using ethyl 1-(2-aminoethyl)-3-{2-[4-(trifluoromethoxy)phenyl]pyridin-4-yl}-1H-pyrazole-5-carboxylate dihydrochloride (0.257 g, 0.521 mmol) and LiOH.H₂O (0.097 g, 2.3 mmol). A beige solid was obtained (0.088 g, 34% yield): ¹H NMR (DMSO-d₆/300 MHz) δ 8.73 (d, 1H), 8.50 (s, 1H), 8.32 (d, 2H), 8.01 (br s, 3H), 7.88 (d, 1H), 7.81 (s, 1H), 7.51 (d, 2H), 4.84 (t, 2H), 3.42-3.38 (m, 2H); HRMS calculated for (M+H) 393.1169, found 393.1189.

### EXAMPLE 46

This example illustrates the production of 1-(2-aminoethyl)-3-{2-[4-(dimethylamino)phenyl]pyridin-4-yl}-1H-pyrazole-5-carboxylic acid trifluoroacetate.

Synthesis was conducted as for the preparation of 1-(2-aminoethyl)-3-pyridin-4-yl-1H-pyrazole-5-carboxylic acid trifluoroacetate using ethyl 1-(2-aminoethyl)-3-{2-[4-(dimethylamino)phenyl]pyridin-4-yl}-1H-pyrazole-5-carboxylate trifluoroacetate (0.238 g, 0.482 mmol) and LiOH.H₂O (0.088 g, 2.1 mmol). A neon orange solid was obtained (0.150 g, 67.0% yield): ¹H NMR (DMSO-d₆/300 MHz) δ 8.64 (d, 1H), 8.46 (s, 1H), 8.07-8.04 (m, 5H), 7.91-7.88 (m, 2H), 6.86 (d, 2H), 4.86 (t, 2H), 3.43-3.37 (m, 2H), 3.03 (s, 6H); HRMS calcd for (M+H) 352.1768, found 352.1770.

### EXAMPLE 47

This example illustrates the production of 1-(2-aminoethyl)-3-{2-[(E)-2-phenylethenyl]pyridin-4-yl}-1H-pyrazole-5-carboxylic acid trifluoroacetate.

Synthesis was conducted as in the preparation of 1-(2-aminoethyl)-3-pyridin-4-yl-1H-pyrazole-5-carboxylic acid trifluoroacetate, using ethyl 1-(2-aminoethyl)-3-{2-[(E)-2-phenylvinyl]pyridin-4-yl}-1H-pyrazole-5-carboxylate dihydrochloride (0.311 g, 0.714 mmol) and LiOH.H₂O (0.120 g, 2.86 mmol). The TFA salt was isolated as a light yellow solid (0.220 g, 68.7% yield): ¹H NMR (DMSO-d₆/300 MHz) δ 8.68 (d, 1H), 8.26 (s, 1H), 8.03 (br s, 3H), 7.89-7.84 (m, 2H), 7.73-7.68 (m, 3H), 7.45-7.36 (m, 4H), 4.89 (t, 2H), 3.41-3.39 (m, 2H); HRMS calculated for (M+H) 335.1503, found 335.1496.

### EXAMPLE 48

This example illustrates the production of 1-(3-aminopropyl)-3-(2-quinolin-3-ylpyridin-4-yl)-1H-pyrazole-5-carboxylic acid dihydrochloride.

Synthesis was conducted as for the preparation of 1-(2-aminoethyl)-3-pyridin-4-yl-1H-pyrazole-5-carboxylic acid trifluoroacetate, using ethyl 1-(3-aminopropyl)-3-(2-quinolin-3-ylpyridin-4-yl)-1H-pyrazole-5-carboxylate dihydrochloride (0.134 g, 0.282 mmol) and LiOH.H₂O (0.047 g, 1.1 mmol). The TFA salt was converted to the HCl salt using methanol and 4N HCl/dioxane. After 0.5 h, the suspension was concentrated and the solid rinsed with diethyl ether to afford a yellow solid (0.086 g, 68% yield): ¹H NMR (DMSO-d₆/300 MHz) δ 9.92 (d, 1H), 9.95 (s, 1H), 9.83 (s, 1H), 8.44-8.41 (m, 2H), 8.17-8.09 (m, 4H), 8.03 (d, 1H), 7.94 (dd, 1H), 7.86 (s, 1H), δ4.69 (t, 2H), 2.90-2.82 (m, 2H), 2.25-2.16 (m, 2H); HRMS calculated for (M+H) 374.1612, found 374.1622.

### EXAMPLE 49

This example illustrates the production of 1-(2-aminoethyl)-3-{2-[4-(hydroxymethyl)phenyl]pyridin-4-yl}-1H-pyrazole-5-carboxylic acid dihydrochloride.

Synthesis was conducted as for the preparation of 1-(2-aminoethyl)-3-pyridin-4-yl-1H-pyrazole-5-carboxylic acid trifluoroacetate, using ethyl 1-(2-aminoethyl)-3-{2-[4-(hydroxymethyl)phenyl]pyridin-4-yl}-1H-pyrazole-5-carboxylate dihydrochloride (0.167 g, 0.379 mmol) and LiOH.H₂O (0.064 g, 1.5 mmol). The TFA salt was converted to the HCl salt using methanol and 4N HCl/dioxane. After 0.5 h, the suspension was concentrated and the solid rinsed with diethyl ether to afford a pale pink solid (0.069 g, 70% yield): ¹H NMR (DMSO-d₆/300 MHz) δ 8.80 (d, 1H), 8.67 (s, 1H), 8.33 (br s, 3H), 8.22-8.19 (m, 3H), 8.00 (s, 1H), 7.55 (d, 2H), 4.89 (t, 2H), 4.62 (m, 2H), 3.40-3.35 (m, 2H); HRMS calculated for (M+H) 339.1452, found 339.1435.

### EXAMPLE 50

This example illustrates the production of 1-(2-aminoethyl)-3-[2-(3-methoxyphenyl)pyridin-4-yl]-1H-pyrazole-5-carboxylic acid trifluoroacetate.

Synthesis was conducted as for the preparation of 1-(2-aminoethyl)-3-pyridin-4-yl-1H-pyrazole-5-carboxylic acid trifluoroacetate, using ethyl 1-(2-aminoethyl)-3-[2-(3-methoxyphenyl)pyridin-4-yl]-1H-pyrazole-5-carboxylate dihydrochloride (0.179 g, 0.407 mmol) and LiOH.H₂O (0.105 g, 2.50 mmol). An off-white solid was obtained (0.168 g, 91.2% yield): ¹H NMR (DMSO-d₆/300 MHz) δ 8.72 (d, 1H), 8.43 (s, 1H), 8.00 (br s, 3H), 7.88 (d, 1H), 7.83 (s, 1H), 7.78-7.74 (m, 2H), 7.44 (dd, 1H), 7.06 (dd, 1H), 4.84 (t, 2H), 3.85 (s, 3H), 3.44-3.38 (m, 2H); HRMS calculated for (M+H) 339.1452, found 339.1469.

### EXAMPLE 51

This example illustrates the production of 1-(2-aminoethyl)-N-hydroxy-3-{2-[4-(hydroxymethyl)phenyl]pyridin-4-yl}-1H-pyrazole-5-carboxamide trifluoroacetate.

Freshly made sodium methoxide (3.48 M, 0.27 mL) was added dropwise to a stirring solution of hydroxylamine hydrochloride (0.039 mL, 0.94 mmol) in methanol (1 mL) maintained at 40° C. The white slurry was cooled to room temperature, and then a solution of ethyl 1-{2-[(tert-butoxycarbonyl)amino]ethyl}-3-{2-[4-(hydroxymethyl)phenyl]pyridin-4-yl}-1H-pyrazole-5-carboxylate (0.400 g, 0.857 mmol) in methanol (5 mL) was added. After stirring for 48 h, 7 mL of 4N HCl (aq) was added and the reaction stirred for 20 h. Purification by Gilson RP HPLC (5-95% acetonitrile/water) afforded a pink solid (0.129 g, 32.1% yield): ¹H NMR (DMSO-d₆/300 MHz) 611.53 (brs, 1H), 8.73 (d, 1H), 8.31 (s, 1H), 8.11-8.01 (m, 5H), 7.75 (d, 1H), 7.52-7.46 (m, 3H), 4.77 (t, 2H), 4.58 (s, 2H), 3.42-3.35 (m, 2H); HRMS calculated for (M+H) 354.1561, found 354.1538.

### EXAMPLE 52

This example illustrates the production of 2-[2-(1H-pyrazol-1-yl)pyridin-4-yl]-5,6,7,8-tetrahydro-4H-pyrazolo[1,5-a][1,4]diazepin-4-one hydrochloride.

A flask was charged with 1. 2-(2-chloropyridin-4-yl)-5,6,7,8-tetrahydro-4H-pyrazolo[1,5-a][1,4]diazepin-4-one (0.251 g, 0.960 mmol), pyrazole (0.325 g, 4.78 mmol), and sodium hydride (0.229 g, 5.73 mmol) in 6 mL anhydrous DMF and stirred under N₂ for 60 h at 138° C. The reaction was then quenched with 1 N HCl (aq) and purified by Gilson RP HPLC (5-95% acetonitrile/water). The appropriate fractions were concentrated and converted to the HCl salt using methanol and 4N HCl/dioxane. The mixture was concentrated to a light yellow solid (0.028 g, 8.2% yield): ¹H NMR (DMSO-d₆/300 MHz) δ 8.64 (d, 1H), 8.48 (d, 1H), 8.35 (br s, 2H), 7.85 (s, 1H), 7.77 (d, 1H), 7.45 (s, 1H), 6.59 (s, 1H), 4.53 (t, 2H), 3.26-3.20 (m, 2H), 2.22-2.16 (m, 2H); HRMS calculated for (M+H) 295.1302, found 295.1285.

### EXAMPLE 53

This example illustrates the production of 2-[2-(1H-imidazol-1-yl)pyridin-4-yl]-5,6,7,8-tetrahydro-4H-pyrazolo[1,5-a][1,4]diazepin-4-one hydrochloride.

Synthesis was conducted as for the production of 2-[2-(1H-pyrazol-1-yl)pyridin-4-yl]-5,6,7,8-tetrahydro-4H-pyrazolo[1,5-a][1,4]diazepin-4-one hydrochloride using 2-(2-chloropyridin-4-yl)-5,6,7,8-tetrahydro-4H-pyrazolo[1,5-a][1,4]diazepin-4-one (0.251 g, 0.960 mmol) and imidazole, sodium derivative (0.431 g, 4.78 mmol). A white solid was obtained (0.074 g, 23% yield): ¹H NMR (DMSO-d₆/300 MHz) δ 10.0 (s, 1H), 8.62 (d, 1H), 8.55 (s, 1H), 8.48 (s, 1H), 8.41 (br s, 1H), 7.99 (d, 1H), 7.90 (s, 1H), 7.65 (s, 1H), 4.53 (t, 2H), 3.28-3.22 (m, 2H), 2.22-2.16 (m, 2H); HRMS calculated for (M+H) 295.1302, found 295.1290.

### EXAMPLE 54

This example illustrates the production of 2-[2-(1H-pyrrol-1-yl)pyridin-4-yl]-5,6,7,8-tetrahydro-4H-pyrazolo[1,5-a][1,4]diazepin-4-one trifluoroacetate.

Synthesis was conducted as for the production of 2-[2-(1H-pyrazol-1-yl)pyridin-4-yl]-5,6,7,8-tetrahydro-4H-pyrazolo[1,5-a][1,4]diazepin-4-one hydrochloride, using 2-(2-chloropyridin-4-yl)-5,6,7,8-tetrahydro-4H-pyrazolo[1,5-a][1,4]diazepin-4-one (0.251 g, 0.960 mmol), pyrrole (0.334 mL, 4.78 mmol), and sodium hydride (0.229 g, 5.73 mmol). The TFA salt was isolated as a black solid (0.128 g, 32.7% yield): ¹H NMR (DMSO-d₆/300 MHz) δ 8.42 (d, 1H), 8.34 (br s, 1H), 8.05 (s, 1H), 7.79 (br s, 2H), 7.67-7.64 (m, 2H), 6.30 (br s, 2H), 4.52 (t, 2H), 3.28-3.22 (m, 2H), 2.22-2.16 (m, 2H); HRMS calculated for (M+H) 294.1349, found 294.1348.

### EXAMPLE 55

This example illustrates the production of 2-[2-(4-methyl-1H-imidazol-1-yl)pyridin-4-yl]-5,6,7,8-tetrahydro-4H-pyrazolo[1,5-a][1,4]diazepin-4-one hydrochloride.

Synthesis was conducted as for the production of 2-[2-(1H-pyrazol-1-yl)pyridin-4-yl]-5,6,7,8-tetrahydro-4H-pyrazolo[1,5-a][1,4]diazepin-4-one hydrochloride, using 2-(2-chloropyridin-4-yl)-5,6,7,8-tetrahydro-4H-pyrazolo[1,5-a][1,4]diazepin-4-one (0.251 g, 0.960 mmol), 4-methylimidazole (0.393 g, 4.78 mmol), and sodium hydride (0.229 g, 5.73 mmol). A brown solid was obtained (0.053 g, 16% yield): ¹H NMR (DMSO-d₆/300 MHz) δ 9.88 (s, 1H), 8.60 (d, 1H), 8.40 (br s, 2H), 8.28 (s, 1H), 7.97 (d, 1H), 7.63 (s, 1H), 4.53 (t, 2H), 3.29-3.22 (m, 2H), 2.36 (s, 3H), 2.22-2.16 (m, 2H); HRMS calculated for (M+H) 309.1458, found 309.1462.

### EXAMPLE 56

This example illustrates the production of 2-[2-(4-phenyl-1H-imidazol-1-yl)pyridin-4-yl]-5,6,7,8-tetrahydro-4H-pyrazolo[1,5-a][1,4]diazepin-4-one trifluoroacetate.

Synthesis was conducted as for the production of 2-[2-(1H-pyrazol-1-yl)pyridin-4-yl]-5,6,7,8-tetrahydro-4H-pyrazolo[1,5-a][1,4]diazepin-4-one hydrochloride, using 2-(2-chloropyridin-4-yl)-5,6,7,8-tetrahydro-4H-pyrazolo[1,5-a][1,4]diazepin-4-one (0.251 g, 0.960 mmol), 4-phenylimidazole (0.690 g, 4.78 mmol), and sodium hydride (0.229 g, 5.73 mmol). The TFA salt was isolated as a white solid (0.060 g, 13% yield): ¹H NMR (DMSO-d₆/300 MHz) 69.17 (s, 1H), 8.76 (s, 1H), 8.57 (d, 1H), 8.37-8.32 (m, 2H), 7.93-7.88 (m, 3H), 7.65 (s, 1H), 7.47 (dd, 2H), 7.36-7.34 (m, 1H), 4.54 (t, 2H), 3.29-3.22 (m, 2H), 2.22-2.16 (m, 2H); HRMS calculated for (M+H) 371.1615, found 371.1626.

### EXAMPLE 57

This example illustrates the production of 2-[2-(4-methyl-1H-pyrazol-1-yl)pyridin-4-yl]-5,6,7,8-tetrahydro-4H-pyrazolo[1,5-a][1,4]diazepin-4-one trifluoroacetate.

Synthesis was conducted as it was for the production of 2-[2-(1H-pyrazol-1-yl)pyridin-4-yl]-5,6,7,8-tetrahydro-4H-pyrazolo[1,5-a][1,4]diazepin-4-one hydrochloride, using 2-(2-chloropyridin-4-yl)-5,6,7,8-tetrahydro-4H-pyrazolo[1,5-a][1,4]diazepin-4-one (0.251 g, 0.960 mmol), 4-methylpyrazole (0.40 mL, 4.8 mmol), and sodium hydride (0.229 g, 5.73 mmol). The TFA salt was isolated as a white solid (0.068 g, 16% yield): ¹H NMR (DMSO-d₆/300 MHz) δ 8.45-8.29 (m, 4H), 7.72 (m, 1H), 7.66 (s, 1H), 7.42 (s, 1H), 4.54 (t, 2H), 3.25-3.20 (m, 2H), 2.20-2.11 (m, 5H); HRMS calculated for (M+H) 309.1458, found 309.1448.

### EXAMPLE 58

This example illustrates the production of 2-[2-(1H-1,2,4-triazol-1-yl)pyridin-4-yl]-5,6,7,8-tetrahydro-4H-pyrazolo[1,5-a][1,4]diazepin-4-one hydrochloride.

Synthesis was conducted as it was for the production of 2-[2-(1H-pyrazol-1-yl)pyridin-4-yl]-5,6,7,8-tetrahydro-4H-pyrazolo[1,5-a][1,4]diazepin-4-one hydrochloride, using 2-(2-chloropyridin-4-yl)-5,6,7,8-tetrahydro-4H-pyrazolo[1,5-a][1,4]diazepin-4-one (0.251 g, 0.960 mmol) and 1,2,4-triazole, sodium derivative (0.435 g, 4.78 mmol). A white solid was obtained (0.157 g, 48.4% yield): ¹H NMR (DMSO-d₆/300 MHz) δ 9.40 (s, 1H), 8.55 (d, 1H), 8.37-8.33 (m, 2H), 8.27 (s, 1H), 7.90 (dd, 1H), 7.50 (s, 1H), 4.53 (t, 2H), 3.25-3.20 (m, 2H), 2.22-2.14 (m, 2H); HRMS calcd for (M+H) 296.1254, found 296.1244.

### EXAMPLE 59

This example illustrates the production of 2-[2-(1H-1,2,3-triazol-1-yl)pyridin-4-yl]-5,6,7,8-tetrahydro-4H-pyrazolo[1,5-a][1,4]diazepin-4-one hydrochloride.

Synthesis was conducted as it was for the production of 2-[2-(1H-pyrazol-1-yl)pyridin-4-yl]-5,6,7,8-tetrahydro-4H-pyrazolo[1,5-a][1,4]diazepin-4-one hydrochloride, using 2-(2-chloropyridin-4-yl)-5,6,7,8-tetrahydro-4H-pyrazolo[1,5-a][1,4]diazepin-4-one (0.251 g, 0.960 mmol), 1,2,3-triazole (0.28 mL, 4.8 mmol), and sodium hydride (0.229 g, 5.73 mmol). A white solid was obtained (0.118 g, 36.0% yield): ¹H NMR (DMSO-d₆/300 MHz) δ 8.89 (s, 1H), 8.61 (d, 1H), 8.52 (s, 1H), 8.37 (br s, 1H), 8.01-7.96 (m, 2H), 7.55 (s, 1H), 4.54 (t, 2H), 3.29-3.22 (m, 2H), 2.22-2.16 (m, 2H); HRMS calcd for (M+H) 296.1254, found 296.1243.

### EXAMPLE 60

This example illustrates the production of ethyl 1-{3-[(tert-butoxycarbonyl)-amino]propyl}-3-(4-methoxyphenyl)-1H-pyrazole-5-carboxylate.

To a cooled (0° C.) solution of ethyl 3-(4-methoxyphenyl)-1H-pyrazole-5-carboxylatein anhydrous DMF (35 mL) was added lithium t-butoxide (1 M in THF, 6.6 mL) dropwise. The reaction stirred for 30 min, and then a solution of tert-butyl 3-bromopropylcarbamate (1.57 g, 6.60 mmol) and sodium iodide (0.989 g, 6.60 mmol) in anhydrous DMF (10 mL) was added dropwise. The reaction was allowed to stir and warm to room temperature for 4 h. The reaction was then poured into water and brine and extracted with ethyl acetate. The organic layers were combined, dried over MgSO₄, filtered, and concentrated. Chromatographic purification (15% ethyl acetate/hexane) afforded a yellow oil (1.13 g, 63.7% yield): ¹H NMR (DMSO-d₆/300 MHz) δ 7.81 (d, 2H), 7.27 (s, 1H), 6.87 (t, 1H), 6.79 (d, 2H), 4.52 (t, 2H), 4.37 (q, 2H), 3.80 (s, 3H), 3.30-2.94 (m, 2H), 1.96-1.91 (m, 2H), 1.39-1.33 (m, 12H); HRMS calculated for (M+H) 404.2180, found 404.2190.

### EXAMPLE 61

This example illustrates the production of 1-{3-[(tert-butoxycarbonyl)-amino]propyl}-3-(4-methoxyphenyl)-1H-pyrazole-5-carboxylic acid.

A solution of ethyl 1-{3-[(tert-butoxycarbonyl)amino]propyl}-3-(4-methoxyphenyl)-1H-pyrazole-5-carboxylate (0.467 g, 1.16 mmol) and LiOH.H₂O (0.097 g, 2.32 mmol) in 12 mL of THF/H₂O (1:1) stirred at room temperature for 3 h. The reaction mixture was concentrated to the aqueous phase, then diluted with 0.1 N HCl and extracted three times with ethyl acetate. The organic layers were combined, dried over MgSO₄, filtered, and concentrated to a pale yellow solid (0.359 g, 82.3% yield): ¹H NMR (DMSO-d₆/300 MHz) δ 7.71 (d, 2H), 7.01-6.94 (m, 3H), 6.88 (s, 1H), 4.62 (t, 2H), 3.80 (s, 3H), 2.94-2.90 (m, 2H), 1.91-1.84 (m, 2H), 1.39 (s, 9H); HRMS calculated for (M+H) 376.1867, found 376.1906.

### EXAMPLE 61

This example illustrates the production of 1-(3-aminopropyl)-3-(4-hydroxyphenyl)-1H-pyrazole-5-carboxylic acid dihydrochloride.

To a cooled (−78° C.) solution of 1-{3-[(tert-butoxycarbonyl)amino]propyl}-3-(4-methoxyphenyl)-1H-pyrazole-5-carboxylic acid (0.262 g, 0.70 mmol) in anhydrous dichloromethane (7 mL) was added boron tribromide (1.0M in CH₂Cl₂, 7.0 mL) dropwise. After 1 h the reaction was carefully quenched with water, then concentrated to the aqueous layer and purified by Gilson RP HPLC (5-95% acetonitrile/water). The appropriate fractions were concentrated. The residue was converted to the HCl salt using 4N HCl/dioxane and methanol. After stirring 1 h, the mixture was concentrated to a white solid (0.238 g, 100% yield): ¹H NMR (DMSO-d₆/300 MHz) δ 8.07 (br s, 3H), 7.67 (d, 2H), 7.18 (s, 1H), 6.83 (d, 2H), 4.59 (t, 2H), 2.84-2.78 (m, 2H), 2.18-2.08 (m, 2H); HRMS calculated for (M+H) 262.1186, found 262.1195.

### EXAMPLE 62

This example illustrates the production of ethyl 1-(3-aminopropyl)-3-(4-methoxyphenyl)-1H-pyrazole-5-carboxylate hydrochloride.

To a flask charged with ethyl 1-{3-[(tert-butoxycarbonyl)amino]propyl}-3-(4-methoxyphenyl)-1H-pyrazole-5-carboxylate (0.588 g, 1.46 mmol) was added 4N HCl/dioxane (5 mL). After 1 h the reaction mixture was filtered and rinsed with diethyl ether to yield a white solid (0.443 g, 89.4% yield): ¹H NMR (DMSO-d₆/300 MHz) δ 8.06 (br s, 3H), 7.81 (d, 2H), 7.32 (s, 1H), 7.00 (d, 2H), 4.61 (t, 2H), 4.37 (q, 2H), 3.80 (s, 3H), 2.87-2.80 (m, 2H), 2.20-2.11 (m, 2H), 1.36 (t, 3H), HRMS calcd for (M+H) 304.1656, found 304.1665.

### EXAMPLE 63

This example illustrates the production of 2-(4-methoxyphenyl)-5,6,7,8-tetrahydro-4H-pyrazolo[1,5-a][1,4]diazepin-4-one.

To a flask charged with ethyl 1-(3-aminopropyl)-3-(4-methoxyphenyl)-1H-pyrazole-5-carboxylate hydrochloride (0.418 g, 1.23 mmol) was added NH₄OH (20 mL) and ethanol (10 mL). After stirring for 18 h, the reaction was filtered, and the white solid rinsed with diethyl ether to afford a white solid (0.243 g, 76.8% yield): ¹H NMR (DMSO-d₆/300 MHz) δ 8.28 (br s, 1H), 7.77 (d, 2H), 7.10 (s, 1H), 6.98 (d, 2H), 4.45 (t, 2H), 3.80 (s, 3H), 3.26-3.21 (m, 2H), 2.20-2.13 (m, 2H); HRMS calculated for (M+H) 258.1242, found 258.1237.

### EXAMPLE 64

This example illustrates the production of 2-(4-hydroxyphenyl)-5,6,7,8-tetrahydro-4H-pyrazolo[1,5-a][1,4]diazepin-4-one.

Synthesis was conducted as it was for the preparation of 1-(3-amino-propyl)-3-(4-hydroxyphenyl)-1H-pyrazole-5-carboxylic acid dihydrochloride using ethyl 1-(3-aminopropyl)-3-(4-methoxyphenyl)-1H-pyrazole-5-carboxylate hydrochloride (0.182 g, 0.70 mmol) and boron tribromide (7.0 mL). Purification by Gilson RP HPLC (5-95% acetonitrile/water) afforded a white solid (0.078 g, 46% yield): ¹H NMR (DMSO-d₆/300 MHz) δ 9.55 (br s, 1H), 8.25 (br s, 1H), 7.65 (d, 2H), 7.03 (s, 1H), 6.80 (d, 2H), 4.44 (t, 2H), 3.26-3.21 (m, 2H), 2.18-2.13 (m, 2H); HRMS calculated for (M+H) 244.1081, found 244.1049.

### EXAMPLE 65

This example illustrates the production of ethyl 1-{3-[(tert-butoxy-carbonyl)amino]propyl}-3-(3-methoxyphenyl)-1H-pyrazole-5-carboxylate.

Synthesis was conducted as it was for the preparation of ethyl 1-{3-[(tert-butoxycarbonyl)amino]propyl}-3-(4-methoxyphenyl)-1H-pyrazole-5-carboxylate, using ethyl 3-(3-methoxyphenyl)-1H-pyrazole-5-carboxylate (2.01 g, 8.17 mmol), lithium t-butoxide (12.3 mL), tert-butyl 3-bromopropylcarbamate (2.92 g, 12.3 mmol) and sodium iodide (1.84 g, 12.3 mmol). Flash chromatography (12% ethyl acetate/hexane) afforded a yellow oil, which was triturated with diethyl ether to yield a pale yellow solid (1.47 g, 45.0% yield): HRMS calculated for (M+H) 404.2180, found 404.2206.

### EXAMPLE 66

This example illustrates the production of 1-{3-[(tert-butoxycarbonyl)-amino]propyl}-3-(3-methoxyphenyl)-1H-pyrazole-5-carboxylic acid.

Synthesis was conducted as it was for the preparation of 1-{3-[(tert-butoxycarbonyl)amino]propyl}-3-(4-methoxyphenyl)-1H-pyrazole-5-carboxylic acid using ethyl 1-{3-[(tert-butoxycarbonyl)amino]propyl}-3-(3-methoxyphenyl)-1H-pyrazole-5-carboxylate (0.610 g, 1.51 mmol) and LiOH.H₂O (0.127 g, 3.02 mmol). An off-white solid was obtained (0.565 g, 100% yield): ¹H NMR (DMSO-d₆/300 MHz) δ 13.47 (br s, 1H), 7.46-7.32 (m, 4H), 6.93-6.86 (m, 2H), 4.56 (t, 2H), 3.83 (s, 3H), 3.01-2.95 (m, 2H), 1.96-1.90 (m, 2H), 1.39 (s, 9H); HRMS calculated for (M+H) 376.1873, found 376.1896.

### EXAMPLE 67

This example illustrates the production of 1-(3-aminopropyl)-3-(3-hydroxyphenyl)-1H-pyrazole-5-carboxylic acid trifluoroacetate.

Synthesis was conducted as it was for the preparation of 1-(3-amino-propyl)-3-(4-hydroxyphenyl)-1H-pyrazole-5-carboxylic acid dihydrochloride, using 1-{3-[(tert-butoxycarbonyl)amino]propyl}-3-(3-methoxyphenyl)-1H-pyrazole-5-carboxylic acid (0.496 g, 1.32 mmol) and boron tribromide (1.0M in CH₂Cl₂, 13.0 mL). The TFA salt was isolated as an off-white solid (0.353 g, 71.3% yield): ¹H NMR (DMSO-d₆/300 MHz) δ 13.62 (br s, 1H), 9.56 (br s, 1H), 7.80 (br s, 3H), 7.27-7.23 (m, 4H), 6.77 (d, 1H), 4.63 (t, 2H), 2.89-2.81 (m, 2H), 2.17-2.07 (m, 2H); HRMS calculated for (M+H) 262.1192, found 262.1223.

### EXAMPLE 68

This example illustrates the production of 2-(3-methoxyphenyl)-5,6,7,8-tetrahydro-4H-pyrazolo[1,5-a][1,4]diazepin-4-one.

Synthesis was conducted as it was for the preparation of ethyl 1-(3-aminopropyl)-3-(4-methoxyphenyl)-1H-pyrazole-5-carboxylate hydrochloride, using ethyl 1-{3-[(tert-butoxycarbonyl)amino]propyl}-3-(3-methoxyphenyl)-1H-pyrazole-5-carboxylate (0.737 g, 1.83 mmol) and 4N HCl/dioxane (10 mL). The resulting colorless oil was subjected to conditions described for the production of 2-(4-methoxyphenyl)-5,6,7,8-tetrahydro-4H-pyrazolo[1,5-a][1,4]diazepin-4-one, sing NH₄OH (12 mL) and ethanol (6 mL). An off-white solid was obtained: ¹H NMR (DMSO-d₆/300 MHz) δ 8.29 (br s, 1H), 7.34-7.45 (m, 3H), 7.23 (s, 1H), 6.90 (dd, 1H), 4.48 (t, 2H), 3.83 (s, 3H), 3.26-3.21 (m, 2H), 2.22-2.14 (m, 2H); HRMS calculated for (M+H) 258.1242, found 258.1232.

### EXAMPLE 69

This example illustrates the production of 2-(3-hydroxyphenyl)-5,6,7,8-tetrahydro-4H-pyrazolo[1,5-a][1,4]diazepin-4-one.

Synthesis was conducted as it was for the preparation of 1-(3-amino-propyl)-3-(4-hydroxyphenyl)-1H-pyrazole-5-carboxylic acid dihydrochloride, using 2-(3-methoxyphenyl)-5,6,7,8-tetrahydro-4H-pyrazolo[1,5-a][1,4]diazepin-4-one (0.325 g 1.26 mmol) and boron tribromide (13.0 mL). Purification by Gilson RP HPLC (5-95% acetonitrile/water) afforded a white solid (0.194 g, 63.3% yield): ¹H NMR (DMSO-d₆/300 MHz) δ 9.45 (brs, 1H), 8.28 (brs, 1H), 7.28-7.19 (m, 3H), 7.08 (s, 1H), 6.75-6.72 (m, 1H), 4.47 (t, 2H), 3.26-3.21 (m, 2H), 2.20-2.16 (m, 2H); HRMS calculated for (M+H) 244.1086, found 244.1115.

### EXAMPLE 70

This example illustrates the production of 1-(3-{[2-(4-bromophenyl)ethyl]-amino}propyl)-3-pyridin-4-yl-1H-pyrazole-5-carboxylic acid hydrochloride.

A single neck round bottom flask was charged with ethyl 3-pyridin-4-yl-1H-pyrazole-5-carboxylate (1.0 g, 4.6 mmol) and 30 mL DMF. The solution was cooled to −40° C. in a dry ice/CH₃CN bath. A 1 M solution of lithium t-butoxide in THF (6.9 mL, 6.9 mmol) was added dropwise over 5 minutes. After stirring at −40° C. for 30 minutes, a solution of 3-[[2-(4-bromophenyl)ethyl](tert-butoxycarbonyl)amino]propyl methanesulfonate (3.0 g, 6.9 mmol) in 10 mL DMF was added dropwise over 5 minutes. After 1 hour the reaction was allowed to warmed to ambient temperature and stirred for 18 hours. The reaction mixture was concentrated in vacuo, and the residue taken up in ethyl acetate. This was washed 3 times with brine, dried over magnesium sulfate, and concentrated to a brown oil. The oil was treated with 20 mL of a 4 N HCl in dioxane solution, and stirred for 30 minutes to remove the protecting group. After concentrating in vacuo the crude mixture was treated with 20 mL of a 2.5 N sodium hydroxide solution. The mixture was heated to 100° C. for 1 hour to hydrolyze the ester. The resulting product mixture was concentrated in vacuo to half the volume, and then chromatographed on a Gilson reverse phase HPLC eluting with an acetonitrile/water gradient (5-70% CH₃CN over 15 minutes) to provide the desired product as a TFA salt. The salt was taken up in methanol, and treated with 10 mL of a 4 N solution of HCl in dioxane to convert to the HCl salt. Crystallization from diethyl ether afforded 1.30 g (56%) of the title compound as a tan solid. LCMS showed a single peak with m/z 429 (M+H). ¹H nmr (DMSO-dr/300 MHz) δ 9.48 (broad s, 2H), 8.94 (d, 2H), 8.46 (d, 2H), 7.90 (s, 1H), 7.50 (d, 2H), 7.22 (d, 2H), 4.71 (t, 2H), 3.20-2.88 (m, 6H), 2.30 (m, 2H), ES⁺ HRMS calculated for M+H 429.0921, observed 429.0934.

### EXAMPLE 71

This example illustrates the production of 1-(3-aminopropyl)-3-[2-(4-methoxyphenyl)pyridin-4-yl]-1H-pyrazole-5-carboxylic acid hydrochloride.

Step 1. Preparation of ethyl 1-(3-aminopropyl)-3-[2-(4-methoxyphenyl)pyridin-4-yl]-1H-pyrazole-5-carboxylate hydrochloride.

This compound was prepared as part of a parallel library. A reaction tube was charged with ethyl 1-{3-[(tert-butoxycarbonyl)amino]propyl}-3-(2-chloropyridin-4-yl)-1H-pyrazole-5-carboxylate (550 mg, 1.35 mmol) and 4-methoxybenzene boronic acid (307 mg, 2.02 mmol). The mixture was purged with N₂, and then 16 mL toluene and 6 mL of a 2M sodium carbonate solution were added. The reaction mixture was again purged with N₂. To this stirring mixture [1,1′-bis(diphenyl phosphino)ferrocene]dichloropalladium II.CH₂Cl₂ (88 mg, 0.11 mmol) was added, and the reaction heated to 80° C. for 18 hours. The water layer was decanted, and the organic layer filtered through celite, and washed with CH₂Cl₂. The filtrate was concentrated in vacuo, and the residue treated with 10 mL of a 4 N HCl in dioxane solution for 1 hour. The product was concentrated in vacuo, washed with diethyl ether, and air dried to afford 673 mg (quantitative) of the desired ester. LCMS showed one major peak with m/z 381 (M+H).

Step 2. Preparation of 1-(3-aminopropyl)-3-[2-(4-methoxyphenyl)pyridin-4-yl]-1H-pyrazole-5-carboxylic acid hydrochloride.

This compound was prepared as part of a parallel library. A reaction tube was charged with ethyl 1-(3-aminopropyl)-3-[2-(4-methoxyphenyl)pyrid in-4-yl]-1H-pyrazole-5-carboxylate hydrochloride (200 mg, 0.44 mmol) and 10 mL of a 2.5 N NaOH solution. The mixture was heated to 100° C. for 30 minutes, and concentrated in vacuo. The product mixture was chromatographed on a Gilson reverse phase HPLC eluting with an acetonitrile/water gradient (5-70% CH₃CN over 15 minutes). The fractions containing the desired product were combined and concentrated. The oil was taken up in methanol and treated with 5 mL of a 4 N HCl in dioxane solution to obtain the HCl salt. The solution was concentrated to dryness, washed with diethyl ether, and air dried to afford 196 mg (quantitative) of the title carboxylic acid. LCMS showed a single peak with m/z 353 (M+H). ES⁺ HR MS calculated for M+H 353.1608, observed 353.1640.

### EXAMPLE 72

This example illustrates the production of 1-(3-aminopropyl)-3-{2-[4-(dimethylamino)phenyl]pyrid in-4-yl}-1H-pyrazole-5-carboxylic acid hydrochloride.

The preparation of 1-(3-aminopropyl)-3-{2-[4-(dimethylamino)phenyl]-pyridin-4-yl}-1H-pyrazole-5-carboxylic acid hydrochloride was carried out in the same manner as that described for the production of 1-(3-aminopropyl)-3-[2-(4-methoxyphenyl)pyridin-4-yl]-1H-pyrazole-5-carboxylic acid hydrochloride, using 4-dimethyl(amino)phenyl boronic acid. Purification afforded the title carboxylic acid as an orange solid. 6% yield over 2 steps. LCMS showed a single peak with m/z 366 (M+H). ES⁺ HR MS calculated for M+H 366.1925, observed 366.1918.

### EXAMPLE 73

This example illustrates the production of 1-(3-aminopropyl)-3-{2-[3-(hydroxymethyl)phenyl]pyridin-4-yl}-1H-pyrazole-5-carboxylic acid hydrochloride.

The preparation of 1-(3-aminopropyl)-3-{2-[3-(hydroxymethyl)phenyl]-pyridin-4-yl}-1H-pyrazole-5-carboxylic acid hydrochloride was carried out in the same manner as that described for the production of 1-(3-aminopropyl)-3-[2-(4-methoxyphenyl)pyridin-4-yl]-1H-pyrazole-5-carboxylic acid hydrochloride, using 3-hydroxymethylphenyl boronic acid. Purification afforded the title carboxylic acid as a brown solid. 5% yield over 2 steps. LCMS showed a single peak with m/z 353 (M+H). ES⁺ HR MS calculated for M+H 353.1608, observed 353.1608.

### EXAMPLE 74

This example illustrates the production of 1-(3-aminopropyl)-3-{2-[4-(trifluoromethoxy)phenyl]pyridin-4-yl}-1H-pyrazole-5-carboxylic acid hydrochloride.

The preparation of 1-(3-aminopropyl)-3-{2-[4-(trifluoromethoxy)-phenyl]pyridin-4-yl}-1H-pyrazole-5-carboxylic acid hydrochloride was carried out in the same manner as that described for the production of 1-(3-aminopropyl)-3-[2-(4-methoxyphenyl)pyrid in-4-yl]-1H-pyrazole-5-carboxylic acid hydrochloride using 4-trifluoromethoxy-benzene boronic acid. Purification afforded the title carboxylic acid as a brown solid. 3% yield over 2 steps. LCMS showed a single peak with m/z 407 (M+H). ES⁺ HR MS calculated for M+H 407.1326, observed 407.1358.

### EXAMPLE 75

This example illustrates the production of 2-[2-(4-methoxyphenyl)-pyridin-4-yl]-5,6,7,8-tetrahydro-4H-pyrazolo[1,5-a][1,4]diazepin-4-one.

This compound was prepared as part of a parallel library. A reaction tube was charged with ethyl 1-(3-aminopropyl)-3-[2-(4-methoxyphenyl)pyridin-4-yl]-1H-pyrazole-5-carboxylate hydrochloride (250 mg, 0.55 mmol), 20 mL ammonium hydroxide, and 10 mL ethanol. The reaction mixture was stirred at room temperature for 18 hours. The mixture was then concentrated in vacuo, washed with water, filtered, and vacuum dried to afford 150 mg (81%) of the title lactam as a tan solid. LCMS showed a single peak with m/z 335 (M+H). ¹H nmr (DMSO-d₆+TFA/300 MHz) δ 8.78 (d, 1H), 8.68 (s, 1H), 8.43 (br s, 1H), 8.26 (d, 1H), 8.09 (d, 2H), 7.93 (s, 1H), 7.23 (d, 2H), 4.59 (m, 2H), 3.89 (s, 3H), 3.26 (m, 2H), 2.21 (m, 2H). ES⁺ HR MS calculated for M+H 335.1503, observed 335.1490.

### EXAMPLE 76

This example illustrates the production of 2-{2-[4-(dimethylamino)-phenyl]pyridin-4-yl}-5,6,7,8-tetrahydro-4H-pyrazolo[1,5-a][1,4]diazepin-4-one trifluoroacetate.

The preparation of 2-{2-[4-(dimethylamino)phenyl]pyridin-4-yl}-5,6,7,8-tetrahydro-4H-pyrazolo[1,5-a][1,4]diazepin-4-one trifluoroacetate was carried out in a manner similar to that described for the preparation of 2-[2-(4-methoxyphenyl)pyridin-4-yl]-5,6,7,8-tetrahydro-4H-pyrazolo[1,5-a][1,4]diazepin-4-one, except that it was chromatographed on a Gilson reverse phase HPLC eluting with an acetonitrile/water gradient (5-70% CH₃CN over 15 minutes). The pure fractions were combined and concentrated to afford the title lactam as a yellow solid (44% yield). LCMS showed a single peak with m/z 348 (M+H). ¹H nmr (DMSO-dr/300 MHz) δ 8.61 (d, 1H), 8.52 (s, 1H), 8.41 (br s, 1H), 8.03 ((d, 2H), 7.98 (d, 1H), 7.84 (s, 1H), 6.89 (d, 2H), 4.57 (t, 2H), 3.25 (m, 2H), 3.05 (s, 6H), 2.21 (m, 2H). mp=223.0-226.7° C. ES⁺ HR MS calculated for M+H 348.1819, observed 348.1790.

### EXAMPLE 77

This example illustrates the production of 2-{2-[3-(hydroxymethyl)-phenyl]pyridin-4-yl}-5,6,7,8-tetrahydro-4H-pyrazolo[1,5-a][1,4]diazepin-4-one trifluoroacetate.

The preparation of 2-{2-[3-(hydroxymethyl)phenyl]pyridin-4-yl}-5,6,7,8-tetrahydro-4H-pyrazolo[1,5-a][1,4]diazepin-4-one trifluoroacetate was carried out in a manner similar to that described for the preparation of 2-[2-(4-methoxyphenyl)pyridin-4-yl]-5,6,7,8-tetrahydro-4H-pyrazolo[1,5-a][1,4]diazepin-4-one, except that it was chromatographed on a Gilson reverse phase HPLC eluting with an acetonitrile/water gradient (5-70% CH₃CN over 15 minutes). The pure fractions were combined and concentrated to afford the lactam as a white solid (26% yield). LCMS showed a single peak with m/z 335 (M+H). ¹H nmr (DMSO-d₆/300 MHz) δ 8.71 (d, 1H), 8.40 (s, 1H), 8.35 (brs, 1H), 8.11 (s, 1H), 8.03 (d, 1H), 7.86 (d, 1H), 7.63 (s, 1H), 7.53-7.41 (m, 2H), 4.61 (s, 2H), 4.54 (t, 2H), 3.24 (m, 2H), 2.19 (m, 2H). ES⁺ HR MS calculated for M+H 335.1503, observed 335.1520.

### EXAMPLE 78

This example illustrates the production of 2-{2-[4-(trifluoromethoxy)-phenyl]pyridin-4-yl}-5,6,7,8-tetrahydro-4H-pyrazolo[1,5-a][1,4]diazepin-4-one.

The preparation of 2-{2-[4-(trifluoromethoxy)phenyl]pyridin-4-yl}-5,6,7,8-tetrahydro-4H-pyrazolo[1,5-a][1,4]diazepin-4-one was carried out in a manner similar to that described for the preparation of 2-[2-(4-methoxyphenyl)pyridin-4-yl]-5,6,7,8-tetrahydro-4H-pyrazolo[1,5-a][1,4]diazepin-4-one. The mixture was then concentrated in vacuo, washed with water, filtered, and vacuum dried to afford the title lactam as an off-white solid (65%). LCMS showed a single peak with m/z 389 (M+H). ¹H nmr (DMSO-d₆/300 MHz) δ 8.70 (d, 1H), 8.40 (s, 1H), 8.33 (m, 3H), 7.82 (d, 1H), 7.62 (s, 1H), 7.49 (d, 2H), 4.53 (m, 2H), 3.24 (m, 2H), 2.19 (m, 2H). ES⁺ HR MS calculated for M+H 389.1220, observed 389.1225.

### EXAMPLE 79

This example illustrates the production of 2-{2-[4-(hydroxymethyl)phenyl]pyridin-4-yl}-5,6,7,8-tetrahydro-4H-pyrazolo[1,5-a][1,4]diazepin-4-one.

Step 1. The preparation of ethyl 1-(3-aminopropyl)-3-{2-[4-(hydroxymethyl)phenyl]pyridin-4-yl}-1H-pyrazole-5-carboxylate hydrochloride was carried out in a manner similar to that described for the production of 1-(3-aminopropyl)-3-[2-(4-methoxyphenyl)pyridin-4-yl]-1H-pyrazole-5-carboxylic acid hydrochloride, step 1 using 4-hydroxymethylphenyl boronic acid. Upon completion of the reaction, ethyl acetate and water were added. The layers were separated, and the organic layer was washed with water, dried over magnesium sulfate, filtered, and concentrated. The resulting residue was treated with excess 4 N HCl/dioxane for 15 minutes at room temperature. The reaction mixture was then chromatographed on a Gilson reverse phase HPLC eluting with an acetonitrile/water gradient (5-70% CH₃CN over 15 minutes). The pure fractions were combined, taken up in MeOH, and treated with 4 N HCl/dioxane to afford the HCl salt of the amine ester as a red solid (19% yield). LCMS showed one major peak with m/z381 (M+H).

Step 2. The preparation of 2-{2-[4-(hydroxymethyl)phenyl]pyridin-4-yl}-5,6,7,8-tetrahydro-4H-pyrazolo[1,5-a][1,4]diazepin-4-one was carried out in a manner similar to that described for the preparation of 2-[2-(4-methoxyphenyl)pyridin-4-yl]-5,6,7,8-tetrahydro-4H-pyrazolo[1,5-a][1,4]diazepin-4-one. The product mixture was concentrated and filtered to afford the lactam as a tan solid (44% yield). LCMS showed a single peak with m/z 335 (M+H). ES⁺ HR MS calculated for M+H 335.1503, observed 335.1520.

### EXAMPLE 80

This example illustrates the production of ethyl 1-(3-aminopropyl)-3-[2-(4 hydroxyphenyl)pyridin-4-yl]-1H-pyrazole-5-carboxylate hydrochloride.

The preparation of ethyl 1-(3-aminopropyl)-3-[2-(4-hydroxyphenyl)pyridin-4-yl]-1H-pyrazole-5-carboxylate hydrochloride was carried out in a manner similar to that described for the production of 1-(3-aminopropyl)-3-[2-(4-methoxyphenyl)pyridin-4-yl]-1H-pyrazole-5-carboxylic acid hydrochloride, step 1 using 4-hydroxyphenyl boronic acid THP ether. Upon completion of the reaction in step 1, ethyl acetate and water were added. The layers were separated, and the organic layer was washed with water, dried over magnesium sulfate, filtered, and concentrated. The residue was treated with excess 4 N HCl/dioxane for 15 minutes. The mixture was concentrated, triturated with ether, and filtered to afford the title amine ester as a tan solid (95% yield). LCMS showed one major peak with m/z 367 (M+H). mp=241.6-244.4° C.

### EXAMPLE 81

This example illustrates the production of 1-(3-aminopropyl)-3-[2-(4-hydroxyphenyl)pyridin-4-yl]-1H-pyrazole-5-carboxylic acid hydrochloride.

The preparation of 1-(3-aminopropyl)-3-[2-(4-hydroxyphenyl)pyridin-4-yl]-1H-pyrazole-5-carboxylic acid hydrochloride was carried out in a manner similar to that described for the production of 1-(3-aminopropyl)-3-[2-(4-methoxyphenyl)-pyridin-4-yl]-1H-pyrazole-5-carboxylic acid hydrochloride, step 2. The title carboxylic acid was obtained as an off-white solid (51% yield).). LCMS showed a single peak with m/z 339 (M+H). ES^{+HR} MS calculated for M+H 339.1452, observed 339.1455.

### EXAMPLE 82

This example illustrates the production of 2-[2-(4-hydroxyphenyl)pyridin-4-yl]-5,6,7,8-tetrahydro-4H-pyrazolo[1,5-a][1,4]diazepin-4-one trifluoroacetate.

The preparation of 2-[2-(4-hydroxyphenyl)pyridin-4-yl]-5,6,7,8-tetrahydro-4H-pyrazolo[1,5-a][1,4]diazepin-4-one trifluoroacetate was carried out in a manner similar to that described for the preparation of 2-[2-(4-methoxyphenyl)pyridin-4-yl]-5,6,7,8-tetrahydro-4H-pyrazolo[1,5-a][1,4]diazepin-4-one. Upon completion of the reaction, the product mixture was concentrated, washed with water, and filtered. The mixture was then chromatographed on a Gilson reverse phase HPLC eluting with an acetonitrile/water gradient (5-70% CH₃CN over 15 minutes). The pure fractions were combined and concentrated to afford the title compound as an off-white solid (41% yield). LCMS showed a single peak with m/z 321 (M+H). ¹H NMR (DMSO-dr/300 MHz) δ 8.72 (d, 1H), 8.63 (s, 1H), 8.42 (br s, 1H), 8.22 (d, 1H), 8.02 (d, 2H), 7.81 (s, 1H), 7.03 (d, 2H), 4.57 (m, 2H), 3.25 (m, 2H), 2.20 (m, 2H). ES⁺ HR MS calculated for M+H 321.1346, observed 321.1368.

### EXAMPLE 83

This examples illustrates the production of 1-(3-{[2-(4-bromophenyl)ethyl]-amino}propyl)-3-{2-[(E)-2-phenylvinyl]pyridin-4-yl}-1H-pyrazole-5-carboxylic acid trifluoroacetate.

Step 1. Preparation of ethyl 1-{3-[[2-(4-bromophenyl)ethyl](tert-butoxycarbonyl)-amino]propyl}-3-(2-chloropyridin-4-yl)-1H-pyrazole-5-carboxylate. A single neck roundbottom flask was charged with ethyl 3-(2-chloropyridin-4-yl)-1H-pyrazole-5-carboxylate (1.0 g, 4.0 mmol) and 30 mL dimethyl formamide under N₂. The solution was cooled to 40° C. in a dry ice/acetonitrile bath. A 1 M solution of lithium t-butoxide in THF (4.8 mL, 4.8 mmol) was added dropwise over 5 minutes. The reaction was stirred for 1 hour at −40° C. A solution of 3-[[2-(4-bromophenyl)ethyl](tert-butoxycarbonyl)amino]propyl methanesulfonate (2.1 g, 4.8 mmol) in 10 mL dimethylformamide was added dropwise over 5 minutes. The resulting reaction mixture was stirred for 1 hour at 40° C., and then stirred for 18 hours at room temperature. The mixture was then concentrated, and the residue taken up in diethyl ether. The solid impurities were filtered off, and washed with diethyl ether. The filtrate was concentrated to a brown oil. LCMS showed a mixture of the ethyl ester and hydrolyzed carboxylic acid. This product mixture was taken on to the next step without further purification.

Step 2. The preparation of ethyl 1-(3-{[2-(4-bromophenyl)ethyl]amino}-propyl)-3-{2-[(E)-2-phenylvinyl]pyridin-4-yl}-1H-pyrazole-5-carboxylate. hydrochloride was carried out in a manner similar to that described in example 2, step 1, using 2-phenylvinyl boronic acid. Upon completion of the reaction, ethyl acetate and water were added. The layers were separated, and the organic layer was washed with water, dried over magnesium sulfated, filtered and concentrated. The residue was treated with excess 4N HCl/dioxane for 30 minutes. The mixture was concentrated, washed with ether, and filtered to afford the ethyl ester as a brown solid (29% yield over 2 steps). LCMS showed one major peak with m/z 559 (M+H). ES⁺ HR MS calculated for M+H 559.1703, observed 559.1679.

Step 3. The preparation of 1-(3-{[2-(4-bromophenyl)ethyl]amino}propyl)-3-{2-[(E)-2-phenylvinyl]pyridin-4-yl}-1H-pyrazole-5-carboxylic acid trifluoroacetate was carried out in a manner similar to that described for the production of 1-(3-aminopropyl)-3-[2-(4-methoxyphenyl)pyridin-4-yl]-1H-pyrazole-5-carboxylic acid hydrochloride, step 2. Upon completion of the reaction, the mixture was concentrated. The residue was then chromatographed on a Gilson reverse phase HPLC eluting with an acetonitrile/water gradient (5-70% CH₃CN over 15 minutes). The pure fractions were combined and concentrated to afford the title compound as an off-white solid (30% yield). LCMS showed a single peak with m/z 531 (M+H). ES⁺ HR MS calculated for M+H 531.1390, observed 531.1411.

### EXAMPLE 84

This examples illustrates the production of ethyl 1-{2-[(tert-butoxy-carbonyl)amino]ethyl}-3-[2-(4-{[tert-butyl (dimethyl)silyl]oxy}phenyl)-pyridin-4-yl]-1H-pyrazole-5-carboxylate.

Ethyl 1-{2-[(tert-butoxycarbonyl)amino]ethyl}-3-(2-chloropyridin-4-yl)-1H-pyrazole-5-carboxylate (5.0 g, 0.013 mol), 4-(t-butyldimethylsilyloxy)phenyl boronic acid (6.4 g, 0.025 mol), sodium carbonate (2.7 g, 0.025 mol), water (12.5 mL), and [1,1′-bis(diphenylphosphino)ferrocene]dichloro palladium (II) complex with dichloromethane (1:1) (1.0 g, 0.0013 mol) in toluene (100 mL) were refluxed for 4 hours. Contents were allowed to cool and partitioned between EtOAc and water. The EtOAc layer was washed with brine, dried over MgSO₄ and concentrated in vacuo. The residue was filtered through a pad of silica gel, eluting with 25% EtOAc/hexanes to give a light amber oil. The oil crystallized under hexanes and was filtered to give the desired product as a white solid, 3.77 g (51% yield). FABHRMS m/z 567.2983 (M+H, C₃₀H₄₃N₄O₅Si requires 567.2997). ¹H NMR (CDCl₃/300 MHz): 8.70 (s, 1H); 8.12 (s, 1H); 7.97 (d, 2H); 7.60 (s, 1H); 7.29 (d, 1H); 6.95 (d, 2H); 4.97 (br, 1H); 4.77 (t, 2H); 4.40 (q, 2H); 3.63 (br, 2H); 1.40 (t, 3H); 1.38 (s, 9H); 1.00 (s, 9H); 0.25 (s, 6H).

Anal. Calculated for C₃₀H₄₂N₄O₅Si: C, 63.58; H, 7.47; N, 9.89. Found: C, 63.51; H, 7.58; N, 9.73.

### EXAMPLE 85

This example illustrates the preparation of ethyl 1-(2-aminoethyl)-3-[2-(4-[tert-butyl(dimethyl)silyl]oxy-phenyl)pyridin-4-yl]-1H-pyrazole-5-carboxylate dihydrochloride.

Ethyl 1-{2-[(tert-butoxycarbonyl)amino]ethyl}-3-[2-(4-{[tert-butyl(dimethyl)-silyl]oxy}phenyl)pyridin-4-yl]-1H-pyrazole-5-carboxylate (1.0 g, 0.0018 mol) and 4N HCl in dioxane were mixed for 2 hours and filtered to give the desired product as a white solid, 875 mg (90% yield). FABHRMS m/z 467.2450 (M+H, C₂₅H₃₅N₄O₃Si requires 467.2473). ¹H NMR (DMSO-d₆+TFA/300 MHz): 8.80 (d, 1H); 8.75 (s, 1H); 8.35 (d, 1H); 8.22 (br, 3H); 8.20-8.10 (m, 3H); 7.13 (d, 2H); 4.90 (t, 2H); 4.40 (q, 2H); 3.40 (q, 2H); 1.39 (t, 3H); 0.95 (s, 9H); 0.23 (s, 6H).

Anal. Calculated for C₂₅H₃₄N₄O₃Si (2 HCl, 1.1 H₂O): C, 53.68; H, 6.88; N, 10.02. Found: C, 53.34; H, 6.98; N, 10.18.

### EXAMPLE 86

This example illustrates the production of 2-[2-(4-hydroxyphenyl)pyridin-4-yl]-6,7-dihydropyrazolo[1,5-a]pyrazin-4(5H)-one.

Ethyl 1-(2-aminoethyl)-3-[2-(4-{[tert-butyl(dimethyl)silyl]oxy}phenyl) pyridin-4-yl]-1H-pyrazole-5-carboxylate dihydrochloride (770 mg, 0.0014 mol), conc ammonium hydroxide (2 mL) and methanol (20 mL) were stirred overnight. Contents were concentrated in vacuo and the residue was slurried in water and filtered to give the desired product as a white solid, 373 mg, (87% yield). FABHRMS m/z 307.1222 (M+H, C₁₇H₁₅N₄O₂ requires 307.1190). ¹H NMR (DMSO-d₆+TFA/300 MHz): 8.75 (d, 1H); 8.63 (s, 1H); 8.40 (s, 1H); 8.21 (d, 1H); 8.00-7.95 (m, 3H); 7.00 (d, 2H); 4.43 (t, 2H); 3.65 (br, 2H).

Anal. Calculated for C₁₇H₁₄N₄O₂ (1.3H₂O): C, 61.92; H, 5.07; N, 16.99. Found: C, 61.79; H, 5.11; N, 16.85.

### EXAMPLE 87

This example illustrates the production of 2-{2-[4-(2-morpholin-4-ylethoxy)phenyl]pyridin-4-yl}-6,7-dihydropyrazolo[1,5-a]pyrazin-4(5H)-one.

2-[2-(4-hydroxyphenyl)pyridin-4-yl]-6,7-dihydropyrazolo[1,5-a]pyrazin-4(5H)-one (500 mg, 0.0016 mol), 4-(2-chloroethyl)morpholine hydrochloride (372 mg (0.002 mol) and potassium carbonate (600 mg, 0.004 mol) were heated in DMF (20 mL) at 80° C. for 3 hours. Contents were allowed to cool, diluted with water (50 mL), cooled to 0° C. and filtered to give the desired product as a white solid, 515 mg (77% yield). FABHRMS m/z 420.2004 (M+H, C₂₃H₂₆N₅O₃ requires 420.2030). ¹H NMR (DMSO-d₆+TFA/300 MHz): 8.81 (d, 1H); 8.66 (s, 1H); 8.43 (s, 1H); 8.23 (d, 1H); 8.15 (d, 2H); 7.94 (s, 1H); 7.28 (d, 2H); 4.55-4.40 (m, 4H); 4.05-3.95 (m, 2H); 3.80-3.50 (m, 8H); 3.30-3.18 (m, 2H).

Anal. Calculated for C₂₃H₂₅N₅O₃ (0.8H₂O): C, 63.60; H, 5.96; N, 16.25. Found: C, 63.67; H, 6.18; N, 16.14.

### EXAMPLE 88

This example illustrates the production of 2-(2-{4-[2-(dimethylamino)ethoxy]-phenyl}pyridin-4-yl)-6,7-dihydropyrazolo[1,5-a]pyrazin-4(5H)-one.

2-(2-{4-[2-(dimethylamino)ethoxy]phenyl}pyridin-4-yl)-6,7-dihydropyrazolo[1,5-a]pyrazin-4(5H)-one was prepared according to the procedure of 2-{2-[4-(2-morpholin-4-ylethoxy)phenyl]pyridin-4-yl}-6,7-dihydropyrazolo[1,5-a]pyrazin-4(5H)-one to give the desired product as a white solid (72% yield). FABHRMS m/z 378.1901 (M+H, C₂₁H₂₄N₅O₂ requires 378.1925). ¹H NMR (DMSO-d₆+TFA/300 MHz): 9.80 (br, 1H); 8.80 (d, 1H); 8.70 (s, 1H); 8.42 (s, 1H); 8.28 (d of d, 1H); 8.12 (d, 2H); 7.96 (s, 1H); 7.28 (d, 2H); 4.50-4.40 (m, 4H); 3.70 (br, 2H); 3.60 (br, 2H); 2.90 (s, 6H). Anal. Calculated for C₂₁H₂₃N₅O₂ (0.6H₂O): C, 64.91; H, 6.15; N, 18.03. Found: C, 64.97; H, 6.28; N, 18.04.

### EXAMPLE 89

This example illustrates the production of Ethyl 1-(2-aminoethyl)-3-{2-[3-(benzyloxy)phenyl]pyridin-4-yl}-1H-pyrazole-5-carboxylate dihydrochloride.

Ethyl 1-{2-[(tert-butoxycarbonyl)amino]ethyl}-3-(2-chloropyridin-4-yl)-1H-pyrazole-5-carboxylate and 3-benzyloxyboronic acid were reacted according to the procedure described for the preparation of ethyl 1-{2-[(tert-butoxycarbonyl)-amino]ethyl}-3-[2-(4-{[tert-butyl(dimethyl)silyl]oxy}phenyl)pyridin-4-yl]-1H-pyrazole-5-carboxylate, to give ethyl 1-(2-t-butoxycarbonylaminoethyl)-3-{2-[3-(benzyloxy-phenyl]pyridin-4-yl}-1H-pyrazole-5-carboxylate as a light amber oil. Ethyl 1-(2-t-butoxycarbonylaminoethyl)-3-[2-[3-(benzyloxy)phenyl]pyridin-4-yl]-1H-pyrazole-5-carboxylate and 4N HCl in dioxane were stirred 3 hours and filtered to give the desired product as a pale yellow solid, 8.0 g (81% yield). FABHRMS m/z 443.2053 (M+H, C₂₆H₂₇N₄O₃ requires 443.2078). ¹H NMR (DMSO-d₆+TFA/300 MHz): 8.88 (d, 1H); 8.80 (s, 1H); 8.10 (s, 1H); 8.05 (br, 3H); 7.78 (s, 1H); 7.65 (d, 1H); 7.55 (t, 1H); 7.45 (d, 1H); 7.40-7.30 (m, 6H); 5.21 (s, 2H); 4.85 (t, 2H); 4.35 (q, 2H); 3.40 (q, 2H); 1.35 (t, 3H).

Anal. Calculated for C₂₆H₂₆N₄O₃ (3HCl, H₂O): C, 54.80; H, 5.48; N, 9.83. Found: C, 55.01; H, 5.84; N, 10.75.

### EXAMPLE 90

This example illustrates the production of 2-{2-[3-(benzyloxy)phenyl]-pyridin-4-yl}-6,7-dihydropyrazolo[1,5-a]pyrazin-4(5H)-one.

2-{2-[3-(benzyloxy)phenyl]pyridin-4-yl}-6,7-dihydropyrazolo[1,5-a]pyrazin-4(5H)-one was prepared according to the procedure for the preparation of 2-[2-(4-hydroxyphenyl)pyridin-4-yl]-6,7-dihydropyrazolo[1,5-a]pyrazin-4(5H)-one, using ethyl 1-(2-aminoethyl)-3-{2-[3-(benzyloxy)phenyl]pyridin-4-yl}-1H-pyrazole-5-carboxylate dihydrochloride, to give the desired product as a white solid (63% yield). FABHRMS m/z 397.1634 (M+H, C₂₄H₂₁N₄O₂ requires 397.1659). ¹H NMR (DMSO-d₆+TFA/300 MHz): 8.81 (d, 1H); 8.62 (s, 1H); 8.40 (s, 1H); 8.21 (d, 1H); 7.92 (s, 1H); 7.80 (s, 1H); 7.70 (d, 1H); 7.63-7.25 (m, 6H); 5.23 (s, 2H); 4.51-4.40 (m, 2H); 3.78-3.60 (m, 2H). Anal. Calculated for C₂₄H₂₀N₄O₂ (H₂O): C, 69.55; H, 5.35; N, 13.52. Found: C, 69.65; H, 5.11; N, 14.50.

### EXAMPLE 91

This example illustrates the production of ethyl 1-(2-aminoethyl)-3-[2-(3-hydroxyphenyl)pyridin-4-yl]-1H-pyrazole-5-carboxylate dihydrochloride.

Ethyl 1-(2-t-butoxycarbonylaminoethyl)-3-{2-[3-(benzyloxy)phenyl]pyridin-4-yl}-1H-pyrazole-5-carboxylate (9.1 g, 0.017 mol), prepared as for ethyl 1-(2-aminoethyl)-3-{2-[3-(benzyloxy)phenyl]pyridin-4-yl}-1H-pyrazole-5-carboxylate dihydrochloride and 10% palladium/carbon (2.0 g) in ethanol (150 mL) were shaken at 55 psi H₂ on a Parr hydrogenator for three and a half days. Contents were filtered through clay and the filtrate was concentrated in vacuo leaving a pale yellow solid (6.4 g). The solid and 4N HCl in dioxane were stirred overnight and filtered to give the desired product as a white solid, 6.0 g (83% yield). HRMS calculated for (M+H) 353.1608, found 353.1630. ¹H NMR (DMSO-d₆+TFA/300 MHz): 8.86 (d, 1H); 8.77 (s, 1H); 8.40 (d, 1H); 8.20 (brs, 3H); 8.12 (s, 1H); 7.59-7.40 (m, 3H); 7.09 (d, 1H); 4.90 (t, 2H), 4.39 (q, 2H); 3.40 (q, 2H); 1.35 (t, 3H).

### EXAMPLE 92

This example illustrates the production of 2-{2-[3-(2-morpholin-4-ylethoxy)-phenyl]pyridin-4-yl}-6,7-dihydropyrazolo[1,5-a]pyrazin-4(5H)-one.

2-{2-[3-(2-morpholin-4-ylethoxy)phenyl]pyridin-4-yl}-6,7-dihydropyrazolo-[1,5-a]pyrazin-4(5H)-one was prepared according to the procedure for 2-{2-[4-(2-morpholin-4-ylethoxy)phenyl]pyridin-4-yl}-6,7-dihydropyrazolo[1,5-a]pyrazin-4(5H)-one, using 2-[2-(3-hydroxyphenyl)pyridin-4-yl]-6,7-dihydropyrazolo[1,5-a]pyrazin-4(5H)-one and chloroethylmorpholine hydrochloride to give the desired as a white solid (55% yield). FABHRMS m/z 420.1996 (M+H, C₂₃H₂₆N₅O₃ requires 420.2030). ¹H NMR (DMSO-d₆+TFA/300 MHz): 8.85 (d, 1H); 8.61 (s, 1H); 8.40 (s, 1H); 8.21 (d, 1H); 7.89 (s, 1H); 7.78 (s, 2H); 7.59 (t, 1H); 7.28 (d, 1H); 4.58-4.40 (m, 4H); 4.08-3.91 (m 2H); 3.84-3.48 (m, 8H); 3.45-3.13 (m, 2H).

Anal. Calculated for C₂₃H₂₅N₅O₃ (0.7H₂O): C, 63.93; H, 6.16; N, 16.21. Found: C, 63.93; H, 5.96; N, 16.42.

### EXAMPLE 93

This example illustrates the preparation of 2-(2-Chloropyridin-4-yl)-2,5,6,7-tetrahydro-4H-pyrazolo[4,3-c]pyridin-4-one.

Step 1. Preparation of 2-Chloro-4-hydrazinopyridine hydrochloride. A solution of 4-amino-2-chloropyridine (1.0 g, 7.78 mmol) in 20% sulfuric acid (20 mL) was cooled to 0° C. and treated with a solution of sodium nitrite (564 mg, 8.17 mmol) in water (3 mL) at a rate such that the reaction temperature did not exceed 10° C. After 15 minutes, the solution was added to a 0° C. suspension of tin(II) chloride in 20% sulfuric acid (20 mL). The frothy suspension was stirred for 15 minutes at 0° C. and then warmed to room temperature over 15 minutes. The mixture was poured into 100 mL of ice water and made basic with concentrated ammonium hydroxide. The product was extracted with diethyl ether and ethyl acetate repeatedly. The organic layers were dried (sodium sulfate) and concentrated to give crude 2-chloro-4-hydrazinopyridine as a yellow solid (830 mg, 5.78 mmol). The solid was dissolved in tetrahydrofuran (5 mL) and diluted with diethyl ether (15 mL). The solution was treated with 1 N HCl in diethyl ether (5.8 mL, 5.8 mmol). The white precipitate was filtered and washed with ether to give 2-chloro-4-hydrazinopyridine hydrochloride as a white solid (995 mg, 5.53 mmol, 71% yield). LC-MS (ES+) MH⁺=144. ¹H NMR (300 MHz, DMSO-d₆) δ 10.0-9.40 (br s, 4H), 8.07 (d, J=6.1, 1H), 6.95 (d, J=1.9, 1H), 6.86 (dd, J=5.8, 2.0, 1H).

Step 2. Preparation of Piperidine-2,4-dione 4-[(2-chloropyridin-4-yl)hydrazone]. A mixture of 2-chloro-4-hydrazinopyridine hydrochloride (961 mg, 5.33 mmol), piperdiene-2.4-dione (Example 1, step 3) (604 mg, 5.33 mmol) and ethanol (20 mL) was refluxed overnight. The reaction mixture was cooled to room temperature, diluted with diethyl ether (20 mL), and filtered. The precipitate was washed with 50% ethanol/diethyl ether and dried to give piperidine-2,4-dione 4-[(2-chloropyridin-4-yl)hydrazone] as an off-white solid (940 mg, 3.94 mmol, 74% yield). LC-MS (ES+) MH⁺=239.

Step 3. Preparation of 2-(2-Chloropyridin-4-yl)-2,5,6,7-tetrahydro-4H-pyrazolo[4,3-c]pyridin-4-one.

A mixture of piperidine-2,4-dione 4-[(2-chloropyridin-4-yl)hydrazone] (863 mg, 3.62 mmol) in dimethylformamide dimethyl acetal (16 mL) was refluxed for 1 hour. The solvent was removed under reduced pressure. The residue was suspended in ethanol/diethyl ether and filtered. The precipitate was washed with 50% ethanol/diethyl ether to give 2-(2-chloropyridin-4-yl)-2,5,6,7-tetrahydro-4H-pyrazolo[4,3-c]pyridin-4-one as an off-white solid (378 mg, 1.52 mmol, 42% yield). The mother liquor was concentrated and purified by flash chromatography (0→10% methanol/ethyl acetate). The resultant oil was triturated with methanol/ether to give another 58 mg (0.23 mmol, 6%) of 2-(2-chloropyridin-4-yl)-2,5,6,7-tetrahydro-4H-pyrazolo[4,3-c]pyridin-4-one. ¹H NMR (300 MHz, DMSO-d₆) δ 9.17 (s, 1H), 8.47 (d, J=5.7, 1H), 8.04 (d, J=1.6, 1H), 7.94 (dd, J=5.5, 1.8, 1H), 7.71 (br s, 1H), 3.44 (td, J=6.5, 2.6, 2H), 2.89 (t, J=6.6, 2H). HRMS calculated for C₁₁H₁₀ClN₄O (MH⁺) 249.0538, found 249.0545.

### EXAMPLE 94

This example illustrates the preparation of 2-(2-quinolin-3-ylpyridin-4-yl)-2,5,6,7-tetrahydro-4H-pyrazolo[4,3-c]pyridin-4-one bis(trifluoroacetate).

The title compound was prepared from 2-(2-chloropyridin-4-yl)-2,5,6,7-tetrahydro-4H-pyrazolo[4,3-c]pyridin-4-one (Example 508) and 3-quinolinylboronic acid by the procedure described for Example 2. ¹H NMR (300 MHz, DMSO-d₆) δ 9.76 (d, J=2.2, 1H), 9.38 (s, 1H), 9.26 (d, J=2.0, 1H), 8.82 (d, J=5.4, 1H), 8.71 (d, J=1.6, 1H), 8.16 (d, J=7.7, 1H), 8.12 (d, J=8.3,1H), 7.96 (dd, J=5.6, 2.0,1H), 7.87 (td, J=7.7, 1.3,1H), 7.76-7.68 (m, 2H), 3.48 (td, J=6.6, 2.4, 2H), 2.95 (t, J=6.6, 2H). HRMS calculated for C₂₀H₁₆N₅O (MH⁺) 342.1349, found 342.1334.

The following examples were made by the same method.

| Exam- | | | |
|---|---|---|---|
| ple | | Calculated | Found |
| No. | Compound Name(s) | (m + H) | (m + H) |
| 95 | 2-[2-(2-fluorophenyl)pyridin-4- | 309.1146 | 309.119 |
| | yl]-2,5,6,7-tetrahydro-4H-pyrazolo[4,3- | | |
| | c]pyridin-4-one trifluoroacetate | | |
| 96 | 2-(2-phenylpyridin-4-yl)-2,5,6,7- | 291.124 | 291.1252 |
| | tetrahydro-4H-pyrazolo[4,3-c]pyridin-4- | | |
| | one trifluoroacetate | | |

### EXAMPLE 97

This example illustrates the preparation of 2-{2-[(E)-2-(4-morpholin-4-ylphenyl)vinyl]pyridin-4-yl}-6,7-dihydropyrazolo[1,5-a]pyrazin-4(5H)-one trifluoroacetate.

mp 290° C. (decomposition); ¹H NMR (300 MHz, DMSO-d₆) δ 8.62 (d, J=6.5 Hz, 1H), 8.39 (2×s, 2H), 7.97-7.83 (m, 2H), 7.70 (s, 1H), 7.57 (d, J=8.6 Hz, 2H), 7.15 (d, J=16.2 Hz, 1H), 7.03 (d, J=16.2 Hz, 2H), 4.50-4.38 (m, 2H), 3.80-3.60 (m, 6H), 3.28-3.19 (m, 4H); m/z402 [M+H]⁺.

### EXAMPLE 98

This example illustrates that MK2 knock-out mice (MK2 (−/−)) are resistant to the formation of K/BN serum-induced arthritis and that compounds that inhibit MK-2 should be effective for the prevention and treatment of TNFα-mediated diseases or disorders.

A strain of mice has been reported that develops symptoms similar to human rheumatoid arthritis. The mice were designated K/BxN mice. See, Wipke, B. T. and P. M. Allen, J. of Immunology, 167:1601-1608 (2001). Serum from the mice can be injected into host animals to provoke a typical R^{a} response. The progression of the R^{a} symptoms in the mice is measured by measuring paw thickness as a function of time.

In the present example, host mice having normal MK-2 production (MK2 (+/+)) were genetically altered by disabling the gene encoding MK-2 to produce mice having no capability of endogenous synthesis of active MK-2 (MK2 (−/−)). Normal host mice (MK2 (+/+)) and MK-2 knock-out mice (MK2 (−/−), were separated into four groups with each group containing both male and female mice. All groups of mice were treated similarly, except that one group (Normal), composed of MK2 (+/+) mice that served as the control group, was not injected with serum from K/BxN mice, while the other three groups were injected with K/BxN serum at day 0. The other three groups of mice were MK2 (+/+), MK2 (−/−), and Anti-TNF. The Anti-TNF group was composed of MK2 (+/+) mice which were also injected at day) with anti-TNF antibody. The paw thickness of all mice was measured immediately after the injections on day 0, and then on each successive day thereafter for 7 days.

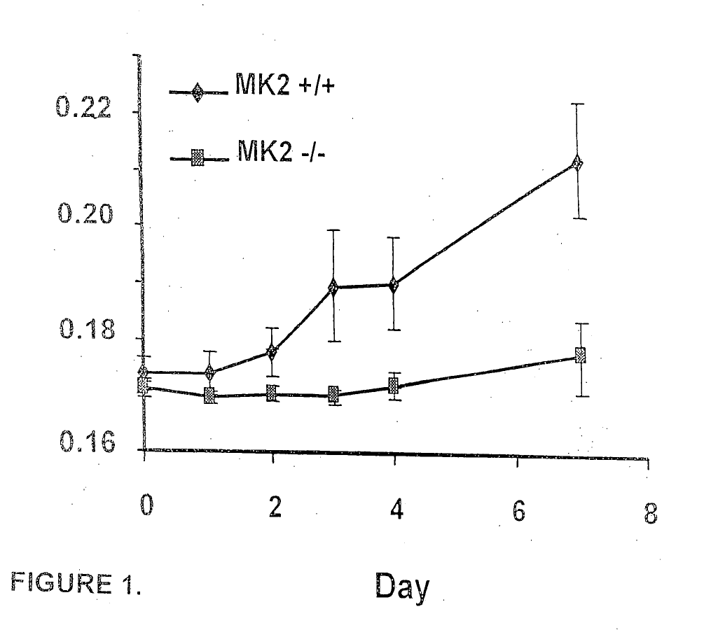

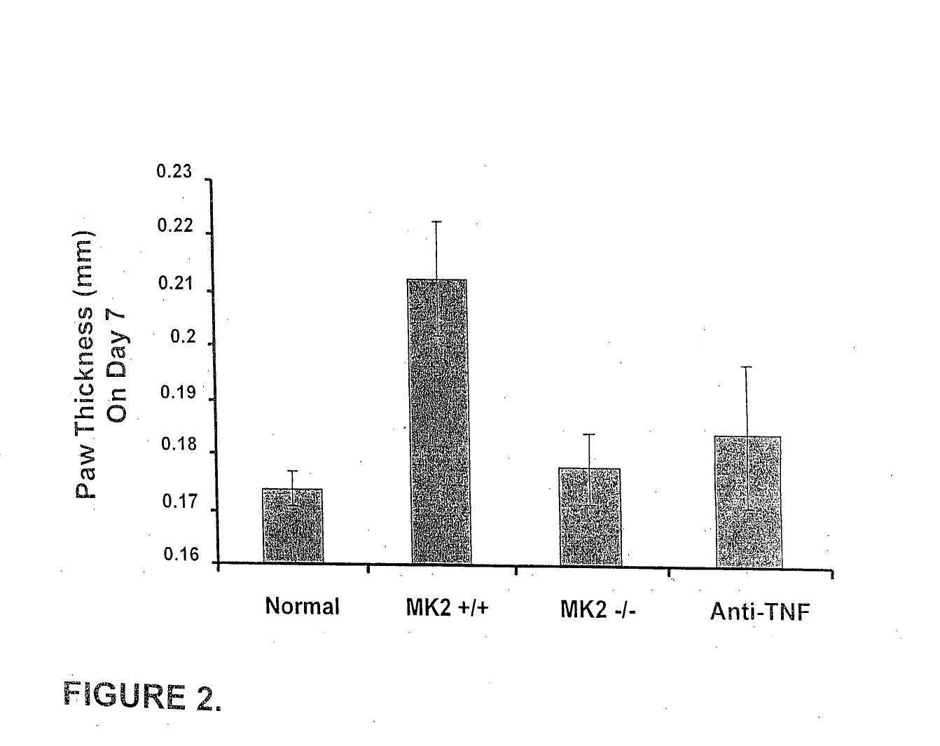

FIG. 1 is a graph that shows paw thickness as a function of time from day 0 to day 7 for MK2 (+/+) and MK2 (−/−) mice, which have received serum injection. It can be seen that paw thickness increased significantly for MK2(+/+) mice, whereas there was substantially no increase in paw thickness for MK2 knock-out mice. This indicated the requirement for a functioning MK2 regulatory system to the inflammatory response caused by the serum challenge. When anti-TNF antibody was administered to the MK2 (+/+) mice along with the serum injection, the swelling response was significantly reduced. This can be seen in FIG. 2, which is a bar chart showing paw thickness at seven days after injection for normal mice, MK2 (+/+) mice receiving serum, MK2 (−/−) mice receiving serum, and MK2 (+/+) mice receiving serum and anti-TNF antibody.

This data shows that the MK2 knock-out mice show no arthritic response to a serum challenge, whereas MK2 (+/+) mice show a normal response. Treatment of MK2 (+/+) mice that receive a serum challenge with anti-TNF antibody reduces the response back to near-normal levels. This illustrates the utility of the MK2 regulatory system as a potential control point for the modulation of TNF production, and indicates that such regulation could serve as a treatment for inflammation—such as that caused by arthritis, for example. It further shows that MK2 inhibition can have a beneficial effect on inflammation, and indicates that administration of an MK2 inhibitor can be an effective method of preventing or treating TNF modulated diseases or disorders.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A compound having the structure: 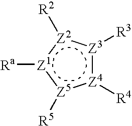
wherein:
Z² and Z³ are nitrogen, Z¹, Z⁴ and Z⁵ are carbon, and join with Z² and Z³ to form a pyrazole ring, or optionally, Z⁴ and Z⁵ are nitrogen, Z¹, Z² and Z³ are carbon and join with Z⁴ and Z⁵ to form a pyrazole ring;
R^{a} is selected from: 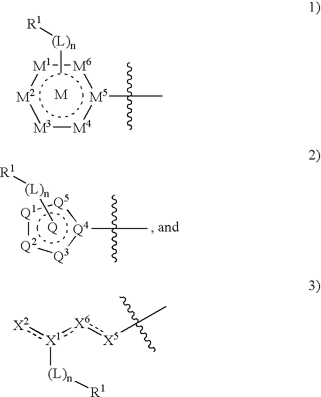
where dashed lines indicate optional single or double bonds;
when R^{a} is ring M and ring M is aromatic, M¹ is carbon and is substituted with (L)ₙR¹, M⁵ is carbon, and each of M², M³, M⁴ and M⁶ is independently selected from carbon and nitrogen and is unsubstituted or substituted with (L)ₙR¹;
when ring M is partially saturated, M¹ is carbon and is mono- or di-substituted with (L)ₙR¹, M⁵ is carbon, and each of M², M³, M⁴ and M⁶ is independently selected from carbon, nitrogen, oxygen and sulfur, and when M², M³, M⁴, or M⁶ is oxygen or sulfur, it is unsubstituted, and when M², M³, M⁴ or M⁶ is carbon or nitrogen, it is optionally unsubstituted; or mono- or di-substituted with (L)ₙR¹;
when R^{a} is ring Q and ring Q is aromatic, Q¹ is selected from carbon and nitrogen, and when Q¹ is carbon, it is substituted with (L)ₙR¹, and when Q¹ is nitrogen, it is unsubstituted, Q⁴ is selected from nitrogen and carbon, and each of Q², Q³ and Q⁵ is-independently selected from nitrogen and carbon, and if carbon, it is substituted with (L)ₙR¹;
optionally when ring Q is aromatic, Q¹ is carbon and is substituted with (L)ₙR¹, Q⁴ is carbon, and one of Q², Q³ and Q⁵ is optionally oxygen or sulfur, and the remainder of Q², Q³ and Q⁵ are independently selected from nitrogen and carbon, and if carbon, are substituted with (L)ₙR¹;
when ring Q is partially saturated, Q¹ is selected from carbon and nitrogen, and if carbon, it is mono- or di-substituted with (L)ₙR¹, and if nitrogen, it is unsubstituted or substituted with (L)ₙR¹, Q⁴ is selected from carbon and nitrogen, but only one of Q¹ and Q⁴ can be nitrogen, each of Q², Q³ and Q⁵ is independently selected from carbon, nitrogen, oxygen and sulfur, and if oxygen or sulfur, it is unsubstituted, and if carbon, it is mono- or di-substituted with (L)ₙR¹, and if nitrogen, it is unsubstituted or substituted with (L)ₙR¹;
when R^{a} is structure 3, it is fully conjugated, X² is selected from oxygen or nitrogen substituted with (L)ₙR¹, X¹ is carbon and is substituted with (L)ₙR¹, and each of X⁵ and X⁶ is independently selected from nitrogen and carbon, and if carbon, it is substituted with (L)ₙR¹;
R¹ is selected from —H, C₁-C₆ alkyl, C₂-C₆ alkenyl, C₂-C₆ alkynyl, C₁-C₆ alkyl-R¹¹, C₂-C₆ alkenyl-R¹¹, C₂-C₆ alkynyl-R¹¹, C₁-C₆ alkyl-(R¹¹)₂, C₂-C₆ alkenyl-(R¹¹)₂, CSR¹¹, amino, CONHR¹¹, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkylC(R¹¹)═N—N(R⁸)(R⁹), (C₁-C₄)alkyl-N═N(R⁷), (C₁-C₄)alkyl-N(R⁷)—N═C(R⁸), nitro, cyano, CO₂R¹¹, O—R¹⁰, C₁-C₄ alkyl-OR¹⁰, COR¹¹, SR¹⁰, SSR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, Si(R¹¹)₃, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;
R⁷, R⁸ and R⁹ are each independently selected from —H, C₁-C₆ alkyl, C₂-C₆ alkenyl, C₂-C₆ alkynyl, C₁-C₄ alkyl-R¹¹, C₁-C₆ alkyl-NHR¹³, C₁-C₆ alkyl-NR¹³R¹⁴, O—R¹⁵, C₁-C₄ alkyl-OR¹⁵, CO₂R¹⁵, C(S)OR¹⁵, C(O)SR¹⁵, C(O)R, C(S)R¹⁷, CONHR¹⁶, C(S)NHR¹⁶ CON(R¹⁶)₂, C(S)N(R¹⁶)₂, SR¹⁵ SOR¹⁷, SO₂R¹⁷, C₁-C₆ alkyl-CO₂R⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, —C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;
R¹⁰ is selected from —H, C₁-C₁₀ alkyl, C₂-C₆ alkenyl, C₂-C₆ alkynyl, C₁-C₆ alkyl-NHR¹³, C₁-C₆ alkyl-NR¹³R¹⁴, C₁-C₄ alkyl-OR¹⁵, CSR¹¹, CO₂R¹⁵, C(S)OR¹⁵, C(O)SR¹⁵, COR¹⁷, C(S)R¹⁷, CONHR¹⁶, C₁-C₄ alkyl-R¹⁶, C₁-C₄ alkyl-NH₂R¹³, C(S)NHR¹⁶, O—R¹⁵, CON(R¹⁶)₂, C(S)N(R¹⁶)₂, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶ C₁-C₆ alkyl-CON(R¹⁶)₂, Si(R¹³)₂R¹⁷, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁵;
R¹¹ is selected from —H, C₁-C₆ alkyl, C₁-C₆ alkoxy, C₂-C₆ alkenyl, C₂-C₆ alkynyl, amino, NHR¹³, NR¹³ R¹⁴, N═NR¹³, C₁-C₆ alkyl-NHR¹³, C₁-C₆ alkyl-NR¹³R¹⁴, O—R¹⁵, C₁-C₄ alkyl-OR¹⁵, SR¹⁵, COR¹³, CO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹, C₁-C₆ alkyl-CON(R¹⁶)₂, C₁-C₆ alkyl-C(S)N(R¹⁶)₂, C₁-C₆ alkyl-SR¹⁵, C₁₀₆ alkyl-SOR¹⁷, C₁-C₆ alkyl-SO₂R¹⁷, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;
R¹² is selected from —H, OH, oxo, C₁-C₁₀ alkyl, C₂-C₁₀ alkenyl, C₂-C₁₀ alkynyl, C₁-C₁₀ alkyl-R¹¹, C₂-C₁₀ alkenyl-R¹¹, C₂-C₁₀ alkynyl-R¹¹, C₁-C₁₀ alkyl-(R¹¹)₂, C₂-C₁₀ alkenyl-(R¹¹)₂, CSR¹¹, hydroxyl C₁-C₆ alkyl-R¹¹, amino C₁-C₄ alkyl-R⁷, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹), C₁-C₁₀ alkyl-NHR⁷, C₁-C₁₀ alkyl-NR⁸R⁹, (C₁-C₁₀)alkyl-N(R⁷)—N(R⁸)(R⁹), (C₁-C₁₀)alkylC(R¹¹)═N—N(R⁸)(R⁹), (C₁-C₁₀)alkyl-N═N(R⁷), (C₁-C₁₀)alkyl-N(R⁷)—N═C(R⁸), SCN, NCS, C₁-C₁₀ alkyl SCN, C₁-C₁₀ alkyl NCS, nitro, cyano, O—R¹⁰, C₁-C₁₀ alkyl-OR¹⁰, COR¹¹, CO₂R¹¹, SR¹⁰, SSR¹⁰, SOR¹¹, SO₂R¹¹, C₁-C₁₀ alkyl-COR¹¹, C₁-C₁₀ alkyl-SR¹⁰, C₁-C₁₀ alkyl-SOR¹¹, C₁-C₁₀ alkyl-SO₂R¹¹, halo, Si(R¹¹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C, —C₁-C₁₀ mono- and bicyclic cycloalkyl are optionally substituted with one or more of the groups defined by R¹⁸;
R¹³ and R¹⁴ are each independently selected from —H, oxo, C₁-C₆ alkyl, C₂-C₆ alkenyl, C₂-C₆ alkynyl, C₁-C₄ alkyl-R²³, C₁-C₆ alkyl-NHRC¹⁹, C₁-C₆ alkyl-NR¹⁹R²⁰, O—R²¹, C₁-C₄ alkyl-OR²¹, CO₂R²¹, COR²¹, C(S)OR²¹, C(O)SR²¹, C(O)R²³, C(S)R²³, CONHR²², C(S)NHR²², CON(R²²)₂, C(S)N(R²²)₂, SR²¹, SOR²³, SO₂R²³, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁵ and R¹⁶ are independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CSR¹¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁷ is selected from —H, C₁-C₆ alkyl, C₂-C₆ alkenyl, C₂-C₆ alkenyl-R¹⁹, C₁-C₆ alkyl-R¹⁹, C₂-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁸ is selected from —H, oxo, OH, C₁-C₁₀ alkyl, C₂-C₁₀ alkenyl, C₂-C₁₀ alkynyl, C₁-C₁₀ alkyl-R²³, C₂-C₁₀ alkenyl-R²³, C₂-C₁₀ alkynyl-R²³, C₁-C₁₀ alkyl-(R²³)₂, C₂-C₁₀ alkenyl-(R²³)₂, CSR²³, amino, NHR¹⁹, NR²⁰R²⁰, N(R¹⁹)—N(R²⁰)(R²⁰), C(R²³)═N—N(R²⁰)(R²⁰), N═N(R¹⁹), N(R¹⁹)—N═C(R²⁰), C(R²³)═N—O(R²¹), ON═C(R²³), C₁-C₁₀ alkyl-NHR¹⁹, C₁-C₁₀ alkyl-NR²OR²⁰, (C₁-C₁₀)alkyl-N(R¹⁹)—N(R²⁰)(R²⁰), (C₁-C₁₀)alkylC(R²³)═N—N(R²⁰)(R²⁰), (C₁-C₁₀)alkyl-N═N(R¹⁹), (C₁-C₁₀)alkyl-N(R¹⁹)—N═C(R²⁰), SCN, NCS, C₁-C₁₀ alkyl SCN, C₁-C₁₀ alkyl NCS, nitro, cyano, O—R²¹, C₁-C₁₀ alkyl-OR²¹, COR²³ CO R²³ SR²¹ SSR²¹, SOR²³, SO₂R²³, C₁-C₁₀ alkyl-COR²³, C₁-C₁₀ alkyl-SR²¹, C₁-C₁₀ alkyl-SOR²³, C₁-C₁₀ alkyl-SO₂R²³, halo, Si(R²³)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁹ and R²⁰ are each independently selected from —H, C₁-C₆ alkyl, C₂-C₆ alkenyl, C₂-C₆ alkynyl, C₁-C₄ alkyl-R²⁹, C₁-C₆ alkyl-NHR²⁵, C₁-C₆ alkyl-NR²⁵R²⁶, O—R²⁷, C₁-C₄ alkyl-OR²⁷, CO₂R²⁷, C(S)OR²⁷, C(O)SR²⁷, C(O)R²⁹ (S)R²⁹, CONHR²⁸, C(S)NHR²⁸, CON(R²⁸)₂, C(S)N(R²⁸)₂, SR²⁷, SOR²⁹, SO₂R²⁹, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁵, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²¹ and R²² are independently selected from —H, C₁-C₁₀ alkyl, C₂-C₆ alkenyl, C₂-C₆ alkynyl, C₁-C₆ alkyl-NHR²⁵, C₁-C₆ alkyl-NR²⁵R²⁶, C₁-C₄ alkyl-OR²⁷, CSR¹¹, CO₂R²⁸, COR²⁹, CONHR²⁸, CON(R²)₂, SOR²⁹, SO₂R²⁹, C₁-C₆ alkyl-CO₂R², C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²³ is selected from —H, C₁-C₆ alkyl, C₂-C₆ alkenyl, C₂-C₆ alkenyl-R²⁵, C₁-C₆ alkyl-R²⁵, C₂-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²R²⁸, C₁-C₆ alkyl-C(S)NHR²⁸, CC—C₆ alkyl-CON(R²⁸)₂, C₁-C₆ alkyl-C(S)N(R²¹)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²⁴ is selected from —H, OH, C₁-C₁₀ alkyl, C₂-C₁₀ alkenyl, C₂-C₁₀ alkynyl, C₁-C₁₀ alkyl-R²⁹, C₂-C₁₀ alkenyl-R²⁹, C₂-C₁₀ alkynyl-R²⁹, C₁-C₁₀ alkyl-(R²⁹)₂, C₂-C₁₀ alkenyl-(R²⁹)₂, CSR²⁹, amino, NHR²⁵, NR²⁶R²⁶, N(R²⁵)—N(R²⁶)(R²⁶), C(R²⁹)═N—N(R²⁶)(R²⁶), N═N(R²⁵), N(R²⁵)—N═C(R²⁶), C(R²⁹)═N—O(R²⁷), ON═C(R²⁹), C₁-C₁₀ alkyl-NHR²⁵, C₁-C₁₀ alkyl-NR²⁶R²⁶, (C₁-C₁₀)alkyl-N(R²⁵)—N(R²⁶) (R²⁶), (C₁-C₁₀)alkylC(R²⁹)═N—N(R²⁶)(R²⁶), (C₁-C₁₀)alkyl-N═N(R²⁵), (C₁-C₁₀)alkyl-N(R²⁵)—N═C(R²⁶), SCN, NCS, C₁-C₁₀ alkyl SCN, C₁-C₁₀ alkyl NCS, nitro, cyano, O—R²⁷, C₁-C₁₀ alkyl-OR²⁷, CO₂R²⁹, COR²⁹, SR²⁷, SSR²⁷, SOR²⁹, SO₂R²⁹, C₁-C₁₀ alkyl-COR²⁹, C₁-C₁₀ alkyl-SR²⁷, C₁-C₁₀ alkyl-SOR²⁹, C₁-C₁₀ alkyl-SO₂R²⁹, halo, Si(R²⁹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²⁵ and R²⁶ are each independently selected from —H, C₁-C₆ alkyl, C₂-C₆ alkenyl, C₂-C₆ alkynyl, C₁-C₄ alkyl-R³⁵, C₁-C₆ alkyl-NHR³, C₁-C₆ alkyl-NR³¹, R³², O—R³³, C₁-C₄ alkyl-OR³³, CO₂R³³, C(S)OR³³, C(O)SR³³, C(O)R³³, C(S)R³⁵, CONHR³⁴, C(S)NHR³⁴, CON(R³⁴)₂, C(S)N(R³⁴)₂, SR³³, SOR³⁵, SO₂R³⁵, C₁-C₆ alkyl-CO₂R³³, —C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R²⁷ and R²⁸ are independently selected from —H, C₁-C₆ alkyl, C₂-C₆ alkenyl, C₂-C₆ alkynyl, C₁-C₆ alkyl-NHR³′, C₁-C₆ alkyl-NR³¹R³², C—C₄ alkyl-OR³³, CSR¹¹, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R²⁹ is selected from —H, C₁-C₆ alkyl, C₂-C₆ alkenyl, C₂-C₆ alkenyl-R³¹, C₁-C₆ alkyl-R³¹, C₂-C₆ alkynyl, amino, NHR³¹, NR³¹R³², C₁-C₆ alkyl-NHR³, C₁-C₆ alkyl-NR³¹R³², O—R³³, C₁-C₄ alkyl-OR³³, SR³³, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C—C₆ alkyl-CONHR³⁴, C₁-C₆ alkyl-C(S)NHR³, C₁-C₆ alkyl-CON(R³⁴)₂, C₁-C₆ alkyl-C(S)N(R³⁴)₂, C₁-C₆ alkyl-SR³³, C₁-C₆ alkyl -SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³⁰ is selected from —H, OH, C₁-C₁₀ alkyl, C₂-C₁₀ alkenyl, C₂-C₁₀ alkynyl, C₁-C₁₀ alkyl-R³⁵, C₂-C₁₀ alkenyl-R³⁵, C₂-C₁₀ alkynyl-R³⁵, C₁-C₁₀ alkyl-(R³⁵)₂, C₂-C₁₀ alkenyl-(R³⁵)₂, CSR³⁵, N═NR³¹, amino, NHR³¹, NR³²R³², N(R³¹)—N(R³²)(R³²), C(R³⁵)═N—N(R³²)(R³²), N═N(R³¹), N(R³¹) N═C(R³²), C(R³⁵)═N—O(R³³), ON═C(R³⁵), C₁-C₁₀ alkyl-NHR³¹, C₁-C₁₀ alkyl-NR³²R³², (C₁-C₁₀)alkyl-N(R³¹)—N(R³²)(R³²), (C₁-C₁₀)alkylC(R³⁵)═N—N(R³²) (R³²), (C₁-C₁₀)alkyl-N═N(R³¹), (C₁-C₁₀)alkyl-N(R³¹)—N═C(R³²), SCN, NCS, C₁-C₁₀ alkyl SCN, C₁-C₁₀ alkyl NCS, nitro, cyano, O—R³³, C_-C₁₀ alkyl-OR³³, COR³⁵, SR³³, SSR³³, SOR³⁵, SO₂R³⁵, C₁-C₁₀ alkyl-COR³⁵, C₁-C₁₀ alkyl-SR³³, C₁-C₁₀ alkyl-SOR³⁵, C₁-C₁₀ alkyl-SO₂R³⁵, halo, Si(R³⁵)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³⁶ is selected from —H, alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;
R², R³, R⁴, R⁵, R³⁷ and R³⁸ are each independently absent, or selected from an R¹ group;
n is 0; and
R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

2. The compound according to claim 1, wherein when Z² and Z³ are both nitrogen, R⁴ is other than pyrrole, or optionally when Z⁴ and Z⁵ are both nitrogen and R^{a} is ring Q, Q² is other than nitrogen.

3. The compound according to claim 1, wherein R^{a} is selected from an M-ring and a Q-ring.

4. The compound according to claim 1, wherein R^{a} is an M-ring.

5. The compound according to claim 4, wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if carbon, the carbon is substituted with (L)ₙR¹.

6. The compound according to claim 1, wherein R^{a} is an M-ring, wherein ring M is an aromatic pyridine or pyrimidine ring, wherein M¹, M³ and M⁴ are carbon and are substituted with (L)ₙR¹, M⁵ is carbon, M² and M⁶ are independently selected from carbon and nitrogen and if carbon, the carbon is substituted with (L)ₙR¹.

7. The compound according to claim 1, wherein:
R^{a} is an M-ring that is an aromatic pyridine or pyrimidine ring;
M¹, M³ and M⁴ are carbon and are substituted with (L)ₙR¹;
M⁵ is carbon;
M² and M⁶ are independently selected from carbon and nitrogen and if carbon, the carbon is substituted with (L)ₙR¹; and
R³ and R⁴ optionally join to form a ring of 5, 6, 7, or 8 atoms, where the atoms in the ring are independently selected from Z³, Z⁴, O, S, C═O, C═S, S═O, SO₂, C that is mono or di-substituted with an R¹ group, and N that is unsubstituted or substituted with an R¹ group.

8. The compound according to claim 1, wherein:
R^{a} is an M-ring that is an aromatic pyridine or pyrimidine;
M¹, M³ and M⁴ are carbon and are substituted with (L)ₙR¹;
M⁵ is carbon;
M² and M⁶ are independently selected from carbon and nitrogen and if carbon, the carbon is substituted with (L)ₙR¹; and
R³ and R⁴ optionally join to form a ring of 6 or 7 atoms, where the atoms in the ring are independently selected from Z³, Z⁴, C═O, C that is mono or di-substituted with an R¹ group, and N that is unsubstituted or substituted with an R¹ group.

9. The compound according to claim 1, wherein:
R^{a} is an M-ring that is an aromatic pyridine or pyrimidine;
M¹, M³ and M⁴ are carbon and are substituted with (L)ₙR¹;
M⁵ is carbon;
M² and M⁶ are independently selected from carbon and nitrogen and if carbon, the carbon is substituted with (L)ₙR¹; and R³ and R⁴ optionally join to form a ring of 6 atoms, where the atoms in the ring are independently selected from Z³, Z⁴, C═O, C that is mono or di-substituted with an R¹ group, and N that is unsubstituted or substituted with an R¹ group.

10. The compound according to claim 1, wherein:
R^{a} is an M-ring that is an aromatic pyridine or pyrimidine ring;
M¹, M³ and M⁴ are carbon and are substituted with (L)ₙR¹;
M⁵ is carbon;
M² and M⁶ are independently selected from carbon and nitrogen and if carbon, the carbon is substituted with (L)ₙR¹; and
R³ and R⁴ optionally join to form a ring that is selected from:

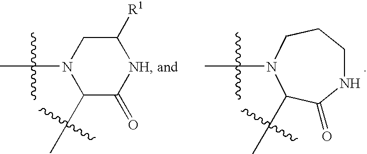

11. The compound according to claim 1, wherein:
R^{a} is an M-ring that is an aromatic pyridine;
M¹, M³, M⁴ and M⁶ are carbon and are substituted with (L)ₙR¹;
M⁵ is carbon; and
M² is nitrogen.

12. The compound according to claim 1, wherein:
R^{a} is an M-ring that is an aromatic pyrimidine:
M¹, M³ and M⁴ are carbon and are substituted with (L)ₙR¹;
M⁵ is carbon; and
M² and M⁶ are nitrogen.

13. The compound according to claim 1, wherein:
R^{a} is an M-ring that is an aromatic pyridine:
M¹, M³, M⁴ and M⁶ are carbon and are substituted with (L)ₙR¹;
M⁵ is carbon;
M² is nitrogen;
R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR⁷, CO₂R⁷, CONHR⁷, N(R⁸)₂, amino C₁-C₄ alkyl, hydroxy C₁-C₄ alkyl, amino, amino C₁-C₄ alkyl-R⁷, halo C₁-C₄ alkyl, C₁-C₆ alkyl-NHR⁷, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;
R⁷ and R⁸ are each independently selected from —H, C₁-C₆ alkyl, C₁-C₄ alkyl-R¹¹, C₁-C₆ alkyl-N(R³)₂, CO₂R⁶, COR⁷, aryl, and arylalkyl, wherein aryl and arylalkyl, are optionally substituted with one or more of the groups defined by R¹⁸;
R⁹ and R¹⁰ are each independently selected from —H, hydroxyl, C₁-C₆ alkyl, C₁-C₆ alkyl-R¹⁷, C₁-C₆ alkyl-NH₂R¹³, CO₂R⁶, COR⁷, C₁-C₆ alkyl-CO₂R⁶, C₁-C₆ alkyl-CONH—R⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;
R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₇-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;
R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R⁸)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;
R¹³ and R¹⁴ are each independently selected from —H, oxo, C₁-C₆ alkyl, COR²³, and aryl;
R¹⁵ and R¹⁶ are each independently selected from —H, aryl, arylalkyl, wherein aryl, arylalkyl, are optionally substituted with one or more of the groups defined by R²⁴;
R¹⁷ is selected from —H, C₁-C₆ alkyl, C₁-C₆ alkyl-R¹⁹, NHR¹⁹, aryl, heteroarylalkyl, and heterocyclylalkyl, wherein aryl is optionally substituted with one or more of the groups defined by R²⁴;
R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;
R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;
R²¹ and R²² are each independently selected from —H and C₁-C₆ alkyl;
R²³ is selected from —H and C₁-C₆ alkyl;
R²⁴ is selected from —H, C₁-C₆ alkyl, C₁-C₆ alkoxy, CO₂R²⁹, halo, and halo C₁-C₄ alkyl;
R²⁹ is selected from —H, and C₁-C₆ alkyl;
R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;
R³⁶ is selected from —H and halo; and
R², R³, R⁴, R³⁷ and R³⁸ are each independently selected from an R¹ group.

14. The compound according to claim 1, wherein:
R^{a} is an M-ring that is an aromatic pyrimidine;
M¹, M³ and M⁴ are carbon and are substituted with (L)ₙR¹;
M⁵ is carbon;
M² and M⁶ are nitrogen;
R¹ is selected from —H, C₁-C₆ alkyl, C₂-C₆ alkenyl, C₂-C₆ alkynyl, hydroxyl, C₁-C₆ alkoxy, C₂-C₆ alkenyl-R¹¹, C₁-C₆ alkoxy-R¹¹, COR¹⁷, CO₂R⁷, CONHR⁷, N(R⁸)₂, amino C₁-C₄ alkyl, hydroxy C₁-C₄ alkyl, amino, amino C₁-C₄ alkyl-R⁷, halo C₁-C₄ alkyl, C₁-C₆ alkyl-NHR⁷, carbonitrile, SR¹⁰, halo, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, C₁-C₆ alkyl-COR¹¹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;
R⁷ and R⁸, are each independently selected from —H, C₁-C₆ alkyl, C₁-C₄ alkyl-R¹¹, C₁-C₆ alkyl-N(R¹³)₂, CO₂R¹⁶, COR¹⁷, aryl, and arylalkyl, wherein aryl and arylalkyl, are optionally substituted with one or more of the groups defined by R¹⁸;
R⁹ and R¹⁰ are each independently selected from —H, hydroxyl, C₁-C₆ alkyl, C₁-C₆ alkyl-R¹⁷, C₁-C₆ alkyl-NH₂R³, CO₂R⁶, COR⁷, C₁-C₆ alkyl-CO₂R⁶, C₁-C₆ alkyl-CONH—R⁶, C₁-C₆ alkyl-CON(R¹⁶)₂, hydroxy C₁-C₄ alkyl, halo C₁-C₄ alkoxy, halo C₁-C₄ alkyl, Si(R¹³)₂R¹⁷, aryl, heteroaryl, heterocyclyl, arylalkyl, and C₁-C₁₀ mono- and bicyclic cycloalkyl, wherein aryl, heteroaryl, heterocyclyl, and arylalkyl, are optionally substituted with one or more of the groups defined by R¹⁸;
R¹¹ is selected from —H, C₁-C₆ alkyl, C₁-C₆ alkoxy, hydroxyl, halo, amino, NHR¹³, N(R¹³)₂, COR¹³, CO₂R¹⁷, halo C₁-C₄ alkyl, aryl, heteroaryl, heterocyclyl, heteroarylalkyl, and heterocyclylalkyl, wherein heterocyclyl, heteroarylalkyl, and heterocyclylalkyl, are optionally substituted with one or more of the groups defined by R¹⁸;
R¹² is selected from —H, hydroxyl, oxo, C₁-C₆ alkyl, hydroxyl C₁-C₆ alkyl-R¹¹, C₁-C₁₀ alkoxy, amino, amino C₁-C₄ alkyl-R⁷, NHR⁷, N(R⁷)₂, C₁-C₆ alkyl-NHR⁷, C₁-C₆ alkyl-NHR⁸R⁹, C₁-C₆ alkyl-N(R³)₂, C₁-C₆ alkyl-R¹¹, C₁-C₆ alkyl-CO₂R⁷R¹¹, C₁-C₆ alkoxy-R¹¹, nitro, O—R¹⁰, C═O, COR¹¹, CO₂R¹¹, SR¹⁰, SOR¹¹, SO₂R¹¹, NHSO₂R¹¹, C₁-C₆ alkyl-SR¹⁰, halo, halo C₁-C₄ alkyl, halo C₁-C₄ alkoxy, hydroxy C₁-C₄ alkyl, hydroxy C₁-C₄ alkoxy, aryl, heteroaryl, heterocyclyl, arylalkyl, heteroarylalkyl, heterocyclylalkyl, and C₁-C₁₀ mono- and bicyclic cycloalkyl, wherein aryl, heteroaryl, heterocyclyl, arylalkyl, heteroarylalkyl, and heterocyclylalkyl, and C₁-C₁₀ mono- and bicyclic cycloalkyl are optionally substituted with one or more of the groups defined by R¹⁸;
R¹³ and R¹⁴ are each independently selected from —H, oxo, C₁-C₆ alkyl, COR²³, and aryl;
R¹⁵ and R¹⁶ are each independently selected from —H, aryl, arylalkyl, wherein aryl, arylalkyl, are optionally substituted with one or more of the groups defined by R²⁴;
R¹⁷ is selected from —H, C₁-C₆ alkyl, C₁-C₆ alkyl-R¹⁹, NHR¹⁹, aryl, heteroarylalkyl, and heterocyclylalkyl, wherein aryl is optionally substituted with one or more of the groups defined by R²⁴;
R¹⁸ is selected from —H, oxo, hydroxyl, C₁-C₁₀ alkyl, C₁-C₁₀ alkoxy, amino, amino C₁-C₆ alkyl, N(R¹⁹)₂, C₁-C₆ alkyl-N(R¹⁹)₂, CO₂R²³, SR²¹, halo, halo C₁-C₄ alkyl, aryl, heteroaryl, and heterocyclyl, wherein aryl, heteroaryl, and heterocyclyl, are optionally substituted with one or more of the groups defined by R²⁴;
R¹⁹ and R²⁰ are each independently selected from —H, C₁-C₆ alkyl, heteroaryl, heterocyclyl, wherein aryl, heteroaryl, and heterocyclyl, are optionally substituted with one or more of the groups defined by R³⁰;
R²¹ and R²² are each independently selected from —H and C₁-C₆ alkyl;
R²³ is selected from —H and C₁-C₆ alkyl;
R²⁴ is selected from —H, C₁-C₆ alkyl, C₁-C₆ alkoxy, CO₂R²⁹, halo, and halo C₁-C₄ alkyl;
R²⁹ is selected from —H, and C₁-C₆ alkyl;
R³⁰ is selected from —H, aryl, heteroaryl, heterocyclyl, alkylaryl, arylalkyl, wherein aryl, heteroaryl, heterocyclyl, alkylaryl, and arylalkyl, are optionally substituted with one or more of the groups defined by R³⁶;
R³⁶ is selected from —H and halo; and
R², R³, R⁴, R³⁷ and R³⁸ are each independently selected from an R¹ group.

15. An MK-2 inhibiting compound that is listed in Table I or Table II.

16. The compound according to claim 15, wherein the compound is selected from the group consisting of:
1-(2-aminoethyl)-3-(2-quinolin-3-ylpyridin-4-yl)-1H-pyrazole-5-carboxylic acid trifluoroacetate,
1-(3-aminopropyl)-3-[2-(3-nitrophenyl)pyridin-4-yl]-1H-pyrazole-5-carboxylic acid dihydrochloride,
6-(aminomethyl)-2-(2-quinolin-3-ylpyridin-4-yl)-6,7-dihydropyrazolo[1,5-a]pyrazin-4(5H)-one,
1-(2-aminoethyl)-3-{2-[(E)-2-phenylethenyl]pyridin-4-yl}-1H-pyrazole-5-carboxylic acid trifluoroacetate,
1-(2-aminoethyl)-3-{2-[4-(hydroxymethyl)phenyl]pyridin-4-yl}-1H-pyrazole-5-carboxylic acid dihydrochloride,
6-(hydroxymethyl)-2-(2-quinolin-3-ylpyridin-4-yl)-6,7-dihydropyrazolo[1,5-a]pyrazin-4(5H)-one, and
1-(3-aminopropyl)-3-(2-quinolin-3-ylpyridin-4-yl)-1H-pyrazole-5-carboxylic acid dihydrochloride, and mixtures thereof.

17. A method of inhibiting MK-2, the method comprising contacting MK-2 with at least one compound having the structure described in claim 1.

18. A method of inhibiting MK-2, the method comprising contacting MK-2 with at least one compound that is selected from the compounds described in claim 15.

19. A method of preventing or treating a TNFα mediated disease or disorder in a subject, the method comprising administering to the subject an effective amount of an MK-2 inhibiting compound having the structure described in claim 1.

20. The method according to claim 19, wherein the subject is one that is in need of such prevention or treatment.

21. The method according to claim 19, wherein the subject is a mammal.

22. The method according to claim 19, wherein the subject is a human.

23. The method according to claim 19, wherein the TNFα mediated disease or disorder is one that is selected from the group consisting of wherein the TNFα mediated disease or disorder is one that is selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

24. The method according to claim 19, wherein the TNFα mediated disease or disorder is selected from the group consisting of: arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, bums, dermatitis, gastrointestinal conditions, inflammatory bowel disease, gastric ulcer, gastric varices, Crohn's disease, gastritis, irritable bowel syndrome, ulcerative colitis, cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye tissue, pulmonary inflammation, viral infections, cystic fibrosis, central nervous system disorders, cortical dementias, and Alzheimer's disease.

25. A method of preventing or treating a TNFα mediated disease or disorder in a subject, the method comprising administering to the subject at least one MK-2 inhibiting compound that is selected from the group consisting of the compounds described in claim 15.

26. A therapeutic composition comprising a compound having the structure described in claim 1.

27. A therapeutic composition comprising at least one MK-2 inhibitory compound that is described in claim 15.

28. A pharmaceutical composition comprising a pharmaceutically acceptable carrier and at least one MK-2 inhibitory compound having the structure described in claim 1.

29. The pharmaceutical composition according to claim 28, wherein the MK-2 inhibitory compound has an IC₅₀ for MK-2 of not over 0.1 mM.

30. A kit comprising a dosage form that includes a therapeutically effective amount of at least one MK-2 inhibitory compound having a structure described in claim 1.
